(19) United States
(12) Patent Application Publication
(10) Pub. No.: US 2023/0407388 A1
(43) Pub. Date: Dec. 21, 2023

(54) Sequencing Templates Comprising Multiple Inserts and Compositions and Methods for Improving Sequencing Throughput

(71) Applicants: Illumina, Inc., San Diego, CA (US); Illumina Cambridge Limited, Cambridge (GB)

(72) Inventors: Tarun Khurana, Fremont, CA (US); Yir-Shyuan Wu, Albany, CA (US); Niall Anthony Gormley, Cambridge (GB); Jonathan Mark Boutell, Hertfordshire (GB)

(73) Assignees: Illumina, Inc., San Diego, CA (US); Illumina Cambridge Limited, Cambridge (GB)

(21) Appl. No.: 18/303,905

(22) Filed: Apr. 20, 2023

Related U.S. Application Data
(63) Continuation of application No. PCT/US2021/055878, filed on Oct. 20, 2021.
(60) Provisional application No. 63/094,422, filed on Oct. 21, 2020.
(60) Provisional application No. 63/256,040, filed on Oct. 15, 2021.

(51) Int. Cl. C12Q 1/6874

(52) U.S. Cl. CPC C12Q 1/6874

(57) ABSTRACT

Described herein is a polynucleotide for use as a sequencing template comprising multiple inserts. Also described herein are method of generating and using these polynucleotides and methods of use of such templates, including analysis of contiguity information. Further, sequencing templates comprising an insert sequence and a copy of the insert sequence can be used to correct for random errors generated during sequencing or amplification or to identify nucleobase damage or other mutation that leads to non-canonical base pairing in a double-stranded nucleic acid. Methods of performing methylation analysis are also described herein.

### CROSS-REFERENCE TO RELATED APPLICATION

This application is a continuation of PCT/US2021/055878, filed Oct. 20, 2021, which claims the benefit of priority of U.S. Provisional Application No. 63/094,422, filed Oct. 21, 2020, and U.S. Provisional Application No. 63/256,040, filed Oct. 15, 2021, the contents of which are each incorporated by reference herein in their entireties for any purpose.

### SEQUENCE LISTING

This application is filed with a Sequence Listing which has been submitted electronically in XML format. Said XML copy, created on Jul. 31, 2023, is named “2023-07-31_01243-0013-00US ST26.xml” and is 94,245 bytes in size. The information in the electronic format of the sequence listing is incorporated herein by reference in its entirety.

### DESCRIPTION

### Field

This application relates to polynucleotides comprising read primer binding sequences, insert sequences derived from a target nucleic acid, a concatenation sequence, and an attachment sequence. Compositions comprising these polynucleotides and methods of generating and sequencing a concatenated nucleic acid sequencing template are also described. In addition, this disclosure relates to methods of preparing sequencing templates comprising multiple inserts. This disclosure also relates to methods of use of such templates, including analysis of contiguity information. Further, sequencing templates comprising two copies of the same insert sequence (i.e., an insert sequence and a copy of an insert sequence) can be used to correct for random errors generated during sequencing or amplification or to identify nucleobase damage or other mutation that leads to non-canonical base pairing in a double-stranded nucleic acid. These sequencing templates comprising an insert sequence and a copy of the insert sequence can also be used for methylation analysis.

### Background

Typically, the read-length on sequencing by synthesis (SBS) platforms is limited to 250-300 base pairs due to phasing/pre-phasing. This read-length limits the throughput of SBS platforms.

Previously, methods were described to improve SBS throughput using polynucleotides comprising multiple inserts. Often these methods relied on orthogonal SBS reactions, for example with different polymerases or substrate combinations or with primer blocking (See WO 2015/0002789 and US 20180312917). However, a need exists for straightforward means to increase sequencing output from a flowcell without need for non-standard reagents to allow cost-effective and user-friendly means of increasing sequencing output.

The present disclosure describes polynucleotides comprising multiple insert sequences from one or more target nucleic acid. These polynucleotides may be generated from multiple DNA libraries. Annealing of a hybridization sequence in one library product to a complement of a hybridization sequence in another library product to form a hybridized adduct can then allow elongation to form the polynucleotide comprising multiple insert sequences. Sequencing of these multiple insert sequences can be performed by sequential SBS elongation reactions based on multiple distinct read primer binding sequences comprised in the polynucleotides.

In addition, conventional short read sequencing methods comprise an initial generation of short separate fragments from intact genomic DNA or RNA. These fragments are generated in a several ways such as physical shearing, enzymatic digestion, or polymerase extension from one or more primers. Template preparation then modifies and appends synthetic adapters to these fragments to enable them to be sequenced. These sequencing templates almost always contain a single fragment from the original sample comprising the sequence of bases in the same order and juxtaposition as in the intact genome. Where a template is double-stranded, the complement of a sequence is associated by hybridization of the two strands. However, when a double-stranded template is denatured, the two complementary strands separate, and a template becomes a single strand comprising a single sequence fragment from the original sample. In this process, any association between the two complementary strands is lost. In addition, in this process of fragmentation and template preparation, any association between two or more fragments that were contiguous in the original unfragmented genome is also lost.

The exception to this rule of loss of contiguity information is found in template preparation methods that employ ligation to join two or more distal fragments together prior to sequencing adapters being appended. One example is “mate-pair” libraries, wherein the ends of a large DNA fragment are joined together forming a circle, then further fragmented followed by recovery of the sub-fragment that spans the co-joined ends. The subsequent template contains two sequences from the original large fragment joined in tandem. Another example is chromatin based conformational capture where distal fragments of DNA in a genome are spatially organized in close proximity due to the structural arrangement of DNA complexed with chromatin in vivo. Ligation of fragments in proximity with one another and subsequent processing generates sequencing templates with tandem inserts that give information about the spatial relevance, and by inference, functional relevance of the individual inserts.

A number of different methods have been developed as potential means of improving preparation of sequencing templates with multiple inserts, such as Duplex Sequencing (Schmitt, et al. Proc. Natl. Acad. Sci. U S. A. 109:14508-14513 (2012), Duplex Proximity Sequencing (Pro-Seq, as described in Pel et al. PLoS One 13:1-19 (2018)), CypherSeq (Gregory et al. Nucleic Acids Res. 44:e22 (2016)), o2n-seq (Wang et al. Nat. Commun. 8, 15335 (2017)), Circle Sequencing (Lou et al., Proc. Natl. Acad. Sci. U S. A. 110:19872-19877 (2013)), and Bot Sequencing (Hoang et al. Proc. Natl. Acad. Sci. U S. A. 113:9846-9851 (2016) and Abascal et al. Nature 593, 405-410 (2021)). However, all these of these methods have shown drawbacks, and none has had universal applicability.

The Concatenating Original Duplex for Error Correction (CODEC) method recently described in Bae et al., bioRxiv, 10.1101/2021.06.11.448110, posted Jun. 12, 2021, involves physically linking both strands of double-stranded DNA for sequencing of a single duplex with a single read pair using specialized CODEC adapter complexes. The CODEC method can be used to identify non-canonical base-pairing that may be due to nucleobase damage or to a change comprised only in one strand of a double-stranded nucleic acid, as well as errors that may have been introduced during PCR amplification or sequencing. However, the CODEC method requires two consecutive ligations that can limit conversion efficiency, and byproducts may also be formed by undesired ligations.

In the absence of innate structural relationships between sequences in the genome, surrogate “association markers” in the form of barcodes may be used. For example, a large fragment of DNA, such as greater than 1000 base pairs, or even greater than 5000 base pairs, can be isolated by dilution, compartmentalization, or immobilization on a surface, and further fragmented wherein each sub-fragment thereafter appends a common barcode sequence. Where many fragments are thus processed in parallel, with each isolated fragment receiving a unique barcode sequence appended to its subsequent subsequences, a pool of all sub-fragments from all fragments can be sequenced in a single experiment, and the subfragments disambiguated by identifying and collating their barcode sequences. This approach enables contiguous sequences within the genome to be associated with one another and can enable the assembly in silico of numerous subfragments into much larger in silico fragments and can help with the phasing of variants in a genome.

In another type of barcoding, unique molecular indices (UMIs) are used for preserving associations between sequences within a genome that physically separate during template preparation and sequencing. The UMIs comprise short barcode sequences appended to fragments of DNA or RNA during template preparation such that individual single molecules each receive a unique barcode. Reading the UMI by sequencing can distinguish individual molecules (such as fragments within a preparation of templates) even when the original sample contained two or more identical fragments, in length and in sequence. UMIs also help identify mistakes (e.g., alterations to the innate genomic sequence) generated and propagated during PCR or other such methods that make copies of original templates. This is useful in experiments for sequencing samples that contain innate variants at low frequencies that would potentially otherwise be difficult to identify in a background of artificial variants created by PCR. In another use of UMIs, a double-stranded fragment can be ligated appended with a double-stranded adapter containing a duplex UMI (i.e., a UMI barcode hybridized to its complement in a double-stranded adapter such that a first and a second strand of the genomic fragment each append a common UMI barcode). In this manner, after separation by denaturation the first strand and second strand can be identified and re-associated by the UMI. Such use of UMIs can help improve the accuracy of sequencing by giving two “reads” of a sequence in the genome, in other words identifying and using the “sense” and “antisense” pair of templates from a fragment to infer the validity of a base call during a sequencing read of either template.

The use of barcodes to associate sequences, either distal or complementary within a genome, is in practice complex because of the constraints around designing and incorporating barcodes within adapters and sequencing reactions. For instance, there is a finite number of permutations for a given length of barcode. In one example, a four base barcode only has two-hundred and fifty-six permutations and not all are functional in practice due to self-complementarity and other sequencing considerations. Similar issues manifest when the barcode is longer but with the added penalty of requiring more cycles of sequencing to read the barcodes.

Adding barcodes to adapters adds complexity to the adapter itself. For instance, adding variations in performance from one adapter to another results in challenges around normalization during library pooling. Complex barcodes also require complex manufacturing, particularly when a barcode and its complement are hybridized in a double-stranded adapter.

The use of in vivo structural associations, such as mate-pairs or chromatin conformational capture, also require complex workflows and is limited in the associations it can identify. For example, a challenge of mate-pairs is the extreme size of large fragments, while a challenge of chromatin conformational capture is chromatin-induced associations.

Disclosed herein are a barcode-free methods that can provide association information about contiguous and complementary sequences within the genome. These methods may utilize a surface to link sequences in tandem within a single template. Methods may also use compartmentalization for generating templates for proximity or haplotype data. When sequenced, the resulting templates can provide information to correct errors in sequencing or identify non-canonical base pairings and also to provide contiguity information for assembly and phasing of genomic information.

Disclosed herein also are methods of detecting methylation status. Conventional methods for detecting methylation status in genomic DNA generally use a chemical or biochemical reaction to convert the bases of interests to a different base. The detection of this conversion is used to infer whether or not the base was methylated. These methods require a sample to be split in two aliquots. One aliquot is treated by the chemistries/biochemistries while the other aliquot remains untreated. Both are then sequenced and compared to one another to deduce the methylation status. One example of such chemistries is bisulfite sequencing, which uses sodium bisulfite conversion of non-methylated C bases to U bases. The uracil nucleotides are then converted to thymine nucleotides during an amplification step such as PCR. Following sequencing of both the treated and untreated sample, a comparison of the reads will indicate, wherein if a C base in the untreated sample is read as a T in the treated sample, that this C base was not methylated in the original sample. However, where a C base in the untreated sample is still read as a C base in the treated sample, then by deduction C base was methylated in the original sample.

A similar strategy is used with the EM-Seq assay as described in Vaisvilas et al., Genome Res. 31(7): 1280-1289 (2021), except that an enzymatic reaction rather than a chemical reaction is used to convert non-methylated C's. A recent publication (Liu et al., Nature Biotechnology 37(4):424-429 (2019)) introduced an alternative chemistry based on borane that converts methylated C nucleotides and does not convert unmethylated C nucleotides. It has a reported advantage over normal C conversion chemistries such as bisulfite sequencing, because the converted genome is mostly still a 4 base genome comprising A, C, G and T as only a small percentage of the genome is methylated (in contrast with bisulfite chemistries where the converted genome is mostly A, G and T).

A common characteristic of current method of methylation analysis is that a sample needs to be split into two aliquots, which are processed and sequenced in parallel. Technologies do exist that directly detect methylation status of bases without needing to split the sample. These methods rely on single-molecule sequencing technologies that use sequencing strategies that can differentiate methylated and unmethylated bases in the original sample. Examples of such technologies include nanopore sequencing (see, for example, “Epigenetics and methylation analysis,” Oxford Nanopore Technologies, downloaded on Oct. 7, 2021 at nanoporetech.com/applications/investigation/epigenetics-and-methylation-analysis) and SMRT sequencing (as described in Flusberg et al., Nat Methods. 7(6): 461-465 (2010)). However, these strategies are disadvantageous for methods where high-throughput sequencing is necessary or where genomes of interest are small in fragment size, such as cell-free DNA.

Described herein are methods where a single aliquot of a methylated sample is treated and sequencing to discern the methylation status of a genome. The methods include those that can discern hydroxymethylated-cytosine from methylated-cytosine. The present methods can decrease sample preparation and sequencing burden and potentially decreases the amount of starting material required for methylation analysis.

### SUMMARY

Described herein are polynucleotides comprising multiple insert sequences. These polynucleotides may be used in methods to allow sequencing of multiple inserts sequences from a target nucleic acid. Also described herein are polynucleotides comprising multiple inserts for use as sequencing templates in methods of error correction and identification of non-canonical base pairing, determining contiguity data, and methylation analysis.

Embodiment 1 is a polynucleotide comprising (a) a 5′ terminal polynucleotide comprising a first read primer binding sequence; (b) a first insert sequence located 3′ of the 5′ terminal polynucleotide, wherein the first insert sequence is derived from a target nucleic acid; (c) a concatenation sequence located 3′ of the first insert sequence comprising a second read primer binding sequence and a hybridization sequence; (d) a second insert sequence located 3′ of the concatenation sequence, wherein the second insert sequence is derived from a discontiguous sequence of the target nucleic acid or from a different target nucleic acid than the first insert sequence; and (e) a 3′ terminal polynucleotide sequence.

Embodiment 2 is a polynucleotide comprising a 3′ terminal polynucleotide comprising a first read primer binding sequence; a first insert sequence 5′ of the 3′ terminal polynucleotide that is derived from a target nucleic acid; a concatenation sequence comprising a second read primer binding sequence that is orthogonal to the first read primer binding sequence, wherein the second read primer binding sequence comprises a hybridization sequence; a second insert sequence 5′ of the concatenation sequence and derived from a discontiguous sequence of the target nucleic acid or from a different target nucleic acid than the first insert sequence; and an attachment polynucleotide at the 5′ end of the polynucleotide and comprising an attachment sequence, wherein the 3′ terminal polynucleotide, the concatenation sequence, and the attachment polynucleotide are not derived from the target nucleic acid.

Embodiment 3 is the polynucleotide of embodiment 1 or 2, wherein the two insert sequences are derived from different target nucleic acids.

Embodiment 4 is the polynucleotide of any of the preceding embodiments, wherein the first insert sequence and the second insert sequence each independently comprise from 40 to 400 nucleotides, 100 to 200 nucleotides, or 150 nucleotides.

Embodiment 5 is the polynucleotide of any of the preceding embodiments, wherein the first read primer binding sequence comprises a first adapter sequence.

Embodiment 6 is the polynucleotide of any of the preceding embodiments, wherein the first read primer binding sequence further comprises the complement of a transposon end sequence.

Embodiment 7 is the polynucleotide of embodiment 5 or 6, wherein the first adapter sequence is the complement of a A14 primer sequence (A14′) or the complement of a B15 primer sequence (B15′).

Embodiment 8 is the polynucleotide of any one of embodiments and 3 to 7, wherein, the 3′ terminal polynucleotide comprises the complement of a P7 primer sequence (P7′) or the complement of a P5 primer sequence (P5′

Embodiment 9 is the polynucleotide of any one of embodiments 2 to 7, wherein the 3′ terminal polynucleotide comprises the complement of a P5 primer sequence (P5′) and the attachment polynucleotide comprises a P7 primer sequence (P7), or the 3′ terminal polynucleotide comprises the complement of a P7 primer sequence (P7′) and the attachment polynucleotide comprises a P5 primer sequence (P5).

Embodiment 10 is the polynucleotide of any one of embodiments 2 to 9, wherein the concatenation sequence comprises (a) the hybridization sequence, and optionally comprises (b) a transposon end sequence 3′ of the hybridization unit and the complement of the transposon end sequence 5′ of the hybridization unit.

Embodiment 11 is the polynucleotide of embodiment 10, wherein the second read primer binding sequence comprises the hybridization sequence and the complement of the transposon end sequence.

Embodiment 12 is the polynucleotide of any one of embodiments 2 to 11, wherein the attachment polynucleotide comprises a second adapter sequence and optionally a transposon end sequence.

Embodiment 13 is the polynucleotide of embodiment 12, wherein the second adapter sequence is an A14 sequence or a B15 sequence.

Embodiment 14 is the polynucleotide of embodiment 13, wherein the first adapter sequence is the complement of an A14 sequence (A14′) and the second adapter sequence is a B15 sequence, or the first adapter sequence is the complement of a B15 sequence (B15′) and the second adapter sequence is an A14 sequence.

Embodiment 15 is the polynucleotide of any one of embodiments 2 to 7 or 9 to 14, wherein the 3′ terminal polynucleotide and/or the attachment polynucleotide each independently comprise at least one of a barcode sequence, a unique molecular identifier (UMI) sequence, a capture sequence, or a cleavage sequence.

Embodiment 16 is the polynucleotide of any one of embodiments 2 to 7 and 9 to 14, wherein the polynucleotide is immobilized on a solid support.

Embodiment 17 is the polynucleotide of embodiment 16, wherein the polynucleotide is immobilized on the solid support via the attachment polynucleotide.

Embodiment 18 is the polynucleotide of embodiment 17, wherein the polynucleotide is immobilized on the solid support via hybridization of the attachment polynucleotide to an attachment polynucleotide complement on the surface of the solid support.

Embodiment 19 is the polynucleotide of embodiment 17, wherein the polynucleotide is immobilized to the solid support via binding of an affinity moiety on the attachment polynucleotide to a binding moiety on the surface of the solid support.

Embodiment 20 is the polynucleotide of any one of embodiments 16 to 19, wherein the solid support is a flow cell or a bead.

Embodiment 21 is the polynucleotide of any one of embodiments 2 to 7 or 9 to 20, wherein the polynucleotide comprises, between the second insert sequence and the attachment polynucleotide, at least one insert unit comprising an insert sequence derived from a discontiguous sequence of the target nucleic acid or from a different target nucleic acid than the other insert sequences at the 5′ end and a concatenation sequence comprising a read primer binding sequence at the 3′ end, wherein the read primer binding sequence is orthogonal to the other read primer binding sequences.

Embodiment 22 is the polynucleotide of embodiment 21, wherein the polynucleotide is hybridized to its complement.

Embodiment 23 is a composition comprising the polynucleotide of any one of embodiments 1, 3-8, or 22 and its complement, wherein the complement comprises (a) a 5′ terminal complement comprising a first complement read primer binding sequence; (b) a complement sequence of the second insert sequence located 3′ of the 5′ terminal complement; (c) a complement concatenation sequence located 3′ of the complement sequence of the second insert sequence comprising: (i) a second complement read primer binding sequence, and (ii) a complement hybridization sequence; (d) a complement sequence of the first insert sequence located 3′ of the complement concatenation sequence; and (e) a 3′ terminal complement.

Embodiment 24 is a composition comprising the polynucleotide of any one of embodiments 2 to 7 or 9 to 22 and its complement, wherein the complement comprises a 3′ terminal complement comprising a first complement read primer binding sequence, wherein the first complement read primer binding sequence is orthogonal to the first and second read primer binding sequences; the complement of the second insert sequence 5′ of the 3′ terminal complement; a complement concatenation sequence 5′ of the complement of the second insert sequence and comprising a 3′ to 5′ second complement read primer binding sequence, wherein the second complement read primer binding sequence is orthogonal to the first and second read primer binding sequences, and to the first complement read primer binding sequence; the complement of the first insert sequence 5′ of the complement concatenation sequence; and a complement attachment polynucleotide at the 5′ end comprising a complement attachment sequence.

Embodiment 25 is the composition of embodiment 24, wherein the first complement read primer binding sequence is complementary to the second adapter sequence and, when present, the transposon end sequence of the attachment polynucleotide; the complement concatenation sequence is complementary to the concatenation sequence; and the complement attachment polynucleotide is complementary to first adapter sequence and, when present, the complement of the transposon end sequence.

Embodiment 26 is the composition of embodiment 24 or 25, wherein the polynucleotide is immobilized on a solid support via the first attachment polynucleotide.

Embodiment 27 is the composition of embodiment 24 or 25, wherein the complement is immobilized on the solid support via the complement attachment polynucleotide.

Embodiment 28 is the polynucleotide of any one of embodiments 2 to 7 or 9 to 22 or the composition of any one of embodiments 24 to 27, wherein the polynucleotide has the structure: 3′-P7′-B15′-ME′-Insert 1-ME-HYB-ME′-Insert 2-ME-A14-P5-5′, wherein ME′ is the complement of a mosaic end sequence (for example, SEQ ID NO: 3).

Embodiment 29 is the polynucleotide or composition of embodiment 28, wherein the complement of the polynucleotide has the structure: 3′-P5′-A14′-ME′-Insert 2-ME-HYB′-ME′-Insert 1-ME-B15-P7-5′.

Embodiment 30 is a transposome complex comprising a transposase; a first transposon comprising the complement of a first read primer binding sequence, wherein the complement of the first read primer binding sequence comprises a 3′ portion comprising a transposon end sequence; and the complement of a first adapter sequence; and a second transposon comprising a 5′ portion comprising the complement of the transposon end sequence; and the complement of a hybridization sequence.

Embodiment 31 is the transposome complex of embodiment 30, wherein the complement of the first adapter sequence is a B15 sequence.

Embodiment 32 is the transposome complex of embodiment 30 or 31, wherein the second transposon comprises a complement attachment sequence 5′ of the first read primer binding sequence, optionally wherein the complement attachment sequence comprises a P7 sequence.

Embodiment 33 is the transposome complex of embodiment 30, wherein the transposome complex has the structure:

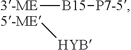

wherein ME is a mosaic end sequence such as SEQ ID NO: 6.

Embodiment 34 is the transposome complex of any one of embodiments 30 to 33, wherein the transposome complex is immobilized on a bead via the first or second transposon.

Embodiment 35 is a transposome complex comprising a transposase; a first transposon comprising an attachment polynucleotide, wherein the attachment polynucleotide comprises a 5′ portion comprising an attachment sequence; a 3′ portion comprising a second read primer binding sequence, comprising a 3′ portion comprising a transposon end sequence; and an adapter; and a second transposon comprising a 5′ portion comprising the complement of the transposon end sequence; and a hybridization sequence.

Embodiment 36 is the transposome complex of embodiment 35, wherein the adapter is an A14 sequence.

Embodiment 37 is the transposome complex of embodiment 35 or 36, wherein the attachment sequence comprises a P5 sequence.

Embodiment 38 is the transposome complex of embodiment 35, wherein the transposome complex has the structure:

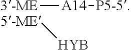

Embodiment 39 is the transposome complex of any one of embodiments 35 to 38, wherein the transposome complex is immobilized to a solid support via the first or second transposon.

Embodiment 40 is the transposome complex of any one of embodiments 35 to 38, wherein the transposome complex is immobilized on a bead.

Embodiment 41 is the transposome complex of any one of embodiments 30 to 40, wherein the transposome complex is immobilized to an affinity binding partner on the solid support or bead via an affinity element connected to a linker attached to the first or second transposon.

Embodiment 42 is a composition or kit comprising more than one transposome complex, such as the transposome complex of any one of embodiments 30 to 41.

Embodiment 43 is a composition or kit comprises a solid support, optionally wherein the optionally support is beads; components for generating transposome complexes, comprising a transposase; oligonucleotides for generating an oligonucleotide duplex, wherein the first oligonucleotide comprises a 3′ transposon end sequence and a 5′ first adapter sequence and the second oligonucleotide comprises a 5′ transposon end sequence and a 3′ second adapter sequence, wherein the 5′ transposon end sequence is complementary to the 3′ transposon end sequence; wherein the first and second adapter sequences are not the same; and a first and second set of primers for adding attachment sequences and hybridization sequences to fragments by PCR, wherein the first set of primers comprises primers for adding a hybridization sequence and a first attachment sequence to fragments; and wherein the second set of primers comprises primers for adding a complement hybridization sequence and a second attachment sequence to fragments; wherein the first and second attachment sequences are not the same.

Embodiment 44 is an adapter composition or kit comprising a first forked adapter complex and a second forked adapter complex, wherein the first forked adapter complex comprises a complement attachment polynucleotide comprising a 5′ portion comprising a complement attachment sequence; and a 3′ portion comprising an adapter; and a hybridization polynucleotide comprising (a) a 5′ portion comprising the complement of a portion of the adapter and hybridized thereto; and (b) the complement of a hybridization sequence, wherein the complement of the hybridization sequence is not complementary to the complement attachment polynucleotide; and the second forked adapter complex comprises an attachment polynucleotide comprising a 5′ portion comprising an attachment sequence; and a 3′ portion comprising the adapter; and a hybridization polynucleotide comprising (a) a portion comprising the complement of a portion of the adapter and hybridized thereto; and (b) a hybridization sequence, wherein the hybridization sequence is not complementary to the attachment polynucleotide.

Embodiment 45 is the adapter composition or kit of embodiment 44, wherein the attachment sequence comprises a P5 primer sequence and the complement attachment sequence comprises a P7 primer sequence.

Embodiment 46 is the adapter composition or kit of embodiment 44 or wherein the complement attachment polynucleotide comprises a B15 sequence and the hybridization polynucleotide comprises a A14 sequence.

Embodiment 47 is the adapter composition or kit of embodiment 46, wherein a first forked adapter complex has the structure:

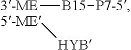

and a second forked adapter complex has the structure:

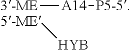

Embodiment 48 is the adapter composition or kit of any one of embodiments 44 to 47, wherein the adapter complexes comprise methylated nucleotides (e.g., include methylated cytosines).

Embodiment 49 is a method of generating a concatenated nucleic acid sequencing template comprising attaching a first read primer binding sequence to the 3′ end of a first insert sequence derived from a first target nucleic acid; attaching a hybridization sequence to the 5′ end of the first insert sequence; attaching the complement of the hybridization sequence to the 3′ end of a second insert sequence derived from a discontiguous region of the first target nucleic acid or from a second target nucleic acid; and annealing the hybridization sequence to the complement of the hybridization sequence to form a hybridized adduct; synthesizing a fully double-stranded concatenated nucleic acid sequencing template from the hybridized adduct; wherein the region between the first and second insert sequences comprises a second read primer binding sequence that comprises the hybridization sequence and is orthogonal to the first read primer binding sequence; thereby generating a concatenated nucleic acid sequencing template.

Embodiment 50 is the method of embodiment 48, wherein the attaching the first read primer binding sequence and the attaching the hybridization sequence comprises contacting the one or more target nucleic acids with a transposome complex, under conditions suitable for tagmentation.

Embodiment 51 is the method of embodiment 49 or 50, wherein the attaching the complement of the hybridization sequence to the 3′ end of a second insert sequence derived from a discontiguous region of the first target nucleic acid or from a second target nucleic acid comprises contacting the one or more target nucleic acids with a transposome complex, under conditions suitable for tagmentation.

Embodiment 52 is the method of embodiment 49, wherein the attaching a first read primer binding sequence to the 3′ end of a first insert sequence and the attaching a hybridization sequence to the 5′ end of the first insert sequence comprise contacting one or more target nucleic acids with a first forked adapter complex of any one of embodiments 44 to 48, under conditions suitable for ligation of the adapter complexes to the ends of the fragments to form fragments ligated at both ends with the first adapter complex and fragments ligated at both ends with the second adapter complex, and denaturing the ligated fragments.

Embodiment 53 is the method of embodiment 49 or 50, wherein attaching the complement of the hybridization sequence to the 3′ end of a second insert sequence comprises contacting the one or more target nucleic acids with a second forked adapter complex, under conditions suitable for ligation of the adapter complexes to the ends of the fragments to form fragments ligated at both ends with the first adapter complex and fragments ligated at both ends with the second adapter complex, and denaturing the ligated fragments.

Embodiment 54 is a method of generating a concatenated nucleic acid sequencing template comprising contacting a first sample comprising a first target nucleic acid with a first transposome complex and a second transposome complex, wherein each transposome complex comprises a transposase; a first transposon comprising a 3′ portion comprising a transposon end sequence and a 5′ portion comprising an adapter sequence; and a second transposon comprising a 5′ portion comprising the complement of the transposon end sequence and hybridized thereto; wherein the adapter sequence in the first transposome complex is the complement of a first adapter sequence and the adapter sequence in the second transposome complex is a second adapter sequence; under conditions sufficient to fragment the first target nucleic acid to generate a first tagged product comprising an insert sequence from the first target nucleic acid tagged at one end with the transposons of the first transposome complex and at the other end with the transposons of the second transposome complex; adding a complement attachment sequence to the 3′ end of the first tagged product and adding the complement of a hybridization sequence to the 5′ end of the first tagged product, optionally by polymerase chain reaction, to form a first modified tagged product; contacting a second sample comprising a second target nucleic acid with the transposome complexes under conditions sufficient to fragment the second target nucleic acid to generate a second tagged product comprising an insert sequence from the second target nucleic acid tagged at one end with the transposons of the first transposome complex and at the other end with the transposons of the second transposome complex; adding an attachment sequence to the 3′ end of the second tagged product and adding a hybridization sequence to the 5′ end of the second tagged product, optionally by polymerase chain reaction, to form a second modified tagged product; annealing the hybridization sequence of the first modified tagged product to the complement of the hybridization sequence in the second modified tagged product to form a hybridized adduct; and synthesizing a fully double-stranded concatenated nucleic acid sequencing template from the hybridized adduct, wherein the concatenated nucleic acid sequence template comprises (a) a first read primer binding sequence 3′ of the insert sequence from the second target nucleic acid, wherein the first read primer binding sequence comprises the first adapter sequence and the complement of the transposon end sequence, and (b) a second read primer binding sequence between the two insert sequences, wherein the second read primer binding sequence comprises the transposon end sequence and the hybridization sequence, and wherein the first read primer binding sequence is orthogonal to the second read primer binding sequence.

Embodiment 55 is a method of generating a concatenated nucleic acid sequencing template comprising contacting a first sample comprising a first target nucleic acid with a first transposome complex, wherein the first transposome complex comprises a transposase; a first transposon comprising a 3′ portion comprising a transposon end sequence and a 5′ portion comprising an attachment sequence and the complement of a first adapter sequence; and a second transposon comprising a 5′ portion comprising the complement of the transposon end sequence and hybridized thereto; under conditions sufficient to fragment the first target nucleic acid to generate a first tagged product comprising an insert sequence from the first target nucleic acid tagged at each end with the transposons of the first transposome complex; adding the complement of a hybridization sequence to the 5′ end of the first tagged product, optionally by polymerase chain reaction, to form a first modified tagged product; contacting a second sample comprising a second target nucleic acid with a second transposome complex, wherein the second transposome complex comprises a transposase; a first transposon comprising a 3′ portion comprising a transposon end sequence and a 5′ portion comprising a second adapter sequence and a complement attachment sequence; and a second transposon comprising a 5′ portion comprising the complement of the transposon end sequence and hybridized thereto; under conditions sufficient to fragment the second target nucleic acid to generate a second tagged product comprising an insert sequence from the second target nucleic acid tagged at each end with the transposons of the second transposome complex; adding the complement of the hybridization sequence to the 5′ end of the second tagged product, optionally by polymerase chain reaction, to form a second modified tagged product; annealing the hybridization sequence of the first modified tagged product to the complement of the hybridization sequence in the second modified tagged product to form a hybridized adduct; and synthesizing a fully double-stranded concatenated nucleic acid sequencing template from the hybridized adduct, wherein the concatenated nucleic acid sequence template comprises (a) a first read primer binding sequence 3′ of the insert sequence from the second target nucleic acid, wherein the first read primer binding sequence comprises the first adapter sequence and the complement of the transposon end sequence, and (b) a second read primer binding sequence between the two insert sequences, wherein the second read primer binding sequence comprises the transposon end sequence and the hybridization sequence, and wherein the first read primer binding sequence is orthogonal to the second read primer binding sequence.

Embodiment 56 is the method of embodiment 54 or 55, wherein the transposome complexes are immobilized on a solid support.

Embodiment 57 is a method of generating a concatenated nucleic acid sequencing template comprising (a) contacting: (i) a first double-stranded polynucleotide comprising a first target nucleic acid with a first restriction enzyme, and (ii) a second double-stranded polynucleotide comprising a second target nucleic acid with a second restriction enzyme; to produce first and second polynucleotides with compatible overhangs, and wherein the restriction enzymes are chosen from type II, type IIS, type IIP, and type IIT restriction enzymes; (b) attaching the compatible overhangs of the first and second polynucleotides using a ligase.

Embodiment 58 is the method of embodiment 57, wherein the contacting step is preceded by: (a) attaching the first restriction enzyme cut site, optionally, by using an adapter, to a first target nucleic acid and generating the first double stranded polynucleotide by primer extension; and (b) attaching the second restriction enzyme cut site, optionally, by using an adapter, to a second target nucleic acid and generating the second double stranded polynucleotide by primer extension.

Embodiment 59 is a method of generating a concatenated nucleic acid sequencing template comprising: (a) shearing or digesting a first source of nucleic acids and a second source of nucleic acids to generate a first library of nucleic acid fragments and a second library of nucleic acid fragments, respectively; (b) attaching a first adapter to each nucleic acid fragment from the first source of nucleic acids and attaching a second adapter to each nucleic acid fragment of the second source of nucleic acids comprising: (i) contacting the nucleic acid fragments with a first polymerase to produce nucleic acid fragments with blunt ends; (ii) phosphorylating 5′-hydroxyl of the nucleic acid fragments with kinase; (iii) adding 3′ adenine to the nucleic acid fragments with a second polymerase; and (iv) ligating the first adapter to each nucleic acid fragment of the first library and ligating the second adapter to each nucleic acid fragment of the second library; (c) mixing and annealing the first and second libraries of nucleic acids, optionally by PCR, wherein (i) the nucleic acids denature at elevated temperatures and (ii) A and A′ sequences hybridize to each other at lower temperatures; and (d) synthesizing a fully double-stranded concatenated nucleic acid sequencing template, optionally by PCR.

Embodiment 60 is the method of any one of embodiments 54 to 59, wherein the method comprises sequencing the concatenated nucleic acid sequence template.

Embodiment 61 is a method of sequencing a concatenated nucleic acid sequencing template comprising sequencing the first insert sequence of a polynucleotide of any one of embodiments 1 to 22 by initiating sequencing with a first read sequencing primer complementary to the first read primer binding sequence; and sequencing the second insert sequence by initiating sequencing with a second read sequencing primer complementary to the second read primer binding sequence.

Embodiment 62 is the method of embodiment 61, wherein a method further comprises sequencing the complement of the second insert sequence by initiating sequencing with a first complement read sequencing primer complementary to the first complement read primer binding sequence; and sequencing the complement of the first insert sequence by initiating sequencing with a second complement read sequencing primer complementary to the second complement read primer binding sequence.

Embodiment 63 is a method of any one of embodiments 49 to 59, wherein compartmentalizing a sample comprising target double-stranded nucleic acid into a plurality of different compartments is performed and generating concatenated nucleic acid sequencing templates is performed within the different compartments.

Embodiment 64 is a polynucleotide comprising (a) a 5′ terminal polynucleotide comprising a first read sequencing primer sequence; (b) an insert sequence derived from a target nucleic acid, wherein the insert sequence is 3′ of the 5′ terminal polynucleotide; (c) a hybridization sequence 3′ of the insert sequence; (d) a copy of the insert sequence 3′ of the hybridization sequence; and (e) a 3′ terminal polynucleotide comprising the complement of a second read sequencing primer sequence.

Embodiment 65 is a polynucleotide comprising (a) a 5′ terminal polynucleotide comprising a first read sequencing primer sequence; (b) a first insert sequence derived from a target nucleic acid, wherein the insert sequence is 3′ of the 5′ terminal polynucleotide; (c) a hybridization sequence 3′ of the insert sequence; (d) a second insert sequence 3′ of the hybridization sequence; and (e) a 3′ terminal polynucleotide comprising the complement of a second read sequencing primer sequence.

Embodiment 66 is a polynucleotide of embodiment 64 or 65, wherein the insert sequences comprise 40 to 400 nucleotides, optionally wherein the insert sequences comprise 1000 or fewer nucleotides.

Embodiment 67 is the polynucleotide of any one of embodiments 64 to 66, wherein the hybridization sequence comprises 10 to 30 nucleotides, optionally wherein one or more nucleotide in the hybridization sequence is a locked nucleic acid.

Embodiment 68 is the polynucleotide of any one of embodiments 64 to 67, wherein the first read sequencing primer sequence and the second read sequencing primer sequence are different.

Embodiment 69 is the polynucleotide of any one of embodiments 64 to 68, wherein the first read sequencing primer sequence and the second read sequencing primer sequence each comprise an A14 sequence or a B15 sequence, or their complements.

Embodiment 70 is the polynucleotide of any one of embodiments 64 to 69, wherein the 3′ terminal polynucleotide comprises the complement of a P5 primer sequence (P5′) and the 5′ terminal polynucleotide comprises a P7 primer sequence (P7 (SEQ ID NO: 8)), or the 3′ terminal polynucleotide comprises the complement of a P7 primer sequence (P7′) and the 5′ terminal polynucleotide comprises a P5 primer sequence (P5 (SEQ ID NO: 7)).

Embodiment 71 is the polynucleotide of any one of embodiments 64 to 70, wherein the 3′ terminal polynucleotide and/or the 5′ terminal polynucleotide each independently comprise at least one of an adapter, a barcode sequence, a unique molecular identifier (UMI) sequence, a capture sequence, or a cleavage sequence.

Embodiment 72 is the polynucleotide of any one of embodiments 64 to 71, wherein the polynucleotide is immobilized on a solid support.

Embodiment 73 is the polynucleotide of embodiment 72, wherein the polynucleotide is immobilized on the solid support via the 5′ terminal polynucleotide.

Embodiment 74 is the polynucleotide of embodiment 73, wherein the polynucleotide is immobilized to the solid support via binding of an affinity moiety on the 5′ terminal polynucleotide to a binding moiety on the surface of the solid support.

Embodiment 75 is the polynucleotide of any one of embodiments 64 to 74, wherein an affinity moiety is attached via a linker to the 5′ terminal polynucleotide.

Embodiment 76 is the polynucleotide of any one of embodiments 64 to wherein the affinity moiety is biotin, desthiobiotin, or dual biotin.

Embodiment 77 is the polynucleotide of any one of embodiments 64 or 66 to 76, wherein the polynucleotide has the structure 5′-P5-A14-Insert-HYB-Insert-B15′-P7′-3′ or 5′-P7-B15-Insert-HYB′-Insert-A14′-P5′-3′, wherein HYB is a hybridization sequence and HYB′ is the complement of a hybridization sequence.

Embodiment 78 is the polynucleotide of any one of embodiments 65 to 77, wherein the polynucleotide has the structure 5′-P5-A14-Insert1-HYB-Insert2-B15′-P7′-3′ or 5′-P7-B15-Insert1-HYB′-Insert2-A14′-P5′-3′; wherein HYB is a hybridization sequence and HYB′ is the complement of a hybridization sequence.

Embodiment 79 is a composition comprising the polynucleotide of any one of embodiments 64 to 78 hybridized to its complement.

Embodiment 80 is a composition comprising the polynucleotide of any one of embodiments 64 to 78 or a composition of embodiment 79 immobilized on the surface of a solid support, wherein the affinity moiety is biotin, desthiobiotin, or dual biotin and the binding moiety is avidin or streptavidin.

Embodiment 81 is the composition of embodiment 80, wherein the solid support is a bead, slide, wall of a vessel, a flow cell, or a nanowell comprised in a flow cell.

Embodiment 82 is a forked adapter comprising two polynucleotide strands comprising (a) a first strand comprising a sequencing primer sequence and (b) a second strand comprising a 3′ hybridization sequence or its complement, wherein the 3′ end of the first strand is fully or partially complementary to the 5′ end of the second strand.

Embodiment 83 is the forked adapter of embodiment 82, wherein the hybridization sequence or its complement is bound to a blocking oligonucleotide that is fully or partially complementary to the hybridization sequence or its complement.

Embodiment 84 is the forked adapter of embodiment 83, wherein the hybridization sequence or its complement is bound to a blocking oligonucleotide that is fully complementary to the hybridization sequence or its complement.

Embodiment 85 is the forked adapter of any one of embodiments 82 to 84, wherein the first strand and/or second strand further comprise at least one of an adapter, a barcode sequence, a unique molecular identifier (UMI) sequence, a sample index sequence, a capture sequence, or a cleavage sequence.

Embodiment 86 is the forked adapter of any one of embodiments 82 to 85, wherein first strand and/or second strand further comprise a P7 or P5 primer sequence, or their complements.

Embodiment 87 is the forked adapter of any one of embodiments 82 to 86, wherein the sequencing primer sequence comprises a B15 sequence (SEQ ID NO: 6) or an A14 sequence (SEQ ID NO: 4), or their complements.

Embodiment 88 is the forked adapter of any one of embodiments 82 to 87, wherein the first strand comprises a 5′ affinity element capable of binding to an affinity binding partner on a solid support or bead.

Embodiment 89 is the forked adapter of embodiment 88, wherein the affinity element is connected via a linker attached to the first strand.

Embodiment 90 is a composition or kit comprising two forked adapters of any one of embodiments 82 to 89, wherein (a) the first forked adapter comprises a first strand comprising a first read sequencing primer sequence and a second strand comprising a complement of a hybridization sequence and (b) the second forked adapter comprises a first strand comprising a second read sequencing primer sequence and a second strand comprising a hybridization sequence.

Embodiment 91 is the composition or kit of embodiment 44-48 or 90, wherein one or both forked adapters comprise a blocking oligonucleotide.

Embodiment 92 is a method of generating one or more concatenated nucleic acid sequencing templates comprising (a) contacting a sample comprising double-stranded nucleic acid fragments each comprising an insert prepared from a target nucleic acid with the composition or kit comprising two forked adapters, wherein one or both forked adapters comprise a blocking oligonucleotide, optionally wherein the first read sequencing adapter sequence comprises a first read primer binding sequence; (b) ligating the forked adapters to the double-stranded fragments to prepared tagged double-stranded fragments; (c) immobilizing the tagged double-stranded fragments on a solid support; (d) denaturing (1) the immobilized tagged double-stranded fragments to produce immobilized single-stranded fragments and (2) the blocking oligonucleotides to unblock hybridization sequences and complements of hybridization sequences; (e) hybridizing two immobilized single-stranded fragments to each other to form a bridge by binding of the hybridization sequence in a first fragment to the complement of a hybridization sequence in a second fragment; and (f) extending from the 3′ ends of each single-stranded fragment to produce a double-stranded concatenated nucleic acid sequencing template comprising inserts from both immobilized single-stranded fragments.

Embodiment 93 is a method of generating one or more concatenated nucleic acid sequencing templates comprising (a) contacting a sample comprising double-stranded target nucleic acid with two pools of transposome complexes in solution; wherein the first pool of transposome complexes comprises (i) a transposase; (ii) a first transposon comprising a 3′ transposon end sequence and a first read sequencing adapter sequence; and (iii) a second transposon comprising a 5′ sequence fully or partially complementary to the 3′ transposon end sequence and a 3′ complement of a hybridization sequence; and wherein the second pool of transposome complexes comprises (i) a transposase; (ii) a first transposon comprising a 3′ transposon end sequence and a second read sequence adapter sequence; and (iii) a second transposon comprising a 5′ sequence fully or partially complementary to the 3′ transposon end sequence and a 3′ hybridization sequence, wherein one or both second transposons comprise a blocking oligonucleotide; (b) tagmenting the double-stranded nucleic acids to produce tagged double-stranded fragments; (c) releasing the transposome complex from the double-stranded fragments; (d) extending and ligating the double-stranded fragments; (e) immobilizing the tagged double-stranded fragments on a solid support; (f) denaturing (1) the immobilized tagged double-stranded fragments to produce immobilized single-stranded fragments and (2) the blocking oligonucleotides to unblock hybridization sequences and complements of hybridization sequences; (g) hybridizing two immobilized single-stranded fragments to each other to form a bridge by binding of the hybridization sequence in a first fragment to the complement of a hybridization sequence in a second fragment; and (h) extending from the 3′ ends of each single-stranded fragment to produce a double-stranded concatenated nucleic acid sequencing template comprising inserts from both immobilized single-stranded fragments.

Embodiment 94 is the method of embodiment 92 or 93, wherein the denaturing is performed with an increase in temperature, change in pH, and/or addition of one or more chaotropic agents.

Embodiment 95 is the method of embodiment 94, wherein the increase in temperature is an increase from 45° C.-55° C. to 85° C.-95° C., optionally wherein the increase in temperature is an increase from 50° C. to 90° C.

Embodiment 96 is the method of any one of embodiments 92 to 95, wherein the one or more chaotropic agents comprise formamide and/or NaOH.

Embodiment 97 is the method of any one of embodiments 92 to 96, wherein the immobilizing is by binding of an affinity moiety (1) comprised in the first and/or second forked adapter or (2) comprised in a tag from a second transposome to one or more binding moieties on the surface of the solid support.

Embodiment 98 is the method of any one of embodiments 92 to 97, wherein the affinity moiety is biotin, desthiobiotin, or dual biotin and the binding moiety is avidin or streptavidin.

Embodiment 99 is the method of any one of embodiments 92 to 98, wherein one or more additional rounds of denaturing, hybridizing, and extending are performed.

Embodiment 100 is the method of any one of embodiments 92 to 99, wherein a first single-stranded fragment comprises an insert and a second single-stranded fragment comprises an insert that is the complement of the insert comprised in the first fragment.

Embodiment 101 is the method of any one of embodiments 92 to 100, wherein a first single-stranded fragment comprises an insert and a second fragment comprises an insert that is not the complement of the insert comprised in the first fragment.

Embodiment 102 is the method of any one of embodiments 92 to 101, wherein hybridizing occurs between single-stranded fragments prepared from double-stranded fragments comprising (1) a first forked adapter ligated at one end of each fragment and a second forked adapter ligated at the other end of each fragment or (2) a tag from a second transposon of a first transposome complex at one end of each fragment and a tag from a second transposon of a second transposome at the other end of each fragment.

Embodiment 103 is the method of any one of embodiments 92 to 102, wherein two immobilized single-stranded fragments do not hybridize to each other to form a bridge in the absence of binding of a hybridization sequence in a first fragment to the complement of a hybridization sequence in a second fragment.

Embodiment 104 is the method of embodiment 103, wherein the hybridizing two immobilized single-stranded fragments to each other to form a bridge does not occur between single-stranded fragments prepared from double-stranded fragments comprising (1) the same forked adapter ligated at both ends of each fragment or (2) a tag from the same transposome complex at both ends of each fragment.

Embodiment 105 is a method of generating one or more concatenated nucleic acid sequencing templates comprising (a) compartmentalizing a sample comprising target double-stranded nucleic acid into a plurality of different compartments; (b) preparing fragments each comprising an insert from the double-stranded nucleic acid within the plurality of different compartments; (c) contacting the plurality of different compartments with a composition or kit of comprising two forked adapters of embodiment 91, wherein one or both forked adapters comprise a blocking oligonucleotide; (d) ligating the forked adapters to the double-stranded fragments to prepared tagged double-stranded fragments within the plurality of different compartments; (e) denaturing (1) the immobilized tagged double-stranded fragments to produce single-stranded fragments and (2) the blocking oligonucleotides to unblock hybridization sequences and complements of hybridization sequences within the plurality of different compartments; (f) hybridizing two single-stranded fragments within the same compartment to each other to form a bridge by binding of the hybridization sequence in a first fragment to the complement of a hybridization sequence in a second fragment; and (g) extending from the 3′ ends of each single-stranded fragment to produce a double-stranded concatenated nucleic acid sequencing template comprising inserts from both single-stranded fragments within the same compartment.

Embodiment 106 is the method of embodiment 105, wherein the target double-stranded nucleic acid comprises double-stranded DNA fragments, and the preparing fragments prepares subfragments of the double-stranded DNA fragments.

Embodiment 107 is the method of embodiment 63, 105 or 107, wherein the compartments are wells, tubes, or droplets.

Embodiment 108 is the method of any one of embodiments 105 to 107, wherein the denaturing is performed with an increase in temperature, change in pH, and/or addition of one or more chaotropic agents.

Embodiment 109 is the method of embodiment 108, wherein the increase in temperature is an increase from 45° C.-55° C. to 85° C.-95° C., optionally wherein the increase in temperature is an increase from 50° C. to 90° C.

Embodiment 110 is the method of embodiment 108 or 109, wherein the one or more chaotropic agents comprise formamide and/or NaOH.

Embodiment 111 is the method of any one of embodiments 105 to 110, wherein one or more additional rounds of denaturing, hybridizing, and extending are performed.

Embodiment 112 is the method of any one of embodiments 105 to 111, wherein a first single-stranded fragment comprises an insert and a second single-stranded fragment comprises an insert that is the complement of the insert comprised in the first fragment.

Embodiment 113 is the method of any one of embodiments 105 to 111, wherein a first single-stranded fragment comprises an insert and a second fragment comprises an insert that is not the complement of the insert comprised in the first fragment.

Embodiment 114 is the method of any one of embodiments 105 to 113, wherein hybridizing occurs between single-stranded fragments prepared from double-stranded fragments comprising a first forked adapter ligated at one end of each fragment and a second forked adapter ligated at the other end of each fragment.

Embodiment 115 is the method of any one of embodiments 105 to 114, wherein single-stranded fragments do not hybridize to each other in the absence of binding of a hybridization sequence in a first fragment to the complement of a hybridization sequence in a second fragment.

Embodiment 116 is the method of embodiment 115, wherein the hybridizing two single-stranded fragments to each other does not occur between single-stranded fragments prepared from double-stranded fragments comprising the same forked adapter ligated at both ends of each fragment.

Embodiment 117 is the method of any one of embodiments 63 or 105 to 116, wherein the compartmentalizing comprises dilution of the sample such that most compartments comprise one or no target double-stranded nucleic acid.

Embodiment 118 is the method of embodiment 117, wherein inserts comprised in the same concatenated sequencing templates were prepared from the same target nucleic acid.

Embodiment 119 is the method of any one of embodiments 63 or 105 to 118, wherein the compartmentalizing separates most different haplotypes into different compartments and the method is used for haplotype phasing.

Embodiment 120 is the method of embodiment 119, wherein the haplotype phasing does not require barcodes.

Embodiment 121 is a solid support comprising two pools of immobilized transposome complexes, wherein (a) the first pool of transposome complexes comprises (i) a transposase; (ii) a first transposon comprising a 3′ transposon end sequence, a first read sequencing adapter sequence, and a 5′ affinity moiety; and (iii) a second transposon comprising a 5′ sequence fully or partially complementary to the 3′ transposon end sequence and a 3′ complement of a hybridization sequence; and (b) the second pool of transposome complexes comprises (i) a transposase; (ii) a first transposon comprising a 3′ transposon end sequence, a second read sequence adapter sequence, and a 5′ affinity moiety; and (iii) a second transposon comprising a 5′ sequence fully or partially complementary to the 3′ transposon end sequence and a 3′ hybridization sequence, wherein each first transposon is immobilized by binding of a 5′ affinity moiety to a binding moiety on the surface of the solid support.

Embodiment 122 is the solid support of embodiment 121, wherein the first or second pool of transposome complexes comprises the transposome complex of any one of embodiments 30 to 42, wherein the first read sequencing adapter sequence comprises a first read primer binding sequence.

Embodiment 123 is the solid support of embodiment 121 or 122, wherein the first and/or second pool of transposomes complexes comprise homodimers and/or heterodimers.

Embodiment 124 is the solid support of embodiment 122 or 123, wherein the solid support is a bead, slide, wall of a vessel, a flow cell, or a nanowell comprised in a flow cell.

Embodiment 125 is the solid support of any one of embodiments 121 to 124, wherein one or more transposons comprises an index sequence and/or a UMI.

Embodiment 126 is the solid support of embodiment 125, wherein a first transposon comprised in a first pool of transposome complexes and/or a first transposon comprised in a second pool of transposome complexes comprise sample indexes.

Embodiment 127 is the solid support of embodiment 126, wherein both a first transposon comprised in a first pool of transposome complexes and a first transposon comprised in a second pool of transposome complexes comprise sample indexes.

Embodiment 128 is the solid support of any one of embodiments 121 to 127, wherein a second transposon comprised in a first pool of transposome complexes and/or a second transposon comprised in a second pool of transposome complexes comprise sample indexes and/or unique molecular identifiers (UMIs).

Embodiment 129 is the solid support of embodiment 128, wherein both a second transposon comprised in a first pool of transposome complexes and a second transposon comprised in a second pool of transposome complexes comprise sample indexes.

Embodiment 130 is the solid support of embodiment 128 or embodiment 129, wherein both a second transposon comprised in a first pool of transposome complexes and a second transposon comprised in a second pool of transposome complexes comprise UMIs.

Embodiment 131 is a method of generating one or more double-stranded concatenated nucleic acid sequencing templates comprising (a) applying a sample comprising a double-stranded nucleic acid immobilized to a solid support; (b) tagmenting the double-stranded nucleic acids to produce tagged double-stranded fragments comprising inserts from the double-stranded nucleic acid, wherein the double-stranded fragments are immobilized to the solid support by binding of the 5′ affinity moieties to a binding moiety on the surface of the solid support; (c) releasing the transposome complex from the double-stranded fragments; (d) extending and ligating the double-stranded fragments; (e) denaturing the double-stranded fragments into single-stranded fragments, wherein single-stranded fragments comprising a 5′ affinity moiety remain immobilized on the solid support; (f) allowing hybridization of a hybridization sequence comprised in a first immobilized single-stranded fragment to a complement of a hybridization sequence comprised in a second immobilized single-stranded fragment thereby forming a bridge; and (g) extending and generating a double-stranded concatenated nucleic acid sequencing template.

Embodiment 132 is the method of embodiment 131, wherein releasing the transposome complex from the double-stranded fragments is performed with SDS.

Embodiment 133 is the method of embodiment 131 or 132, wherein allowing hybridization comprises cooling the solid support and/or applying a hybridization buffer.

Embodiment 134 is the method of embodiment 133, wherein the cooling comprises reducing the temperature of the solid support to 60° C. or cooler.

Embodiment 135 is the method of embodiment 133 or 134, wherein the hybridization buffer comprises a high salt concentration, optionally wherein the high salt concentration is 750 mM NaCl.

Embodiment 136 is the method of any one of embodiments 131 to 135, wherein the denaturing comprises heating the solid support or applying a chemical denaturant.

Embodiment 137 is the method of embodiment 136, wherein the denaturing comprises increasing the temperature of the solid support to 90° C. or warmer.

Embodiment 138 is the method of any one of embodiments 131 to 137, wherein extending comprises providing polymerase, dNTPs, and extension buffer.

Embodiment 139 is the method of any one of embodiments 131 to 138, further comprising additional rounds of allowing hybridization and extending and generating a double-stranded concatenated nucleic acid sequencing template.

Embodiment 140 is the method of embodiment 131 to 139, wherein hybridization of a hybridization sequence comprised in a first immobilized single-stranded fragment to a complement of a hybridization sequence comprised in a second immobilized single-stranded fragment only occurs when the first and second fragment are at a proximity to each other on the surface of the solid support that is closer than the length of the longer of the first or second fragment.

Embodiment 141 is the method of embodiment 131 to 140, wherein the first immobilized fragment and the second immobilized fragment are immobilized in close proximity on the solid support, wherein the close proximity allows binding of 4 or more, 5 or more, 6 or more, 7 or more, 8 or more, 9 or more, or or more nucleotides comprised in the hybridization sequence comprised in the first immobilized fragment to nucleotides comprised in the complement of the hybridization sequence comprised in the second immobilized fragment.

Embodiment 142 is the method of any one of embodiments 131 to 141, wherein the first immobilized fragment and the second immobilized fragment are immobilized within 20 to 500 nanometers of each other on the surface of the solid support.

Embodiment 143 is the method of any one of embodiments 93 to 121 or 131 to 142, wherein the sample comprises multiple double-stranded nucleic acids.

Embodiment 144 is the method of embodiment 143, wherein both the first and the second immobilized fragments are prepared from the same double-stranded nucleic acid, and the double-stranded concatenated nucleic acid sequencing template comprises two inserts from the same double-stranded nucleic acid.

Embodiment 145 is the method of embodiment 144, wherein the two inserts are from two contiguous sequences comprised in the same double-stranded nucleic acid.

Embodiment 146 is the method of embodiment 144, wherein the two inserts are from two proximal sequences comprised in the same double-stranded nucleic acid, wherein the proximal sequences are separated by 100 or less nucleotides, 200 or less nucleotides, 300 or less nucleotides, 400 or less nucleotides, 500 or less nucleotides, 700 or less nucleotides, or 1,000 or less nucleotides in the double-stranded nucleic acid.

Embodiment 147 is the method of embodiment 146, wherein an area of the solid support comprises multiple double-stranded concatenated nucleic acid sequencing template that share common insert sequences from proximal sequences comprised in the same double-stranded nucleic acid.

Embodiment 148 is a double-stranded concatenated nucleic acid sequencing template prepared by the method of any one of embodiments 131 to 147, wherein the structure of the template comprises (a) 5′-P545-A14-ME-Insert1-ME′-HYB-ME-Insert2-ME′-B15′-i7′-P7′-3′; (b) 5′-P5-A14-ME-Insert1-ME′46-HYB-i8′-ME-Insert2-ME′-B15′-P7′-3′; or (c) 5′-P545-A14-ME-Insert1-ME′46-HYB-i8′-ME-Insert2-ME′-B15′-i7′-P7′-3′, or their complements.

Embodiment 149 is the method of any one of embodiments 131 to 148, further comprising (a) releasing double-stranded concatenated nucleic acid sequencing templates from the solid support; and (b) sequencing the templates to determine insert sequences comprised in the templates.

Embodiment 150 is the method of embodiment 149, wherein the releasing comprising enzymatic digestion or chemical cleavage.

Embodiment 151 is the method of embodiment 149 or 150, further comprising amplifying the templates after releasing and before sequencing.

Embodiment 152 is a method of generating one or more concatenated nucleic acid sequencing templates comprising (a) compartmentalizing a sample comprising target double-stranded nucleic acid into a plurality of different compartments; (b) tagmenting the double-stranded nucleic acids to produce tagged double-stranded fragments comprising inserts from the double-stranded nucleic acid within the plurality of different compartments, wherein the tagmenting is performed with two pools of transposome complexes, wherein the first pool of transposome complexes comprises (i) a transposase; (ii) a first transposon comprising a 3′ transposon end sequence and a first read sequencing adapter sequence; and (iii) a second transposon comprising a 5′ sequence fully or partially complementary to the 3′ transposon end sequence and a 3′ complement of a hybridization sequence; and wherein the second pool of transposome complexes comprises (i) a transposase; (ii) a first transposon comprising a 3′ transposon end sequence and a second read sequence adapter sequence; and (iii) a second transposon comprising a 5′ sequence fully or partially complementary to the 3′ transposon end sequence and a 3′ hybridization sequence; (b) denaturing the tagged double-stranded fragments to produce single-stranded fragments; (c) hybridizing two single-stranded fragments within the same compartment to each other by binding of the hybridization sequence in a first fragment to the complement of a hybridization sequence in a second fragment; and (d) extending from the 3′ ends of each single-stranded fragment to produce a double-stranded concatenated nucleic acid sequencing template comprising inserts from both single-stranded fragments.

Embodiment 153 is the method of embodiment 152, wherein double-stranded concatenated nucleic acid sequencing templates are only produced from hybridizing of two single-stranded fragments present in the same compartment.

Embodiment 154 is the method of embodiment 152 or 153, wherein the hybridization sequence and/or its complement is bound to a blocking oligonucleotide that is fully or partially complementary to the hybridization sequence or its complement, and the denaturing comprises denaturing the blocking oligonucleotide to unblock the hybridization sequence and/or its complement.

Embodiment 155 is the method of any one of embodiments 152 to 154, wherein the transposome complexes are in solution.

Embodiment 156 is the method of any one of embodiments 152 to 155, wherein the compartments are wells, tubes, or droplets.

Embodiment 157 is the method of any one of embodiments 152 to 156, wherein the denaturing is performed with an increase in temperature, change in pH, and/or addition of one or more chaotropic agents.

Embodiment 158 is the method of embodiment 157, wherein the increase in temperature is an increase from 45° C.-55° C. to 85° C.-95° C., optionally wherein the increase in temperature is an increase from 50° C. to 90° C.

Embodiment 159 is the method of embodiment 157 or 158, wherein the one or more chaotropic agents comprise formamide and/or NaOH.

Embodiment 160 is the method of any one of embodiments 152 to 159, wherein one or more additional rounds of denaturing, hybridizing, and extending are performed.

Embodiment 161 is the method of any one of embodiments 152 to 160, wherein a first single-stranded fragment comprises an insert and a second fragment comprises an insert that is not the complement of the insert comprised in the first fragment.

Embodiment 162 is the method of any one of embodiments 152 to 161, wherein the compartmentalizing comprises dilution of the sample such that most compartments comprise one or no target double-stranded nucleic acid.

Embodiment 163 is the method of embodiment 162, wherein inserts comprised in the same concatenated sequencing templates were prepared from the same target nucleic acid.

Embodiment 164 is the method of any one of embodiments 63 or 152 to 163, wherein the compartmentalizing separates most different haplotypes into different compartments and the method is used for haplotype phasing.

Embodiment 165 is the method of embodiment 164, wherein the haplotype phasing does not require barcodes.

Embodiment 166 is the method of any one of embodiments 93 to 121 or 131 to 165, further comprising amplifying the templates.

Embodiment 167 is the method of any one of embodiments 49-55, 57-59, 93 to 121, or 131 to 166, further comprising sequencing the templates.

Embodiment 168 is the method of embodiment 167, wherein sequencing is performed using sequencing primers that bind to A14, B15, and/or a hybridization sequence (HYB).

Embodiment 169 is the method of embodiment 167 or 168, wherein sequencing comprises dark cycles wherein data are not being recorded for a portion of the sequencing.

Embodiment 170 is the method of embodiment 169, wherein the data not being recorded are sequence data associated with the 3′ transposon end sequence or its complement.

Embodiment 171 is the method of any one of embodiments 167 to 170, further comprising (a) evaluating sequences of inserts comprised in the same template; and (b) determining proximity data for sequences comprised in the double-stranded nucleic acid based on inserts that are comprised in the same template.

Embodiment 172 is the method of embodiment 171, wherein the proximity data are determinations that insert sequences (or their complements) were comprised in the same target nucleic acid.

Embodiment 173 is the method of any one of embodiments 167 to 172, further comprising (a) evaluating sequencing results from multiple sequences of a given insert prepared from different templates; and (b) determining instances of non-canonical base pairing based on the sequencing data from (i) the insert and its complement comprised in the same concatenated sequencing template; and/or (ii) the insert comprised in multiple concatenated sequencing templates.

Embodiment 174 is the method of any one of embodiments 167 to 173, further comprising evaluating sequencing results from multiple sequences of a given insert prepared from different templates; and correcting errors in sequencing results for this insert based on the sequencing data from (i) the insert and its complement comprised in the same concatenated sequencing template; and/or (ii) the insert comprised in multiple concatenated sequencing templates.

Embodiment 175 is a method of identifying modified cytosines comprised in an insert sequence comprised in a concatenated sequencing template, comprising (a) preparing a double-stranded concatenated sequencing template, wherein each strand comprises an insert sequence and a copy of the insert sequence and the two strands are complementary to each other; (b) subjecting the double-stranded concatenated sequencing template to a condition for altering modified and/or unmodified cytosines; (c) preparing amplicons of each strand of the double-stranded concatenated sequencing template; (d) sequencing amplicons and evaluating sequencing results for the insert sequence and the copy of the insert sequence in the amplicons produced from each strand; and (e) determining positions of modified cytosines comprised in the insert sequence based on the sequences of each strand of the double-stranded concatenated sequencing template.

Embodiment 176 is the method of embodiment 175, wherein the modified cytosines are methylated or hydroxymethylated cytosines.

Embodiment 177 is the method of embodiment 175 or 176, wherein the concatenated sequencing templates are prepared by the method of any one of embodiments 93 to 121 or 131 to 165.

Embodiment 178 is the method of embodiment 177, wherein extension to produce the double-stranded concatenated sequencing template is performed with a reaction solution comprising methylated-dCTP.

Embodiment 179 is the method of any one of embodiments 175 to 178, wherein uracils comprised in the concatenated sequencing templates are converted to thymines when preparing amplicons.

Embodiment 180 is the method of any one of embodiments 175 to 179, wherein modified cytosines or unmodified cytosines are altered, optionally wherein modified cytosines are altered by TET-Assisted Pyridine Borane Sequencing (TAPS) treatment or unmodified cytosines are altered by sodium bisulfate or enzymatic treatment.

Embodiment 181 is the method of embodiment 180, wherein modified cytosines are altered and the positions of modified cytosines are determined by the presence of (T,C) in the insert sequence and the copy of the insert sequence, respectively, and the positions of unmodified cytosines are determined by the presence of (C,C) in the insert sequence and the copy of the insert sequence, respectively, and wherein the modified and unmodified cytosines are paired with G's in the complementary strand.

Embodiment 182 is the method of embodiment 180, wherein unmodified cytosines are altered and the positions of modified cytosines are determined by the presence of (C,T) in the insert sequence and the copy of the insert sequence, respectively, and the positions of unmodified cytosines are determined by the presence of (T,T) in the insert sequence and the copy of the insert sequence, respectively, and wherein the modified and unmodified cytosines are paired with G's in the complementary strand.

Embodiment 183 is the method of embodiment 180, wherein the method differentiates positions of methylated cytosines from hydroxymethylated cytosines.

Embodiment 184 is the method of embodiment 183, wherein the subjecting each strand to a condition for altering modified and/or unmodified cytosines comprises (a) reacting each strand with β-glycosyltransferase; (b) reacting each strand with a DNA methyltransferase (DNMT); and (c) reacting each strand with a condition that converts unmodified cytosines to uracils.

Embodiment 185 is the method of embodiment 184, wherein (a) the positions of methylated cytosines are determined by the presence of (C,C) in the insert sequence and the copy of the insert sequence, respectively; (b) the positions of hydroxymethylated cytosines are determined by the presence of (C,T) in the insert sequence and the copy of the insert sequence, respectively; and (c) the positions of unmodified cytosines are determined by the presence of (T,T) in the insert sequence and the copy of the insert sequence, respectively; and wherein the methylated, hydroxymethylated, and unmodified cytosines are paired with G's in the complementary strand.

Embodiment 186 is the method of embodiment 183, wherein the subjecting each strand to a condition for altering modified and/or unmodified cytosines comprises (a) reacting each strand with a DNMT; and (b) reacting each strand with a condition that converts methylated cytosines to dihydroxyuracil (^{DH} U).

Embodiment 187 is the method of embodiment 186, wherein (a) the positions of methylated cytosines are determined by the presence of (T,T) in the insert sequence and the copy of the insert sequence, respectively; (b) the positions of hydroxymethylated cytosines are determined by the presence of (T,C) in the insert sequence and the copy of the insert sequence, respectively; and (c) the positions of unmodified cytosines are determined by the presence of (C,C) in the insert sequence and the copy of the insert sequence, respectively; and wherein the methylated, hydroxymethylated, and unmodified cytosines are paired with G's in the complementary strand.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides an overview of how a polynucleotide comprising 2 insert sequences can increase sequencing throughput for a flow cell. Sequencing is performed with the read 1 (R1) sequencing primer followed the read 2 (R2) sequencing primer. Then, turnaround is performed and sequencing is performed with the read 3 (R3) sequencing primer followed by the read 4 (R4) sequencing primer.

FIG. 2 shows sequencing of a representative polynucleotide with 2 insert sequences, wherein the polynucleotide comprises P5′ and P7 sequences and a hybridization (HYB) sequence. The polynucleotide is first sequenced using a Read 1 sequencing primer that hybridizes to the 3′ polynucleotide (comprising a P5′ sequence) of the polynucleotide followed by a Read 2 sequencing primer that hybridizes to the HYB sequence. Turnaround is performed. Then the polynucleotide is sequenced using a Read 3 sequencing primer that hybridizes to the 3 polynucleotide (comprising a P7′ sequence) and a Read 4 sequencing primer that hybridizes to the complement of a hybridization sequence (HYB′).

FIG. 3 shows sequencing of a representative polynucleotide with two insert sequences, generated from Library A or Library B. The polynucleotide is first sequenced using a Read 1 sequencing primer that hybridizes to the 3′ polynucleotide (comprising a P5′ sequence) followed by a Read 2 sequencing primer that hybridizes to the HYB sequence and an SBS sequence. The SBS sequence aids in binding of the sequencing primer, for example, an SBS sequence may comprise ME or ME′). Turnaround is performed. Then the polynucleotide is sequenced using a Read 3 sequencing primer that hybridizes to the 3′ polynucleotide (comprising a P7′ sequence) followed by a Read 4 sequencing primer that hybridizes to the complement of a hybridization sequence (HYB′) and SBS sequence. The representative polynucleotide also shows that the two insert sequences may come from 2 separate libraries, Library A and Library B.

FIGS. 4A-4B show an overview of sequencing of a standard Illumina pair-end library comprising one insert compared to the sequence of polynucleotide comprising two insert sequences. (A) With a standard Illumina pair-end library, 150-cycle sequencing by synthesis (SBS) sequencing is performed for the forward strand with the Read 1 sequencing (seq) primer (SEQ ID NO: 22) that hybridizes to A14′ and ME′. Then a paired-end turn around is performed, and 150-cycle sequencing by SBS is performed for the reverse strand with the Read 2 seq primer (SEQ ID NO: 23) that hybridizes to B15′ and ME′. (B) With a pair-end library of polynucleotides comprising two insert sequences, 150-cycle SBS sequencing is performed for the forward strand with the Read 1-A sequencing (first read) primer that hybridizes to A14′ and ME′. Then the SBS-synthesized strand can be denatured, and the Read 1-B seq primer (second read) is hybridized to the HYB and ME′. Paired-end turn around is performed and 150-cycle sequencing by SBS sequencing is performed for the reverse strand for each of the two insert sequences of the polynucleotide using the Read 2-A sequencing (third read) primer that hybridizes to B15′ and ME′ and then the Read 2-B sequencing (fourth read) primer that hybridizes to HYB′ and ME′. In this way, the sequences of two insert sequences from a target nucleic acid are acquired using the same area of the flowcell as the standard method.

FIGS. 5A-5C show steps in a standard Nextera Flex workflow that results in a sequencing-ready fragment comprising a single insert sequence from a target nucleic acid (genomic DNA or gDNA).

FIGS. 6A-6E show a general overview of preparation of a tandem read library with transposomes to incorporate A14 and B15 sequences (A), followed by PCR to add either P5 and HYB (H) sequences (B) or HYB′ (H′) and P7′ (C). Boxed library products in (D) are capable of forming a hybridization adduct (via HYB/HYB′ hybridization) with another library product to allow extension. At least 1/9th of the extended product is anticipated to be sequenceable product (E).

FIGS. 7A-7B shows a method wherein a P5-HYB′ forked library is formed in one tube using bead-based tagmentation and a P7-HYB forked library is formed in another tube using solution-based tagmentation (A). The library products can form a hybridized adduct based on hybridization of HYB and HYB′ and polynucleotides can be generated via extension (B).

FIGS. 8A-8B show preparation of library products via bead-linked transposomes (BLTs) in tube 1 (type 1 BLTs with anchoring to the bead by P5) and tube 2 (type 2 BLTs with anchoring to the bead by P7). P7 can be anchored to beads using single desthiobiotin, which can be easily removed off streptavidin-coated beads using a release buffer (A). Therefore, the P7-HYB library can be selectively released off the beads and allowed to hybridize to P5-HYB′ library on the bead type 1 (B). After extension, a concatenated nucleic acid sequencing template is generated.

FIGS. 9A-9B show a simple single-tube workflow based on bead-linked-transposons that allows generated of two libraries, wherein one library product comprises HYB′ and the other library product comprises HYB (A). A process of denaturing, hybridization, and extension results in preparation of concatenated nucleic acid sequencing template (B).

FIG. 10 shows a representative Truseq method to generate 2 library products that can be used to generate polynucleotides comprising 2 inserts that can be used for sequencing. The SBS sequence is a sequence that may bind to a sequencing primer, for example the SBS sequence may comprise a sequence complementary to a known sequencing primer. The “SBS” in this figure generically refers to either a SBS sequence or a sequence fully or partially complementary to a SBS sequence (e.g., SBS or SBS′).

FIG. 11 shows Bioanalyzer results on the size of a tandem library (i.e., a polynucleotide comprising two insert sequences) generated via a Truseq method compared to the two library products (P5-HYB′ and P7-HYB) used to generate the tandem library.

FIG. 12 shows 2 libraries generated via a Truseq method, wherein the attachment polynucleotide and the hybridization polynucleotide of each forked adapters comprise SBS sequences. As shown in FIG. 12, “SBS” can generically refer to either a SBS or SBS' sequence (i.e., the tandem SBS sequences in FIG. 12 may comprise SBS/SBS' sequences that are fully or partially complementary).

FIG. 13 shows 2 libraries generated via a Truseq method, wherein the attachment polynucleotide of each forked adapter comprises either A14 and ME or B15 and ME.

FIGS. 14A and 14B show thumbnail images of data from sequencing of a polynucleotide comprising two insert sequences with a Read 1-A seq primer (first read primer 1, (A)) and a Read 1-B seq primer (second read primer, (B)).

FIGS. 15A-F shows an exemplary method of preparing a tandem insert library using ligation. FIG. 15A (SEQ ID NOS: 41, 42, 43, and 30) shows an exemplary first starting library a BtgZI cut site. FIG. 15B (SEQ ID NOS: 44, 45, 46, and 31) shows an exemplary second starting library with a BglII cut site. Each of the two starting libraries are digested with respective restriction enzymes to generate compatible overhangs (FIGS. 15C-D) (SEQ ID NOS: 41, 43, 44, and 46-48). Streptavidin magnetic beads are used to clean up the digested DNA and the digested DNA are ligated together (FIG. 15E) (SEQ ID NOS: 49-54). Each new piece of DNA has unique adapters that mitigates issues with fork handle complementarities. Primers Reads 1, 2, 3, and 4 are used to sequence the new library (FIG. 15F). Exemplary P5 and P7 sequences are shown in black highlights and white text.

FIGS. 16A-B show an exemplary method of preparing a tandem insert library with two different ends. FIG. 16A shows an exemplary workflow to produce a first library using an adapter with a BtgZI cut site and a P5-Read 1 site. FIG. 16B shows an exemplary workflow to produce a second library using an adapter with a BglII cut site and a P7-Read 2 site. Both libraries are made double stranded by primer extension using one primer.

FIG. 17 shows an exemplary method of preparing a tandem insert library using a strand overlap extension (SOE) method. DNA 1 and DNA 2 represent inputs for exemplary first and second libraries. DNA 1 and DNA 2 are prepared separately so that each resulting tandem insert library has DNA appended to a unique adapter. Each library is sheared to produce DNA fragments, and are processed with polymerase to remove damaged DNA ends that result from the shearing process. The DNA fragments are treated with polymerase to generate blunt end DNA duplexes, and with kinase to phosphorylate the 5′OH of the DNA fragments. Then, a polymerase is used to add an adenine to the 3′ ends of each duplex and the DNA fragments are ligated to the adapters. The first library is ligated with a P5-Read 1/A adapter (adapter 1). The second library is ligated with a P7-Index-Read 2/A′ adapter (adapter 2 or 3). The libraries are cleaned up to select for 150-200 base pair fragments. The libraries are mixed and added to a PCR reaction. The DNA fragments denature at elevated temperatures and reanneal at lower temperatures. This results in the A and A′ complementary sequences to hybridize to each other. A polymerase extends the strands to form the tandem insert polynucleotide. ER=end repair. A-tail=adenine tail. Tag=an exemplary index in a barcode sequence. P5=P5 primer sequence. P7=P7 primer sequence. In some embodiments, a tag is added adjacent to P7. In some embodiments, a tag is added adjacent to P5.

FIG. 18 shows an exemplary library fragment with two inserts separated by an adapter sequence. As shown, four sequencing reads are possible. Reads 1 and 4 give paired end data from the first insert. Reads 2 and 3 give paired end data from the second insert. P5=P5 primer sequence. P7=P7 primer sequence.

FIG. 19 shows an exemplary tandem insert library fragment with inserts from two separate genomes, E. coli and human, or two separate amplicons from the same genome. The two inserts are separate by an adapter sequence. As shown, four sequencing reads are possible. For example, Reads 1 and 4 give paired end data from the E. coli inserts. Reads 2 and 3 give paired end data from the human inserts. P5=P5 primer sequence. P7=P7 primer sequence.

FIG. 20A-D show sequencing data for a tandem insert library produced using the ligation method shown in FIGS. 15A-F. FIG. 20A=Read 1. FIG. 20B=Read 2. FIG. 20C=Read 3. FIG. 20D=Read 4.

FIGS. 21A-B show sequencing data for a tandem insert library produced using the ligation method shown in FIGS. 15A-F. Percent base-calls at each cycle number or a read are shown. Each insert exhibits correct base composition for the genome in question. FIG. 21A=Reads 1 and 4 for E. coli inserts. FIG. 21B=Reads 2 and 3 for human inserts.

FIG. 22 shows a tandem insert library fragment producing using the SOE method shown in FIG. 17. Instead of using sheared genomic DNA fragments, monotemplates were used in this experiment—a PhiX amplicon was used for Insert 1 and an E. coli amplicon was used for Insert 2. Adapters were ligated to the monotemplates and the tandem insert library was produced using the SOE method as shown in FIG. 17. Reads 1 and 4 give paired end data from the PhiX amplicon. Reads 2 and 3 give paired end data from the E. coli amplicon. P5=P5 primer sequence. P7=P7 primer sequence.

FIGS. 23A-D show sequencing data for a tandem insert library produced using the SOE method shown in FIG. 17. FIG. 23A=Read 1. FIG. 23B=Read 2. FIG. 23C=Read 3. FIG. 23D=Read 4.

FIGS. 24A-C show sequencing data for a tandem insert library produced using the SOE method shown in FIG. 17. FIG. 24A (SEQ ID NOS: 55-58) shows the expected sequences for Reads 1, 2, 3, and 4 from a tandem insert library polynucleotide. The double slash marks “II” indicate that the DNA sequence shown belongs to a single polynucleotide template. FIGS. 24B-C show the observed Read 1 (FIG. 24B) (SEQ ID NOS: 36 and 59-62) and Read 2 sequences (FIG. 24C) (SEQ ID NOS: 37 and 63-66).

FIG. 25 provides a summary of forked adapters that may be used to prepare sequencing templates comprising multiple inserts from a target nucleic acid. The first oligonucleotide of a first forked adapter (the “first adapter”) may comprise a 3′ end comprising a transposon end sequence and a 5′ end comprising an adapter, such as a first read sequencing adapter sequence (P5.R1). The first adapter may also comprise a second oligonucleotide comprising a 5′ end comprising the complement of the transposon end sequence comprised in the first oligonucleotide and a 3′ end comprising the complement of a hybridization sequence (X′). The first adapter may also comprise a third oligonucleotide that is a blocking oligonucleotide (X′B) capable of binding to X′. In parallel fashion, the first oligonucleotide of a second forked adapter (the “second adapter”) may comprise a first oligonucleotide comprising a 3′ end comprising a transposon end sequence and a 5′ end comprising an adapter, such as a second read sequencing adapter sequence (P7.R2). The second adapter may also comprise a second oligonucleotide comprising a 5′ end comprising the complement of the transposon end sequence comprised in the first oligonucleotide and a 3′ end comprising a hybridization sequence (X). The second adapter may also comprise a third oligonucleotide that is a blocking oligonucleotide (X′B′) capable of binding to X. The blocking oligonucleotides serve to block hybridization of X′ in the first forked adapter to the X in the second forked adapter until the blocking oligonucleotides are removed. The first adapter and second adapter together may be used in methods to prepare a sequencing template comprising two inserts, as described herein. Bio=biotin, which can be used as an affinity moiety.

FIGS. 26A-26D show combinations of different first and second forked adapters that may be used in the present methods, along with a representation of how similar fragments may be prepared using transposomes in solution. (A) The second oligonucleotide of both the first and second forked adapters are bound to blocking oligonucleotides. (B) The second oligonucleotide of the first forked adapter is bound to a blocking oligonucleotide. (C) The second oligonucleotide of the second forked adapter is bound to a blocking oligonucleotide. (D) Two pools of transposomes in solution may be used to tagment target nucleic acid into fragments in solution. After inactivation (such as with SDS) and extension and ligation with an extension-ligation mixture (ELM), similar tagged fragments may be prepared as shown in A-C for ligation of forked adapter.

FIGS. 27A-27C show different tagged fragments that may be generated by ligation or tagmentation in solution with a mix of the first forked adapter and second forked adapter shown in FIGS. 26A-26D. (A) A fragment tagged with a first forked adapter at one end and a second forked adapter ligated at the other end. (B) A fragment tagged with a first forked adapter at both ends. (C) A fragment tagged with a second forked adapter ligated at both the first and second ends. The expected ratio of tagged fragments would be 50% (A): 25% (B): 25% (C).

FIGS. 28A-28C show how different types of tagged fragments (using methods with the representative first and second adapters shown in FIG. 25 or with the method of FIG. 26D) would or would not hybridize after being immobilized on the surface of a solid support. For ease of illustration, the left and right solid support shown present two different views of the same surface on a solid support; the nucleic acid fragments would all extend upwards from the same surface on a solid support with hybridized fragments forming a bridged configuration. (A) A double-stranded fragment comprising an insert is immobilized to a surface of a solid support and denatured, thus producing two single-stranded fragments. A first single-stranded fragment comprising a ligated first oligonucleotide of the first forked adapter (P5.R1) at one end and a ligated second oligonucleotide of the second forked adapter at the other end (X) can hybridize to a second single-stranded fragment comprising a ligated second strand of the first forked adapter (X′) at one end and a ligated first oligonucleotide of the second forked adapter at the other end (P7.R2). These two fragments may likely be complements of each other (i.e., were two single strands comprised in the same double-stranded fragment), because both strands from a double-stranded fragment will likely be immobilized close to each other after the double-stranded fragment is denatured (shown). The two fragments can also be sequences that are not complements of each other (not shown). This hybridization of two single-stranded fragments occurs via binding of the hybridization sequence (X) to the complement of the hybridization sequence (X′). After the hybridization of the two fragments by X/X′, elongation can be performed from the 3′ ends of the ligated sequences. (B) Single-stranded tagged fragments with ligated first/second oligonucleotides from the first forked adapter at both ends cannot hybridize to each other (since they both comprise an X′ sequence at one end). (C) Single-stranded tagged fragments with ligated first/second oligonucleotides from the second forked adapter at both ends cannot hybridize to each other at the hybridization sequence (since they both comprise an X sequence at one end). Accordingly, 100% of single-stranded fragments with the same insert that are capable of hybridizing to each other at the hybridization sequence are those prepared from a double-stranded fragment with one forked adapter at a first end and a second forked adapter at a second end.

FIG. 29 shows a double-stranded concatenated sequencing template comprising two inserts in each strand prepared using forked adapter. In this representative example, both inserts are copies of the same insert sequence of Strand A or Strand A′ (shown). In other examples, the two insert sequences in each strand of a double-stranded concatenated sequencing template may be different from each other (not shown).

FIG. 30 shows methods of denaturing (to separate strands of the double-stranded fragment and remove blocking oligonucleotides) and annealing of immobilized single-stranded fragments. When used after ligation of forked adapters, these methods can prepare concatenated sequencing templates comprising two inserts in each strand. As both strands of a double-stranded fragment will be constrained and likely to bind in the same area of a solid support, this method would often produce concatenated sequencing templates comprising two copies of the same insert sequence (such as A′/A′ and A/A). Whereas concatenated sequencing templates can be prepared from single-stranded fragments comprising different adapters (such as A/A′, B/B′, and D/D′), concatenated sequencing templates (produced from two single-stranded fragments generated from one double-stranded fragment) will not be prepared from single-stranded fragments that comprise the same adapters at both ends (such as C/C′ and E/E′).

FIG. 31 shows a method of preparing concatenated sequencing templates using tubes or wells as compartments. The f1, f2, and f3 refer to different relatively large fragments that can then be converted into subfragments.

FIG. 32 shows a method of preparing concatenated sequencing templates using droplets as compartments.

FIG. 33 shows a method of preparing concatenated sequencing templates for haplotype phasing using compartments. A sample is subjected to limiting dilution in compartments, which leads to a very low likelihood that two chromosomes of different haplotypes end up in the same compartment. In this example, Chr1-Hap1 and Chr2-Hap1 are comprised in one compartment and Chr1-Hap2 and Chr2-Hap2 are comprised in a different compartment. The box shown with the checked arrow comprise concatenated sequencing templates that can be generated after the process of denaturing, reannealing, and extending. The box shown with the “X” arrow indicates concatenated sequencing templates that cannot be generated (because these chromosomes were comprised in different compartments). Concatenated sequencing templates can only comprise inserts sequences from chromosomes that were comprised in the same compartment, and these templates are comprised in the box shown with the checked arrow. The dashed ovals in the box shown with the checked arrow represent concatenated sequencing templates that constitute the original haplotypes. The other concatenated sequencing templates in the box shown with the checked arrow (i.e., those not in dashed ovals) comprise inserts that originated from different chromosomes.

FIG. 34 shows transposomes that may be used to prepare sequencing templates comprising two or more inserts. A first and a second transposome each comprise a forked adapter. As used herein, a “first oligo” or “first strand” may refer to a first transposon that is comprised in a forked adapter, and a “second oligo” or “second strand” may refer to a second transposon that is comprised in a forked adapter. The forked adapter of the first transposome comprises a first strand comprising a 3′ transposon end sequence (such as ME, SEQ ID NO: 6) and a first read sequencing adapter sequence (P5.R1) and a second strand comprising a 5′ complement of a transposon end sequence (such as ME′, SEQ ID NO: 3) and a 3′ complement of a hybridization sequence (X′). The forked adapter of the second transposome comprises a first strand comprising a 3′ transposon end sequence and a second read sequencing adapter sequence (P7.R2) and a second strand comprising a 5′ complement of a transposon end sequence and a 3′ hybridization sequence (X). This representative example shows two pools of transposomes wherein each pool is a homodimer (denoted with two checked transposons or two striped transposons). As described herein, transposomes may also comprise heterodimers.

FIG. 35 shows a solid support having immobilized transposomes (as shown in further detail in FIG. 34) immobilized on its surface. B=biotin, which is used as an affinity moiety to bind transposomes to the surface of a solid support.

FIG. 36 shows steps of tagmentation using the solid support shown in FIG. 35. A double-stranded nucleic acid is added to the solid support. Next, fragments are prepared by tagmentation. Transposases are removed using SDS and washing. Finally, extension and ligation are performed using an extension ligation mix (ELM) buffer. This example shows tagmentation by only one pair of transposomes.

FIG. 37 shows bridging of fragments produced by transposomes. A double-stranded DNA may comprise the sequence A in the sense strand and A′ in the antisense strand. The bridges may be between a first transposome and a second transposome, or a first transposome and a first transposome, or a second transposome and a second transposome. Such permutations will occur in a ratio of respectively.

FIG. 38 shows immobilized fragments after release of transposomes and denaturing of fragments. The single-stranded fragments may have been prepared from a first transposome and a second transposome (50%), or a first transposome and a first transposome (25%), or a second transposome and a second transposome (25%). Accordingly, fragments have either X or X′ on their free end, based on which transposome prepared each fragment.

FIG. 39 shows representative single-stranded fragments and whether they can hybridize with each other to form a bridge. A X/X′ set of sequences in two different single-stranded fragments can hybridize (producing 100% of hybridizations), a X′/X′ set of sequences cannot hybridize (0%), and a X/X set of sequences cannot hybridize (0%). Accordingly, 100% bridged single-stranded fragments are prepared from binding of an X sequence in one fragment to an X′ in another fragment (i.e., binding of a hybridization sequence to its complement).

FIG. 40 shows formation (or not) of concatenated sequencing templates comprising two copies of an insert sequence. A double-stranded concatenated sequencing template is formed comprising two copies of the A-strand in tandem in the sense strand and two copies of the A′-strand in tandem in the antisense strand after hybridization of the X/X′ sequences (100%), while no concatenated sequencing template is formed between single-stranded fragments that both comprise a X′ (0%) or both comprise a X sequence (0%). The resulting double-stranded concatenated sequencing template may comprise P5 or P5′ at one end and P7 or P7′ at the other end.

FIG. 41 shows bridges that may be formed when a double-stranded nucleic acid is tagmented by transposomes to prepare two bridged inserts. In this representative example, the double-stranded nucleic acid comprising sequences A and B in the sense strand and sequences A′ and B′ in the antisense strand. Exemplary options for tagging of the two bridged fragments with different adapter sequences from the first and/or second forked adapters comprised in transposomes are shown.

FIG. 42 shows exemplary hybridizations between single-stranded fragments to produce concatenated sequencing templates. These hybridizations can occur between fragments that comprise an insert and its complement sequence (such as A/A′ or B/B′) or between fragments that comprise two different inserts (such as A/B, A′/B, A/B′, and A′/B′). Some hybridizations will all produce sequenceable concatenated sequencing templates (after extension) with P5/P5′ at one end and P7/P7′ at the other end. Other hybridizations will produce some nonsequenceable concatenated sequencing templates (after extension). Nonsequenecable concatenated sequencing templates could include those with P5/P5′ at both ends or P7/P7′ at both ends, and these representative templates are outlined with dashed boxes.

FIG. 43 shows two bridged inserts prepared from only transposomes comprising the second forked adapter or from only transposomes comprising the first forked adapter.

FIG. 44 shows that single-stranded fragments with an adapter from the second forked adapter at both ends cannot hybridize together, and single-stranded fragments with an adapter from the first forked adapter at both ends cannot hybridize together. This lack of hybridization is because a X sequence cannot hybridize with another X sequence, and similarly a X′ sequence cannot hybridize with another X′ sequence.

FIG. 45 shows representative examples wherein a group of 5 bridged inserts can lead to a variety of hybridizing between fragments comprising different insert sequences. Though not shown in the figure, fragments with sense and antisense of the same sequence (such as A and A′) can also hybridize. While not all pairing would produce sequenceable concatenated sequencing templates (after extension) with different adapters at the ends of the templates, many combinations would. Exemplary concatenated sequencing templates generated from hybridized single-stranded fragments are shown in the boxes.

FIGS. 46A-46C show sequencing templates that include sample indexes. (A) Transposome complexes comprising sample indexes i5 on the first strand of the forked adapter comprised in the first transposome complex and an i7 on the first stand of the forked adapter comprised in the second transposome complex, along with a sequencing template that may be prepared using these transposomes. (B) Transposome complexes comprising sample indexes i8 on the second strand of the forked adapter comprised in the first transposome complex and an i6 on the second stand of the forked adapter comprised in the second transposome complex, along with a sequencing template that may be prepared using these transposomes. (C) A representative sequencing template that may be prepared when the first and second strand of the first and second transposomes comprise sample indexes.

FIG. 47 shows how dark cycles may be used to avoid sequencing of ME sequences after binding of primers to A14, B 15 ′, or X sequences used as primer binding sites for concatenated sequencing templates. Binding of primers is shown with arrows that indicate the direction of the sequencing read.

FIG. 48 shows a representative double-stranded concatenated sequencing template comprising an insert and a copy of an insert in each strand, wherein the insert sequences comprise methylated cytosines (NC) and hydroxymethylated cytosines (INC), which may be referred to herein as modified cytosines. One single-stranded template comprises the sense insert (S) and a copy of it (S-copy), while the other single-stranded template comprising the antisense insert (S′) and a copy of it (S′-copy). The S-copy and S′-copy do not comprise modified cytosines. Underlined A, T, and G positions indicate that non-cytosine nucleotides.

FIG. 49 shows results from treatment of the template shown in FIG. 48 with a treatment that converts non-methylated cytosines to uracils (such as sodium bisulfite).

FIGS. 50A-50C show the top strand (A) and bottom strand (B) of a double-stranded concatenated sequencing template as shown in FIG. 25 before and after PCR to prepare amplicons, as well as analysis of sequencing results (C).

FIG. 51 shows results from treatment of the template shown in FIG. 48 with a treatment that converts modified cytosines (methylated and hydroxymethylated cytosines) to dihydroxyuracils (DH U, such as with a TAPS method).

FIGS. 52A-52C show the top strand (A) and bottom strand (B) of a double-stranded concatenated sequencing template as shown in FIG. 51 before and after PCR to prepare amplicons, as well as analysis of sequencing results (C).

FIG. 53 shows a sequencing template prepared with extension performed in the presence of methylated-dCTP. The S-copy and S′-copy can comprise methylated cytosines when prepared by this method.

FIG. 54 shows results after treatment of the sequencing template shown in FIG. 53 with a treatment that converts non-methylated cytosines to uracils.

FIGS. 55A-55C show the top strand (A) and bottom strand (B) of a double-stranded concatenated sequencing template as shown in FIG. 54 before and after PCR to prepare amplicons, as well as analysis of sequencing results (C).

FIG. 56 shows results after treatment of the sequencing template shown in FIG. 53 with a treatment that converts non-methylated cytosines to uracils.

FIGS. 57A-57C show the top strand (A) and bottom strand (B) of a double-stranded concatenated sequencing template as shown in FIG. 54 before and after PCR to prepare amplicons, as well as analysis of sequencing results (C).

FIG. 58 shows a representative step comprised in a method for performing methylation analysis to differentiate unmodified cytosines, methylated cytosines, and hydroxymethylated cytosines using 0-glucosyltransferase treatment followed by DNA methyltransferase 1 (DNMT1) treatment.

FIG. 59 shows method of converting non-methylated cytosines in the sequencing template prepared in FIG. 58 to uracils.

FIGS. 60A-60C show the top strand (A) and bottom strand (B) of a double-stranded concatenated sequencing template as shown in FIG. 59 before and after PCR to prepare amplicons, as well as analysis of sequencing results (C).

FIG. 61 shows a representative step comprised in a method for performing methylation analysis to differentiate cytosines, methylated cytosines, and hydroxymethylated cytosines using DNA methyltransferase 1 (DNMT1) and conversion of methylated cytosines to DH U.

FIGS. 62A-62C show the top strand (A) and bottom strand (B) of a double-stranded concatenated sequencing template as shown in FIG. 61 before and after PCR to prepare amplicons, as well as analysis of sequencing results (C).

### DESCRIPTION OF THE SEQUENCES

Table 1 provides a listing of certain sequences referenced herein.

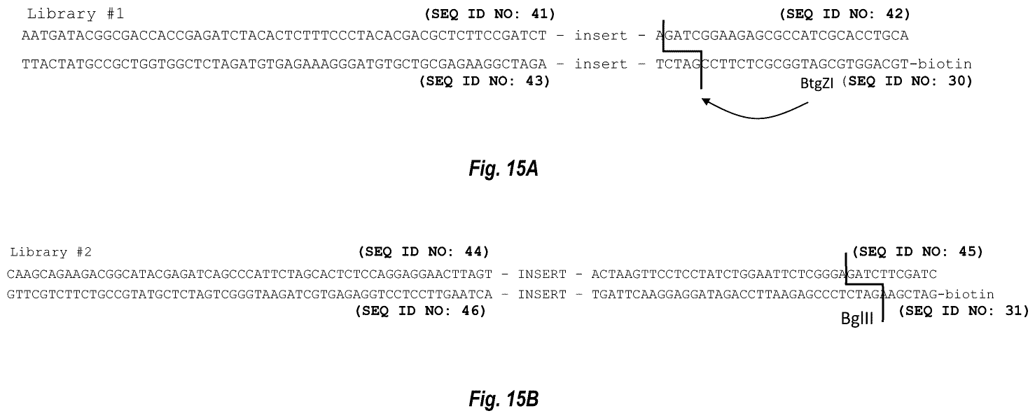

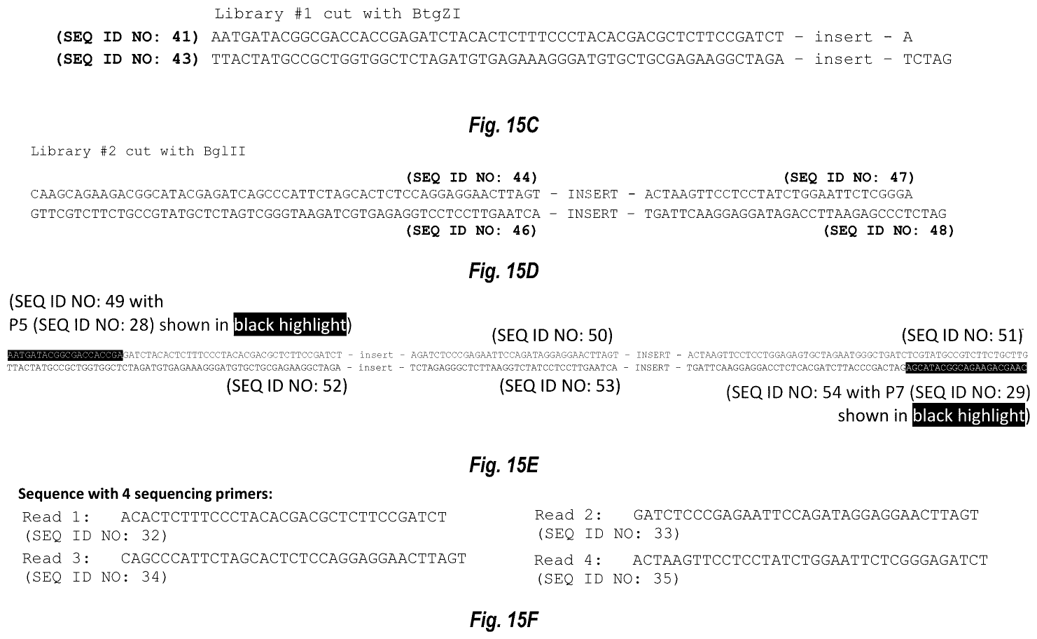

TABLE 1
Description of the Sequences

| Description | Sequences | SEQ ID NO |
|---|---|---|
| Exemplary A14-ME | TCGTCGGCAGCGTCAGATGTGTATAAGAGACAG | 1 |
| Exemplary B15-ME | GTCTCGTGGGCTCGGAGATGTGTATAAGAGACAG | 2 |
| Exemplary ME′ | -phos-CTGTCTCTTATACACATCT | 3 |
| Exemplary A14 | TCGTCGGCAGCGTC | 4 |
| Exemplary B15 | GTCTCGTGGGCTCGG | 5 |
| Exemplary ME | AGATGTGTATAAGAGACAG | 6 |
| Exemplary P5 | AATGATACGGCGACCACCGAGAUCTACAC | 7 |
| Exemplary P7 | CAAGCAGAAGACGGCATACGAG*AT | 8 |
| (G* = modified | | |
| guanine, e.g., an | | |
| 8-oxo-guanine) | | |
| 20P7-B8-ME | CAGAAGACGGCATACGAGATGGGCTCGGAGATGTGTATAAG | 9 |
| | AGACAG | |
| p-18ME′HYB′ | Phos/TGTCTCTTATACACATCTCTCTCTTCTCTCCTTCTT | 10 |
| | CTCTCT | |
| p-18ME′HYB | Phos/TGTCTCTTATACACATCTAGAGAGAAGAAGGAGAGA | 11 |
| | AGAGAG | |
| desthio20P7-B8-ME | /5deSBioTEG/CAGAAGACGGCATACGAGATGGGCTCGGA | 12 |
| | GATGTGTATAAGAGACAG | |
| Exemplary HYB | AGAGAGAAGAAGGAGAGAAGAGAG | 13 |
| (HYB1) | | |
| HYB2 (updated | GAGTAAGTGGAAGAGATAGGAAGG | 14 |
| HYB sequence | | |
| comprising a | | |
| C/G lock on the | | |
| 5′ end of the | | |
| HYB sequence) | | |
| Exemplary P5- | 5′ AATGATACGGCGACCACCGAGATCTACACTCTTTCCCT | 15 |
| SBS sequence | ACACGACGCTCTTCCGATC*T | |
| (* = | | |
| phosphorothioate | | |
| bond) | | |
| Exemplary SBS- | /5Phos/GATCGGAAGAGCCCTTCCTATCTCTTCCACTTAC | 16 |
| HYB′ sequence | TC | |
| Exemplary P7- | 5′CAAGCAGAAGACGGCATACGAGATACATCGGTGACTGGA | 17 |
| SBS sequence | GTTCAGACGTGTGCTCTTCCGATC*T | |
| (* = | | |
| phosphorothioate | | |
| bond) | | |
| Exemplary SBS- | /5Phos/GATCGGAAGAGCGAGTAAGTGGAAGAGATAGGAA | 18 |
| HYB sequence | GG3′ | |
| Exemplary SBS | TCTTTCCCTACACGACGCTCTTCCGATC | 19 |
| sequence | | |
| Exemplary SBS | /5Phos/GATCGGAAGAGC | 20 |
| sequence | | |
| Exemplary SBS | ACATCGGTGACTGGAGTTCAGACGTGTGCTCTTCCGATC | 21 |
| sequence | | |
| Read 1 | TCTTTCCCTACACGACGCTCTTCCGATCT | 22 |
| sequencing | | |
| primer | | |
| Read 2 | CGGTCTCGGCATTCCTGCTGAACCGCTCTTCCGATCT | 23 |
| sequencing | | |
| primer | | |
| Adapter of | /5 Phos/GATCGGAAGAGCGAGTAAGTGGAAGAGATAGGA | 24 |
| P7/HYB2 | AGG3′ | |
| adapter set | | |
| Adapter of | 5′ CAAGCAGAAGACGGCATACGAGATACATCGGTGACTGG | 25 |
| P7/HYB2 | AGTTCAGACGTGTGCTCTTCCGATC*T | |
| adapter set (* = | | |
| phosphorothioate | | |
| bond) | | |
| Adapter of | /5 Phos/GATCGGAAGAGCCCTTCCTATCTCTTCCACTTA | 26 |
| P5/HYB2′ | CTC 3′ | |
| adapter set | | |
| Adapter of | 5′ AATGATACGGCGACCACCGAGATCTACACTCTTTCCCT | 27 |
| P5/HYB2′ | ACACGACGCTCTTCCGATC*T | |
| adapter set | | |
| Exemplary P5 | AATGATACGGCGACCACCGA | 28 |
| Exemplary P7 | CAAGCAGAAGACGGCATACGA | 29 |
| Exemplary | TGCAGGTGCGATGGCGCTCTTCCGATCT | 30 |
| sequence with | | |
| BtgZI cut site in | | |
| FIG. 15A | | |
| Exemplary | GATCGAAGATCTCCCGAGAATTCCAGATAGGAGGAACTTAG | 31 |
| sequence with | T | |
| BglII cut site in | | |
| FIG. 15B | | |
| Exemplary Read | ACACTCTTTCCCTACACGACGCTCTTCCGATCT | 32 |
| 1 sequence | | |
| Exemplary Read | GATCTCCCGAGAATTCCAGATAGGAGGAACTTAGT | 33 |
| 2 sequence | | |
| Exemplary Read | CAGCCCATTCTAGCACTCTCCAGGAGGAACTTAGT | 34 |
| 3 sequence | | |
| Exemplary Read | ACTAAGTTCCTCCTATCTGGAATTCTCGGGAGATCT | 35 |
| 4 sequence | | |
| Exemplary Read | CCTATGATGTTTATCCTTTGGATGGTCGCCATGA | 36 |
| 1 sequence | | |
| Exemplary Read | GACCTTCGCCCGTTTTTTACGGTGCCCGCCAATCG | 37 |
| 2 sequence | | |
| Exemplary Read | TTATCCGCCATATTCGCGATGAATCACATGATCAC | 38 |
| 3 sequence | | |
| Exemplary Read | TCAATAGTCACACAGTCCTTGACGGTATAATAACC | 39 |
| 4 sequence | | |
| A2 | TCACTCAAGAACAGC | 40 |

### DESCRIPTION OF THE EMBODIMENTS

Described herein are polynucleotides comprising multiple insert sequences, wherein the insert sequences are derived from one or more target nucleic acid. These polynucleotides may comprise a concatenation sequence and multiple primer sequences. This application also describes methods of generating these polynucleotides and uses of these polynucleotides. The presence of multiple insert sequences within a given polynucleotide can increase the output of the sequencing platforms by increasing the number of reads that are produced per flowcell.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in the Definitions section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless stated otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The section headings used herein are for organizational purposes only and are not to be construed as limiting the subject matter described.

### I. Definitions

“Hybridization sequence” or “HYB,” as used herein, refers to a sequence that can hybridize to a complementary hybridization sequence. Hybridization of HYB in one library product to a HYB′ in another library product can lead to a hybridization adduct, wherein the two library products anneal to each other via hybridization of HYB/HYB′.

As used herein, a “concatenated nucleic acid sequencing template” refers to a double-stranded composition of a polynucleotide and its complement. A concatenated nucleic acid sequencing template can be generated by association of two library products by hybridization of HYB/HYB′ followed by extension to generate a double-stranded template.

“Insert sequence,” as used herein, refers to a region of a target nucleic acid that is comprised in a polynucleotide. A polynucleotide may comprise multiple insert sequences.

“Stacked reads” or “tandem reads,” as used herein, relates to sequencing reads of multiple insert sequences that are generated from a single polynucleotide. These sequencing reads may be sequential. For example, a polynucleotide comprising 2 or more insert sequences and 2 or more primer sequences can be used to generate tandem reads. A “tandem reads library,” as used herein, refers to a library of polynucleotides comprising multiple insert sequences that can be used to generate tandem reads.

“SBS,” as used herein refers to a sequence that is incorporated into a polynucleotide to improve binding of a read primer. In embodiments wherein polynucleotides are made from library products produced by tagmentation, SBS may be a mosaic end sequence and SBS' may be the complement of a mosaic end sequence, such as ME and ME′. SBS and SBS' sequences may also be comprised in adapters when library products are produced using Truseq methods (Illumina).

### II. Polynucleotides Comprising Multiple Insert Sequences

Described herein are polynucleotides that comprise multiple insert sequences, wherein each insert comprises a portion of one or more target nucleic acid. A single polynucleotide comprising multiple insert sequences allows for sequencing of multiple regions of the one or more target nucleic acid in the same region of a flowcell. In this way, more regions of the one or more target nucleic acid can be sequenced without the need for a larger flowcell.

In some embodiments, the polynucleotides are generated from 2 separate library products based on hybridizing of a HYB in one library product to a HYB′ sequence in the other library product to form a hybridized adduct, followed by elongation to produce a concatenated nucleic acid sequencing template.

These polynucleotides may also comprise additional sequences, such as one or more primer sequences, a concatenation sequences, attachment polynucleotides.

In some embodiments, a polynucleotide comprises a 3′ terminal polynucleotide comprising a first read primer binding sequence; a first insert sequence 5′ of the 3′ terminal polynucleotide that is derived from a target nucleic acid; a concatenation sequence comprising a second read primer binding sequence that is orthogonal to the first read primer binding sequence, wherein the second read primer binding sequence comprises a hybridization sequence; a second insert sequence 5′ of the concatenation sequence and derived from a discontiguous sequence of the target nucleic acid or from a different target nucleic acid than the first insert sequence; and an attachment polynucleotide at the 5′ end of the polynucleotide and comprising an attachment sequence, wherein the 3′ terminal polynucleotide, the concatenation sequence, and the attachment polynucleotide are not derived from the target nucleic acid.

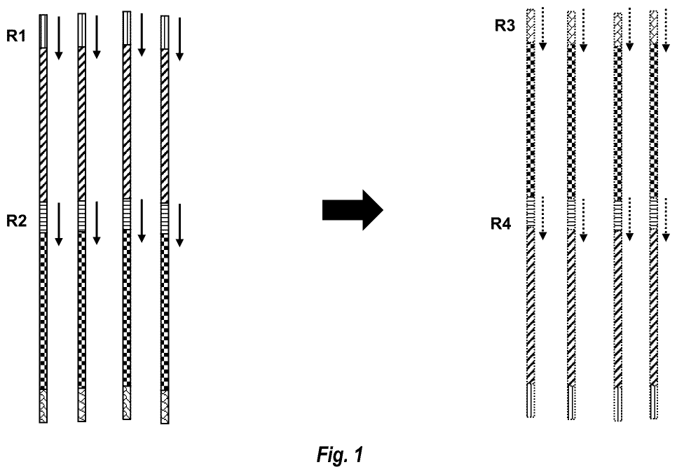

FIG. 1 presents an overview of these polynucleotides, showing how sequencing of an exemplary polynucleotide with 4 primer sequences allows for sequencing of 2 distinct insert sequences.

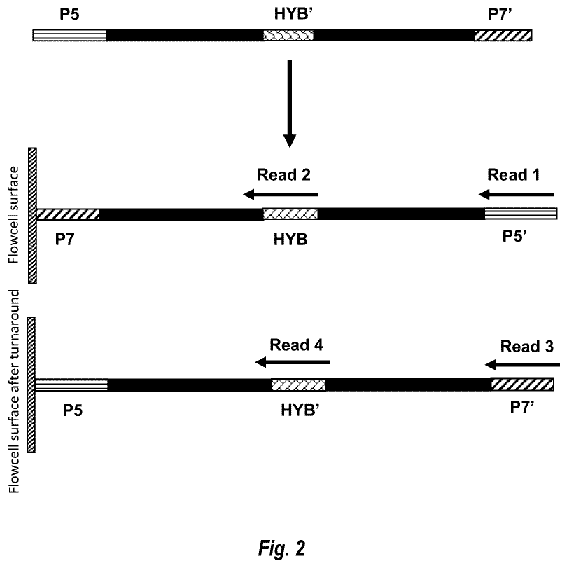

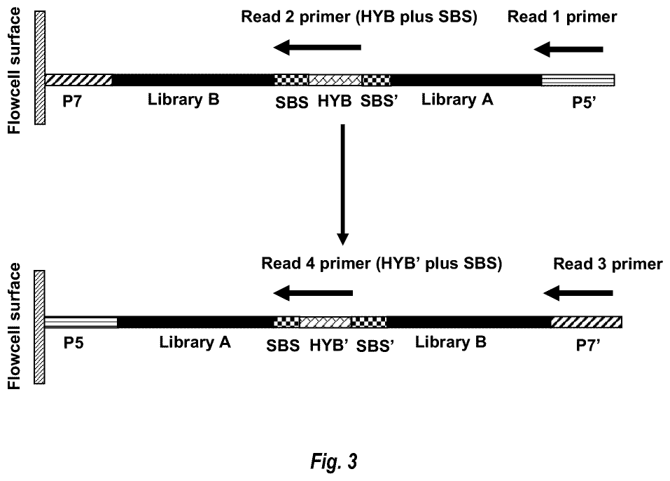

FIG. 2 shows the structure of an exemplary polynucleotide, wherein the concatenation sequence comprises a second read primer binding sequence (Read 2) comprising a hybridization sequence (HYB), a first read primer binding sequence (Read 1) that binds a 3′ polynucleotide comprising a P5′ sequence, and an attachment sequence that comprises a P7 sequence. As shown in FIG. 3, the different inserts in a polynucleotide may be generated from different libraries.

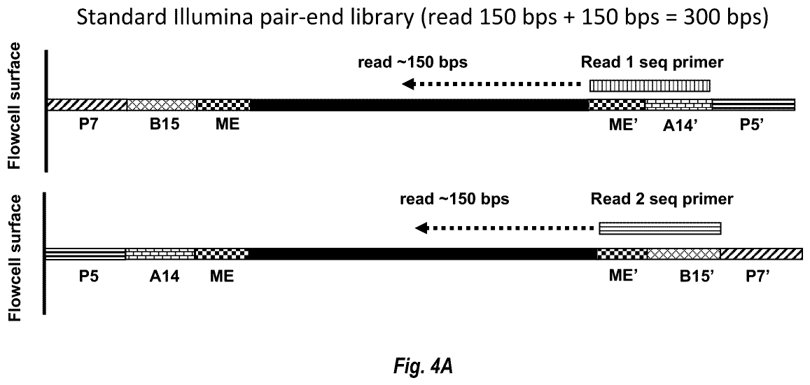

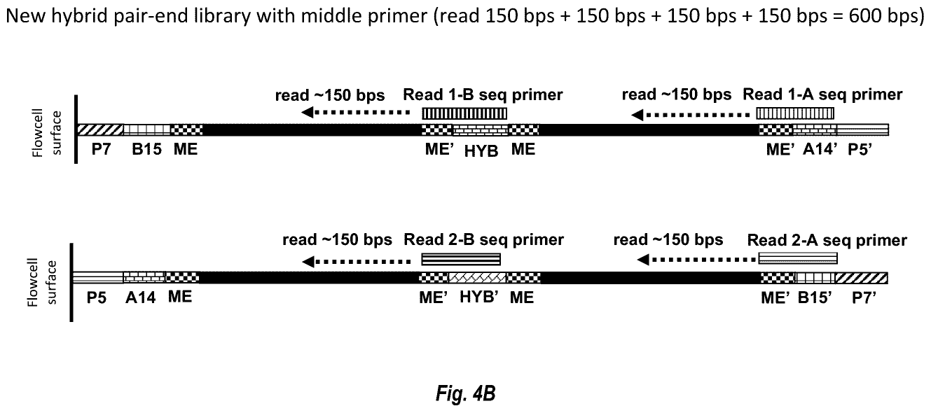

Polynucleotides with multiple insert sequences can allow a greater amount of sequence to be generated from a flowcell compared to a standard Illumina pair-end library, as shown in FIG. 4A versus FIG. 4B. In FIGS. 4A and 4B, the same amount of flow cell surface was used in both cases, so twice as much sequence was generated for the same area of the flow cell surface using the polynucleotide comprising two insert sequences compared to a polynucleotide comprising a single insert.

Also described herein are polynucleotides that may be used as sequencing templates. These sequencing templates may be used with any standard sequencing methods known in the art.

In some embodiments, polynucleotides comprise more than one insert sequence. “Insert sequence” or “insert,” as used herein, refers to a region of a target nucleic acid, such as a double-stranded nucleic acid, that is comprised in a polynucleotide. A polynucleotide may comprise multiple insert sequences. In some embodiments, a polynucleotide comprises two insert sequences. In some embodiments, a polynucleotide comprises three, four, or five insert sequences. A polynucleotide comprising more than one insert that can be used as a sequencing template may be referred to herein as a “concatenated nucleic acid sequencing template” or “concatenated sequencing template.”

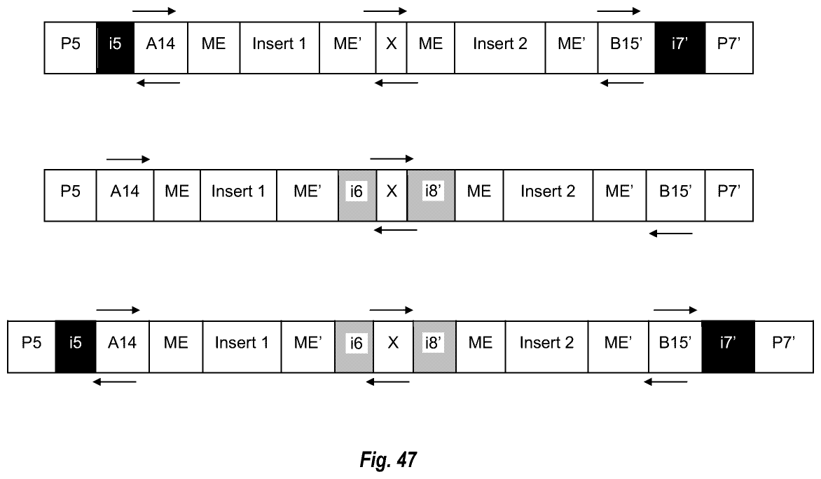

In some embodiments, polynucleotides comprise a hybridization sequence or the complement of a hybridization sequence. “Hybridization sequence” or “HYB,” as used herein, refers to a sequence that can hybridize to a complementary hybridization sequence. For example, hybridization of HYB in one fragment (such as a library product) to a HYB′ (the complement of a hybridization sequence) in another fragment can lead to a hybridization adduct or a bridge, wherein the two fragments anneal to each other via hybridization of HYB/HYB′. In some embodiments, HYB comprises sufficient nucleotides to attach two single-stranded fragments together when HYB hybridizes to HYB′. In some embodiments, a HYB sequence comprised in a concatenated sequencing template may used as a primer binding site, as shown in FIG. 47.

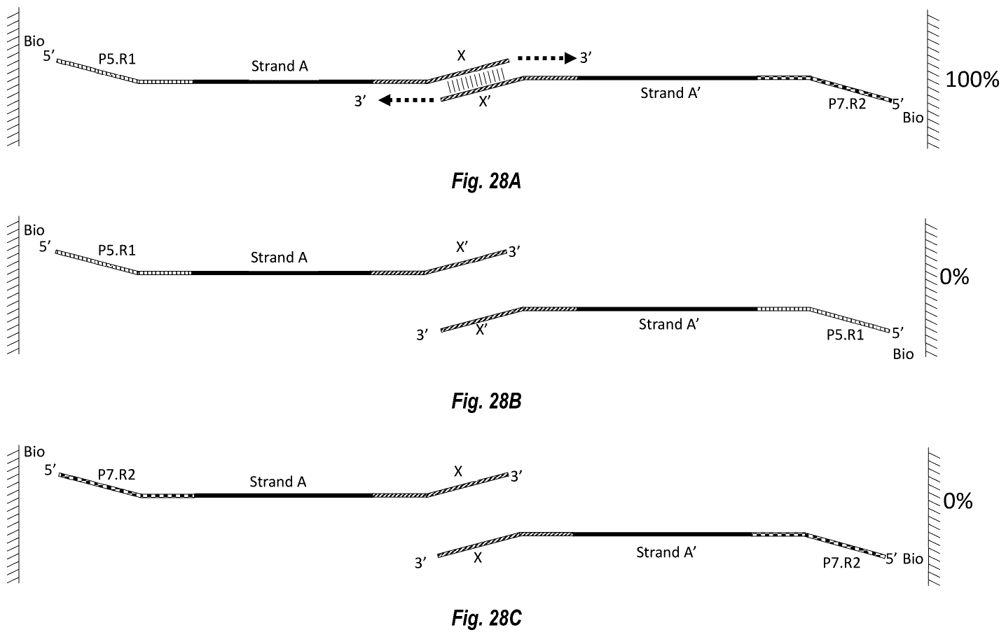

In some embodiments, a HYB or HYB′ comprises 10-30 nucleotides. In some embodiments, binding of the HYB in a first single-stranded nucleic acid fragment to the HYB′ in a second single-stranded nucleic acid fragment is sufficient to “bridge” the two fragments (as described in methods herein with examples shown in FIGS. 28A and 39). The nucleotides comprised in a HYB or HYB′ may be naturally occurring or artificial or modified nucleotides. In some embodiments, HYB or HYB′ comprising artificial or modified nucleotides may require fewer nucleotides in these sequences to allow bridging between two single-stranded fragments.

In some embodiments, one or more nucleotide in the HYB or HYB′ is a locked nucleic acid or a bridged nucleic acid. As used herein, a “locked nucleic acid” or “LNA” refers to a modified RNA nucleotide in which the ribose moiety is modified with an extra bridge connecting the 2′ oxygen and 4′ carbon. In some embodiments, LNAs confer heightened structural stability in the HYB or HYB′ sequence, thus increasing the hybridization melting temperature (Tm) of the HYB/HYB′ interaction. For example, HYB or HYB′ sequences comprising one or more LNAs may only comprise relatively short sequences (such as 10-20 nucleotides), yet still confer sufficiently strong binding to allow formation of bridges between a first single-stranded fragment comprising a HYB and a second single-stranded fragment comprising a HYB′.

In some embodiments, the polynucleotide comprises two or more inserts. As described herein, these inserts may be copies of the same sequence from a target nucleic acid or separate sequences from a target nucleic acid. As used herein, a “chimeric template” refers to a template comprising different inserts.

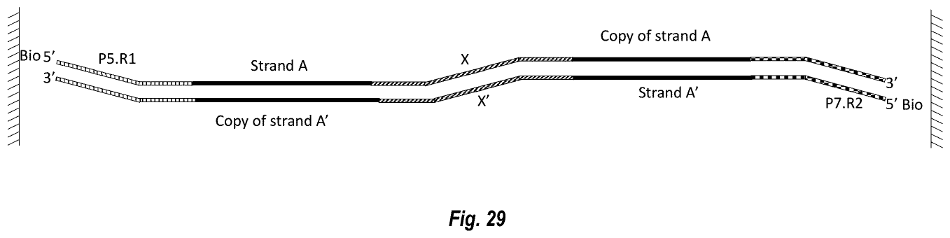

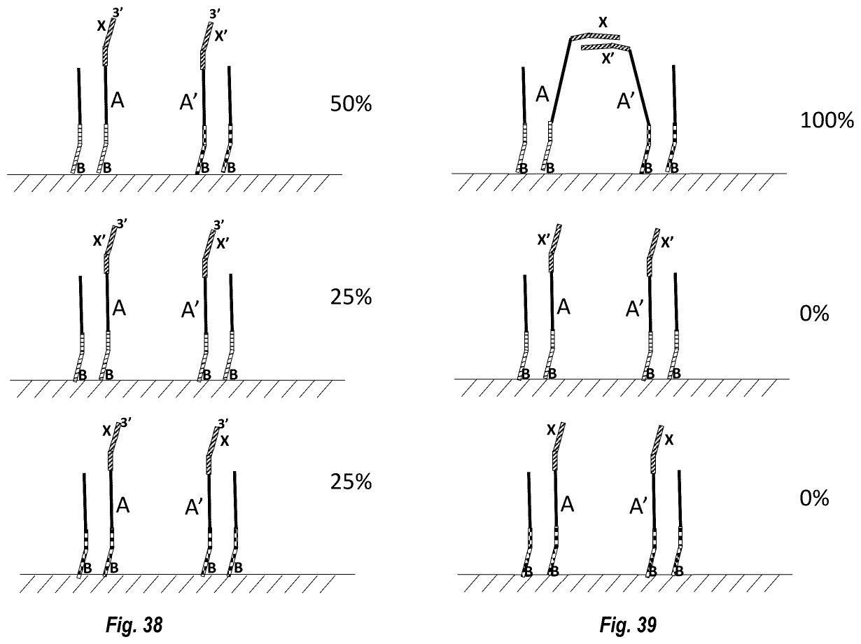

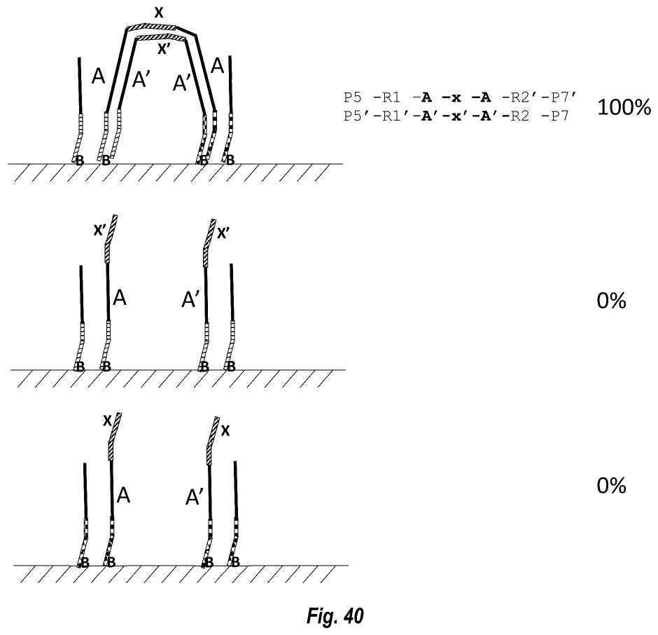

A wide variety of different polynucleotides comprising two inserts will be described herein, such as those in FIG. 29 and FIG. 40. In addition to more than one insert and a hybridization sequence (or its complement), the present polynucleotides may also comprise a variety of other types of inserts.

For example, a polynucleotide may comprise one or more sequencing primer sequences. Such sequencing primer sequences may be used for binding primers to initiate sequencing when the polynucleotides are used as sequencing templates. In some embodiments, a polynucleotide comprises a first read sequencing primer sequence and/or a second read sequencing primer sequence. As used herein “first read sequencing primer sequence” and “second read sequencing primer sequences” refer to sequences that can bind to a primer that may be used in different sequencing reads. These terms do not limit to any specific sequence, and, for example, a first read sequencing primer sequence may be used to initiate a second sequencing read in a given experiment and a second read sequencing primer may be used to initiate a first sequencing read in a given experiment. Such primer sequences may vary based on the sequencing platform that a user plans to utilize, and such primer sequences would be well-known in the art, such as A14 (SEQ ID NO: 4) and B15 sequences (SEQ ID NO: 5).

In some embodiments, the first read sequencing primer sequence and the second read sequencing primer sequence are different. In some embodiments, the first read sequencing primer sequence and the second read sequencing primer sequence each comprise an A14 sequence or a B15 sequence, or their complements. In some embodiments, the 3′ terminal polynucleotide comprises the complement of a P5 primer sequence (P5′) and the 5′ terminal polynucleotide comprises a P7 primer sequence (P7, SEQ ID NO: 48), or the 3′ terminal polynucleotide comprises the complement of a P7 primer sequence (P7′) and the 5′ terminal polynucleotide comprises a P5 primer sequence (P5, SEQ ID NO: 7).

In some embodiments, the 3′ terminal polynucleotide and/or the 5′ terminal polynucleotide each independently comprise at least one of an adapter, a barcode sequence, a unique molecular identifier (UMI) sequence, a capture sequence, or a cleavage sequence. In other words, polynucleotides may comprise additional sequences of use in methods that a user wants to perform, such as sequencing.

Using methods described herein, one insert in a polynucleotide may be prepared from a fragment comprising a portion of a sense strand of a target nucleic acid and the other insert is prepared by elongation from a fragment comprising a portion of an antisense strand of a target nucleic acid. Using methods described herein, one insert may be prepared from a fragment comprising a portion of an antisense strand of a target nucleic acid and the other insert is prepared by elongation from a fragment comprising a portion of a strand of a target nucleic acid.

In some embodiments, a polynucleotide comprises two insert sequences that are copies of each other. In some embodiments, a polynucleotide comprises a 5′ terminal polynucleotide comprising (a) a first read sequencing primer sequence; (b) an insert sequence derived from a target nucleic acid, wherein the insert sequence is 3′ of the 5′ terminal polynucleotide; (c) a hybridization sequence 3′ of the insert sequence; (d) a copy of the insert sequence 3′ of the hybridization sequence; and (e) a 3′ terminal polynucleotide comprising the complement of a second read sequencing primer sequence. In some embodiments, this polynucleotide may be a sequencing template. While the two copies of the insert (i.e., the insert sequence and the copy of the insert sequence) may be expected to be identical, sequencing results may indicate that they are not. For example, the two copies of the insert may be different based on a mismatch mutation in the target nucleic acid or based on introduction of an error during PCR amplification.

In some embodiments, a polynucleotide comprises two insert sequences that are not copies of each other. In some embodiments, the two insert sequences may be different. In some embodiments, the two insert sequences comprised in a polynucleotide were prepared from different regions of a target nucleic acid. In some embodiments, a polynucleotide comprises (a) a 5′ terminal polynucleotide comprising a first read sequencing primer sequence; (b) a first insert sequence derived from a target nucleic acid, wherein the insert sequence is 3′ of the 5′ terminal polynucleotide; (c) a hybridization sequence 3′ of the insert sequence; (d) a second insert sequence 3′ of the hybridization sequence; and (e) a 3′ terminal polynucleotide comprising the complement of a second read sequencing primer sequence. As described herein for methods with immobilized transposomes, such templates with two different insert sequences can be used to determine contiguity data.

The two inserts comprised in a polynucleotide may be the same of different sizes. In some embodiments, inserts that are copies comprise the same number of nucleotides. In some embodiments, the insert sequences comprise 40 to 400 nucleotides, optionally wherein the insert sequences comprise 1000 or fewer nucleotides. In some embodiments, a paired sequencing read protocol may be performed for a larger insert, such as one comprising more than 500 nucleotides.

In some embodiments, a polynucleotide is immobilized on a solid support. In some embodiments, the polynucleotide is immobilized on the solid support via the 5′ terminal polynucleotide (such as in the embodiment shown in FIG. 29). In some embodiments, a polynucleotide is immobilized to the solid support via binding of an affinity moiety on the 5′ terminal polynucleotide to a binding moiety on the surface of the solid support. In some embodiments, an affinity moiety is attached via a linker to the 5′ terminal polynucleotide. In some embodiments, the affinity moiety is biotin, desthiobiotin, or dual biotin.

In some embodiments, a polynucleotide has the structure: 5′-P5-A 14-Insert-HYB-Insert-B15′-P7′-3′; or

5′-P7-B15-Insert-HYB′-Insert-A14′-P5′-3′, wherein HYB is a hybridization sequence and HYB′ is the complement of a hybridization sequence. In some embodiments, the two insert sequences are copies of the same sequence that are identical or two sequences that have greater than 95% sequence homology. Potential reasons for differences in two copies of an insert sequences are described herein, such as non-canonical base pairing or random errors introduced during sequencing. Figure shows a representative double-stranded polynucleotide that comprises two complementary concatenated sequencing templates. One template comprises two A inserts, while the complementary strand comprises two A′ inserts.

In some embodiments, a polynucleotide has the structure: 5′-P5-A 14-Insert-HYB-Insert-B15′-P7′-3′; or

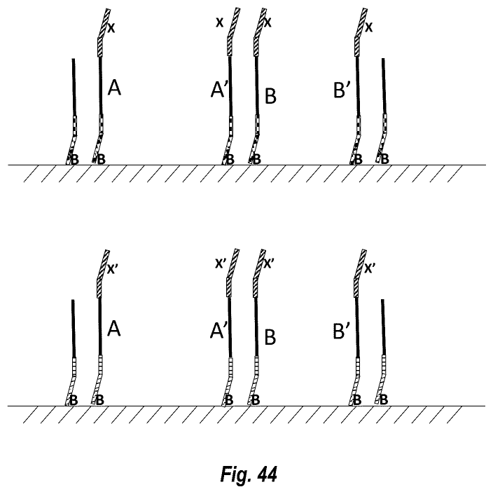

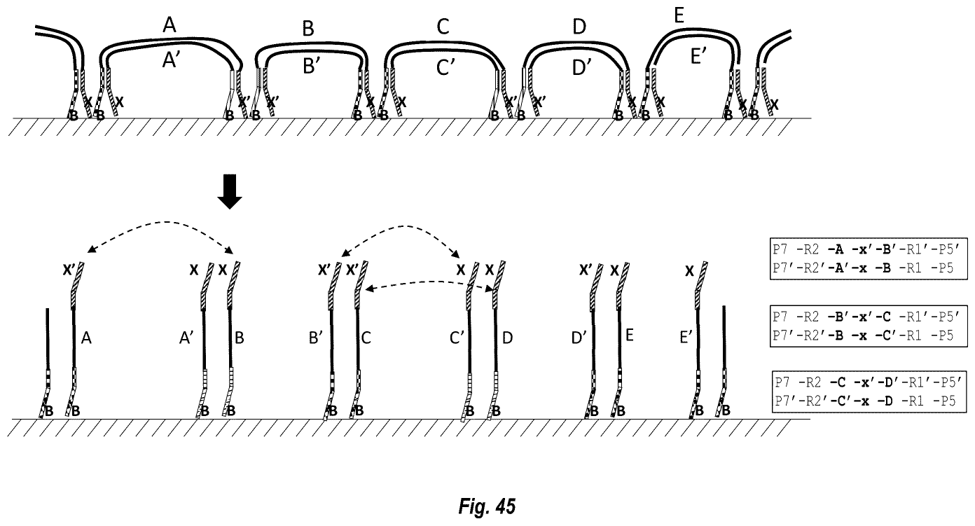

5′-P7-B15-Insert1-HYB′-Insert2-A14′-P5′-3′, wherein HYB is a hybridization sequence and HYB′ is the complement of a hybridization sequence. In some embodiments, Insert 1 and Insert 2 comprise different sequences with little or no sequence homology. FIG. 45 shows representative means of bridging that can be used to generate two complementary polynucleotides each comprising two different sequences.

In some embodiments, a composition comprises a polynucleotide hybridized to its complement. In some embodiments, a polynucleotide hybridized to its complement may be termed a double-stranded concatenated sequencing template. In some embodiments, a double-stranded concatenated sequencing template is immobilized to the surface of a solid support by both of its 5′ ends.

In some embodiments, a polynucleotide or a composition comprising a polynucleotide and its complement is immobilized on the surface of a solid support, wherein the affinity moiety is biotin, desthiobiotin, or dual biotin and the binding moiety is avidin or streptavidin.

A wide range of different solid support may be used for immobilization. In some embodiments, the solid support is a bead, slide, wall of a vessel, a flow cell, or a nanowell comprised in a flow cell.

In some embodiments, a linker for attaching an affinity moiety to a polynucleotide is a cleavable linker. In some embodiments, a user can release a polynucleotide from a solid support at a desired time by cleaving this cleavable linker.

A. Target Nucleic Acid

Target nucleic acids used herein can be composed of DNA, RNA or analogs thereof. The source of the target nucleic acids can be genomic DNA, messenger RNA, or other nucleic acids from native sources. In some cases, the target nucleic acids that are derived from such sources can be amplified prior to use in a method or composition herein.

Exemplary biological samples from which target nucleic acids can be derived include, for example, those from a mammal such as a rodent, mouse, rat, rabbit, guinea pig, ungulate, horse, sheep, pig, goat, cow, cat, dog, primate, human or non-human primate; a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, such as Escherichia coli, staphylococci or Mycoplasma pneumoniae; an archae; a virus such as Hepatitis C virus or human immunodeficiency virus; or a viroid. Target nucleic acids can be derived from a homogeneous culture or population of the above organisms or alternatively from a collection of several different organisms, for example, in a community or ecosystem. Nucleic acids can be isolated using methods known in the art including, for example, those described in Sambrook et al, Molecular Cloning: A Laboratory Manual, 3rd edition, Cold Spring Harbor Laboratory, New York (2001) or in Ausubel et al, Current Protocols in Molecular Biology, John Wiley and Sons, Baltimore, Md. (1998), each of which is incorporated herein by reference.

In some embodiments, target nucleic acids can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid. For example, PCR amplification produces fragments having a size defined by the length of the fragment between the flanking primers used for amplification.

A population of target nucleic acids, or amplicons thereof, can have an average strand length that is desired or appropriate for a particular application of the methods or compositions set forth herein. For example, the average strand length can be less than 100,000 nucleotides, 50,000 nucleotides, 10,000 nucleotides, nucleotides, 1,000 nucleotides, 500 nucleotides, 100 nucleotides, or 50 nucleotides. Alternatively or additionally, the average strand length can be greater than 10 nucleotides, 50 nucleotides, 100 nucleotides, 500 nucleotides, 1,000 nucleotides, 5,000 nucleotides, 10,000 nucleotides, 50,000 nucleotides, or 100,000 nucleotides. The average strand length for population of target nucleic acids, or amplicons thereof, can be in a range between a maximum and minimum value set forth above. It will be understood that amplicons generated at an amplification site (or otherwise made or used herein) can have an average strand length that is in a range between an upper and lower limit selected from those exemplified above.

In some embodiments, the target nucleic acids have a relatively short average strand length, such as less than 200 nucleotides, less than 150 nucleotides, less than 100 nucleotides, less than 75 nucleotides, less than 50 nucleotides, or less than 36 nucleotides. Sequencing of target nucleic acids with relatively short average strand length are not limited by read-length, and increasing the number of reads could significantly increase sequencing output. Examples of sample types with relatively short average strand length are cell-free DNA (cfDNA) and exome sequencing sample.

In some embodiments, the target nucleic acids are cell-free DNA (cfDNA) from a maternal blood sample. In some embodiments, the cfDNA is extracted from a maternal plasma sample. In some embodiments, the cfDNA is for noninvasive prenatal testing (NIPT).

In some embodiments, the target nucleic acids are exomes. In some embodiments, exomes are prepared via targeted resequencing. In some embodiments, exomes are prepared by whole-genome enrichment. In some embodiments, exomes are prepared by hybridization-based enrichment.

In some embodiments, the target nucleic acids are DNA and RNA. Separate libraries of RNA and DNA can be prepared to generate hybrid DNA/RNA polynucleotides. In some embodiments, polynucleotides comprise one or more insert comprising RNA and one or more insert comprising DNA. Such polynucleotides comprising RNA insert(s) and DNA insert(s) can be termed “hybrid polynucleotides” and allow multiple readouts to be generated from a single sequencing run. In some embodiments, polynucleotides comprising RNA and DNA inserts have a dual sample index to allow for self-normalizing. In some embodiments, the minimum of DNA or RNA in the starting libraries dictates the amount of hybrid polynucleotides generated.

Any of a variety of known amplification techniques can be used to increase the amount of template sequences present for use in a method set forth herein. Exemplary techniques include, but are not limited to, polymerase chain reaction (PCR), rolling circle amplification (RCA), multiple displacement amplification (MDA), or random prime amplification (RPA) of nucleic acid molecules having template sequences. It will be understood that amplification of target nucleic acids prior to use in a method or composition set forth herein is optional. As such, target nucleic acids will not be amplified prior to use in some embodiments of the methods and compositions set forth herein. Target nucleic acids can optionally be derived from synthetic libraries. Synthetic nucleic acids can have native DNA or RNA compositions or can be analogs thereof. Solid-phase amplification methods can also be used, including for example, cluster amplification, bridge amplification or other methods set forth below in the context of array-based methods.

In some embodiments, the polynucleotides disclosed herein can be sequenced using any suitable nucleic acid sequencing platform to determine the nucleic acid sequence of the target sequence. In some respects, sequences of interest are correlated with or associated with one or more congenital or inherited disorders, pathogenicity, antibiotic resistance, or genetic modifications. Sequencing may be used to determine the nucleic acid sequence of a short tandem repeat, single nucleotide polymorphism, gene, exon, coding region, exome, or portion thereof. As such, the methods and compositions described herein relate to methods useful in, but not limited to, cancer and disease diagnosis, prognosis and therapeutics, DNA fingerprinting applications (e.g., DNA databanking, criminal casework), metagenomic research and discovery, agrigenomic applications, and pathogen identification and monitoring.

In some embodiments, a sample used to prepare sequencing templates comprises double-stranded nucleic acid. This double-stranded nucleic acid may be referred to as target nucleic acid. In some embodiments, a double-stranded nucleic acid may be added to a solid support comprising immobilized transposomes. In some embodiments, a double-stranded nucleic acid may be fragmented and combined with a mixture of forked adapters.

In some embodiments, a sample comprises multiple double-stranded nucleic acids.

A biological sample used in accordance with the present disclosure can be any type that comprises target nucleic acids. However, the sample need not be completely purified, and can comprise, for example, nucleic acid mixed with protein, other nucleic acid species, other cellular components, and/or any other contaminant. In some embodiments, the biological sample comprises a mixture of nucleic acid, protein, other nucleic acid species, other cellular components, and/or any other contaminant present in approximately the same proportion as found in vivo. For example, in some embodiments, the components are found in the same proportion as found in an intact cell. In some embodiments, the biological sample has a 260/280 absorbance ratio of less than or equal to 2.0, 1.9, 1.8, 1.7, 1.6, 1.5, 1.4, 1.3, 1.2, 1.1, 1.0, 0.9, 0.8, 0.7, or 0.60. In some embodiments, the biological sample has a 260/280 absorbance ratio of at least 2.0, 1.9, 1.8, 1.7, 1.6, 1.5, 1.4, 1.3, 1.2, 1.1, 1.0, 0.9, 0.8, 0.7, or 0.60. Because the methods provided herein allow nucleic acid to be bound to solid supports, other contaminants can be removed merely by washing the solid support after surface bound tagmentation occurs. The biological sample can comprise, for example, a crude cell lysate or whole cells. For example, a crude cell lysate that is applied to a solid support in a method set forth herein, need not have been subjected to one or more of the separation steps that are traditionally used to isolate nucleic acids from other cellular components. Exemplary separation steps are set forth in Sambrook et al., Molecular Cloning: A Laboratory Manual, 2d Edition, 1989, and Short Protocols in Molecular Biology, ed. Ausubel, et al, hereby incorporated by reference.

In some embodiments, the sample that is applied to the solid support has a 260/280 absorbance ratio that is less than or equal to 1.7.

Thus, in some embodiments, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising nucleic acid.

In some embodiments, the sample is blood. In some embodiments, the sample is a cell lysate. In some embodiments, the cell lysate is a crude cell lysate. In some embodiments, the method further comprises lysing cells in the sample after applying the sample to a solid support to generate a cell lysate.

In some embodiments, the sample is a biopsy sample. In some embodiments, the biopsy sample is a liquid or solid sample. In some embodiments, a biopsy sample from a cancer patient is used to evaluate sequences of interest to determine if the subject has certain mutations or variants in predictive genes.

In some embodiments, the sample comprises a target double-stranded DNA. In some embodiments, the DNA is genomic DNA. In some embodiments, the DNA is cell-free DNA (cfDNA). In some embodiments, the DNA is circulating tumor DNA (ctDNA).

In some embodiments, the DNA is double-stranded cDNA that is prepared from RNA. In some embodiments, the RNA is mRNA. In some embodiments, the RNA comprises coding, untranslated region (UTR), introns, and/or intergenic sequences.

B. 3′ Terminal Polynucleotide

In some embodiments, the 3′ terminal polynucleotide comprises a first read primer binding sequence.

In some embodiments, the 3′ terminal polynucleotide comprises at least one of a barcode sequence, a unique molecular identifier (UMI) sequence, a capture sequence, or a cleavage sequence. In some embodiments, the 3′ terminal polynucleotide and/or the attachment polynucleotide each independently comprise at least one of a barcode sequence, a unique molecular identifier (UMI) sequence, a capture sequence, or a cleavage sequence.

In some embodiments, the 3′ terminal polynucleotide comprises a ME′, B15′, and/or P7′ sequence. In some embodiments, the 3′ terminal polynucleotide comprises a ME′, B15′, and P7′ sequence.

In some embodiments, the 3′ terminal polynucleotide comprises the complement of a P5 primer sequence (P5′) and the attachment polynucleotide comprises a P7 primer sequence (P7). In some embodiments, the 3′ terminal polynucleotide comprises the complement of a P7 primer sequence (P7′) and the attachment polynucleotide comprises a P5 primer sequence (P5).

In some embodiments, the 3′ terminal polynucleotide comprises a ME′-B15′-P7′ sequence.

C. Insert Sequences

Insert sequences comprised in a polynucleotide comprise sequences from a target nucleic acid. As such, the polynucleotides described herein can be used for a number of purposes, such as to generate tandem reads when sequencing.

Polynucleotide described herein comprise more than one insert sequence. In some embodiments, a polynucleotide comprises 2, 3, 4, 5, 6, 7, 8, 9, 10, or more insert sequences. In some embodiments, a polynucleotide comprises two insert sequences. In some embodiments, a polynucleotide comprises three insert sequences.

Insert sequences may be derived from one or more target nucleic acid.

In some embodiments, a polynucleotide comprises multiple insert sequences that are derived from multiple target nucleic acids.

In some embodiments, a polynucleotide may comprise multiple insert sequences that are all derived from the same target nucleic acid. In some embodiments, multiple insert sequences are derived from discontiguous sequences of the target nucleic acid. By discontiguous sequences, it is meant that the multiple insert sequences in a polynucleotide do not adjoin each other in the original target nucleic acid. In some embodiments, the multiple insert sequences are from random regions of the target nucleic acid. In some embodiments, the methods for generating the present polynucleotides do not select for specific insert sequences.

In some embodiments, multiple insert sequences each comprise from 40 to 400 nucleotides, or each comprise from 100 to 200 nucleotides, or each comprise 150 nucleotides. In some embodiments, a first insert sequence and a second insert sequence each comprise from 40 to 400 nucleotides, or each comprise from 100 to 200 nucleotides, or each comprise 150 nucleotides.

In some embodiments, a polynucleotide comprises more than two insert sequences. In some embodiments, a polynucleotide comprises, between the second insert sequence and the attachment polynucleotide, at least one insert unit comprising an insert sequence derived from a discontiguous sequence of the target nucleic acid or from a different target nucleic acid than the other insert sequences at the 5′ end and a concatenation sequence comprising a read primer binding sequence at the 3′ end, wherein the read primer binding sequence is orthogonal to the other read primer binding sequences.

In embodiments where a polynucleotide comprises more than two insert sequences, the polynucleotide may comprise multiple different concatenation sequences, wherein each concatenation sequence comprises a primer sequence, and wherein the primer sequences comprised in different concatenation sequences are different. In some embodiments, one or more primer sequences comprise a hybridization sequence, wherein hybridization sequences are different in different primer sequences.

For example, to generate a polynucleotide comprising three insert sequences, two different HYB/HYB′ sequence pairs can be used, such as HYB1/HYB1′ and HYB2/HYB2′. To generate the polynucleotide with three inserts, HYB1/HYB1′ can be used to link insert 1 and insert 2, and HYB2/HYB2′ can be used to link insert 2 and insert 3. A forked adapter for insert 1 could comprise P5 and HYB1, an adapter for insert 2 could comprise HYB1′ and HYB2, and an adapter for insert 3 could comprise HYB2′ and P7′.

Insert sequences can be generated by a number of methods to generate nucleic acid fragments, such as tagmentation or fragmentation.

D. Adapter Sequences

In some embodiments, the polynucleotide may comprise one or more adapter sequence.

Adapter sequences may comprise one or more functional sequences or components selected from the group consisting of primer sequences, anchor sequences, universal sequences, spacer regions, index sequences, capture sequences, barcode sequences, cleavage sequences, sequencing-related sequences, and combinations thereof. In some embodiments, an adapter sequence comprises a primer sequence. In other embodiments, an adapter sequence comprises a primer sequence and an index or barcode sequence. A primer sequence may also be a universal sequence. This disclosure is not limited to the type of adapter sequences that could be used and a skilled artisan will recognize additional sequences that may be of use for library preparation and next generation sequencing. A universal sequence is a region of nucleotide sequence that is common to two or more nucleic acid fragments. Optionally, the two or more nucleic acid fragments may also have regions of sequence differences. A universal sequence that may be present in different members of a plurality of nucleic acid fragments can allow for the replication or amplification of multiple different sequences using a single universal primer that is complementary to the universal sequence.

In some embodiments, the first read primer binding sequence comprises a first adapter sequence. In some embodiments, the first adapter sequence is the complement of a A14 primer sequence (A14′) or the complement of a B15 primer sequence (B15′).

In some embodiments, an adapter sequence comprises an SBS or SBS' sequence. In some embodiments, a SBS or SBS' sequence may comprise all or part of a standard sequence comprised in oligonucleotides used in Truseq workflows, such that standard sequence primers can be used. In some embodiments, SBS may be a mosaic end sequence and SBS' may be the complement of a mosaic end sequence, such as ME and ME′.

In some embodiments, a SBS or SBS' sequence may comprise A14-ME or B15-ME, or their complements. SEQ ID NOs: 15-21 show some exemplary SBS or SBS' sequences or adapters comprising SBS or SBS' sequences.

In some embodiments, SBS and SBS' are all or partially complementary sequences that can form an adapter duplex. In some embodiments, SBS and SBS' are partially complementary. In some embodiments, SBS and SBS' are fully complementary. In some embodiments, SBS and/or SBS' comprise a 13-base pair sequence. In some embodiments, the adapter duplex comprises P5-HYB′ and P7-HYB in addition to SBS or SBS′. In this way, for example, when two library fragments are stacked together (i.e., in tandem together) to generate polynucleotides with two inserts, the resulting polynucleotide can be sequenced with standard sequencing primers.

In some embodiment, an adapter sequence has a melting temperature of 65° C. or higher for binding to a sequencing primer. In some embodiments, an adapter sequence binds a sequencing primer such that the binding is not lost with temperatures used for sequencing. In some embodiments, the adapter sequence comprises significant (greater than 10%) of each of A, T, C, and G. In some embodiments, the G/C content of the adapter sequence is 40%-60%. In some embodiments, the G/C content of the adapter sequence is 30% or greater and 70% or less. In some embodiments, the G/C content of the adapter sequence is between 40% or greater and 50% or less or 50% or greater or 60% or less.

In some embodiments, the attachment polynucleotide comprises a second adapter sequence. In some embodiments, the second adapter sequence is an A14 sequence or a B15 sequence.

In some embodiments, the first adapter sequence is the complement of an A14 sequence (A14′) and the second adapter sequence is a B15 sequence. In some embodiments, the first adapter sequence is the complement of a B15 sequence (B15′) and the second adapter sequence is an A14 sequence.

In some embodiments, adapter sequences are transferred to the 5′ ends of a nucleic acid fragment by a tagmentation reaction.

E. Concatenation Sequence

In some embodiments, a concatenation sequence comprises a second read primer binding sequence that is orthogonal to the first read primer binding sequence, wherein the second read primer binding sequence comprises a hybridization sequence. In some embodiments, the hybridization sequence is HYB′. In some embodiments, the second read primer binding sequence comprises a hybridization sequence (HYB) and the complement of an SBS' sequence (ME′), as shown in FIG. 4B. In some embodiments, the fourth read primer binding sequence comprises the complement of a hybridization sequence (HYB′) and the complement of a SBS sequence (SBS′), as shown in FIG. 4B.

In some embodiments, the concatenation sequence comprises a transposon end sequence 3′ of the hybridization sequence and a complement of the transposon end sequence 5′ of the hybridization sequence.

In some embodiments, the concatenation sequence comprises ME′, HYB′, and/or ME. In some embodiments, the concatenation sequence comprises ME′, HYB′, and ME. In some embodiments, the concatenation sequence is ME′-HYB′-ME.

In some embodiments, the second read primer binding sequence comprises the complement of a hybridization sequence and a complement of the transposon end sequence. In some embodiments, the second read primer binding sequence comprises HYB′ or ME′. In some embodiments, the second read primer binding sequence comprises HYB′ and ME′. In some embodiments, the second read primer binding sequence is HYB′-ME′.

F. Immobilization and Attachment Polynucleotide

In some embodiments, the polynucleotide is immobilized on a solid support.

In some embodiments, the polynucleotide is immobilized on the solid support via an attachment polynucleotide. In some embodiments, the attachment polynucleotide comprises an attachment sequence.

In some embodiments, the attachment polynucleotide comprises an attachment sequence. In some embodiments, the attachment sequence is a nucleic acid sequence that hybridizes to a transposon in a transposome complex and that is immobilized on a solid support, such as a slide, flow cell, or bead. In some embodiments, the attachment sequence functions to attach a transposome complex to a solid support. In some embodiments, the attachment sequence functions to attach a polynucleotide to a solid support. In some embodiments, the attachment sequence is P5.

In some embodiments, the polynucleotide is immobilized on the solid support via hybridization of the attachment polynucleotide to an attachment polynucleotide complement on the surface of the solid support. In some embodiments, the polynucleotide is immobilized to the solid support via binding of an affinity moiety on the attachment polynucleotide to a binding moiety on the surface of the solid support.

In some embodiments, the solid support is a flow cell or a bead.

In some embodiments, the attachment polynucleotide comprises at least one of a barcode sequence, a unique molecular identifier (UMI) sequence, a capture sequence, or a cleavage sequence.

In some embodiments, the attachment polynucleotide comprises a second adapter sequence. In some embodiments, the second adapter sequence is A14 or B15.

In some embodiments, the attachment polynucleotide comprises a transposon end sequence. In some embodiments, the transposon end sequence is ME.

In some embodiments, the attachment sequence is P5, the second adapter sequence is A14, and/or the transposon end sequence is ME. In some embodiments, the attachment polynucleotide comprises P5, A14, and/or ME. In some embodiments, the attachment polynucleotide comprises P5, A14, and ME. In some embodiments, the attachment polynucleotide comprises P5-A14-ME.

G. Samples Indexes and UMIs

In some embodiments, polynucleotides comprise, in addition to a hybridization sequence (or its complement) and at least 2 inserts, a primer sequence, an index sequence, a barcode sequence, a purification tag, or any combination thereof. In some embodiments, polynucleotides comprise sample indexes and/or unique molecular identifiers (UMIs). In some embodiments, one or more of these sequences are incorporated into polynucleotides using forked adapters that are ligated to double-stranded fragments or using forked adapters that are comprised within in transposomes that are incorporated into double-stranded fragments during tagmentation. Alternatively, additional sequences may be added to polynucleotides (such as concatenated sequencing templates) after they have been generated, such as with PCR.

Unique molecular identifiers (UMIs) are sequences of nucleotides applied to or identified in nucleic acid molecules that may be used to distinguish individual nucleic acid molecules from one another. UMIs may be sequenced along with the nucleic acid molecules with which they are associated to determine whether the read sequences are those of one source nucleic acid molecule or another. The term “UMI” may be used herein to refer to both the sequence information of a polynucleotide and the physical polynucleotide per se. UMIs are similar to barcodes, which are commonly used to distinguish reads of one sample from reads of other samples, but UMIs are instead used to distinguish nucleic acid template fragments from another when many fragments from an individual sample are sequenced together. UMIs may be defined in many ways, such as described in WO 2019/108972 and WO 2018/136248, which are incorporated herein by reference.

In some embodiments, two sample indexes are used to prepare unique dual indexes (UDIs). In some embodiments, a sample index is an i5-i8 sequence. Alternatively, i6 and i8 sequences may be used as UMIs.

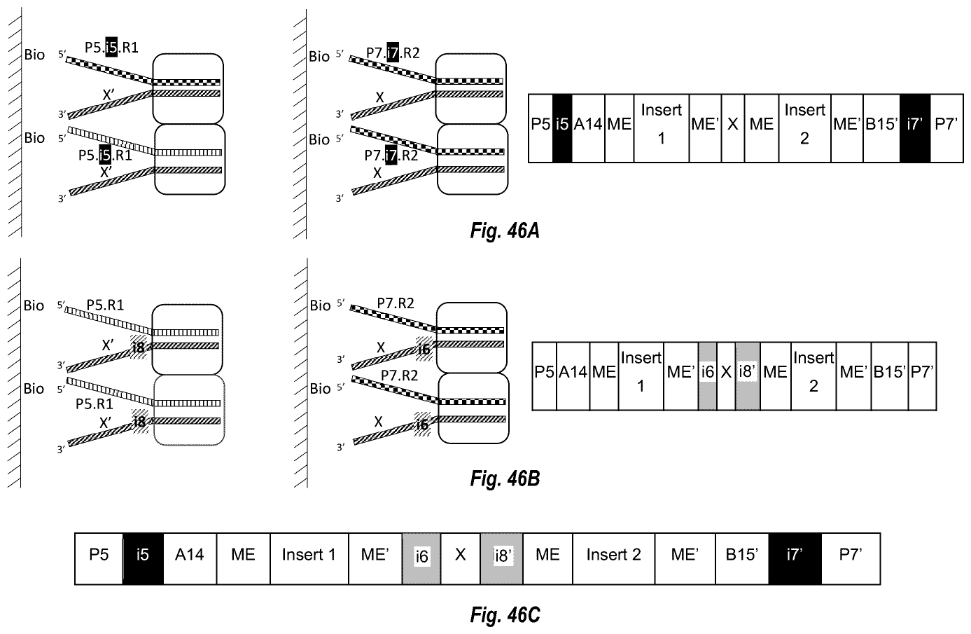

While UMIs are useful for removing PCR duplicates in double-stranded nucleic acids and for detection of low-frequency variants, UDIs are useful for mitigating sample misassignment due to index hopping in library sequencing and demultiplexing. UDIs, such as unique i5 and i7 index sequences, can be added to the ends of target nucleic acids so that both ends contain a UDI. UDIs can be used with patterned flow cells, such as Illumina's NovaSeq 6000 system (See, e.g., WO 2018/204423, WO 2018/208699, WO 2019/055715, and WO 2016/176091; which are incorporated by reference herein in their entireties). In some embodiments described herein, such as those shown in FIGS. 46A and 46B, transposons comprised in different pools of transposome complexes are designed to prepare polynucleotides incorporate UDIs or UMIs during tagmentation and obviate the need for a separate PCR step to incorporate UDIs or UMIs. Exemplary polynucleotides comprising UDIs (such as i5 and i7) or UMIs (such as i6 or i8) are shown in FIGS. 46A-46C.

H. Compositions Comprising a Polynucleotide and its Complement

In some embodiments, a composition comprises a polynucleotide and its complement. In some embodiments, a polynucleotide is hybridized to its complement. In some embodiments, a polynucleotide and its complement are comprised in a double-stranded composition.

In some embodiments, a composition comprises a polynucleotide and its complement, wherein the complement comprises a 3′ terminal complement comprising a first complement read primer binding sequence, wherein the first complement read primer binding sequence is orthogonal to the first and second read primer binding sequences; the complement of the second insert sequence of the 3′ terminal complement; a complement concatenation sequence 5′ of the complement of the second insert sequence and comprising a 3′ to 5′ second complement read primer binding sequence, wherein the second complement read primer binding sequence is orthogonal to the first and second read primer binding sequences, and to the first complement read primer binding sequence; the complement of the first insert sequence 5′ of the complement concatenation sequence; and a complement attachment polynucleotide at the 5′ end comprising a complement attachment sequence.

In some embodiments, a composition comprises a polynucleotide and a complement, wherein either the polynucleotide or the complement is immobilized on a solid support. In some embodiments, a composition comprises a polynucleotide that is immobilized on a solid support via the first attachment polynucleotide. In some embodiments, the complement is immobilized on the solid support via the complement attachment polynucleotide.

In some embodiments, the complement attachment polynucleotide comprises an attachment sequence. In some embodiments, the attachment sequence comprised in the complement attachment polynucleotide is P7.

In some embodiments, the complement attachment polynucleotide comprises a ME-B15-P7 sequence. In some embodiments, the complement attachment sequence comprises P7. In some embodiments, the complement concatenation sequence comprises ME-HYB-ME′. In some embodiments, the second read complement primer sequence comprises HYB-ME′. In some embodiments, the 3′ terminal polynucleotide complement comprises P5′-A14′-ME′. In some embodiments, the first read complement read primer binding sequence comprises A14′-ME′. In some embodiments, the complement hybridization sequence comprises HYB.

I. Structures of a Polynucleotide or a Composition

A polynucleotide may have a variety of structures. In some embodiments, a composition comprises a polynucleotide, or its complement, of one of the following structures.

In some embodiments, the polynucleotide has the structure: 3′-P7′-B15′-ME′-Insert 1-ME-HYB-ME′-Insert 2-ME-A14-P5-5′.

In some embodiments, the complement of the polynucleotide has the structure: 3′-P5′-A14′-ME′-Insert 2-ME-HYB′-ME′-Insert 1-ME-B15-P7-5′.

J. Kits Comprising a Polynucleotide

In some embodiments, a kit or composition comprises a first transposome complex and a second transposome complex, wherein the first transposome complex comprises a transposon comprising the complement of a hybridization sequence and the second transposome complex comprises a transposon comprising a hybridization sequence.

In some embodiments, a composition or kit comprises a solid support, optionally wherein the optionally support is beads; components for generating transposome complexes, comprising a transposase; oligonucleotides for generating an oligonucleotide duplex, wherein the first oligonucleotide comprises a 3′ transposon end sequence and a 5′ first adapter sequence and the second oligonucleotide comprises a 5′ transposon end sequence and a 3′ second adapter sequence, wherein the 5′ transposon end sequence is complementary to the 3′ transposon end sequence; wherein the first and second adapter sequences are not the same; and a first and second set of primers for adding attachment sequences and hybridization sequences to fragments by PCR, wherein the first set of primers comprises primers for adding a hybridization sequence and a first attachment sequence to fragments; and wherein the second set of primers comprises primers for adding a complement hybridization sequence and a second attachment sequence to fragments; wherein the first and second attachment sequences are not the same.

In some embodiments, a kit or composition comprises one or more forked adapter complex. In some embodiments, a kit or composition comprises a first forked adapter complex and a second forked adapter complex.

In some embodiments, a kit or composition comprises one or more assembled adapter duplexes. In some embodiments, a kit or composition comprises an assembled adapter duplex comprising a first adapter duplex and a second adapter duplex.

In some embodiments, a kit or composition comprises a forked adapter complex and an assembled adapter duplex.

In some embodiments, a kit or composition comprises assembled enzyme and transposons.

In some embodiments, a kit or composition comprises purified oligonucleotides.

### III. Methods of Preparing Polynucleotides Comprising Multiple Insert Sequences

A variety of methods can be used to generate the polynucleotides described herein.

A. Methods Comprising a Transposition Reaction

In some embodiments, a polynucleotide is prepared via a method comprising a transposition reaction.

A transposition reaction is a reaction wherein one or more transposons are inserted into target nucleic acids at random sites or almost random sites. Components in a transposition reaction include a transposase (or other enzyme capable of fragmenting and tagging a nucleic acid as described herein, such as an integrase) and a transposon element that includes a double-stranded transposon end sequence that binds to the transposase (or other enzyme as described herein), and an adapter sequence attached to one of the two transposon end sequences. One strand of the double-stranded transposon end sequence is transferred to one strand of the target nucleic acid and the complementary transposon end sequence strand is not (a non-transferred transposon sequence). The adapter sequence can include one or more functional sequences or components (e.g., primer sequences, anchor sequences, universal sequences, spacer regions, or index tag sequences) as needed or desired.

Transposon based technology can be utilized for fragmenting DNA, for example, as exemplified in the workflow for NEXTERA™ FLEX DNA sample preparation kits (Illumina, Inc.), wherein target nucleic acids, such as genomic DNA, are treated with transposome complexes that simultaneously fragment and tag (“tagmentation”) the target, thereby creating a population of fragmented nucleic acid molecules tagged with unique adapter sequences at the ends of the fragments.

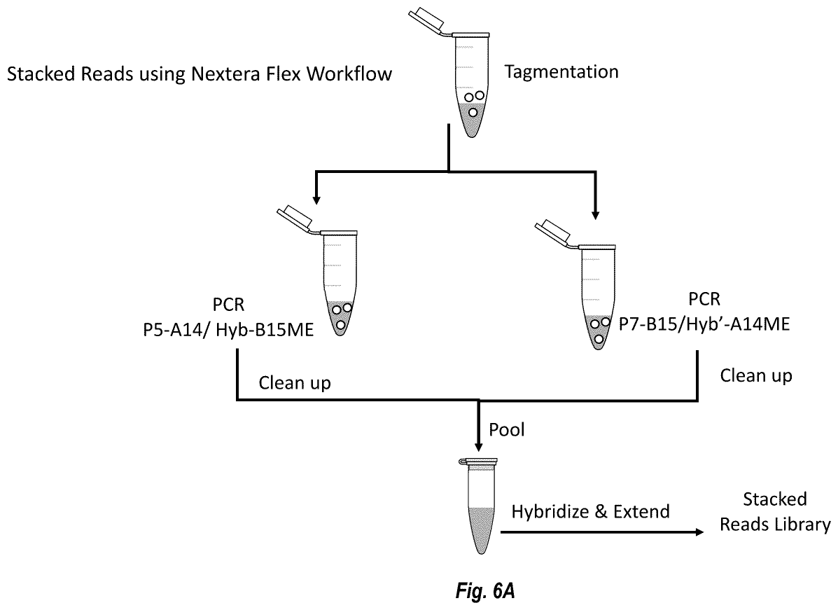

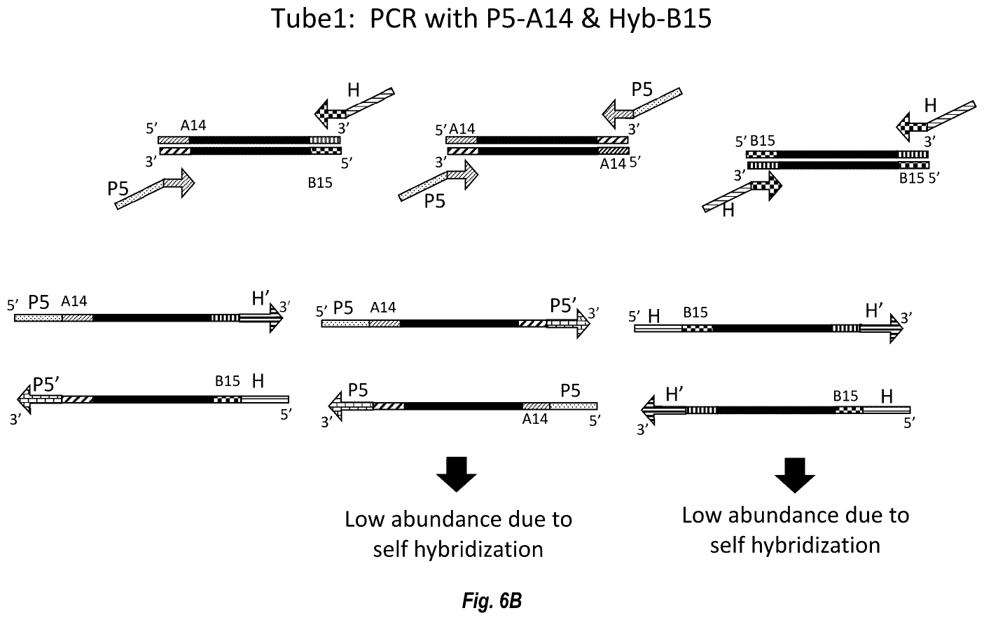

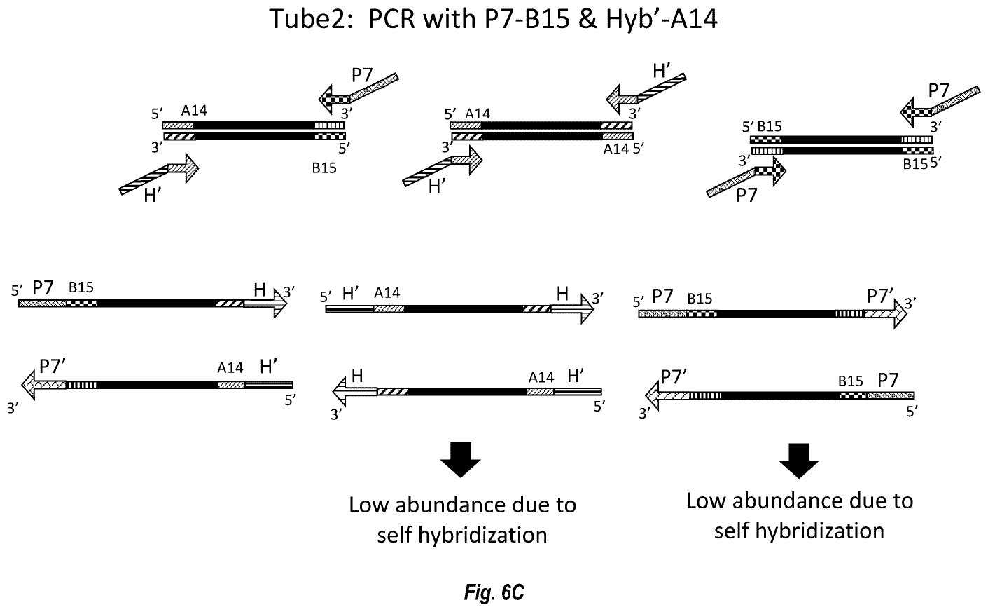

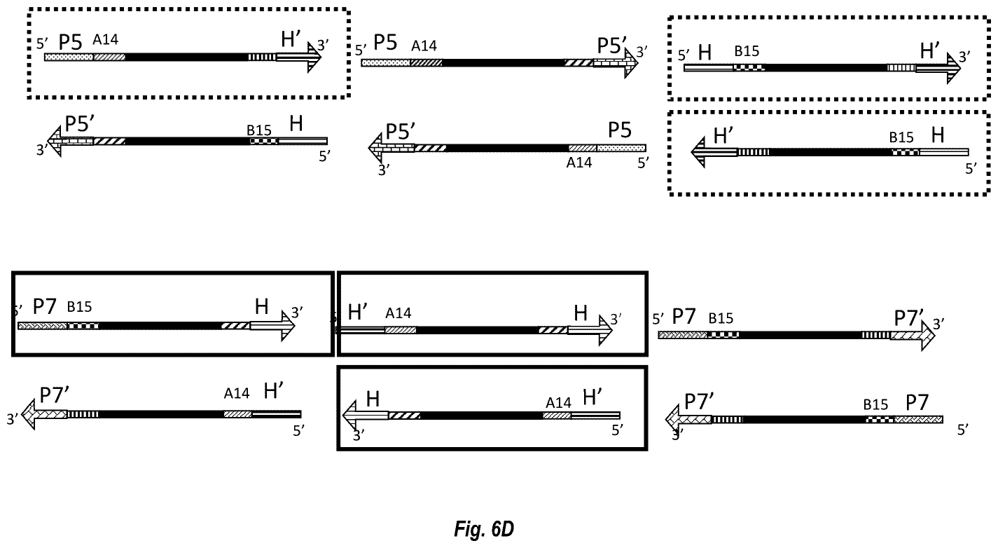

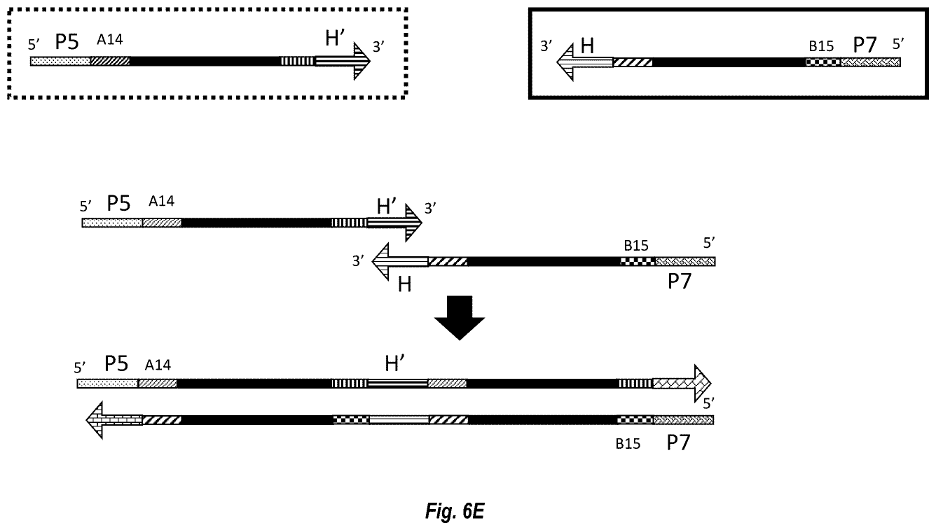

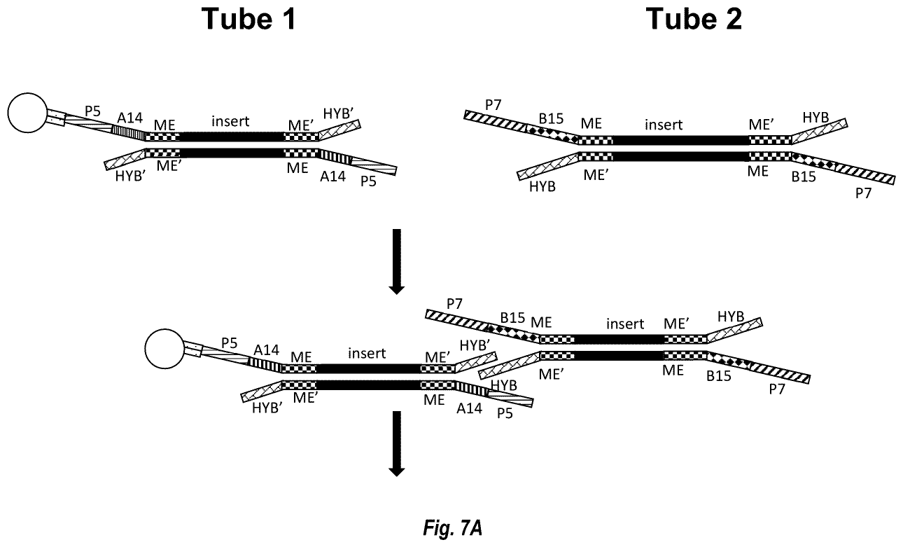

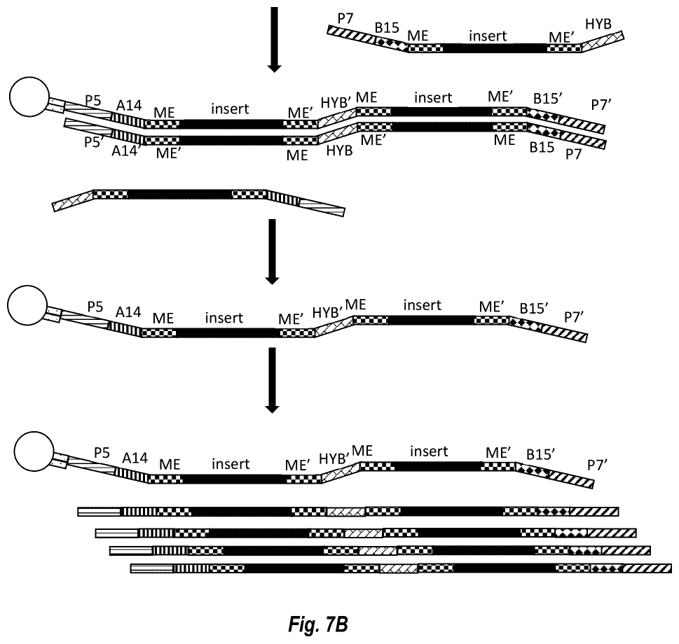

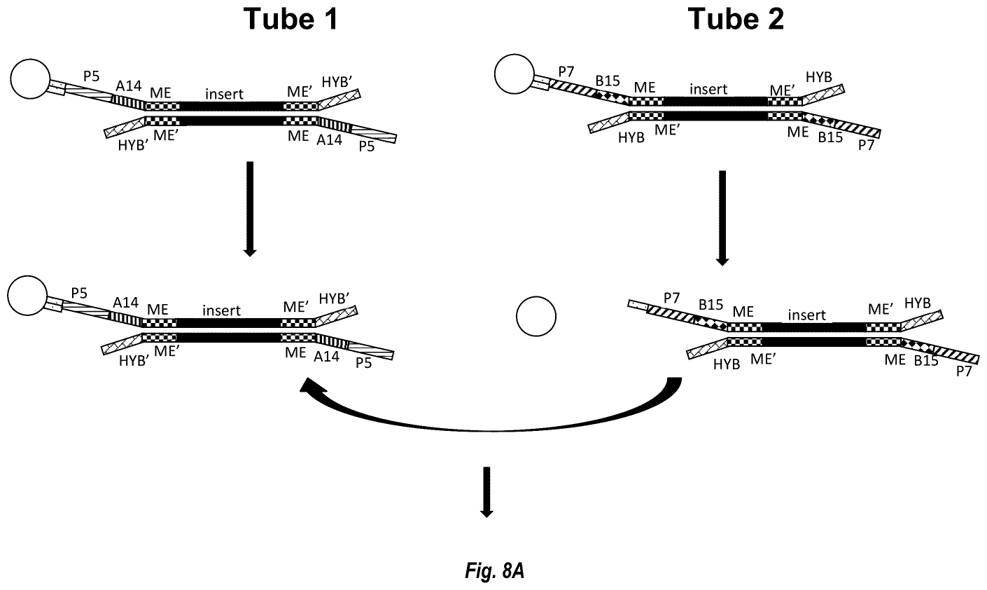

FIGS. 6A-9B present a variety of approaches for generating library products comprising HYB or HYB′ sequences using transposition reactions. In some embodiments, bead-linked transposomes (BLTs) are used. In some embodiments, the reactions, transposomes in solution are used.

A “transposome complex” is comprised of at least one transposase (or other enzyme as described herein) and a transposon recognition sequence. In some such systems, the transposase binds to a transposon recognition sequence to form a functional complex that is capable of catalyzing a transposition reaction. In some aspects, the transposon recognition sequence is a double-stranded transposon end sequence. The transposase binds to a transposase recognition site in a target nucleic acid and insert sequences the transposon recognition sequence into a target nucleic acid. In some such insertion events, one strand of the transposon recognition sequence (or end sequence) is transferred into the target nucleic acid, resulting in a cleavage event. Exemplary transposition procedures and systems that can be readily adapted for use with the transposases.

Exemplary transposases that can be used with certain embodiments provided herein include (or are encoded by): Tn5 transposase, Sleeping Beauty (SB) transposase, Vibrio harveyi, MuA transposase and a Mu transposase recognition site comprising R1 and R2 end sequences, Staphylococcus aureus Tn552, Ty1, Tn7 transposase, Tn/O and IS10, Mariner transposase, Tc1, P Element, Tn3, bacterial insertion sequences, retroviruses, and retrotransposon of yeast. More examples include IS5, Tn10, Tn903, IS911, and engineered versions of transposase family enzymes. The methods described herein could also include combinations of transposases, and not just a single transposase.

In some embodiments, the transposase is a Tn5, Tn7, MuA, or Vibrio harveyi transposase, or an active mutant thereof. In other embodiments, the transposase is a Tn5 transposase or a mutant thereof. In other embodiments, the transposase is a Tn5 transposase or a mutant thereof. In other embodiments, the transposase is a Tn5 transposase or an active mutant thereof. In some embodiments, the Tn5 transposase is a hyperactive Tn5 transposase, or an active mutant thereof. In some aspects, the Tn5 transposase is a Tn5 transposase as described in PCT Publ. No. WO2015/160895, which is incorporated herein by reference. In some aspects, the Tn5 transposase is a hyperactive Tn5 with mutations at positions 54, 56, 372, 212, 214, 251, and 338 relative to wild-type Tn5 transposase. In some aspects, the Tn5 transposase is a hyperactive Tn5 with the following mutations relative to wild-type Tn5 transposase: E54K, M56A, L372P, K212R, P214R, G251R, and A338V. In some embodiments, the Tn5 transposase is a fusion protein. In some embodiments, the Tn5 transposase fusion protein comprises a fused elongation factor Ts (Tsf) tag. In some embodiments, the Tn5 transposase is a hyperactive Tn5 transposase comprising mutations at amino acids 54, 56, and 372 relative to the wild type sequence. In some embodiments, the hyperactive Tn5 transposase is a fusion protein, optionally wherein the fused protein is elongation factor Ts (Tsf). In some embodiments, the recognition site is a Tn5-type transposase recognition site (Goryshin and Reznikoff, J. Biol. Chem., 273:7367, 1998). In one embodiment, a transposase recognition site that forms a complex with a hyperactive Tn5 transposase is used (e.g., EZ-Tn5™ Transposase, Epicentre Biotechnologies, Madison, Wis.). In some embodiments, the Tn5 transposase is a wild-type Tn5 transposase.

In some embodiments, the transposome complex comprises a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adapter sequence in each monomer and the second population has a different adapter sequence in each monomer.

In some embodiments, the transposase complex comprises a transposase (e.g., a Tn5 transposase) dimer comprising a first and a second monomer. In some aspects, each monomer comprises a first transposon, a second transposon, and an attachment polynucleotide, where the first transposon includes a transposon end sequence at its 3′ end (also referred to as a 3′ transposon end sequence) and an adapter sequence at its 5′ end (also referred to as a 5′ adapter sequence); the second transposon includes a transposon end sequence at its 5′ end (also referred to as a 5′ transposon end sequence) and an adapter sequence at its 3′ end (also referred to as a 3′ adapter sequence); and the attachment polynucleotide includes an attachment adapter sequence hybridized to the 5′ adapter sequence of the first transposon, a primer sequence, and a linker. In some embodiments, the 5′ transposon end sequence of the second transposon is at least partially complementary to the 3′ transposon end sequence of the first transposon. In some embodiments, the attachment adapter sequence of the attachment polynucleotide is at least partially complementary to the adapter sequence of the first transposon. In some embodiments, the linker of the attachment polynucleotide includes a binding element.

1. Transposome Complexes

In some embodiments, a transposome complex comprises a first transposon comprising the complement of a first read primer binding sequence, wherein the complement of the first read primer binding sequence comprises: a 3′ portion comprising a transposon end sequence; the complement of a first adapter sequence; and a second transposon comprising a 5′ portion comprising the complement of the transposon end sequence; and the complement of a hybridization sequence. In some embodiments, the first read primer binding sequence comprises a first read sequencing adapter sequence.

In some embodiments, the 3′ transposon end sequence comprises a mosaic end (ME) sequence and the 5′ transposon end sequence comprises an ME′ sequence.

In some embodiments, the complement of the first adapter sequence is a B15 sequence.

In some embodiments, the first read primer binding sequence is ME′-B15′.

In some embodiments, the second transposon comprises a complement attachment sequence 5′ of the first read primer binding sequence. In some embodiments, the complement attachment sequence comprises a P7 sequence.

In some embodiments, the transposome complex has a structure of:

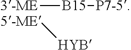

In some embodiments, a transposome complex comprises a transposase; a first transposon comprising an attachment polynucleotide, wherein the attachment polynucleotide comprises a 5′ portion comprising an attachment sequence; a 3′ portion comprising a second read primer binding sequence, comprising a 3′ portion comprising a transposon end sequence; and an adapter; and a second transposon comprising a 5′ portion comprising the complement of the transposon end sequence; and a hybridization sequence.

In some embodiments, adapter is an A14 sequence. In some embodiments, the attachment sequence comprises a P5 sequence.

In some embodiments, the transposome complex has a structure of:

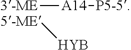

In some embodiments, the first and second transposons as described herein are annealed to each other, and the first transposon is annealed to the attachment polynucleotide. The annealed polynucleotides are then loaded onto a transposase, such as a Tn5 transposase, thereby forming a transposome complex, which is then contacted with and bound to a solid support, such as a bead. In some embodiments, the annealed transposons are bound to a solid support such as a bead and a transposase is then complexed with the transposons, thereby creating a transposome that is bound to a solid support.

2. End Sequences

In some embodiments, the first transposon includes a 3′ transposon end sequence and the second transposon includes a 5′ transposon end sequence. In some embodiments, the 5′ transposon end sequence is at least partially complementary to the 3′ transposon end sequence. In some embodiments, the complementary transposon end sequences hybridize to form a double-stranded transposon end sequence that binds to the transposase (or other enzyme as described herein). In some embodiments, the transposon end sequence is a mosaic end (ME) sequence. Thus, in some embodiments, the 3′ transposon end sequence is an ME sequence and the 5′ transposon end sequence is an ME′ sequence.

3. Adapter Sequences

As discussed above in Section II.D, in any of the embodiments of the method described herein, the first transposon includes a 5′ adapter sequence and the second transposon includes a 3′ adapter sequence. In some embodiments, the attachment polynucleotide includes an attachment adapter sequence hybridized to the 5′ adapter sequence. In some embodiments, the attachment adapter sequence is at least partially complementary to the 5′ adapter sequence. In some embodiments, the adapter sequence is an A14 sequence or a B15 sequence. Thus, in some embodiments, the 5′ adapter sequence is an A14 sequence and the attachment adapter sequence is an A14′ sequence. In some embodiments, the 3′ adapter sequence is a B15′ sequence.

In any of the embodiments, the adapter sequence or transposon end sequences, including A14-ME, ME, B15-ME, ME′, A14, B15, and ME are provided below:

A14-ME:
(SEQ ID NO: 1)
5′-TCGTCGGCAGCGTCAGATGTGTATAAGAGACAG-3′
B15-ME:
(SEQ ID NO: 2)
5′-GTCTCGTGGGCTCGGAGATGTGTATAAGAGACAG-3′
ME′:
(SEQ ID NO: 3)
5′-phos-CTGTCTCTTATACACATCT-3′
A14:
(SEQ ID NO: 4)
5′-TCGTCGGCAGCGTC-3′
B15:
(SEQ ID NO: 5)
5′-GTCTCGTGGGCTCGG-3′
ME:
(SEQ ID NO: 6)
AGATGTGTATAAGAGACAG

4. Immobilized Transposomes and Solid Supports

In some embodiments, the transposome complex is immobilized to a solid support via the first or second transposon. In some embodiments, the transposome complex is immobilized on a bead. In some embodiments, the transposome complex is immobilized on a bead via the first or second transposon.

The terms “solid surface,” “solid support,” and other grammatical equivalents refer to any material that is appropriate for or can be modified to be appropriate for the attachment of the transposome complexes. As will be appreciated by those in the art, the number of possible substrates is multitude. Possible substrates include, but are not limited to, glass and modified or functionalized glass, plastics (including acrylics, polystyrene and copolymers of styrene and other materials, polypropylene, polyethylene, polybutylene, polyurethanes, TEFLON, etc.), polysaccharides, polyhedral organic silsesquioxane (POSS) materials, nylon or nitrocellulose, ceramics, resins, silica, or silica-based materials including silicon and modified silicon, carbon, metals, inorganic glasses, plastics, optical fiber bundles, beads, paramagnetic beads, and a variety of other polymers.

In some embodiments, the transposome complex is immobilized on the solid support via a binding element (and optional linker). In some embodiments, the solid support is a bead, a paramagnetic bead, a flowcell, a surface of a microfluidic device, a tube, a well of a plate, a slide, a patterned surface, or a microparticle. In some embodiments, the solid support comprises or is a bead. In one embodiment, the bead is a paramagnetic bead. In some embodiments, the solid support comprises a plurality of solid supports. In some embodiments, transposome complexes are immobilized on a plurality of solid supports. In some embodiments, the plurality of solid supports comprises a plurality of beads. In some embodiments, the plurality of transposome complexes are immobilized on the solid support at a density of at least 10³, 10⁴, 10⁵, 10⁶ complexes per mm². In some embodiments, the solid support is a bead or a paramagnetic bead, and there are greater than 10,000, 20,000, 30,000, 40,000, 50,000, or 60,000 transposome complexes bound to each bead.

Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextran such as Sepharose, cellulose, nylon, cross-linked micelles and TEFLON, as well as any other materials outlined herein for solid supports. In certain embodiments, the microspheres are magnetic microspheres or beads, for example paramagnetic particles, spheres or beads. The beads need not be spherical; irregular particles may be used. Alternatively or additionally, the beads may be porous. The bead sizes range from nanometers, e.g., 100 nm, to millimeters, e.g., 1 mm, with beads from 0.2 micron to 200 microns being preferred, and from 0.5 to 5 micron being particularly preferred, although in some embodiments smaller or larger beads may be used. The bead may be coated with a binding partner, for example the bead may be streptavidin coated. In some embodiments, the beads are streptavidin coated paramagnetic beads, for example, Dynabeads MyOne streptavidin C1 beads (Thermo Scientific catalog #65601), Streptavidin MagneSphere Paramagnetic particles (Promega catalog #Z5481), Streptavidin Magnetic beads (NEB catalog #514205) and MaxBead Streptavidin (Abnova catalog #U0087). The solid support could also be a slide, for example a flowcell or other slide that has been modified such that the transposome complex can be immobilized thereon.

In some embodiments, the binding partner is present on the solid support or bead at a density of from 1000 to 6000 pmol/mg, or 2000 to 5000 pmol/mg, or 3000 to 5000 pmol/mg, or 3500 to 4500 pmol/mg.

In some embodiments, the solid surface is the inner surface of a sample tube. In some embodiments, the solid surface is a capture membrane. In one example, the capture membrane is a biotin-capture membrane (for example, available from Promega Corporation). In some embodiments, the capture membrane is filter paper. In some embodiments of the present disclosure, solid supports comprised of an inert substrate or matrix (e.g. glass slides, polymer beads etc.) which has been functionalized, for example by application of a layer or coating of an intermediate material comprising reactive groups which permit covalent attachment to molecules, such as polynucleotides. Examples of such supports include, but are not limited to, polyacrylamide hydrogels supported on an inert substrate such as glass, particularly polyacrylamide hydrogels as described in WO2005/065814 and US2008/0280773, the contents of which are incorporated herein in their entirety by reference. The methods of tagmenting (fragmenting and tagging) DNA on a solid surface for the construction of a tagmented DNA library are described in WO2016/189331 and US2014/0093916A1, which are incorporated herein by reference in their entireties. In some embodiments, the transposome complex described herein is immobilized to a solid support via the binding element. In some such embodiments, the solid support comprises streptavidin as the binding partner and the binding element is biotin.

In some embodiments, transposome complexes are immobilized on a solid support, such as a bead, at a particular density or density range. In some embodiments, the density of complexes on a solid support refers to the concentration of transposome complexes in solution during the immobilization reaction. The complex density assumes that the immobilization reaction is quantitative. Once the complexes are formed at a particular density, that density remains constant for the batch of surface-bound transposome complexes. The resulting beads can be diluted, and the resulting concentration of complexes in the diluted solution is the prepared density for the beads divided by the dilution factor. Diluted bead stocks retain the complex density from their preparation, but the complexes are present at a lower concentration in the diluted solution. The dilution step does not change the density of complexes on the beads, and therefore affects library yield but not insert (fragment) size. In some embodiments, the density is between 5 nM and 1000 nM, or between 5 and 150 nM, or between 10 nM and 800 nM. In other embodiments, the density is 10 nM, or 25 nM, or 50 nM, or 100 nM, or 200 nM, or 300 nM, or 400 nM, or 500 nM, or 600 nM, or 700 nM, or 800 nM, or 900 nM, or 1000 nM. In some embodiments, the density is 100 nM. In some embodiments, the density is 300 nM. In some embodiments, the density is 600 nM. In some embodiments, the density is 800 nM. In some embodiments, the density is 100 nM. In some embodiments, the density is 1000 nM.

In some embodiments, the composition includes a solid support and a transposome complex immobilized to the solid support. In some embodiments, the transposome complex includes a transposase, a first transposon, an attachment polynucleotide, and a second transposon. In some embodiments, the first transposon includes a 3′ transposon end sequence and a 5′ adapter sequence. In some embodiments, the attachment polynucleotide includes an attachment adapter sequence hybridized to the 5′ adapter sequence and a binding element. In some embodiments, the second transposon comprises a 5′ transposon end sequence and a 3′ adapter sequence. In some embodiments, the transposome complex is immobilized to the solid support through the attachment polynucleotide. In some embodiments, the attachment polynucleotide further comprises a primer sequence.

In some embodiments, the binding element comprises or is an optionally substituted biotin. In some embodiments, the binding element is connected to the attachment polynucleotide via a linker. In some embodiments, the binding element comprises or is a biotin linker. In some embodiments, the binding element comprises or is a 3′, 5′, or internal biotin.

Some embodiments of the transposome complex described herein include an attachment polynucleotide. As used herein, the attachment polynucleotide is a polynucleotide that hybridizes to a transposon on one end and binds to a surface on a second end. Thus, the transposome complex described herein is immobilized to a solid support through the attachment polynucleotide. In some embodiments, an attachment polynucleotide includes an attachment adapter sequence hybridized to the adapter sequence of the first transposon or the adapter sequence of the second transposon, a primer sequence, and a linker. In some embodiments, the linker includes a binding element.

As described herein the attachment adapter sequence may be at least partially complementary to the adapter sequence of the first or second transposon. In some embodiments, the attachment adapter sequence hybridizes to the 5′ adapter sequence. In embodiments when the attachment adapter sequence hybridizes to the 5′ adapter sequence, where the 5′ adapter sequence is an A14 sequence, the attachment adapter sequence is an A14′ sequence. In some embodiments, the attachment adapter sequence hybridizes to the 3′ adapter sequence. In embodiments when the attachment adapter sequence hybridizes to the 3′ adapter sequence, where the 3′ adapter sequence is a B15′ sequence, the attachment adapter sequence is a B15 sequence. In any of these embodiments, the attachment adapter sequence may be fully complementary to the adapter sequence of the first or second transposon or partially complementary to the adapter sequence of the first or second transposon.

In some embodiments, the attachment polynucleotide contains a primer sequence. In some embodiments, the primer sequence is a P5 primer sequence or a P7 primer sequence or a complement thereof (e.g., P5′ or P7′). The P5 and P7 primers are used on the surface of commercial flow cells sold by Illumina, Inc., for sequencing on various Illumina platforms. The primer sequences are described in U.S. Pat. Publ. No. 2011/0059865, which is incorporated herein by reference in its entirety. Examples of P5 and P7 primers, which may be alkyne terminated at the 5′ end, include the following:

P5:
(SEQ ID NO: 7)
AATGATACGGCGACCACCGAGAUCTACAC
P7:
(SEQ ID NO: 8)
CAAGCAGAAGACGGCATACGAG*AT

and derivatives thereof. In some examples, the P7 sequence includes a modified guanine at the G* position, e.g., an 8-oxo-guanine. In other examples, the * indicates that the bond between the G* and the adjacent 3′ A is a phosphorothioate bond. In some examples, the P5 and/or P7 primers include unnatural linkers. Optionally, one or both of the P5 and P7 primers can include a poly T tail. The poly T tail is generally located at the 5′ end of the sequence shown above, e.g., between the 5′ base and a terminal alkyne unit, but in some cases can be located at the 3′ end. The poly T sequence can include any number of T nucleotides, for example, from 2 to 20. While the P5 and P7 primers are given as examples, it is to be understood that any suitable primers can be used in the examples presented herein. The index sequences having the primer sequences, including the P5 and P7 primer sequences serve to add P5 and P7 for activating the library for sequencing. While the P5 and P7 primers are given as examples, it is to be understood that any suitable amplification primers can be used in the examples presented herein.

As used herein, one example of a linker is a moiety that covalently connects a binding element to the end of the nucleotide portion of the attachment polynucleotide and may be used to immobilize the attachment polynucleotide to a solid support. The linker may be a cleavable linker, for example, a linker capable of being cleaved to remove the attachment polynucleotide, and thus the transposome complex or tagmentation product from the solid support. A cleavable linker as used herein is a linker that may be cleaved through chemical or physical means, such as, for example, photolysis, chemical cleavage, thermal cleavage, or enzymatic cleavage. In some embodiments the cleavage may be by biochemical, chemical, enzymatic, nucleophilic, reduction sensitive agent or other means. Cleavable linkers may comprise a moiety selected from the group consisting of: a restriction endonuclease site; at least one ribonucleotide cleavable with an RNAse; nucleotide analogues cleavable in the presence of certain chemical agent(s); photo-cleavable linker unit; a diol linkage cleavable by treatment with periodate (for example); a disulfide group cleavable with a chemical reducing agent; a cleavable moiety that may be subject to photochemical cleavage; and a peptide cleavable by a peptidase enzyme or other suitable means. Cleavage may be mediated enzymatically by incorporation of a cleavable nucleotide or nucleobase into the cleavable linker, such as uracil or 8-oxo-guanine.

In some embodiments, the linker described herein may be covalently and directly attached the attachment polynucleotide, for example, forming a —O— linkage, or may be covalently attached through another group, such as a phosphate or an ester. Alternatively, the linker described herein may be covalently attached to a phosphate group of the attachment polynucleotide, for example, covalently attached to the 3′ hydroxyl via a phosphate group, thus forming a —O—P(O)₃— linkage.

A binding element, as used herein, is a moiety that can be used to bind, covalently or non-covalently, to a binding partner. In some aspects, the binding element is on the transposome complex and the binding partner is on the solid support. In some embodiments, the binding element can bind or is bound non-covalently to the binding partner on the solid support, thereby non-covalently attaching the transposome complex to the solid support. In some embodiments, the binding element is capable of binding (covalently or non-covalently) to a binding partner on a solid support. In some aspects, the binding element is bound (covalently or non-covalently) to a binding partner on the solid support, resulting in an immobilized transposome complex.

In such embodiments, the binding element comprises or is, for example, biotin, and the binding partner comprises or is avidin or streptavidin. In other embodiments, the binding element/binding partner combination comprises or is FITC/anti-FITC, digoxigenin/digoxigenin antibody, or hapten/antibody. Further suitable binding pairs include, but not limited to, desthiobiotin-avidin, dithiobiotin-avidin, iminobiotin-avidin, biotin-avidin, dithiobiotin-succinilated avidin, iminobiotin-succinilated avidin, biotin-streptavidin, and biotin-succinilated avidin. In some embodiments, the binding element is a biotin and the binding partner is streptavidin.

In some embodiments, the binding element can bind to the binding partner via a chemical reaction or is bound covalently by reaction with the binding partner on the solid support, thereby covalently attaching the transposome complex to the solid support. In some aspects, the binding element/binding partner combination comprises or is amine/carboxylic acid (e.g., binding via standard peptide coupling reaction under conditions known to one of ordinary skill in the art, such as EDC or NHS-mediated coupling). The reaction of the two components joins the binding element and binding partner through an amide bond. Alternatively, the binding element and binding partner can be two click chemistry partners (e.g., azide/alkyne, which react to form a triazole linkage).

In some embodiments, the attachment polynucleotide further includes additional sequences or components, such as a universal sequence, a spacer region, an anchor sequence, or an index tag sequence, or a combination thereof. A universal sequence is a region of nucleotide sequence that is common to two or more nucleic acid fragments. Optionally, the two or more nucleic acid fragments also have regions of sequence differences. A universal sequence that may be present in different members of a plurality of nucleic acid fragments can allow for the replication or amplification of multiple different sequences using a single universal primer that is complementary to the universal sequence.

Variations of the transposome complex, including the transposase, the transposons, and the attachment polynucleotide may be realized. For example, variations in configuration, design, hybridization, structural elements, and overall arrangement of the transposome complex may be realized. The disclosure and drawings provided herein provide several variations, but it is understood that additional variations within the scope of the disclosure may be readily realized.

In some embodiments, one or more library product used to generate a polynucleotide is produced by bead-based tagmentation. In some embodiments, one or more library product used to generate a polynucleotide is produced by solution-based tagmentation.

B. Truseq Methods

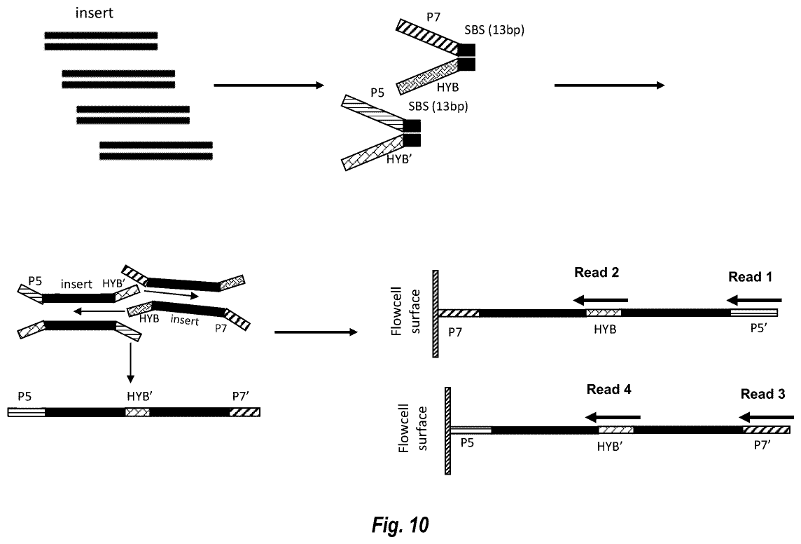

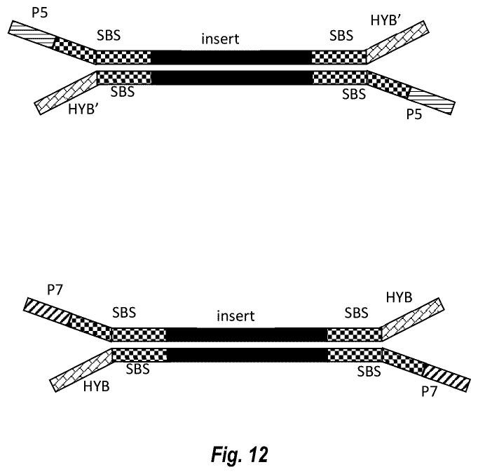

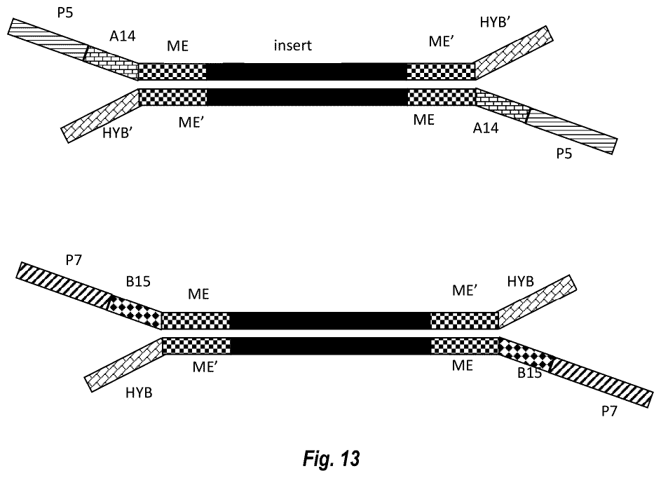

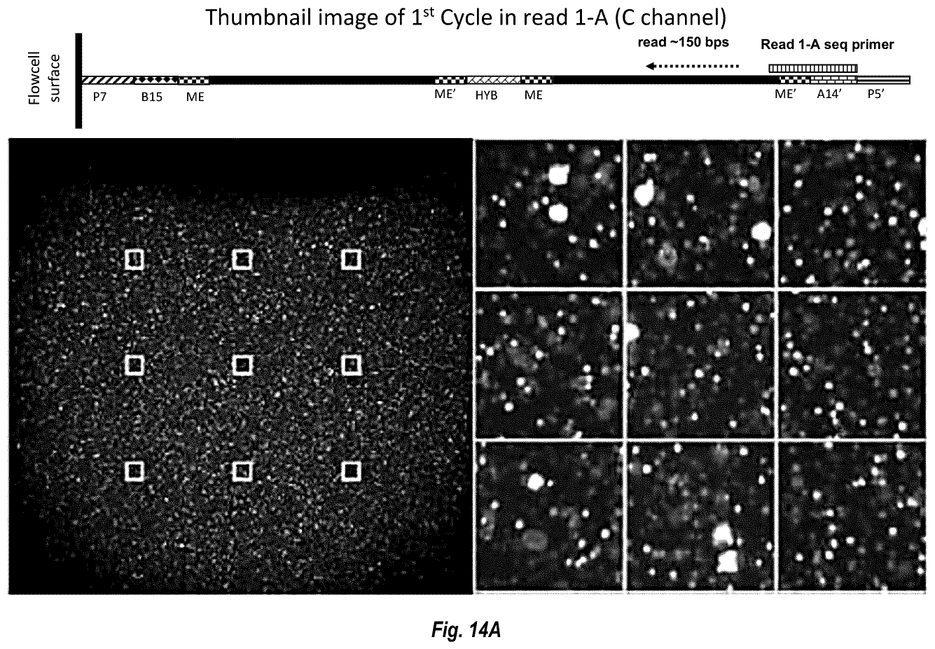

Forked adapter-based technology can be utilized for generating polynucleotides, for example, as exemplified in the workflow for Truseq sample preparation kits (Illumina, Inc.). FIGS. 10, 12, and 13 present a variety of approaches for generating library products comprising HYB or HYB′ sequences using Truseq methods.

In some embodiments, an adapter composition or kit comprises a first forked adapter complex and a second forked adapter complex, wherein the first forked adapter complex comprises: a complement attachment polynucleotide comprising a 5′ portion comprising a complement attachment sequence; and a 3′ portion comprising an adapter; and a hybridization polynucleotide comprising (a) a 5′ portion comprising the complement of a portion of the adapter and hybridized thereto; and (b) the complement of a hybridization sequence, wherein the complement of the hybridization sequence is not complementary to the complement attachment polynucleotide; and the second forked adapter complex comprises an attachment polynucleotide comprising a 5′ portion comprising an attachment sequence; and a 3′ portion comprising the adapter; and a hybridization polynucleotide comprising (a) a 5′ portion comprising the complement of a portion of the adapter and hybridized thereto; and (b) a hybridization sequence, wherein the hybridization sequence is not complementary to the attachment polynucleotide.

In some embodiments, the attachment sequence comprises a P5 primer sequence and the complement attachment sequence comprises a P7 primer sequence.

In some embodiments, the complement attachment polynucleotide comprises a B15 sequence and the hybridization polynucleotide comprises a A14 sequence.

In some embodiments, the first forked adapter complex has the structure:

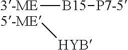

In some embodiments, the second forked adapter complex has the structure:

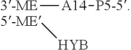

In some embodiments, the adapter complexes comprise methylated nucleotides (e.g., include methylated cytosines).

C. Methods Comprising Ligation

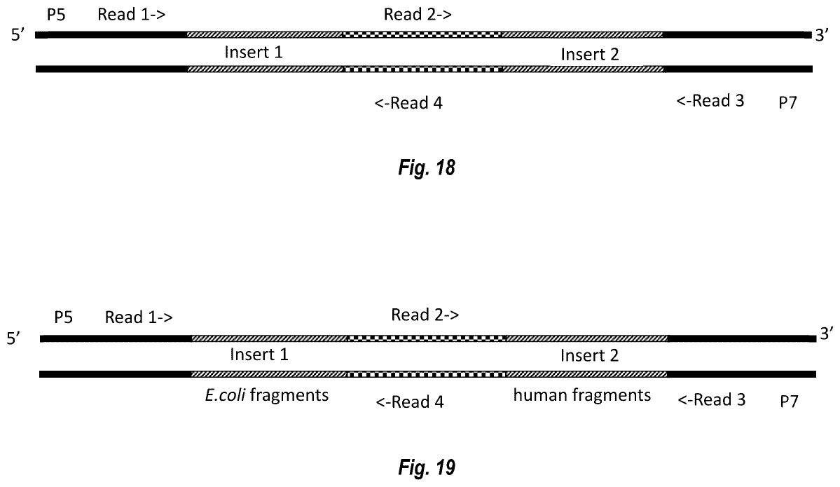

In some embodiments, a library of polynucleotides is prepared via a method comprising a ligation step (FIGS. 15A-F) such that each polynucleotide contains two inserts separated by an adapter sequence (FIGS. 18-19). Each starting polynucleotide has one insert. Starting polynucleotides from two or more libraries are treated with restriction enzymes to produce polynucleotides with compatible overhangs such that the polynucleotides may be ligated together in a variety of desired configurations to produce a new library of polynucleotides. The overhangs circumvent any issues that may arise due to fork adapter handle complementarities. In some embodiments, the new library is prepared from two starting libraries.

In some embodiments, the overhangs are produced using restriction enzymes and restriction enzyme recognition sites. In some embodiments, the enzyme is a type II, type IIS, type IIP, or type IIT restriction enzyme. In some embodiments, the enzyme is BtgZI. In some embodiments, the enzyme is BgLII. In some embodiments, the overhangs are ligated together using a ligase.

In some embodiments, the polynucleotides are attached to a binding element, such as biotin. In some embodiments, the digested ends of polynucleotides are removed by applying a binding partner, such as streptavidin magnetic beads.

FIGS. 15A-F show an exemplary ligation method of preparing a tandem insert library. In some embodiments, the tandem insert library is sequenced using multiple reads. In some embodiments, Read 1 and Read 4 give paired end data from the first insert. In some embodiments, Read 2 and Read 3 give paired end data from the second insert.

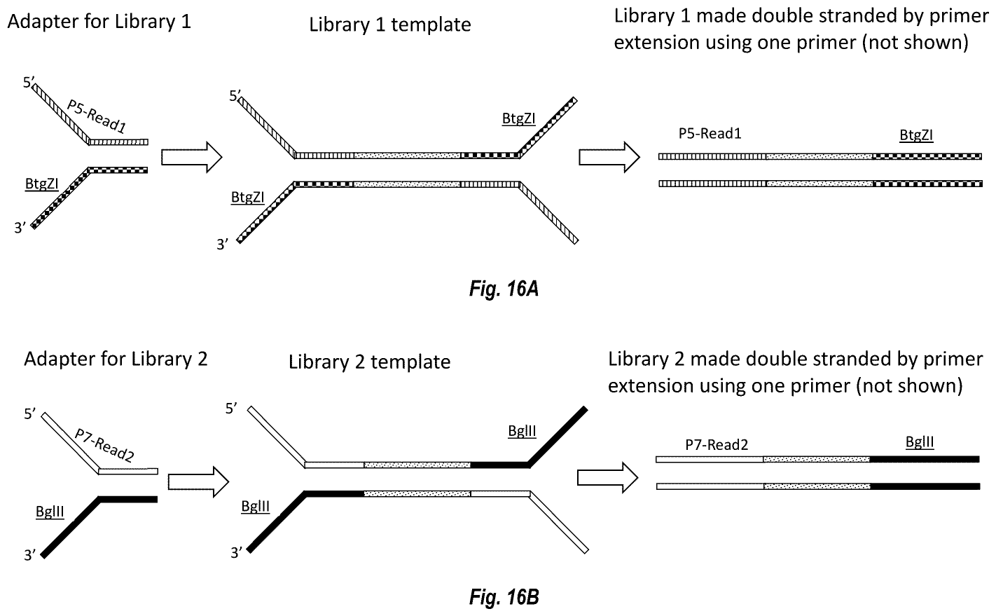

In some embodiments, forked adapters are ligated to inserts to used to generate polynucleotides with different ends (FIGS. 16A-B). In some embodiments, the forked adapter for a first library comprises (1) P5 and Read 1 on its first strand; and (2) a BtgZI restriction enzyme recognition site on its second strand. In some embodiments, the forked adapter for a second library comprises (1) P7 and Read 2 on its first strand; and (2) a BglII restriction enzyme recognition site on its second strand. In some embodiments, primer extension is used to generate polynucleotides that are double-stranded along the entire length of each polynucleotide, i.e., without forked configurations (FIGS. 16A-B).

D. Methods Comprising Strand Overlap Extension (SOE)

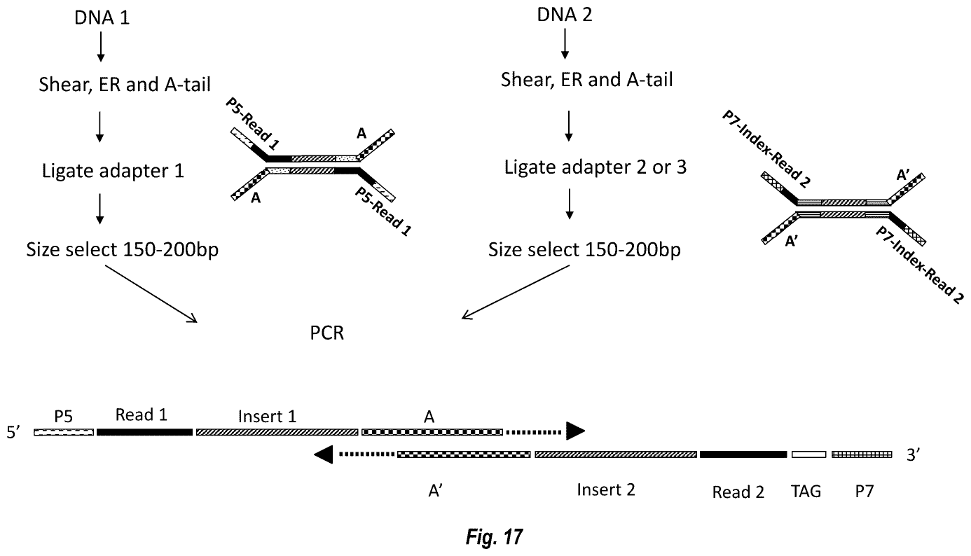

In some embodiments, a library of polynucleotides is prepared via a method comprising strand overlap extension (SOE) (FIGS. 17-18) such that each polynucleotide contains two inserts separated by an adapter sequence (FIGS. 17-18). In some embodiments, the adapter sequence is a concatenation sequence, defined herein as a hybridization sequence that may comprise one or more primer binding sequences. Each starting polynucleotide has one insert. Starting polynucleotides from two or more libraries are ligated with adapters. In some embodiments, these adapters are forked adapters or Y adapters. Forked adapters are designed such that every starting library has a unique adapter sequence attached to its polynucleotides. These adapter sequences provide complementary sequences for annealing in a variety of desired configurations to produce a new library of polynucleotides (FIG. 17). In some embodiments, the new library is prepared from two starting libraries. In some embodiments, the new library is prepared from three or more starting libraries.

For example, a first library contains polynucleotides that have a first adapter sequence at one end and a second adapter sequence on the other end. In these embodiments, the first or the second adapter sequence bears a 3′ sequence that is complementary to the 3′ end sequence of a third adapter sequence in a second library. The mixing of the two libraries together by denaturation and reannealing allows the complementary ends from both libraries to hybridize. In these embodiments, a polymerase extension reaction extends the complementary regions to full length, thus generating dual-insert polynucleotides.

FIGS. 17-18 show an exemplary SOE method of preparing a tandem insert library. In some embodiments, a starting library DNA is sheared to produce DNA fragments. A polymerase is used to remove damaged DNA ends as well as extend the DNA strands to generate blunt end duplexes. A kinase is used to phosphorylate the 5′-hydroxyl of the DNA strands. Then, a polymerase is used to add a single adenine base to the 3′ ends of each duplex. With this adenine overhang (the “A-tail” in FIG. 17), each end of a DNA fragment may be ligated to the single thymine overhang of an adapter. After ligation of the DNA fragments with the adapters, the libraries are cleaned up to select for 150-200 base pair fragments, and are mixed and prepared for a PCR reaction. The DNA strands denature at elevated temperatures and reanneal at lower temperatures. This allows the A and A′ complementary adapter sequences to hybridize with each other. The polymerase in the PCR reaction then extends the strands to form the tandem insert polynucleotide.

In many embodiments, the adapter may comprise a variety of sequences in a variety of combinations. In some embodiments, the adapter is a forked adapter that may include a P5, Read 1, tag, and/or A sequence. In some embodiments, the adapter is a forked adapter that may include a P7, Index, Read 2, tag, and/or A′ sequence.

In some embodiments, the tandem insert library is sequenced using multiple reads. In some embodiments, Read 1 and Read 4 give paired end data from the first insert. In some embodiments, Read 2 and Read 3 give paired end data from the second insert.

### IV. Methods of Generating a Concatenated Nucleic Acid Sequencing Template

This application also discloses methods of generating a concatenated nucleic acid sequencing template. Multiple insert sequences can be sequenced from a concatenated nucleic acid sequencing template. In other words, a concatenated nucleic acid sequencing template can be used for generating tandem reads.

In some embodiments, a concatenated nucleic acid sequencing template is generated via formation of a hybridized adduct. As used herein, a “hybridized adduct” refers to a hybridization sequence annealed to a complement of a hybridization sequence. In some embodiments, a fully double-stranded concatenated nucleic acid sequencing template is generated after formation of a hybridized adduct.

In some embodiments, a method of generating a concatenated nucleic acid sequencing template comprises: attaching a first read primer binding sequence to the 3′ end of a first insert sequence derived from a first target nucleic acid; attaching a hybridization sequence to the 5′ end of the first insert sequence; attaching the complement of the hybridization sequence to the 3′ end of a second insert sequence derived from a discontiguous region of the first target nucleic acid or from a second target nucleic acid; and annealing the hybridization sequence to the complement of the hybridization sequence to form a hybridized adduct; synthesizing a fully double-stranded concatenated nucleic acid sequencing template from the hybridized adduct; wherein the region between the first and second insert sequences comprises a second read primer binding sequence that comprises the hybridization sequence and is orthogonal to the first read primer binding sequence; thereby generating a concatenated nucleic acid sequencing template.

In some embodiments, the attaching the first read primer binding sequence and the attaching the hybridization sequence comprises contacting the one or more target nucleic acids with a transposome complex under conditions suitable for tagmentation.

In some embodiments, the attaching the complement of the hybridization sequence to the 3′ end of a second insert sequence derived from a discontiguous region of the first target nucleic acid or from a second target nucleic acid comprises contacting the one or more target nucleic acids with a transposome complex of under conditions suitable for tagmentation.

In some embodiments, the attaching a first read primer binding sequence to the 3′ end of a first insert sequence and the attaching a hybridization sequence to the 5′ end of the first insert sequence comprise contacting one or more target nucleic acids with a first forked adapter complex under conditions suitable for ligation of the adapter complexes to the ends of the fragments to form fragments ligated at both ends with the first adapter complex and fragments ligated at both ends with the second adapter complex, and denaturing the ligated fragments.

In some embodiments, the attaching the complement of the hybridization sequence to the 3′ end of a second insert sequence comprises contacting one or more target nucleic acids with a second forked adapter complex under conditions suitable for ligation of the adapter complexes to the ends of the fragments to form fragments ligated at both ends with the first adapter complex and fragments ligated at both ends with the second adapter complex, and denaturing the ligated fragments.

In some embodiments, a method of generating a concatenated nucleic acid sequencing template comprises contacting a first sample comprising a first target nucleic acid with a first transposome complex and a second transposome complex, wherein each transposome complex comprises:
a transposase;
a first transposon comprising a 3′ portion comprising a transposon end sequence and a 5′ portion comprising an adapter sequence; and a second transposon comprising a 5′ portion comprising the complement of the transposon end sequence and hybridized thereto;
wherein the adapter sequence in the first transposome complex is the complement of a first adapter sequence and the adapter sequence in the second transposome complex is a second adapter sequence;
under conditions sufficient to fragment the first target nucleic acid to generate a first tagged product comprising an insert sequence from the first target nucleic acid tagged at one end with the transposons of the first transposome complex and at the other end with the transposons of the second transposome complex;
adding a complement attachment sequence to the 3′ end of the first tagged product and adding the complement of a hybridization sequence to the 5′ end of the first tagged product, optionally by polymerase chain reaction, to form a first modified tagged product;
contacting a second sample comprising a second target nucleic acid with the transposome complexes under conditions sufficient to fragment the second target nucleic acid to generate a second tagged product comprising an insert sequence from the second target nucleic acid tagged at one end with the transposons of the first transposome complex and at the other end with the transposons of the second transposome complex;
adding an attachment sequence to the 3′ end of the second tagged product and adding a hybridization sequence to the 5′ end of the second tagged product, optionally by polymerase chain reaction, to form a second modified tagged product;
annealing the hybridization sequence of the first modified tagged product to the complement of the hybridization sequence in the second modified tagged product to form a hybridized adduct; and
synthesizing a fully double-stranded concatenated nucleic acid sequencing template from the hybridized adduct, wherein the concatenated nucleic acid sequence template comprises:
(a) a first read primer binding sequence 3′ of the insert sequence from the second target nucleic acid, wherein the first read primer binding sequence comprises the first adapter sequence and the complement of the transposon end sequence, and
(b) a second read primer binding sequence between the two insert sequences, wherein the second read primer binding sequence comprises the transposon end sequence and the hybridization sequence, and
wherein the first read primer binding sequence is orthogonal to the second read primer binding sequence.

In some embodiments, a method of generating a concatenated nucleic acid sequencing template comprises:
contacting a first sample comprising a first target nucleic acid with a first transposome complex, wherein the first transposome complex comprises:
a transposase;
a first transposon comprising a 3′ portion comprising a transposon end sequence and a portion comprising an attachment sequence and the complement of a first adapter sequence; and
a second transposon comprising a 5′ portion comprising the complement of the transposon end sequence and hybridized thereto;
under conditions sufficient to fragment the first target nucleic acid to generate a first tagged product comprising an insert sequence from the first target nucleic acid tagged at each end with the transposons of the first transposome complex; and adding the complement of a hybridization sequence to the 5′ end of the first tagged product, optionally by polymerase chain reaction, to form a first modified tagged product;
contacting a second sample comprising a second target nucleic acid with a second transposome complex, wherein the second transposome complex comprises: a transposase;
a first transposon comprising a 3′ portion comprising a transposon end sequence and a 5′ portion comprising a second adapter sequence and a complement attachment sequence; and
a second transposon comprising a 5′ portion comprising the complement of the transposon end sequence and hybridized thereto;
under conditions sufficient to fragment the second target nucleic acid to generate a second tagged product comprising an insert sequence from the second target nucleic acid tagged at each end with the transposons of the second transposome complex; adding the complement of the hybridization sequence to the 5′ end of the second tagged product, optionally by polymerase chain reaction, to form a second modified tagged product;
annealing the hybridization sequence of the first modified tagged product to the complement of the hybridization sequence in the second modified tagged product to form a hybridized adduct; and
synthesizing a fully double-stranded concatenated nucleic acid sequencing template from the hybridized adduct, wherein the concatenated nucleic acid sequence template comprises:
(a) a first read primer binding sequence 3′ of the insert sequence from the second target nucleic acid, wherein the first read primer binding sequence comprises the first adapter sequence and the complement of the transposon end sequence, and
(b) a second read primer binding sequence between the two insert sequences, wherein the second read primer binding sequence comprises the transposon end sequence and the hybridization sequence, and
wherein the first read primer binding sequence is orthogonal to the second read primer binding sequence.

In some embodiments, the transposome complexes are immobilized on a solid support.

### V. Methods of Preparing Sequencing Templates Using Forked Adapters

In some embodiments, forked adapters may be used to prepare sequencing templates comprising more than one insert.

In some embodiments, the adapter may be a forked adapter, also known as a Y-adapter. Forked adapter-based technology can be utilized for generating polynucleotides, for example, as exemplified in the workflow for TruSeq™ sample preparation kits (Illumina, Inc.). Reagents from the workflow for TruSight® Oncology kits (Illumina, Inc.) may also be used to assemble forked adapters. In some embodiments, a forked adapter comprises a HYB or HYB′ sequence.

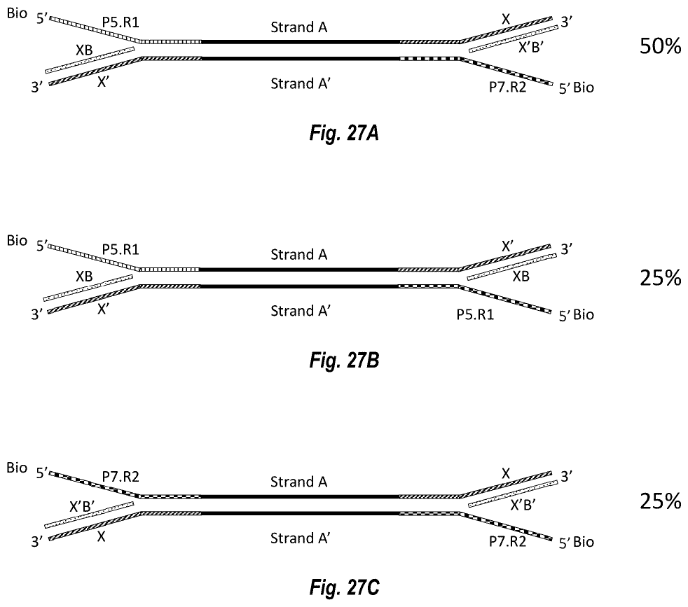

As used herein, a “forked adapter” refers to an adapter comprising two strands of nucleic acid, wherein the two strands each comprise a region that is complementary to the other strand and a region that is not complementary to the other strand. In some embodiments, the two strands of nucleic acid in the forked adapter are annealed together before ligation, with the annealing based on complementary regions. In some embodiments, the complementary regions each comprise 12 nucleotides. In some embodiments, a forked adapter is ligated to both strands at the end of a double-stranded DNA fragment. In some embodiments, a forked adapter is ligated to one end of a double-stranded DNA fragment. In some embodiments, a forked adapter is ligated to both ends of a double-stranded DNA fragment. In some embodiments, the forked adapters on opposite ends of a fragment are different (as shown in FIG. 27A). In some embodiments, one strand of the forked adapter is phosphorylated at it 5′ to promote ligation to fragments. In some embodiments, one strand of the forked adapter has a phosphorothioate bond directly before a 3′ T. In some embodiments, the 3′ T is an overhang (i.e., not paired with a nucleotide in the other strand of the forked adapter). In some embodiments, the 3′ T overhang can basepair with an A-tail present on a library fragment. In some embodiments, the phosphorothioate bond blocks exonuclease digestion of the 3′ T overhang.

In some embodiments, each forked adapter comprises a first oligonucleotide and a second oligonucleotide that are partially hybridized to each other to form a double-stranded section and a single stranded section.

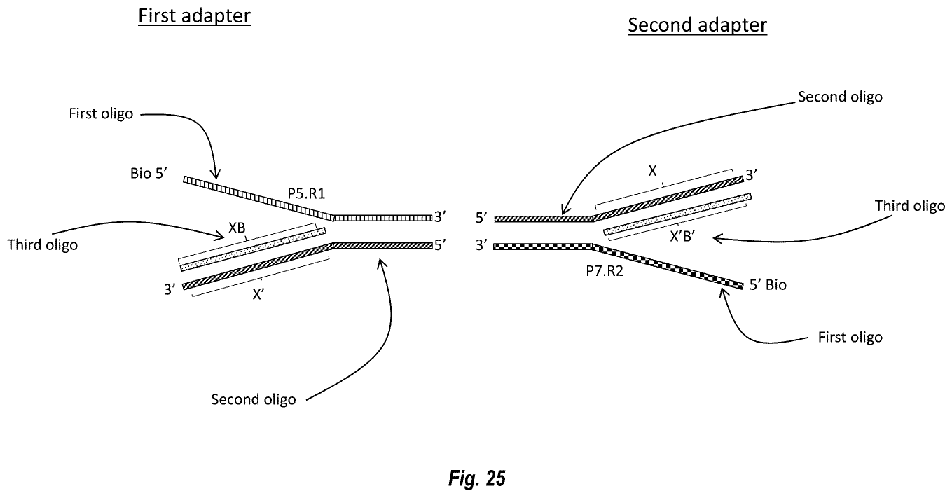

FIG. 25 shows a pair of forked adapters (i.e., a first adapter and a second adapter) that may be used to prepare sequencing templates. In some embodiments, the first strand of each forked adapter comprises an adapter, such as a sequencing primer sequence. In some embodiments, the second strand of each forked adapter comprises either a hybridization sequence (X) or the complement of a hybridization sequence (X′).

In order to block a hybridization sequence (X) and its complement (X′) from binding to each other at undesired times, blocking oligonucleotides can be employed. In some embodiments, blocking oligonucleotides comprise one or more modification such that they are not targets of tagmentation. In other words, the blocking oligonucleotides may be designed to be resistant to transposases and thus avoid cleavage of the double-stranded nucleic acid formed by hybridization of a blocking oligonucleotide to a hybridization sequence or its complement. In some embodiments, a blocking oligonucleotide comprises a phosphorothioate backbone.

In some embodiments, a blocking oligonucleotide comprises the complement of all or part of the sequence one wants to block from hybridizing. Thus, in some embodiments, a blocking oligonucleotide may be all or part of an X or X′ sequence. As used herein, a “blocking oligonucleotide” refers to an oligonucleotide that can be used to inhibit binding of two sequences to each other, until the blocking oligonucleotide bound to at least one of the two sequences is removed. In some embodiments, a blocking oligonucleotide comprises a sequence that is fully or partially complementary to all or part of either the hybridization sequence (X or HYB) or its complement (X′ or HYB′). For example, a blocking oligonucleotide (X′B′) to block a HYB sequence (X in FIG. 25) may comprise all or part of a HYB′ sequence, and a blocking oligonucleotide (XB) to block a HYB′ sequence (X′ in FIG. 25) may comprise all or part of a HYB sequence.

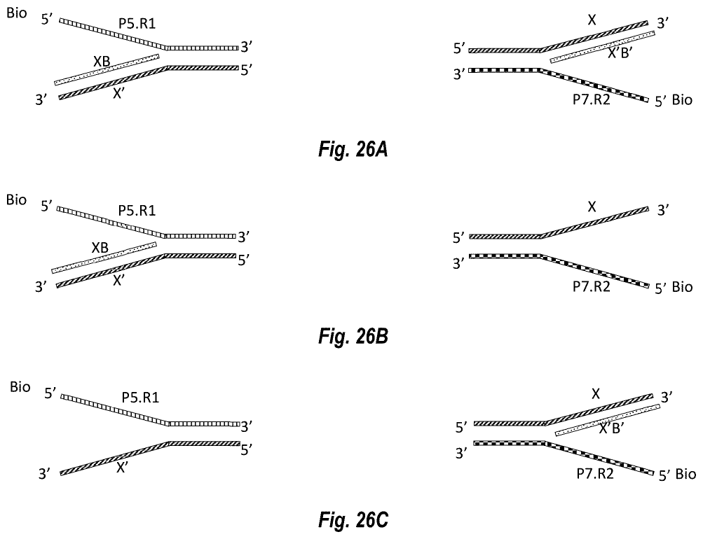

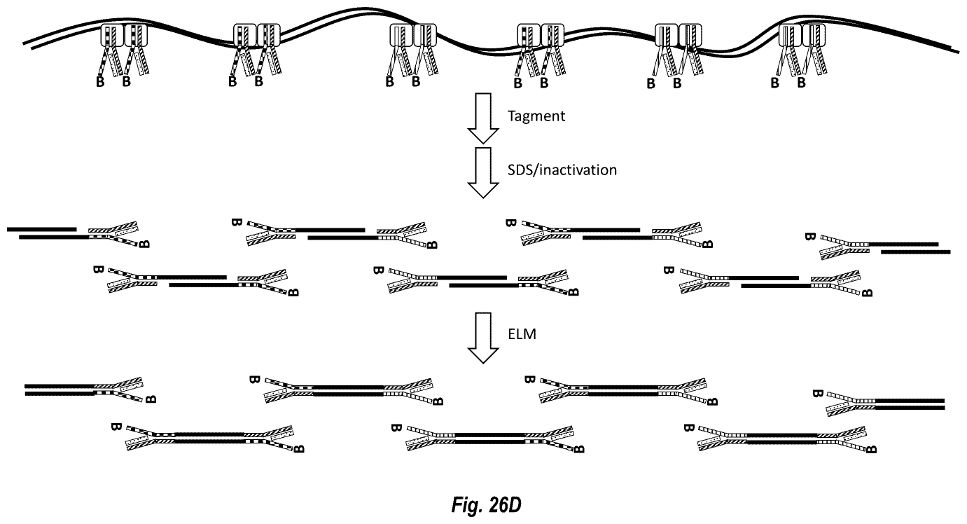

In the case of the forked adapters shown in FIG. 26, one or more blocking oligonucleotide can serve to block binding of a X sequence in one forked adapter to a X′ sequence in the other forked adapter.

In some embodiments, a blocking oligonucleotide (XB) is bound to the X′ sequence. In some embodiments, a blocking oligonucleotide (X′B′) is bound to the X sequence. In some embodiments, a blocking oligonucleotide is bound to both the X and X′ sequences. The blocking oligonucleotide may be fully or partially complementary to either an X or an X′ sequence. In some embodiments, the blocking oligonucleotide binds to the full X or X′ sequence. In some embodiments, the blocking oligonucleotide binds to a portion of the X or X′ sequence.

One or both forked adapters may also comprise an affinity moiety on the 5′ end of the first strand of the forked adapter. In some embodiments, such as that shown in FIG. 26, both the first strand of the first forked adapter and the first strand of the second forked adapter comprise an affinity moiety at the 5′ end of the strand. In some embodiments, the affinity moiety is biotin, desthiobiotin, or dual biotin. In some embodiments, the affinity moiety is a biotin (i.e., the first strand of one or both forked adapters are biotinylated). In some embodiments, the affinity moiety binds to a binding moiety on a surface of a solid support. In some embodiments, the binding moiety is avidin or streptavidin, which binds to an avidin or streptavidin on the surface of a solid support. A range of affinity moieties that can bind to binding moieties are known to those skilled in the art, and a user may choose any pair of an affinity/binding moiety of their choice.

In some embodiments, the binding moiety serves to immobilize tagged fragments (prepared by ligation of forked adapters to fragments) on a solid support. In some embodiments, single-stranded fragments ligated to at least one first strand of a forked adapter will be immobilized on the solid support. In some embodiments, immobilized fragments can be washed and blocking oligonucleotides can be removed, without the fragments being released from the surface of the solid support.

In some embodiments, a first strand of a forked adapter comprises a 5′ affinity element capable of binding to an affinity binding partner on a solid support or bead. Such an affinity element may be biotin, as shown by the “Bio” in the first and second adapters shown in FIG. 25.

In some embodiments, the affinity element is connected via a linker attached to the first strand. In some embodiments, this linker is a cleavable linker.

In some embodiments, the affinity moiety is linked to the first strand of a forked adapter by a linker. In some embodiments, the linker is a cleavable linker. In some embodiments, a user can release sequencing templates prepared from immobilized fragments from a solid support at a desired time by cleaving a cleavable linker between the affinity moiety and the first strand of the forked adapter. In some embodiments, amplicons of sequencing templates may be prepared on the surface of the solid support, in which case the amplicons may be sequenced without requiring release of sequencing templates from the surface.

In some embodiments, the hybridization sequence (HYB) and the complement of the hybridization sequence (HYB′) can hybridize to each other. However, in some cases, this could potentially lead to dimerization between different forked adapters based on binding of HYB in one forked adapter to a HYB′ in another forked adapter. Such adapter dimerization could decrease the ability to ligate the forked adapters to the end of fragments of nucleic acid.

In some embodiments, a blocking oligonucleotide is employed to block binding of HYB to HYB′ between different forked adapters until a user wants this binding to occur. In some embodiments, the hybridization sequence or its complement is bound to a blocking oligonucleotide that is fully or partially complementary to the hybridization sequence or its complement.

FIGS. 26A-26C show a variety of different forked adapters embodiments. A blocking oligonucleotide may be bound to the second strand of both the first and second forked adapter (FIG. 26A). Alternatively, a blocking oligonucleotide may be bound to only the second strand of a first forked adapter (FIG. 26B) or to only the second strand of the second forked adapter. As long as either the hybridization sequence (X) or the complement of the hybridization sequence (X′) is bound by a blocking oligonucleotide, the blocking oligonucleotide will block annealing of forked adapter to each other via association of X to X′. Similar methods can be performed with transposome complexes in solution, as shown in FIG. 26D.

In some embodiments, a forked adapter comprising two polynucleotide strands comprises (a) a first strand comprising a sequencing primer sequence; and (b) a second strand comprising a 3′ hybridization sequence or its complement, wherein the 3′ end of the first strand is fully or partially complementary to the 5′ end of the second strand. In other words, the two strands of a forked adapter may hybridize together in a certain region, while the two strands are separate in another region. The sequence of the first and second strand may be different or all or partially non-complementary in the region wherein the two strands are separate, while the first and second strand may be the same and fully or partially complementary in the region wherein the two strands are hybridized together.

As is well-known in the field, additional sequences of interest can be comprised in forked adapters, such as UMIs and sample indexes. In other words, forked adapters are not limited to the types of sequences shown in FIG. 25, but forked adapters may comprise one or more additional types of sequences, such as UMIs or sample indexes.

In some embodiments, the first strand and/or second strand further comprise at least one of an adapter, a barcode sequence, a unique molecular identifier (UMI) sequence, a sample index sequence, a capture sequence, or a cleavage sequence.

In some embodiments, the sequencing primer sequence comprised in a first strand of a forked adapter comprises a B15 sequence or an A14 sequence, or their complements. In some embodiments, the first strand of a forked adapter further comprises a P7 or P5 primer sequence, or their complements. Such embodiments are shown in FIG. 25, wherein the first strand of a first adapter comprises a P5 sequence and a first read sequencing adapter sequence (P5.R1) and the first strand of a second adapter comprises a P7 sequence and a second read sequencing adapter sequence (P7.R2).

In some embodiments, a forked adapter is comprised in a mixture with another non-identical forked adapter. In some embodiments, a mixture comprises a first forked adapter and a second forked adapter that are different.

In some embodiments, a composition or kit comprises two forked adapters, wherein (a) the first forked adapter comprises a first strand comprising a first read sequencing primer sequence and a second strand comprising a complement of a hybridization sequence and (b) the second forked adapter comprises a first strand comprising a second read sequencing primer sequence and a second strand comprising a hybridization sequence. In some embodiments, one or both forked adapter comprised in a kit or composition comprise a blocking oligonucleotide.

A mixture of forked adapters may be ligated to double-stranded nucleic acid fragments. These fragments may be prepared from DNA (such as genomic DNA or cDNA prepared from RNA) using well-known techniques in the art, such as physical means using acoustics, nebulization, centrifugal force, needles, or hydrodynamics. Enzymatic means of preparing fragments are also well-known, such as DNase treatment.

When a mixture comprising a first forked adapter and a second forked adapter is combined double-stranded nucleic acid fragments under conditions for ligating, the predicted ratio would be 50% of fragments would be tagged with a first forked adapter at one end and a second forked adapter at a second end (FIG. 27A), 25% of fragments would be tagged with a first forked adapter at both ends (FIG. 27B), and 25% of fragments would be tagged with a second forked adapter at both ends (FIG. 27C). In some embodiments, the ligation products shown in FIGS. 27A-27C may be produced by a ligation reaction prepared in solution. In other words, the tagged fragments shown in FIGS. 27A-27C may be prepared in solution.

In some embodiments, tagged fragments prepared in solution by ligation of forked adapters can then be immobilized on the surface of a solid support.

In some embodiments, a method of generating one or more concatenated nucleic acid sequencing templates comprises contacting a sample comprising double-stranded nucleic acid fragments each comprising an insert prepared from a target nucleic acid with a composition or kit comprising two forked adapters, wherein one or both forked adapters comprise a blocking oligonucleotide. In some embodiments, after contacting the sample with the two forked adapters, the method comprises ligating the forked adapters to the double-stranded fragments to prepare tagged double-stranded fragments and immobilizing the tagged double-stranded fragments on a solid support.

In some embodiments, double-stranded fragments are applied to a solid support after ligation with forked adapters. In some embodiments, both the 5′ ends of tagged double-stranded fragments comprise an affinity moiety (based on ligation of the first strand of a forked adapter comprising an affinity moiety) that can bind to a binding moiety on the surface of a solid support. In some embodiments, binding of the affinity moiety to the binding moiety immobilizes fragments on the solid support, such that they will not be released from the support by temperature changes that can allow release of a blocking oligonucleotide bound to a hybridization sequence or its complement.

After immobilizing double-stranded fragments on the surface of a solid support, a method can comprise denaturing (1) the immobilized tagged double-stranded fragments to produce immobilized single-stranded fragments and (2) the blocking oligonucleotides to unblock hybridization sequences and complements of hybridization sequences. In some embodiments, the denaturing is performed with an increase in temperature, change in pH, and/or addition of one or more chaotropic agents. In some embodiments, for example, a single temperature change can mediate denaturing of the two strands of double-stranded fragments and release of the blocking oligonucleotide. In some embodiments, wherein the increase in temperature associated with denaturing is an increase from 45° C.-55° C. to 85° C.-95° C., optionally wherein the increase in temperature is an increase from 50° C. to 90° C. In some embodiments, the one or more chaotropic agents comprise formamide and/or NaOH.

In some embodiments, a first single-stranded fragment comprises an insert, and a second single-stranded fragment comprises an insert that is the complement of the insert comprised in the first fragment. In some embodiments, a first single-stranded fragment comprises an insert, and a second fragment comprises an insert that is not the complement of the insert comprised in the first fragment. In some embodiments, hybridizing occurs between single-stranded fragments prepared from double-stranded fragments comprising a first forked adapter ligated at one end of each fragment and a second forked adapter ligated at the other end of each fragment. In some embodiments, two immobilized single-stranded fragments do not hybridize to each other to form a bridge in the absence of binding of a hybridization sequence in a first fragment to the complement of a hybridization sequence in a second fragment. In some embodiments, hybridizing two immobilized single-stranded fragments to each other to form a bridge does not occur between single-stranded fragments prepared from double-stranded fragments comprising the same forked adapter ligated at both ends of each fragment.

In some embodiments, the surface of the solid support is washed after the denaturing, and the blocking oligonucleotides will be removed by the wash, while the single-stranded fragments remain immobilized due to the interaction between the 5′ affinity moiety on the fragments with the binding moiety of the surface of the solid support. In some embodiments, the immobilizing of double-stranded or single-stranded fragments is by binding of an affinity moiety from the first and/or second forked adapter to one or more binding moieties on the surface of the solid support. In some embodiments, the affinity moiety is biotin, desthiobiotin, or dual biotin and the binding moiety is avidin or streptavidin.

Since the single-stranded fragments are prepared from double-stranded fragments that were already immobilized on a single surface on a solid support, complementary single-stranded fragments from a double-stranded fragment are likely to be in close proximity (as shown in FIG. 28A, wherein the left and right surface of a solid support show different views of the same surface). The denaturing of the blocking oligonucleotides means that the hybridization sequence and its complement (X and X′ in FIG. 28A) are now available to bind each other.

Next, the method comprises hybridizing two immobilized single-stranded fragments to each other to form a bridge by binding of the hybridization sequence in a first fragment to the complement of a hybridization sequence in a second fragment and extending from the 3′ ends of both single-stranded fragments to produce a double-stranded concatenated nucleic acid sequencing template wherein each strand of the template comprises inserts (or their complements) from both immobilized single-stranded fragments (as shown in FIG. 29).

In some embodiments, a single-stranded fragment prepared from a double-stranded fragment ligated with a first strand of a first forked adapter (such as shown in FIG. 25) at a first end and the second strand of a second forked adapter can bind to another single-stranded fragment prepared from a double-stranded fragment ligated with a first strand of a first forked adapter at a first end and the second strand of a second forked adapter by association of the hybridization sequence (X) in a first fragment to the complement of the hybridization sequence (X′) in a second fragment (FIG. 28A).

In some embodiments, one or more additional rounds of denaturing, hybridizing, and extending are performed. In this way, the method can proceed in making sequencing templates until single-stranded fragments do not have appropriate other single-stranded fragments with which to form bridges (and concatenated sequencing templates) via HYB/HYB′ binding.

In some embodiments, both single-stranded fragments prepared from a double-stranded fragment are immobilized on the surface of the same solid support. In some embodiments, the method is performed with a single surface on a solid support, so that all fragments are immobilized on the same solid support. The left and right surfaces (shown with attachment of the first and second fragments) presented in FIGS. 28A-28C represent two different views of the same surface on a solid support.

In some embodiments, release of blocking oligonucleotides generates “free” hybridization sequence that can bind to their complement sequences. In some embodiments, the hybridization sequence comprised in one single-stranded fragment can bind to a complement of the hybridization sequence in another single-stranded fragment. Such binding may generate a “bridge” as shown in FIG. 28A.

After elongation, a concatenated sequencing template can comprise two inserts that are copies of each other, as shown in FIG. 29.

Single-stranded fragments with identical ligated adapters cannot hybridize to each other. For example, two fragments tagged with X′ cannot pair to each other at the hybridization sequence (FIG. 28B) and two fragments tagged with X cannot pair with each other at the hybridization sequence (FIG. 28C).

Accordingly, no sequencing templates comprising two inserts can be prepared from fragments that comprise the same adapters (as indicated by the 0% shown in FIGS. 28B and 28C). While the two insert sequences could hybridize to each other (sequences Strand A and Strand A′ in FIGS. 28A-28C), hybridization directly between these sequences would not allow extension after the hybridizing, because such a pairing between Strand A and Strand A′ would be followed by 3′ sequences that are not complementary (X/X′).

In this way, 100% of sequencing templates comprising two copies of an insert are prepared from fragments that comprised different adapters (FIG. 28A). This aspect is important, since a first forked adapter can comprise different sequences than the second forked adapter. For example, a first forked adapter may comprise a first read sequencing adapter sequence (P5.R1) while a second forked adapter may comprise a second read sequencing adapter sequence (P7.R2), as shown in FIG. 28A.

Accordingly, a full-length concatenated sequencing template can be prepared after elongation comprising two copies of the same insert sequences and appropriate adapters that may be needed for the desired sequencing platform, as shown in FIG. 29. In other words, one skilled in the art can design the forked adapter in such a way that the resulting sequencing template comprising desired adapter sequences for their preferred sequencing platform.

Since double-stranded fragments are first immobilized on the solid support and then denatured, there is a high probability that two single-stranded fragments denatured from the same double-stranded fragment will be immobilized in close proximity to each other on the surface. This ordering of steps means that the two single-stranded fragments from the same double-stranded fragment (wherein one fragment comprises a Strand A sequence and the other fragment comprises a Strand A′ sequence, as shown in FIG. 28A) will likely be able to interact with each other. This aspect increases the likelihood that sequencing templates prepared by the present methods will comprise two copies of the same sequence from the target nucleic acid (one from Strand A and one from the complement of Strand A′ prepared by elongation). As described herein, such sequencing templates with two copies of the same insert sequence (arising from complementary strands of the target nucleic acid) allow for error correction or identification of base pair mismatches between the strand and anti-sense strand of a target nucleic acid. Such base pair mismatches may be uncommon and otherwise difficult to resolve with standard sequencing.

Alternatively, single-stranded fragments comprising unrelated insert sequences and complementary adapters can also hybridize into bridges and then generate concatenated sequencing templates. Concatenated sequencing templates with two different inserts can serve to increase the sequencing depth by allowing additional sequence reads as compared to sequencing with standard sequencing templates that comprise a single insert.

A. Methods of Compartmentalization for Evaluating Proximity Data

Any method described herein may be used with compartmentalization. In some embodiments, compartmentalization allows for generating proximity data, such as whether different inserts were comprised in the same target nucleic acid. When the same target nucleic acid is a chromosome, compartmentalization may be used for methods of haplotype phasing as described herein.

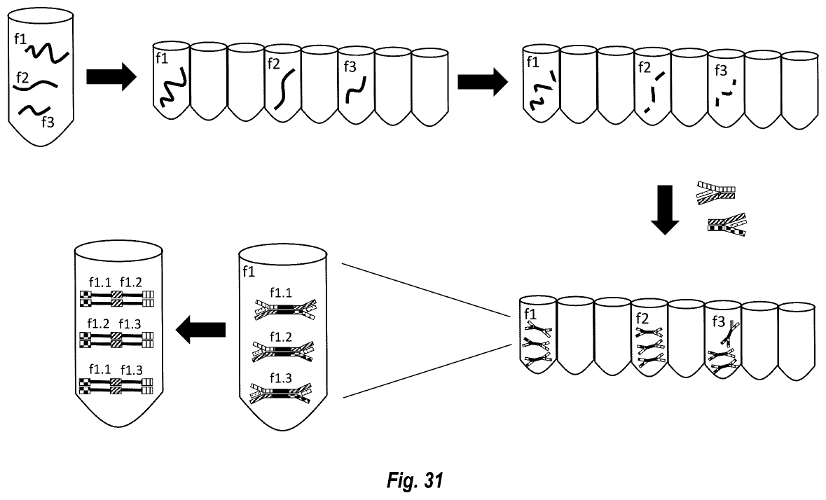

In some embodiments, compartmentalization is used with the present methods using forked adapters or transposomes to evaluate proximity data. In some embodiments, compartments may be used with dilution to limit the number of available target nucleic acids. In some embodiments, each compartment generally comprises one or no target nucleic acid after dilution (as shown in FIG. 31). Accordingly, fragments prepared in a given compartment are generally those prepared from the same target nucleic acid. In this way, inserts comprised in the same concatenated sequencing templates prepared by these methods can be inferred to have originated from the same target nucleic acid.

In some embodiments, the compartments are wells, tubes, or droplets. For example, FIG. 31 shows a method with wells, and FIG. 32 shows a method with droplets. A wide range of different wells, tubes, and droplets would be known to one skilled in the art and any type may be used in the present methods.

“Droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. In another example, a droplet may be bounded by filler fluid and the atmosphere. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, combinations of such shapes, and various shapes formed during droplet operations, such as merging or splitting or formed as a result of contact of such shapes with one or more surfaces of a droplet actuator. For examples of droplet fluids that may be subjected to droplet operations using the approach of the present disclosure, see Eckhardt et al., International Patent Pub. No. WO/2007/120241, entitled, “Droplet-Based Biochemistry,” published on Oct. 25, 2007, the entire disclosure of which is incorporated herein by reference. U.S. Pat. No. 10,975,371 teaches a wide variety of applications of droplets and droplet actuators and is incorporated herein in its entirety.

In some embodiments, fragments may be prepared within compartments using two pools of forked adapters: one pool comprising forked adapters comprising a hybridization sequence (i.e., the second adapter of FIG. 25) and the other pool comprising forked adapters comprising the complement of the hybridization sequence (i.e., the first adapter of FIG. 25).

In some embodiments, a method of generating one or more concatenated nucleic acid sequencing templates comprises compartmentalizing a sample comprising target double-stranded nucleic acid into a plurality of different compartments and preparing fragments each comprising an insert from the double-stranded nucleic acid within the plurality of different compartments. The method may then comprise contacting the plurality of different compartments with a composition or kit comprising two forked adapters, wherein one or both forked adapters comprise a blocking oligonucleotide, and ligating the forked adapters to the double-stranded fragments to prepared tagged double-stranded fragments within the plurality of different compartments.

In some embodiments, the method may then comprise denaturing (1) the immobilized tagged double-stranded fragments to produce single-stranded fragments and (2) the blocking oligonucleotides to unblock hybridization sequences and complements of hybridization sequences within the plurality of different compartments, and hybridizing two single-stranded fragments within the same compartment to each other to form a bridge by binding of the hybridization sequence in a first fragment to the complement of a hybridization sequence in a second fragment. In some embodiments, the method may comprise extending from the 3′ ends of each single-stranded fragment to produce a double-stranded concatenated nucleic acid sequencing template comprising inserts from both single-stranded fragments within the same compartment.

In some embodiments, the target double-stranded nucleic acid comprises double-stranded DNA fragments, and the preparing fragments prepares subfragments of the double-stranded DNA fragments. In other words, the target double-stranded nucleic acid may be fragmented into relatively large fragments, which are then fragmented into subfragments in compartments. This is shown in FIGS. 31 and 32, wherein the f1 fragment is fragmented into subfragments 1.1, 1.2, and 1.3.

Since single-stranded fragments are not immobilized in this method, concatenated sequencing templates are likely prepared comprising two different insert sequences. In some embodiments, a first single-stranded fragment comprises an insert and a second fragment comprises an insert that is not the complement of the insert comprised in the first fragment.

In some embodiments, the hybridizing occurs between single-stranded fragments prepared from double-stranded fragments comprising a first forked adapter ligated at one end of each fragment and a second forked adapter ligated at the other end of each fragment.

In some embodiments, single-stranded fragments do not hybridize to each other to form a bridge in the absence of binding of a hybridization sequence in a first fragment to the complement of a hybridization sequence in a second fragment. In some embodiments, the hybridizing two single-stranded fragments to each other to form a bridge does not occur between single-stranded fragments prepared from double-stranded fragments comprising the same forked adapter ligated at both ends of each fragment.

B. Haplotype Phasing

“Haplotype phasing,” as used herein, refers to identifying alleles that are co-located on the same chromosome. Sequencing data generally consists of unphased genotypes, and such data cannot differentiate which of the two parental chromosomes, or haplotypes, a particular allele falls on.

Methods of compartmentalization (such as for use in preparing whole-genome haplotyping) are well-known in the art, such as those taught in Amini et al., Nat Genet. 46(12):1343-9 (2014); Kaper F, et al. Proc. Natl. Acad. Sci. USA. 110(14):5552-5557 (2013); Kitzman J O, et al. Nat. Biotechnol. 29(1):59-63 (2011); Peters B A, et al. Nature. 487(7406):190-195 (2012); Fan H C, et al. Nat. Biotechnol. 29(1):51-57 (2011); Levy S, et al. PLoS Biol. 5(10):e254 (2007); Duitama J, et al. Nucleic Acids Res. 40(5):2041-2053 (2012); Suk E K, et al. Genome Res. 21(10):1672-1685 (2011), each of which is incorporated by reference in its entirety herein.

In some embodiments, compartmentalizing separates different haplotypes into different compartments and the method is used for haplotype phasing. In some embodiments, target nucleic acids, such as double-stranded DNA, are aliquoted into multiple compartments by limiting dilution such that an individual compartment contains a limited number of DNA molecules whereby any position of the genome is likely to be represented by haploid DNA in a compartment.

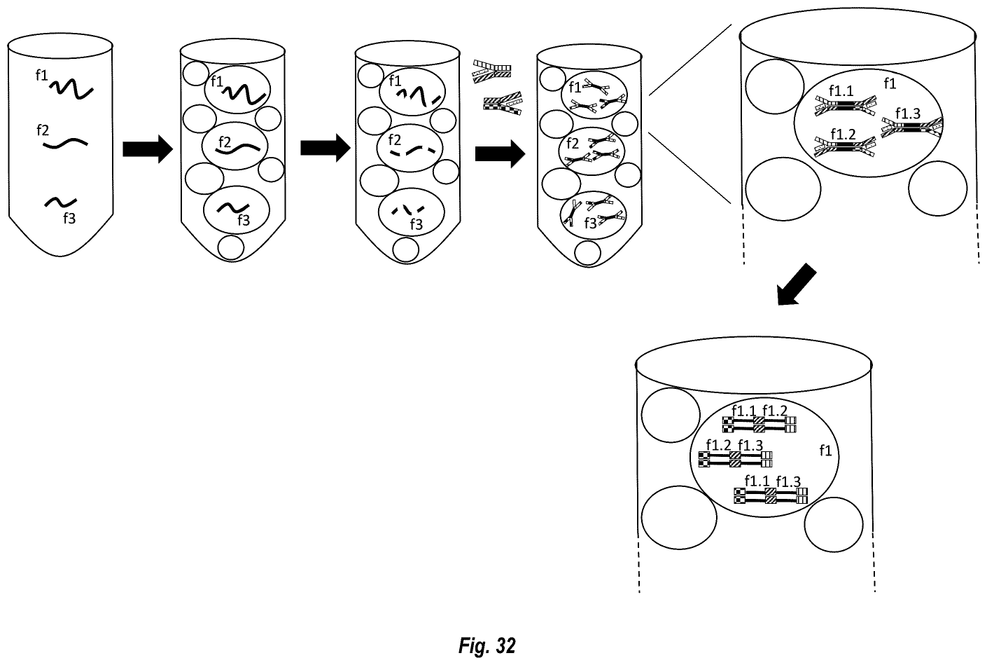

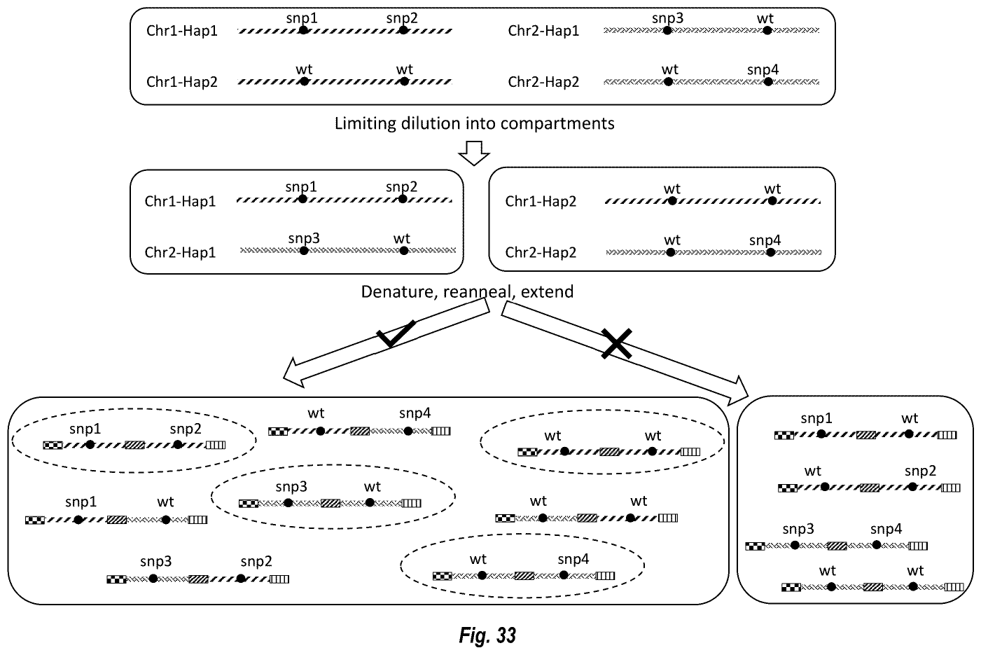

In some embodiments, the limiting dilution reduces the chance that both haplotypes (such as Chr1-Hap1 and Chr2-Hap2 in FIG. 33) are in the same compartment, but the method does not require that only a single chromosome be comprised in a compartment. In other words, the dilution may be to the point that the chance is negligible that two haploid copies of the same chromosome would be comprised in the same compartment (for example less than 5% or less than 1%), but compartments may often comprise more than one chromosome (wherein the more than one chromosome are generally not haploid copies of the same chromosome).

Such a method is shown in FIG. 33, wherein chromosomes are subjected to limiting dilution into compartments, followed by preparation of single-stranded fragments, and then hybridization and extension to prepare concatenated sequencing templates within individual compartments.

In the example shown in FIG. 33, Chr1-Hap1 ends up in a compartment with Chr2-Hap1, but Chr1-Hap2 ends up in a compartment with Chr2-Hap2. Since concatenated sequencing templates are prepared with compartments, these templates can only comprise inserts of chromosomes that were in the same compartment (shown as the box with the checked arrow). Other combinations (shown in the box with the “X” arrow) cannot be formed because these haplotypes were not comprised in the same compartment in this example.

When this method is performed with a sample from an organism with a known genome, the presence of inserts from different chromosomes in the same concatenated sequencing template (because these different chromosomes were comprised in the same compartment during the method) can be resolved from the sequencing data. By analysis to determine the chromosomes that were in the same compartment, information on the alleles comprised in a haploid copy can be determined. In some embodiments, the method does not require barcodes. Instead, the present use of concatenated sequencing templates prepared in compartments allows for analysis of which insert sequences were comprised in a haploid copy without requiring barcodes.

### VI. Methods of Preparing Sequencing Templates Comprising Multiple Inserts Using Transposomes in Solution

In some embodiments, tagmentation is performed in solution to prepare tagged double-stranded fragments. These tagged double-stranded fragments may be used for preparing sequencing templates comprising multiple inserts similarly to methods described above for ligation of forked adapters. In some embodiments, tagged double-stranded fragments are prepared in solution using two pools of transposomes, and the tagged double-stranded fragments are then immobilized on a solid support. In some embodiments, the immobilizing is performed by binding of an affinity moiety that was incorporated in tagged fragments during tagmentation to a binding moiety on a solid support. FIG. 26D shows embodiments of preparing tagged double-stranded fragments in solution using tagmentation, and these tagged double-stranded fragments may be used for preparing concatenated sequencing templates as described above for methods using forked adapters.

In some embodiments, a method of generating one or more concatenated nucleic acid sequencing templates comprises (a) contacting a sample comprising double-stranded target nucleic acid with two pools of transposome complexes in solution; wherein the first pool of transposome complexes comprises a transposase; a first transposon comprising a 3′ transposon end sequence and a first read sequencing adapter sequence; and a second transposon comprising a 5′ sequence fully or partially complementary to the 3′ transposon end sequence and a 3′ complement of a hybridization sequence; and wherein the second pool of transposome complexes comprises a transposase; a first transposon comprising a 3′ transposon end sequence and a second read sequence adapter sequence; and a second transposon comprising a 5′ sequence fully or partially complementary to the 3′ transposon end sequence and a 3′ hybridization sequence.

In some embodiments, one or both second transposons comprise a blocking oligonucleotide. Such blocking oligonucleotides are described above for methods with forked adapters, and the blocking oligonucleotides may be used to inhibit binding of a hybridization sequence comprised in one pool of transposome complexes to the complement of the hybridization sequence in the other pool of transposome complexes.

In some embodiments, the method comprises tagmenting the double-stranded nucleic acids to produce tagged double-stranded fragments; releasing the transposome complex from the double-stranded fragments; and extending and ligating the double-stranded fragments.

In some embodiments, the tagged double-stranded fragments are immobilized on a solid support. In some embodiments, this immobilization is performed by binding of a 5′ affinity moiety comprised in a tag to a binding moiety on the solid support.

In some embodiments, the method then comprises denaturing (1) the immobilized tagged double-stranded fragments to produce immobilized single-stranded fragments and (2) the blocking oligonucleotides to unblock hybridization sequences and complements of hybridization sequences. In some embodiments, after the denaturing, the method comprises hybridizing two immobilized single-stranded fragments to each other to form a bridge by binding of the hybridization sequence in a first fragment to the complement of a hybridization sequence in a second fragment and extending from the 3′ ends of each single-stranded fragment to produce a double-stranded concatenated nucleic acid sequencing template comprising inserts from both immobilized single-stranded fragments.

In some embodiments, the double-stranded concatenated nucleic acid sequencing template comprises an insert sequence and a copy of the insert sequence. In some embodiments, the double-stranded concatenated nucleic acid sequencing template comprises two insert sequences that are different from each other.

The hybridizing of a hybridization sequence in one single-stranded template to the complement of the hybridization sequence in another single-stranded template and extension to prepare concatenated sequencing templates can be performed as described above for forked adapter methods. Essentially, once tagged double-stranded fragments in solution are prepared (either by ligation of forked adapters or by tagmentation in solution), the later steps of immobilizing and preparing bridges and then concatenated sequencing templates can be performed by similar steps.

In some embodiments, hybridizing occurs between single-stranded fragments prepared from double-stranded fragments comprising a tag from a second transposon of a first transposome complex at one end of each fragment and a tag from a second transposon of a second transposome at the other end of each fragment.

In some embodiments, the hybridizing two immobilized single-stranded fragments to each other to form a bridge does not occur between single-stranded fragments prepared from double-stranded fragments comprising a tag from the same transposome complex at both ends of each fragment.

### VII. Methods of Preparing Sequencing Templates Comprising Multiple Inserts Using Solid Supports with Immobilized Transposomes

In some embodiments, sequencing templates comprising multiple inserts are prepared using transposomes immobilized on a solid support. In some embodiments, the solid support is a bead, slide, wall of a vessel, a flow cell, or a nanowell comprised in a flow cell.

As used herein, a “transposome complex” or a “transposome” is comprised of at least one transposase (or other enzyme as described herein) and a transposon recognition sequence. In some such systems, the transposase binds to a transposon recognition sequence to form a functional complex that is capable of catalyzing a transposition reaction. In some respects, the transposon recognition sequence is a double-stranded transposon end sequence. The transposase binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting in a cleavage event. Exemplary transposition procedures and systems can be readily adapted for use with the transposases.

A “transposase” means an enzyme that is capable of forming a functional complex with a transposon end-containing composition (e.g., transposons, transposon ends, transposon end compositions) and catalyzing insertion or transposition of the transposon end-containing composition into a double-stranded target nucleic acid. A transposase as presented herein can also include integrases from retrotransposons and retroviruses.

Transposon based technology can be utilized for fragmenting DNA, wherein target nucleic acids, such as genomic DNA, are treated with transposome complexes that simultaneously fragment and tag the target (“tagmentation”), thereby creating a population of fragmented nucleic acid molecules tagged with unique adapter sequences at the ends of the fragments. Tagmentation includes the modification of DNA by a transposome complex comprising transposase enzyme complexed with one or more tag (such as adapter sequences) comprising transposon end sequences (referred to herein as transposons). Tagmentation thus can result in the simultaneous fragmentation of the DNA and ligation of the adapters to the 5′ ends of both strands of duplex fragments.

A transposition reaction is a reaction wherein one or more transposons are inserted into target nucleic acids at random sites or almost random sites. Components in a transposition reaction may include a transposase (or other enzyme capable of fragmenting and tagging a nucleic acid as described herein, such as an integrase) and a transposon element that includes a double-stranded transposon end sequence that binds to the enzyme, and an adapter sequence attached to one of the two transposon end sequences. One strand of the double-stranded transposon end sequence is transferred to one strand of the target nucleic acid and the complementary transposon end sequence strand is not (i.e., a non-transferred transposon sequence). The adapter sequence can comprise one or more functional sequences (e.g., primer sequences) as needed or desired.

The term “transposon end” refers to a double-stranded nucleic acid DNA that exhibits only the nucleotide sequences (the “transposon end sequences”) that are necessary to form the complex with the transposase or integrase enzyme that is functional in an in vitro transposition reaction. In some embodiments, a transposon end is capable of forming a functional complex with the transposase in a transposition reaction. As non-limiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands. Although the term “DNA” is used throughout the present disclosure in connection with the composition of transposon ends, it should be understood that any suitable nucleic acid or nucleic acid analogue can be utilized in a transposon end.

The term “transferred strand” refers to the transferred portion of both transposon ends. Similarly, the term “non-transferred strand” refers to the non-transferred portion of both “transposon ends.” The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transposon is a forked adapter transposon. A forked adapter transposon comprises two strands. In some embodiments, the second strand of the forked adapter transposon comprises an adapter sequence and a sequence fully or partially complementary to the first strand of the first forked adapter transposon. The sequence with full or partial complementarity in the first and second strands allow for the two strands to hybridize together and form the forked structure.

In some embodiments, more than one type of transposome complexes is immobilized on the surface of a solid support. In some embodiments, fragments can be prepared with different tags based on use of different transposomes.

In some embodiments, a solid support comprises two pools of immobilized transposome complexes. In some embodiments, a first pool of transposome complexes comprises (a) a transposase; (b) a first transposon comprising a 3′ transposon end sequence, a first read sequencing adapter sequence, and a 5′ affinity moiety; and (c) a second transposon comprising a 5′ sequence fully or partially complementary to the 3′ transposon end sequence and a 3′ complement of a hybridization sequence. In some embodiments, a second pool of transposome complexes comprises (a) a transposase; (b) a first transposon comprising a 3′ transposon end sequence, a second read sequence adapter sequence, and a 5′ affinity moiety; and (c) a second transposon comprising a 5′ sequence fully or partially complementary to the 3′ transposon end sequence and a 3′ hybridization sequence. In some embodiments, each first transposon is immobilized by binding of a 5′ affinity moiety to a binding moiety on the surface of the solid support.

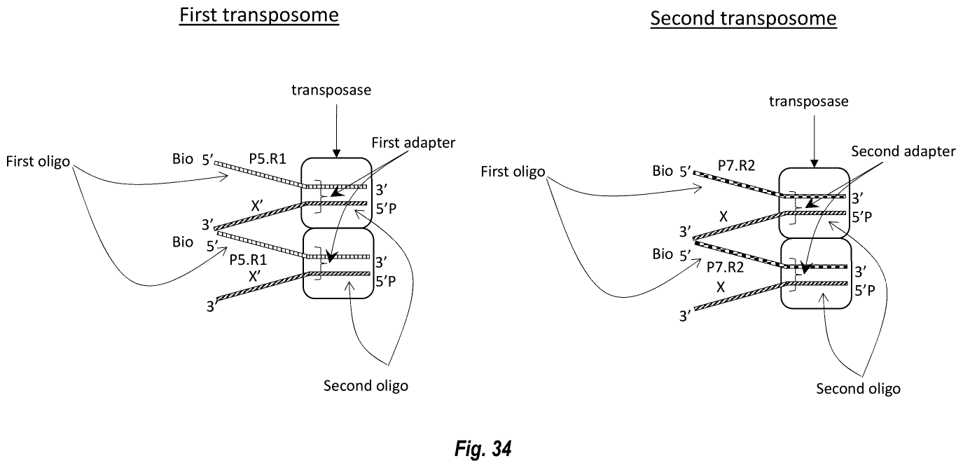

In some embodiments, a first pool of immobilized transposome complexes comprises first forked adapter comprising a first oligonucleotide comprising P5.R1 and a second oligonucleotide comprising a X′ (complement of a hybridization sequence). In some embodiments, a second pool of immobilized transposome complexes comprises a second forked adapter comprising a first oligonucleotide comprising P7.R2 and a second oligonucleotide comprising a X (hybridization sequence). Such an exemplary embodiment is shown in FIG. 34.

In some embodiments, a transposome complex comprises a dimer of two molecules of a transposase. In some embodiments, transposome complexes comprise homodimers and/or heterodimers.

In some embodiments, a transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. As used herein, “homodimers” refers to a transposome dimer that comprises the same transposon sequences at both sites. In some embodiments, the compositions and methods described herein employ a population of transposome complexes assembled by contacting a first forked adapter with a transposase to prepare a first transposome complex and contacting a second forked adapter with a transposase to assemble a second transposome complex and then pooling together the first and second transposome complexes. In some embodiments, a pool of transposome complexes comprises homodimers comprising a first forked adapter and homodimers comprising a second forked adapter.

In some embodiments, a transposome complex is a heterodimer, wherein two molecules of a transposase are each bound to a different forked adapter comprising a first and second transposon (e.g., the sequences of the two transposons bound to each monomer of a transposome complex are different, forming a “heterodimer”).

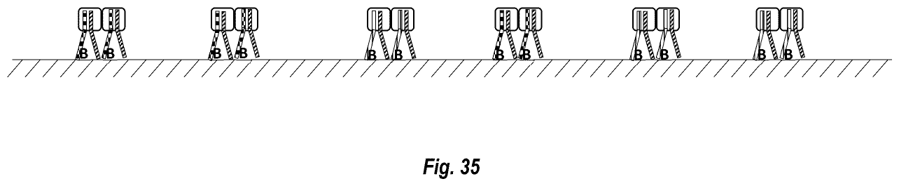

In some embodiments, the compositions and methods described herein employ a population of transposome complexes assembled by pooling a first forked adapter and a second forked adapter together with transposases to assemble the pool of transposome complexes. After this pooling, the predicted ratio of assembled transposome complexes would be 25% transposome complexes that are homodimers comprising the first forked adapter, 25% transposome complexes that are homodimers comprising the second forked adapter, and 50% transposome complexes that are heterodimers comprising the first forked adapter and the second forked adapter. In some embodiments, the first and/or second pool of transposome complexes are homodimers or heterodimers. In some embodiments, the first and the second pool of transposome complexes are homodimers or heterodimers. Exemplary homodimers, heterodimers, and solid supports comprising immobilized homodimers and their methods of use are disclosed in U.S. Pat. No. 9,683,230, which is incorporated herein in its entirety. FIG. 35 shows an exemplary solid support comprising two pools of homodimers, wherein all homodimers are immobilized on the surface of a solid support. A pool of two homodimers or a pool comprising heterodimers may be used to generate tagged double-stranded fragments wherein at least some fragments comprise a tag from a transposome complex comprised in a first pool at one end and a tag from a transposome complex comprised in a second pool at the other end.

In some embodiments, one or more transposons comprise at least one of an adapter, a barcode sequence, a unique molecular identifier (UMI) sequence, a capture sequence, or a cleavage sequence. In other words, transposons may comprise additional sequences of use in methods that a user wants to perform, such as sequencing. In some embodiments, one or more transposons comprises an index sequence and/or a UMI. In some embodiments, one or more transposons comprises an index sequence and a UMI. Transposons comprising UMIs and their methods of use are described in WO 2019/108972, WO 2018/136248, WO2016176091, and WO202014437, each of which is incorporated in its entirety herein.

In some embodiments, a first transposon comprised in a first pool of transposome complexes and/or a first transposon comprised in a second pool of transposome complexes comprise sample indexes. In some embodiments, both a first transposon comprised in a first pool of transposome complexes and a first transposon comprised in a second pool of transposome complexes comprise sample indexes. In a representative example, an embodiment may include a first transposon comprising i5 that is comprised in a first pool of transposome complexes and a first transposon comprising i7 that is comprised in a second pool of transposome complexes, as shown in FIG. 46A.

In some embodiments, a second transposon comprised in a first pool of transposome complexes and/or a second transposon comprised in a second pool of transposome complexes comprise sample indexes and/or UMIs. In some embodiments, both a second transposon comprised in a first pool of transposome complexes and a second transposon comprised in a second pool of transposome complexes comprise sample indexes.

In some embodiments, both a second transposon comprised in a first pool of transposome complexes and a second transposon comprised in a second pool of transposome complexes comprise UMIs. In a representative example, an embodiment may include a second transposon comprising i8 that is comprised in a first pool of transposome complexes and a second transposon comprising i6 that is comprised in a second pool of transposome complexes, wherein i6 and i8 function as UMIs, as shown in FIG. 46B.

In some embodiments, the first and second transposons comprised in both a first pool and a second pool of transposomes may comprise either a sample index sequence or a UMI. When such transposomes are used in the present methods, a polynucleotide such as shown in FIG. 46C may be produced.

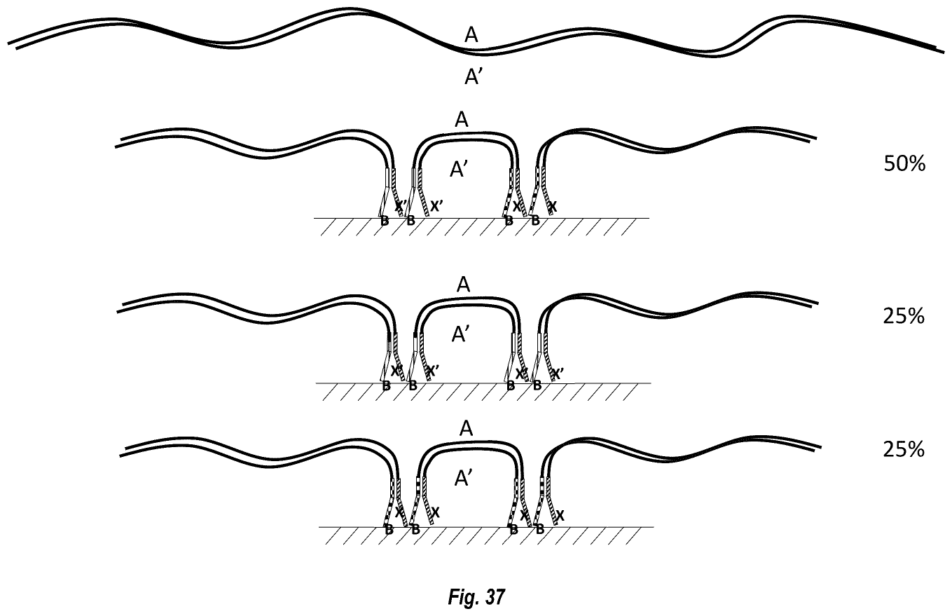

In some embodiments, a method of generating one or more double-stranded concatenated nucleic acid sequencing templates (as shown in FIG. 37) comprises applying a sample comprising a double-stranded nucleic acid immobilized to a solid support and tagmenting the double-stranded nucleic acids to produce tagged double-stranded fragments comprising inserts from the double-stranded nucleic acid, wherein the double-stranded fragments are immobilized to the solid support by binding of the 5′ affinity moieties to a binding moiety on the surface of the solid support. In some embodiments, the 5′ affinity moiety is comprised in the first transposon (i.e., the first strand of a forked adapter comprised in a transposome complex).

In some embodiments, transposome complexes are then released from the double-stranded fragments. In some embodiments, releasing the transposome complex from the double-stranded fragments is performed with SDS and washing.

In some embodiments, the method comprises extending and ligating the double-stranded fragments after releasing the transposome complexes. In some embodiments, extending and ligating comprises providing polymerase, dNTPs, and extension buffer (ELMT).

In some embodiments, the method comprises denaturing the extended and ligated double-stranded fragments into single-stranded fragments, wherein single-stranded fragments comprising a 5′ affinity moiety remain immobilized on the solid support as shown in FIG. 38. In some embodiments, the denaturing comprises heating the solid support or applying a chemical denaturant. In some embodiments, the denaturing comprises increasing the temperature of the solid support to 90° C. or warmer.

In some embodiments, the method comprises allowing hybridization of a hybridization sequence comprised in a first immobilized single-stranded fragment to a complement of a hybridization sequence comprised in a second immobilized single-stranded fragment thereby forming a bridge. In some embodiments, allowing hybridization comprises cooling the solid support and/or applying a hybridization buffer. In some embodiments, the cooling comprises reducing the temperature of the solid support to 60° C. or cooler. In some embodiments, the hybridization buffer comprises a high salt concentration, optionally wherein the high salt concentration is 750 mM NaCl.

In some embodiments, a hybridization sequence (X or HYB) comprised in a first single-stranded fragment can hybridize to the complement of a hybridization sequence (X′ or HYB′) comprised in a second single-stranded fragment. In some embodiments, the hybridization sequence and/or its complement is bound to a blocking oligonucleotide that is fully or partially complementary to the hybridization sequence or its complement, and the denaturing comprises denaturing the blocking oligonucleotide to unblock the hybridization sequence and/or its complement. Such blocking oligonucleotides can function as described above for forked adapters, wherein association of a hybridization sequence to its complement is blocked until the blocking oligonucleotide is denatured. In some embodiments, a forked adapter comprised in a transposome comprises 3 oligonucleotides, wherein 2 oligonucleotides comprise the first and second transposon of the forked transposon and the third oligonucleotide is a blocking oligonucleotide. In some embodiments, a blocking oligonucleotide (such as XB or X′B′) is hybridized to the forked adapter transposon at the 3′ended single stranded section of the second transposon. This blocking oligonucleotide may be hybridized to either, or both, the first and second adapter of a forked adapter transposon. In some embodiments, a blocking oligonucleotide prevents a first forked adapter transposon and second forked adapter transposon from hybridizing to one another via the 3′ complementary section of the second oligonucleotides. In some embodiments, the blocking oligonucleotide comprises nucleotides that are not a target for tagmentation.

In some embodiments, binding of a HYB comprised in a first immobilized single-stranded fragment to a HYB′ comprised in a second immobilized single-stranded fragment may be termed “bridging” (similarly to how this term is used in methods using forked adapters).

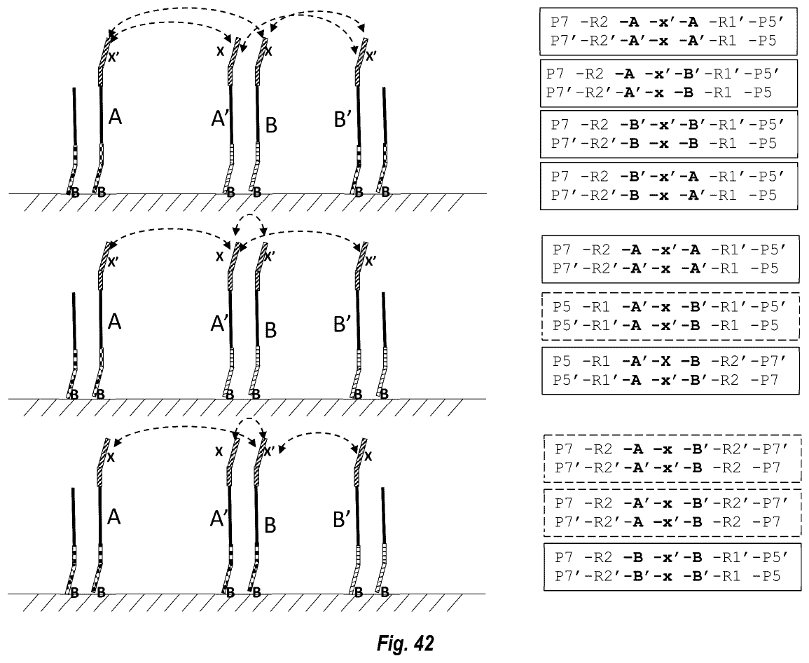

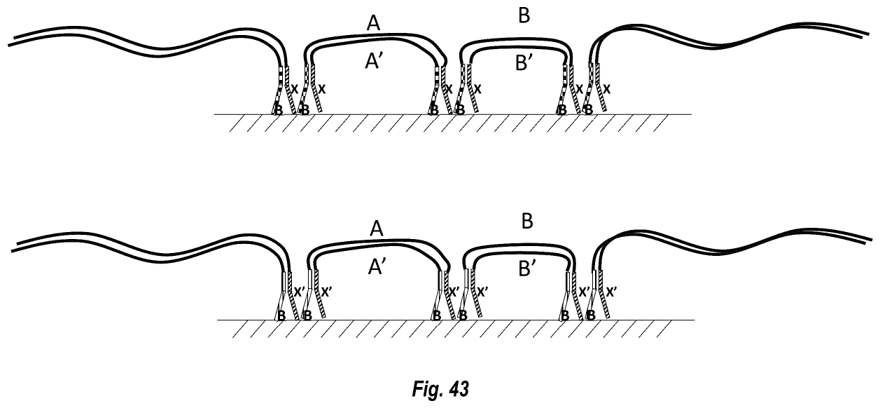

In some embodiments, a fragment comprising a X sequence can hybridize to a X′ sequence in other fragment (as shown in FIGS. 42 and 45). In some embodiments, fragments that comprise adapters incorporated from only the forked adapter comprised in the second transposome or from only the forked adapter comprised in the first transposome cannot bridge together (as shown in FIGS. 43 and 44).

In some embodiments, after bridging of two single-stranded fragments, a method comprises extending and generating a double-stranded concatenated nucleic acid sequencing template.

In some embodiments, a method comprises additional rounds of allowing hybridization and extending and generating a double-stranded concatenated nucleic acid sequencing template. In other words, the step of allowing bridging between two immobilized single-stranded fragments can be repeated until no more double-stranded concatenated nucleic acid sequencing templates can be prepared. The number of double-stranded concatenated nucleic acid sequencing templates prepared may be limited by the number of single-stranded fragments immobilized in close proximity with complementary HYB/HYB′ sequences. Once no more single-stranded fragments can partner with other single-stranded fragments, no more additional concatenated sequencing templates can be prepared.

In some embodiments, concatenated sequencing templates prepared using immobilized transposomes comprise two copies of the same insert. In some embodiments, a high ratio of DNA to transposomes leads to a high proportion of concatenated sequencing templates comprising two copies of the same insert. In some embodiments, DNA is pre-fragmented into short fragments less than 1000 bp in length before tagmentation by immobilized transposomes to produce a high proportion of concatenated sequencing templates comprising two copies of the same insert. Under such conditions, the outcome will be predominantly single-stranded fragments comprising sense and antisense complementary sequences that hybridize together, such that extension produces a concatenated sequencing template comprising two copies of the same insert.

In some embodiments, concatenated sequencing templates comprise two inserts that are not copies of each other. In some embodiments, the inserts comprised in a concatenated sequencing template are different. In some embodiments, concatenated sequencing templates comprising two different inserts are used to generate proximity data using the methods outlined below.

A. Fragmenting of Proximal or Contiguous Regions of a Double-Stranded Nucleic Acid by Spatially Localized Transposomes

Binding of double-stranded nucleic acids to transposases comprised in transposome complexes is random, but a given double-stranded nucleic acid would be fragmented by transposomes that are immobilized in a specific area of the surface of the solid support. This aspect of the method is outlined in FIG. 45, wherein regions A-E are ordered in one double-stranded nucleic acid and thus produce bridged fragments when tagmented. This double-stranded nucleic acid imposes a spatial limitation, wherein once a first region of the double-stranded nucleic acid is bound to a transposome complex in a given region of the surface, the rest of the double-stranded nucleic acid is only free to bind to transposome complexes in this region. The ability to preserve genomic connectivity information based on the location of fragments on the surface of a solid support with immobilized transposomes is disclosed in U.S. Pat. No. 10,246,746, which is incorporated by reference herein in its entirety.

In sum, different fragments from the same double-stranded nucleic acid can be tagmented and immobilized across neighboring transposome complexes, as shown in FIG. 45. Thus, fragments comprising inserts prepared from a double-stranded nucleic acid will be immobilized in a spatial relationship based on how close or far these inserts sequences were in the double-stranded nucleic acid before tagmentation.

B. Proximity of Immobilized Single-Stranded Fragments for Bridging

Because single-stranded nucleic acids prepared using immobilized transposomes are immobilized before forming bridges between a HYB in a first single-stranded fragment and a HYB′ in a second single-stranded fragment, the first and second fragments that join in a bridge must be immobilized in close proximity on the surface of the solid support. For example, the first and second fragments may be the sense and antisense strands produced from the same double-stranded fragment. This is shown in FIGS. 38 and 39, wherein complementary single-stranded fragments from a double-stranded fragment immobilized at both ends may be denatured and then may reanneal to each other when hybridization is allowed. As shown in FIG. 40, hybridizing of single-stranded inserts (such as those comprising A and A′) can lead to generation of a concatenated sequencing template after extension. In contrast, no template will be prepared between two fragments both comprising X′ or both comprising X.

In some embodiments, single-stranded fragments prepared from different double-stranded fragments may be in close enough proximity to hybridize to each other for bridging. In essence, both the first and second single-stranded fragment are tethered to the surface of the solid support at their 5′ ends, so the free 3′ ends of each fragment (comprising HYB or HYB′) must be able to reach each other to interact. If the 3′ ends of two immobilized fragments cannot reach each other because they are immobilized too far apart on the surface of the solid support, a HYB/HYB′ bridge cannot be formed between these two fragments.

Accordingly, if the distance between two immobilized fragments is greater than the length of the longer fragment, there is no way for these fragments to interact, as their HYB/HYB′ sequences could not overlap. In some embodiments, hybridization of a hybridization sequence comprised in a first immobilized single-stranded fragment to a complement of a hybridization sequence comprised in a second immobilized single-stranded fragment only occurs when the first and second fragment are at a proximity to each other on the surface of the solid support that is closer than the length of the longer of the first or second fragment.

In some embodiments, a sufficient number of nucleotides comprised in a HYB in a first single-stranded fragment must be able to hybridize to a HYB′ in a second single-stranded fragment. If no nucleotides between the HYB in a first single-stranded fragment and a HYB′ in a second single-stranded fragment can hybridize with each other, then these two fragments cannot produce a bridge. In some embodiments, the first immobilized fragment and the second immobilized fragment are immobilized in close proximity on the solid support, wherein the close proximity allows binding of 4 or more, 5 or more, 6 or more, 7 or more, 8 or more, 9 or more, or 10 or more nucleotides comprised in the hybridization sequence comprised in the first immobilized fragment to nucleotides comprised in the complement of the hybridization sequence comprised in the second immobilized fragment.

In some embodiments, the first immobilized fragment and the second immobilized fragment are immobilized within 20 to 500 nanometers of each other on the surface of the solid support. In some embodiments, the first immobilized fragment and the second immobilized fragment are immobilized within 20 to 300 nanometers of each other on the surface of the solid support. In some embodiments, immobilized single-stranded fragments that are within 500 nanometers are fewer may be able to bridge with each other via binding of a HYB in one fragment to a HYB′ in the other fragment. In some embodiments, two immobilized fragments from sequences that were adjacent in a double-stranded nucleic acid may be adjacent on the surface of the solid support without a different fragment being immobilized between them.

In some embodiments, a sample comprises multiple different double-stranded nucleic acids. In some embodiments, spatially localized fragments are prepared from the same double-stranded nucleic acid. In some embodiments, both the first and the second immobilized fragments are prepared from the same double-stranded nucleic acid, and the double-stranded concatenated nucleic acid sequencing template comprises two inserts from the same double-stranded nucleic acid.

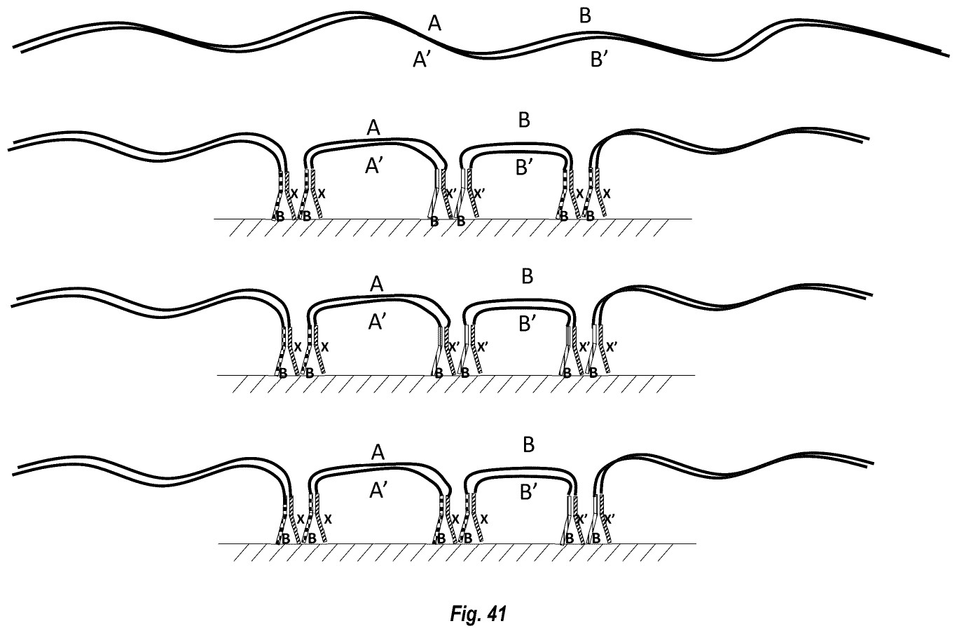

In some embodiments, the two inserts are from two contiguous sequences comprised in the same double-stranded nucleic acid (such as the bridged fragments shown in FIG. 41). For example, FIG. 42 shows single-stranded fragments comprising an A or A′ insert bridging with themselves or bridging with single-stranded fragments comprising a B or B′ sequence, wherein both the A/A′ and B/B′ fragments are prepared from neighboring sequences in the same double-stranded nucleic acid. Such pairings will be based on hybridization of a X sequence in one fragment to a X′ sequence in another fragment. After extension, a double-stranded concatenated sequencing template may be prepared. At least some of the concatenated sequencing templates will be sequenceable based on the presence of P5/P5′ at one end and P7/P7′ at the other end (as shown in the boxes outlined with a solid line in FIG. 42). Other concatenated sequencing templates that may be produced will not generally be sequenceable as they have the same complementary adapter sequences at both ends of templates (such as P5/P5′ or P7/P7′, as shown in templates in the dashed boxes in FIG. 42).

When a sequencing template is released from the solid support and sequenced, the presence of A and B inserts in a single-stranded template (and A′ and B′ inserts in another single-stranded template) can be used to indicate that A and B sequences are in close proximity in the same double-stranded nucleic acid. For example, the A and B sequences may be determined to have been in the same target nucleic acid.

FIG. 43 shows bridged tagmentation reactions that occur randomly with identical transposomes (i.e., comprising the same transposons). As shown in FIG. 44, the resulting single-stranded fragments will not be able to hybridize and bridge with one another, because the resulting single stranded fragments comprise only X (top panel) or X′ (bottom panel) sequences. In the absence of some single-stranded fragments comprising X and some single-stranded fragments comprising X′, no bridging would be expected with no generation of double-stranded concatenated sequencing templates.

In some embodiments, the concentration of double-stranded nucleic acid in a sample applied to the solid support is low enough to generally avoid single-stranded fragments from different double-stranded nucleic acid polynucleotides being in close enough proximity to bridge together. In this way, most fragments that bridge together (and allow for preparation of double-stranded concatenated sequencing templates) are those from double-stranded fragments prepared from the same double-stranded nucleic acid polynucleotide and not from another double-stranded polynucleotide in the same sample. In this way, concatenated sequencing templates that comprise fragments from unrelated double-stranded nucleic acids can generally be avoided when using methods with immobilized transposomes if the user prefers.

In some embodiments, the two inserts comprised in a first single-stranded fragment and a second single-stranded fragment that form a bridge between their HYB/HYB′ are from non-contiguous regions of the same nucleic acid. In some embodiments, the two inserts in a first single-stranded fragment and a second single-stranded fragment that form a HYB/HYB′ bridge are from two proximal sequences comprised in the same double-stranded nucleic acid. In some embodiments, the proximal sequences are separated by 100 or less nucleotides, 200 or less nucleotides, 300 or less nucleotides, 400 or less nucleotides, 500 or less nucleotides, 700 or less nucleotides, or 1,000 or less nucleotides in the double-stranded nucleic acid. Such relatively small distances between proximal sequences leads to a high likelihood that single-stranded fragments from these sequences may be able to bridge with each other and generate concatenated nucleic acid sequencing templates.

In some embodiments, an area of the solid support comprises multiple double-stranded concatenated nucleic acid sequencing template that share common insert sequences from proximal sequences comprised in the same double-stranded nucleic acid. Using the example nucleic acid shown in FIG. 45, the spatial relationship of fragments A-E can be resolved using sequencing data from the concatenated sequencing templates that may be prepared. FIG. 45 shows possible pairing using a 1-dimensional illustration, but one must appreciate that these interactions happen on a 2-dimensional plane (X,Y). Further, the fragments may be localized on the surface because a nucleic acid bound to an initial transposome could be twisted back on itself multiple times in a serpentine arrangement before binding to other transposomes. Accordingly, the final pairing of sequences may be based on this serpentine arrangement of single-stranded fragments on the surface.

In some embodiments, the proximity of sequences (such as A-E in FIG. 45) can be resolved by analysis of which fragments comprising these sequences can bridge to form concatenated sequencing templates.

In some embodiments, fragments that are closer on the surface of the solid support (because they were prepared from fragments that were in close proximity in the double-stranded nucleic acid that was tagmented) will bridge together with a higher frequency than those that are further away. Accordingly, neighboring fragments will generally bridge with the highest frequency to form concatenated sequencing templates (excluding reannealing of single-stranded fragment prepared with the same insert including their insert sequences as shown in FIG. 39, which will not produce a concatenated sequencing template and reannealing of single-stranded fragment prepared with the same insert by bridging of the hybridization sequencing in one fragment to its complement in the other as shown in FIG. 40) based on the serpentine arrangement on the surface of single-stranded fragments produced from a given double-stranded nucleic acid. As the distance between two sequences in a double-stranded nucleic acid that was fragmented increases, the distance between single-stranded fragments comprising these sequences as inserts on the surface of the solid support will generally increase as well, as shown in FIG. 45. Thus, the frequency of generated concatenated sequencing templates comprising two different inserts (or their complements) will allow analysis of proximity information in the double-stranded nucleic acid that is tagmented.

Neighboring sequences will be estimated to have greater frequency of being comprised in the same concatenated sequencing template as compared to sequences that were farther apart, and this frequency will decrease as the distance between the fragments increases. It follows then that any two sequences that are separated by too large a distance in the double-stranded nucleic acid that is tagmented will not be able to bridge and form a concatenated sequencing template. The lack of these concatenated sequencing templates in sequencing data can thus be interpreted as too far a distance to form bridges between single-stranded fragments comprising a given pair of inserts.

FIG. 45 shows how bridged fragments prepared with immobilized transposomes can lead to denatured single-stranded fragments that can hybridize to each other based on binding of X to X′. The bridging of single-stranded fragments (which can then generate concatenated sequencing templates) can be used to “walk” down the sequence of the double-stranded nucleic acid that was tagmented. Thus, the compiled sequencing data of the pool of concatenated sequencing templates formed on the surface can be used to form a representation of the double-stranded nucleic acid that is tagmented.

Single-stranded fragments formed from the same double-stranded fragment (such as those comprising A and A′ in FIG. 40) can bridge with each other and then form a concatenated sequencing template comprising two copies of the same insert sequence. Such concatenated sequencing templates comprising two copies of the same insert can be used for error correction, identification of mutations that are only present in a single strand, and methylation analysis, as described herein.

In some embodiments, gaps in the nucleic acid sequence left after the tagmentation event may be filled using an extending step. In general, an extending step is followed by a ligating step. Extending and/or ligating are performed using appropriate conditions. In some embodiments, the buffer used is an extension-ligation mix buffer (e.g., extension-ligation mix buffer 3, ELM3). A polymerase such as T4 DNA pol Exo- (New England BioLabs, Catalog #M0203S) or Ttaq608 may be used in said extending and/or ligating step.

C. Representative Structures of Sequencing Templates Prepared Using Immobilized Transposomes

A user can design transposons comprising forked adapters to incorporate sequences of interest (such as adapters, primer binding sites, etc.). These sequences of interest can be selected by the user based on, for example, what sequencing platform they prefer to use and the requirements for sequencing templates on this platform.

Representative first and second forked adapters that may be comprised in transposomes for preparing sequencing templates described herein are shown in FIGS. 46A and 46B. FIGS. 46A-46C also show the structures of representative sequencing templates that may be produced with such transposomes.

In some embodiments, a sequencing template prepared using immobilized transposomes has a structure of:
5′-P545-A14-ME-Insert1-ME′-HYB-ME-Insert2-ME′-B15′-i7′-P7′-3′; a13395
5′-P5-A14-ME-Insert1-ME′-i6-HYB-i8′-ME-Insert2-ME′-B15′-P7′-3′; or
5′-P545-A14-ME-Insert1-ME′-i6-HYB-i8′-ME-Insert2-ME′-B15′47′-P7′-3′, or their complements.

D. Amplification

In some embodiments, the method comprises amplifying the generated double-stranded sequencing templates after releasing them from the surface of the solid support and before sequencing.

In some embodiments, sequencing templates are amplified using cluster amplification methodologies as exemplified by the disclosures of U.S. Pat. Nos. 7,985,565 and 7,115,400, the contents of each of which is incorporated herein by reference in its entirety. The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules. Each cluster or colony on such an array is formed from a plurality of identical immobilized polynucleotide strands and a plurality of identical immobilized complementary polynucleotide strands. The arrays so-formed are generally referred to herein as “clustered arrays.” The products of solid-phase amplification reactions such as those described in U.S. Pat. Nos. 7,985,565 and 7,115,400 are so-called “bridged” structures formed by annealing of pairs of immobilized polynucleotide strands and immobilized complementary strands, both strands being immobilized on the solid support at the 5′ end, in some embodiments via a covalent attachment. Cluster amplification methodologies are examples of methods wherein an immobilized nucleic acid template is used to produce immobilized amplicons. Other suitable methodologies can also be used to produce immobilized amplicons from sequencing templates produced according to the methods provided herein. For example, one or more clusters or colonies can be formed via solid-phase PCR whether one or both primers of each pair of amplification primers are immobilized.

In other embodiments, sequencing templates are amplified in solution. For example, in some embodiments, the nucleic acid fragments are cleaved or otherwise liberated from the solid support and amplification primers are then hybridized in solution to the liberated molecules. In other embodiments, amplification primers are hybridized to the nucleic acid fragments for one or more initial amplification steps, followed by subsequent amplification steps in solution. Thus, in some embodiments an immobilized nucleic acid template can be used to produce solution-phase amplicons.

It will be appreciated that any of the amplification methodologies described herein or generally known in the art can be utilized with universal or target-specific primers to amplify the sequencing templates. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354, which is incorporated herein by reference in its entirety. The above amplification methods can be employed to amplify one or more nucleic acids of interest. For example, PCR, including multiplex PCR, SDA, TMA, NASBA and the like can be utilized to amplify the sequencing templates. In some embodiments, primers directed specifically to the nucleic acid of interest are included in the amplification reaction.

### VIII. Methods of Preparing Sequencing Templates Comprising Multiple Inserts Using Transposomes in Solution Within Compartments

Methods of evaluating proximity data of sequences within a double-stranded nucleic acid may also be performed with compartments, using compartments as described above for methods with forked adapters. In some embodiments, the compartments are wells, tubes, or droplets.

In some embodiments, transposomes within compartments are in solution. In some embodiments, transposomes are not immobilized on a solid support when preparing sequencing templates in compartments.

In some embodiments, since double-stranded fragments are not immobilized before preparing single-stranded fragments, methods with transposomes in compartments generally prepare concatenated sequencing templates comprising two different inserts. This is because the selection pressure of having the two single-stranded fragments prepared from the same double-stranded fragment in close proximity of a solid support is lost when the fragments are not immobilized and instead tagmentation happens in a solution-phase.

In some embodiments, two pools of transposomes may be used. In some embodiments, a first transposome and a second transposome as shown in FIG. 34 may be used.

In some embodiments, a method of generating one or more concatenated nucleic acid sequencing templates comprises compartmentalizing a sample comprising target double-stranded nucleic acid into a plurality of different compartments and tagmenting the double-stranded nucleic acids to produce tagged double-stranded fragments comprising inserts from the double-stranded nucleic acid within the plurality of different compartments.

In some embodiments, the tagmenting is performed with two pools of transposome complexes. In some embodiments, the first pool of transposome complexes comprises (a) a transposase; (b) a first transposon comprising a 3′ transposon end sequence and a first read sequencing adapter sequence; and (c) a second transposon comprising a 5′ sequence fully or partially complementary to the 3′ transposon end sequence and a 3′ complement of a hybridization sequence. In some embodiments, the second pool of transposome complexes comprises (a) a transposase; (b) a first transposon comprising a 3′ transposon end sequence and a second read sequence adapter sequence; and (c) a second transposon comprising a 5′ sequence fully or partially complementary to the 3′ transposon end sequence and a 3′ hybridization sequence. In some embodiments, tagmentation prepares tagged double-stranded fragments. In some embodiments, a first single-stranded fragment comprises an insert and a second fragment comprises an insert that is not the complement of the insert comprised in the first fragment.

In some embodiments, the method comprises denaturing the tagged double-stranded fragments to produce single-stranded fragments, hybridizing two single-stranded fragments within the same compartment to each other by binding of the hybridization sequence in a first fragment to the complement of a hybridization sequence in a second fragment, and extending from the 3′ ends of each single-stranded fragment to produce a double-stranded concatenated nucleic acid sequencing template comprising inserts from both single-stranded fragments. In some embodiments, templates are released from compartments before further processing.

In some embodiments, double-stranded concatenated nucleic acid sequencing templates are only produced from hybridizing of two single-stranded fragments present in the same compartment. In other words, only single-stranded fragments in the same compartment can hybridize together, and single-stranded fragments in different compartments are not available to associate with each other. In some embodiments, the compartmentalizing comprises dilution of the sample such that most compartments comprise one or no target double-stranded nucleic acid. In this way, insert sequences that are comprised in the same concatenated sequencing template are likely to have been comprised in the same target nucleic acid.

In this way, a user can identify that two sequences comprised in the same concatenated sequencing template originated from the same target nucleic acid. Such ability to identify sequences that originated from the same target nucleic acid can help to the sequences that comprise a given target nucleic acid.

In some embodiments, wherein the compartmentalizing separates different haplotypes into different compartments and the method is used for haplotype phasing. In other words, a user could evaluate sequences comprised in the same concatenated sequencing template and determine that these sequences were comprised in the same haplotype. In some embodiments, the haplotype phasing does not require barcodes.

In some embodiments, the hybridization sequence and/or its complement is bound to a blocking oligonucleotide that is fully or partially complementary to the hybridization sequence or its complement, and the denaturing comprises denaturing the blocking oligonucleotide to unblock the hybridization sequence and/or its complement. Such blocking oligonucleotides are described above for methods with forked adapters. In some embodiments, one or more blocking oligonucleotides inhibit association of first transposomes with second transposomes in solution. In other words, the timing of association of the hybridization sequence and its complement can be controlled to happen only after single-stranded tagged fragments are prepared.

In some embodiments, the denaturing is performed with an increase in temperature, change in pH, and/or addition of one or more chaotropic agents. In some embodiments, the increase in temperature is an increase from 45° C.-55° C. to 85° C.-95° C., optionally wherein the increase in temperature is an increase from 50° C. to 90° C. In some embodiments, the one or more chaotropic agents comprise formamide and/or NaOH.

In some embodiments, one or more additional rounds of denaturing, hybridizing, and extending are performed. In other words, rounds of denaturing, hybridizing, and extending may be repeated until there are no single-stranded fragments available for hybridizing with other single-stranded fragments.

In some embodiments, the method further comprising amplifying the templates.

### IX. Methods of Sequencing a Concatenated Nucleic Acid Sequence Template

In some embodiments, a method comprises sequencing a concatenated nucleic acid sequence template. In some embodiments, tandem reads are generated by sequencing a concatenated nucleic acid sequence template.

In some embodiments, the sequences of different inserts are generated sequentially. In some embodiments, a method of sequencing a concatenated nucleic acid sequencing template comprises sequencing the first insert sequence and sequencing the second insert sequence.

In some embodiments, a method of sequencing a concatenated nucleic acid sequencing template comprises sequencing the first insert sequence of a polynucleotide by initiating sequencing with a first read sequencing primer complementary to the first read primer binding sequence; and sequencing the second insert sequence by initiating sequencing with a second read sequencing primer complementary to the second read primer binding sequence. An exemplary method is presented in FIG. 2, wherein the “Read 1” sequencing primer is used to sequence the first insert sequence (located between the P5′ and HYB sequences in the polynucleotide) and the “Read 2” sequencing primer is used to sequence the second insert sequence (located between the HYB′ and P7′ sequences in the polynucleotide).

In some embodiments, the first and second insert sequences may be generated from separate libraries (“Library A” and “Library B,” as shown in FIG. 3).

In some embodiments, a method of sequencing a concatenated nucleic acid sequencing template comprises sequencing the complement of the second insert sequence and then sequencing the complement of the first insert sequence.

In some embodiments, a method of sequencing a concatenated nucleic acid comprises sequencing the complement of the second insert sequence by initiating sequencing with a first complement read sequencing primer complementary to the first complement read primer binding sequence; and sequencing the complement of the first insert sequence by initiating sequencing with a second complement read sequencing primer complementary to the second complement read primer binding sequence.

In some embodiments, more than two insert sequences or more than two complements of insert sequences from a polynucleotide may be sequenced.

The polynucleotides comprising multiple insert sequences described herein can be sequenced according to any suitable sequencing methodology, such as direct sequencing or next generation sequencing, including sequencing by synthesis, sequencing by ligation, sequencing by hybridization, sequencing based on detection of released protons can use an electrical detector and associated techniques that are commercially available from Ion Torrent (Guilford, CT, a Life Technologies subsidiary), nanopore sequencing and the like. In some embodiments, the DNA fragments are sequenced on a solid support, such as a flow cell. Exemplary SBS procedures, fluidic systems, and detection platforms that can be readily adapted for use with polynucleotides comprising multiple insert sequences of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

The methods described herein are not limited to any particular type of sequencing instrumentation used.

### X. Methods of Use of Sequencing Templates Comprising Multiple Inserts

In some embodiments, sequencing templates comprising multiple inserts are used to determine the sequences of two or more inserts from a double-stranded nucleic acid.

In some embodiments, sequencing templates comprising two or more inserts are used to produce multiple copies of the sequence of an insert from a double-stranded nucleic acid. Although each sequence from an insert comprised in such a template would be expected to have the same sequence, it is well-known a variety of different artifacts can lead to an incorrect sequence. For example, an error that is introduced into an amplicon produced from a sequencing template during amplification can cause a discrepancy in a sequence that is not related to a different in the double-stranded nucleic acid used to prepare inserts.

A. Sequencing

In some embodiments, a method comprises releasing generated double-stranded concatenated nucleic acid sequencing templates from the solid support and sequencing the templates to determine insert sequences comprised in the templates. In some embodiments, the releasing comprising enzymatic digestion or chemical cleavage. Such means of releasing sequencing templates from the surface of a solid support are well-known in the art.

The incorporated materials of U.S. Pat. Nos. 7,985,565 and 7,115,400 describe methods of solid-phase nucleic acid amplification which allow amplification products to be immobilized on a solid support in order to form arrays comprised of clusters or “colonies” of immobilized nucleic acid molecules.

Exemplary SBS procedures, fluidic systems and detection platforms that can be readily adapted for use with amplicons produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

In some embodiments, sequencing is performed after amplifying. In some embodiments, amplification is not performed before sequencing. A number of different sequencing methods are known to those skilled in the art, such as those described in U.S. Pat. Nos. 9,683,230 and 10,920,219, each of which is incorporated by reference herein in its entirety.

In some embodiments, the sequencing fragments are deposited on a flow cell. In some embodiments, the sequencing fragments are hybridized to complementary primers grafted to the flow cell or surface. In some embodiments, the sequences of the sequencing fragments are detected by array sequencing or next-generation sequencing methods, such as sequencing-by-synthesis.

The P5 and P7 primers are used on the surface of commercial flow cells sold by Illumina, Inc., for sequencing on various Illumina platforms. Such primer sequences are described in U.S. Patent Publication No. 2011/0059865 A1, which is incorporated herein by reference in its entirety. While the P5 and P7 primers are given as examples, it is to be understood that any suitable amplification primers can be used in the examples presented herein.

In some embodiments, a sequencing primer used for sequencing comprises a sequence fully or partially complementary to one or more unique primer binding sequences comprised in the sequencing template. In some embodiments, a sequencing primer comprises at least an A2 sequence (SEQ ID NO: 40), at least an A14 sequence (SEQ ID NO: 4), or at least a B15 sequence (SEQ ID NO: 5), or their complements.

In some embodiments, sequencing is performed using sequencing primers that bind to A14, B15, and/or a hybridization sequence (HYB). FIG. 47 presents some representative combinations of primers that may be used to sequence templates described herein.

An advantage of certain methods set forth herein is that they provide for rapid and efficient detection of a plurality of target nucleic acid in parallel. Accordingly, the present disclosure provides integrated systems capable of preparing and detecting nucleic acids using techniques known in the art. Thus, an integrated system of the present disclosure can include fluidic components capable of delivering amplification reagents and/or sequencing reagents to one or more nucleic acid fragments, the system comprising components such as pumps, valves, reservoirs, fluidic lines, and the like. A flow cell can be configured and/or used in an integrated system for detection of target nucleic acids. Exemplary flow cells are described, e.g., in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

B. Dark Cycles in Sequencing

In some embodiments, a custom sequencing recipe can be prepared to comprise dark cycles (also known as dark regions), which are used to skip the recording of a particular sequence. As used herein, a “dark cycle” refers to a method wherein the sequencing chemistry of a particular sequence is carried out, but the sequencing is not imaged by the sequencer. WO 2012055929 and WO 2010127304 describe dark cycles, and each of these is incorporated by reference herein. Dark cycles can be used to mitigate phasing/prephasing issues relating to repeatedly sequencing low diversity sequences, such as a library of ME sequences, that may globally worsen the sequencing result. After the dark cycles, the imaging of sequences is resumed so that the insert sequences comprised in sequencing templates are recorded.

A custom sequencing protocol can include an appropriate number of dark cycles to span the length of the sequence to be skipped over. In other words, the number of dark cycles can be based on the number of bases intended to be skipped over. For example, if the sequence to be skipped over is an ME sequence, which is 19 bases long, 19 dark cycles are used. In some embodiments, the sequence to be skipped over is an ME sequence or its complement. In embodiments with a 19-nucleotide long ME, the number of dark cycles is 19. With a ME having a different number of nucleotides, the dark cycle is generally the number of nucleotides. In some embodiments, a user can skip the entire ME. In some embodiments, a user can skip most of the ME domain and sequence part of it, ignoring those nucleotides comprised in the ME that are sequenced.

In some embodiments, the sequencing method comprises dark cycles wherein data are not being recorded for a portion of the sequencing method. In some embodiments, the data not being recorded are sequence data associated with the 3′ transposon end sequence. In some embodiments, the sequence data not being recorded is an ME sequence. In some embodiments, the dark cycles comprise 19 cycles.

In some embodiments, sequencing comprises dark cycles wherein data are not being recorded for a portion of the sequencing. In some embodiments, the data not being recorded are sequence data associated with a transposon end sequence or its complement (ME or ME′).

Examples of where binding of a sequencing primer to a sequencing primer sequence (i.e., a primer binding site) is shown in the arrows on top of the representative polynucleotides in FIG. 47. After binding of sequencing primer to an A14, B15′, or X sequence, dark cycles may be used to avoid sequencing of some or all of the ME sequences.

In some embodiments, the sequencing method does not comprise dark cycles. In these embodiments, custom primers are used to obviate the need for dark cycles. In some embodiments, the custom primers may be bridged primers that comprise a sequence that aligns with ME, wherein the ME sequence is not imaged.

C. Error Correction or Identification of Mutations Present in a Single Strand of a Double-Stranded Nucleic Acid

In some embodiments, concatenated sequencing templates comprising two copies of the same insert can be used for error correction and identification of mutations that are only present in a single strand. This is because, in essence, a read of a single concatenated sequencing template is equivalent to reading both strands of a double-stranded nucleic acid that is tagmented. Thus, preparing and sequencing concatenated sequencing templates can increase the sequencing depth. Increased sequencing depth can be crucial for discovering rare somatic mutations present in, for example, a patient with a solid tumor to increase the chance of identifying the mutation.

In some embodiments, results from sequencing of the concatenated sequencing templates described herein allows for error correction. Such errors can include correcting for random errors introduced during amplification or sequencing itself

In some embodiments, results from sequencing of the concatenated sequencing templates described herein allows for identification of mutations or other base pair differences that are present only in one strand of a double-stranded nucleic acid.

Different means of preparing such concatenated sequencing templates comprising two copies of the same insert are described herein, such as extension after bridging of single-stranded fragments prepared using ligation of forked adapters (as shown in FIG. 29) or with using tagmentation with transposomes comprising forked adapters (as shown in FIG. 40).

In some embodiments, a difference between two copies of a sequence in a concatenated sequencing template is due to an error (such as a mistake introduced by sequencing or amplifying).

In some embodiments, the method comprises evaluating sequencing results from multiple sequences of a given insert prepared from different templates and correcting errors in sequencing results for this insert. In some embodiments, correcting the error is based on the sequencing data from the insert and its complement comprised in the same concatenated sequencing template and/or the insert comprised in multiple concatenated sequencing templates.

In some embodiments, a difference between two copies of a sequence in concatenated sequencing template is due to mutation that was only present in a single-strand of the double-stranded nucleic acid that is tagmented. Such a mutation present in only one strand may be termed “non-canonical base pairing” and may be due to nucleobase damage or mutation. Such non-canonical base pairings can generally be difficult to evaluate, and the present method may improve on identification of such base pairings.

In some embodiments, a method comprises evaluating sequencing results from multiple sequences of a given insert prepared from different templates. In some embodiments, determining instances of non-canonical base pairing based on the sequencing data from the insert and its complement comprised in the same concatenated sequencing template; and/or the insert comprised in multiple concatenated sequencing templates.

D. Determining Proximity or Contiguity Information

In some embodiments, a method comprises evaluating sequences of inserts comprised in the same template and determining proximity data for sequences comprised in the double-stranded nucleic acid based on inserts that are comprised in the same template.

As shown in FIG. 45, the present method can be used “walk” down a double-stranded nucleic acid (such as that shown in FIG. 45), with bridging and generation of concatenated sequencing templates from single-stranded fragments produced by denaturing double-stranded fragments prepared from a double-stranded nucleic acid. As described above, the number and frequency of concatenated sequencing templates comprising a given pair of inserts can be used to determine contiguity data on the double-stranded nucleic acid.

### XI. Methods of Methylation Analysis Using Concatenated Sequencing Templates

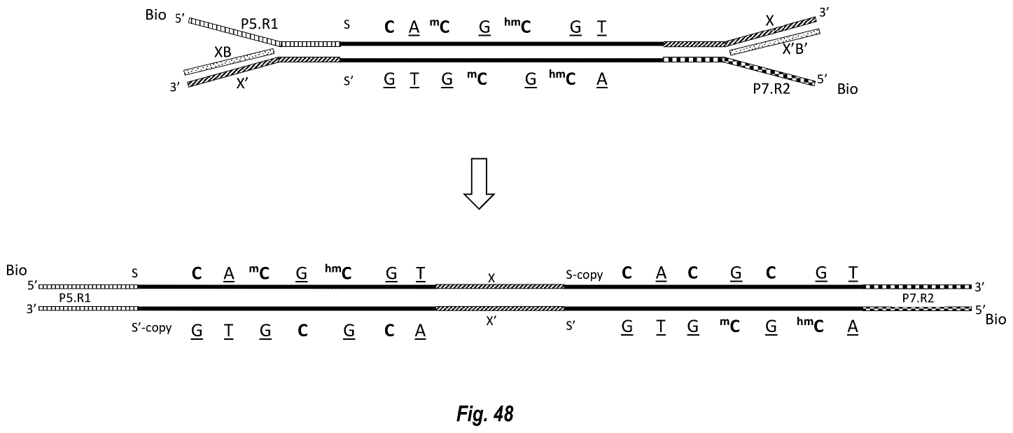

In some embodiments, concatenated sequencing templates comprising an insert sequence and a copy of the same insert may be used for methylation analysis. These sequences may be described above as concatenated sequences with “two copies” of an insert sequence, however, a copy of an insert sequence would not comprise modified nucleotides (such as modified cytosines) in the absence of conditions to promote them. This aspect is shown in FIG. 48, wherein the S and S′ insert sequences comprise methylated cytosines and hydroxymethylated cytosines, but the S-copy and the S′-copy do not. Thus, while the sequences of S and S-copy are the same and S′ and S′-copy are the same, the methylation status of S and S-copy may be different and the methylation status of S′ and S′-copy may be different.

As used herein, “methylation analysis” refers to evaluating whether cytosines in a given insert from a target nucleic acid are methylated or hydroxymethylated. As used herein, “modified cytosines” refers to methylated or hydroxymethylated cytosines, “unmodified cytosines” refers to cytosines that are not methylated. In some embodiments, the methylated cytosine is 5-methylcytosine (5mC), and the hydroxymethylated cytosine is 5-hydroxymethylcytosine (5hmC).

Means of performing methylation analysis are generally known in the art, but these methods may rely on comparison of two different aliquots of a sample (one aliquot treated with an agent to alter modified or unmodified cytosines and the other aliquot untreated). Standard sequencing analysis for methylation analysis can then be performed to identify modified cytosines, often by evaluating mismatch between treated and untreated aliquots and/or evaluating differences in the sequence results from complementary sequences from a target nucleic acid.

The present methods instead use double-stranded concatenated sequencing templates prepared from a sample comprising target nucleic acid without requiring two separate aliquots of a sample. Further, the present methods have an insert sequence and a copy of insert sequence linked together in a single-stranded concatenated sequencing template and differences between these two sequences can be used for methylation analysis. The analysis of these linked sequences will be more straightforward than analysis of unlinked sequences and require only a single sample.

In some embodiments, the two complementary strands of a double-stranded concatenated sequencing template are amplified (such as with cluster amplification) and sequenced on a flowcell, which allows for a base coding analysis to identify modified and unmodified cytosines, as described herein. In some embodiments, the amplification replaces uracils that are incorporated into sequencing templates with thymines, as uracils will stall polymerases used for SBS sequencing. In some embodiments, the replacement of uracils with thymines during amplification is based on the presence of dTTP in the cluster amplification mix (and absence of dUPT in the cluster amplification mix).

The present application discloses a wide variety of different ways that one skilled in the art may choose to perform such analysis, as shown in FIGS. 48-62C. The choice of a particular method depends on whether a user wants to convert cytosines or convert methylated cytosines. Also, a user may choose a method to differentiate methylated cytosines, hydroxymethylated cytosines, and unmodified cytosines from each other, or a user may choose to only differentiate modified cytosines from unmodified cytosines.

In some embodiments, after conversion of cytosines or modified cytosines to uracils or dihydroxyuracil (DH U), a PCR reaction converts the uracils or ^{DH}U's to thymines. In this way, a T/G mismatch (instead of a standard C/G match) in complementary sequences can be evaluated as a position that comprised either a cytosine or modified cytosine, as will be discussed below.

In some embodiments, a method of identifying modified cytosines comprised in an insert sequence comprised in a concatenated sequencing template comprises preparing a double-stranded concatenated sequencing template, wherein each strand comprises an insert sequence and a copy of the insert sequence and the two strands are complementary to each other and subjecting each strand to a condition for altering modified and/or unmodified cytosines. A variety of approaches will be described herein, but one skilled in the art could choose any method to alter either modified or unmodified cytosines. In some embodiments, altering either modified or unmodified cytosines allows a user to identify positions of modified or unmodified cytosines in a target nucleic acid, as will be described herein for some representative methods.

An exemplary double-stranded concatenated sequencing template, wherein each strand comprises an insert sequence and a copy of the insert sequence and the two strands are complementary to each other, that may be used for the present method is shown in FIG. 48 (comprising a S insert and a S-copy in one strand and a S′ insert and a S′-copy in the other strand).

In some embodiments, the method further comprises preparing amplicons of each single-stranded concatenated sequencing template and sequencing amplicons and evaluating sequencing results for the insert sequence and the copy of the insert sequence in the amplicons produced from each strand. In some embodiments, the method comprises determining positions of modified cytosines comprised in the insert sequence based on the sequences of each strand of the double-stranded concatenated sequencing template.

In figures shown herein, one strand may be referred to as a “top strand” and another as “bottom strand” to indicate that these are complementary single-stranded templates that are comprised together in a double-stranded concatenated sequencing template.

In some embodiments, the concatenated sequencing templates are prepared by a method described herein. Alternatively, other methods of preparing concatenated sequencing templates may be used, such those described in the CODEC method (described in Bae et al., bioRxiv, 10.1101/2021.06.11.448110, posted Jun. 12, 2021), followed by the presently described methylation analysis.

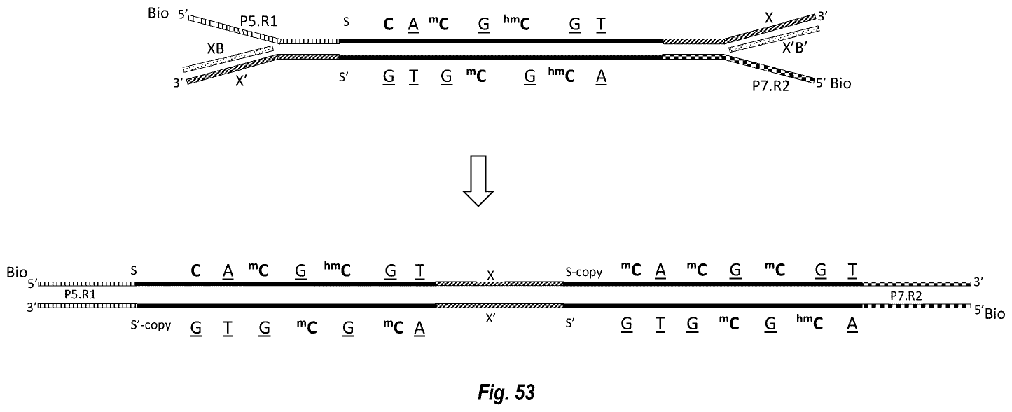

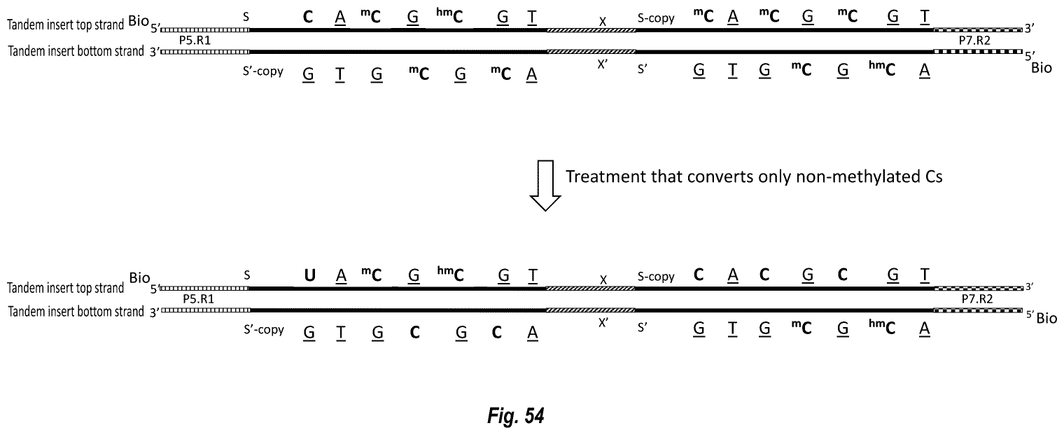

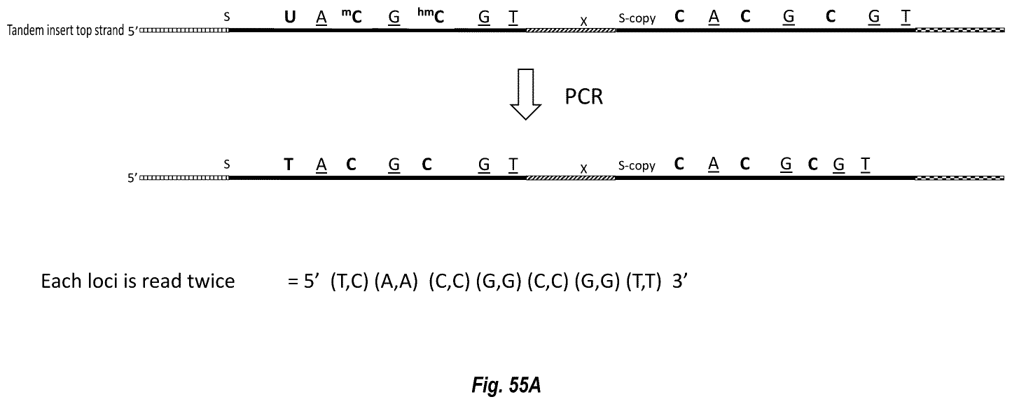

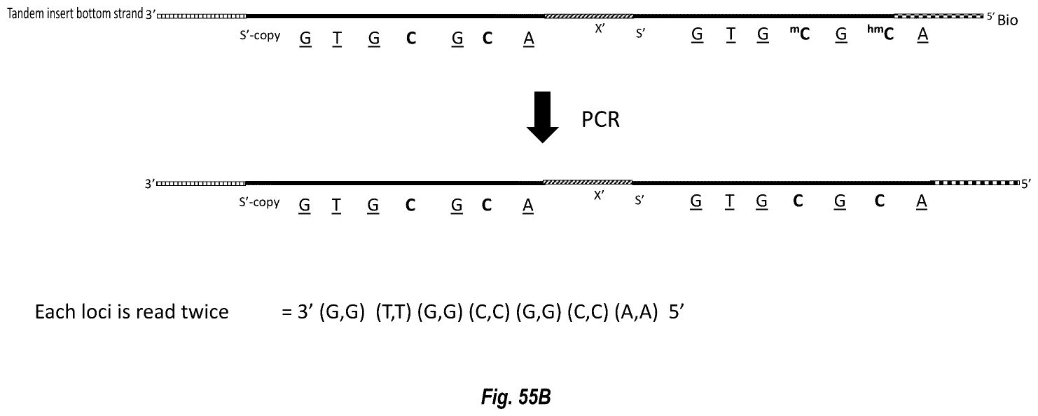

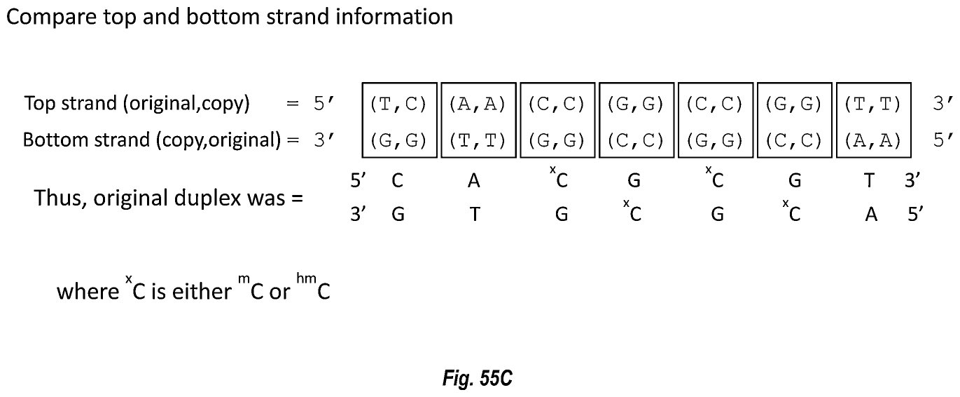

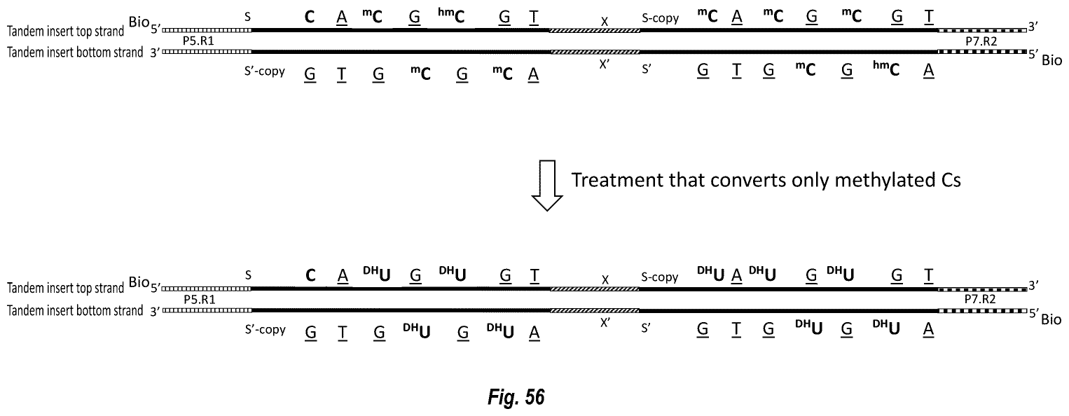

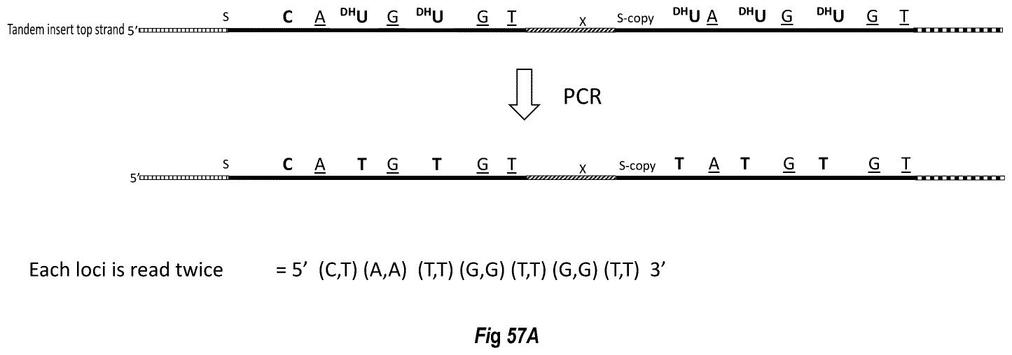

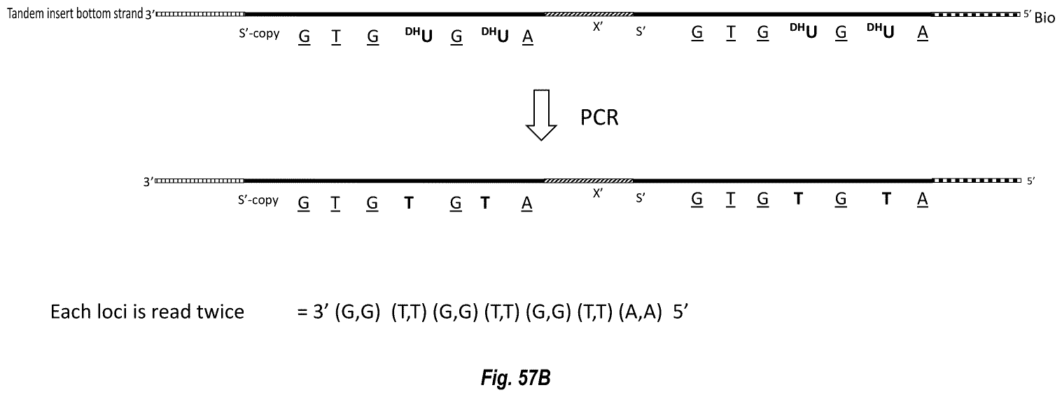

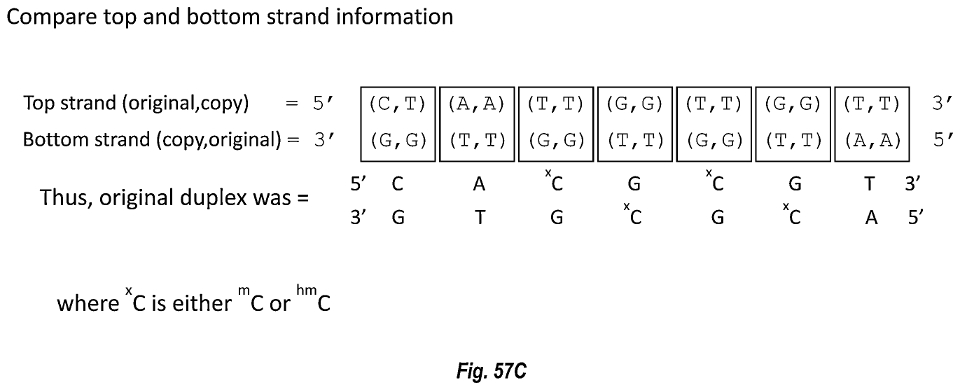

In some embodiments, extension to produce the double-stranded concatenated sequencing template is performed with a reaction solution comprising methylated-dCTP, as shown in FIG. 53. In some embodiments, extension is performed with a reaction solution comprising methylated-dCTP to allow for preserving methylated cytosines in a copy of an insert sequence (such as shown in the S′-copy and S-copy in FIG. 53). This extension with methylated-dCTP can be paired with methods that convert only unmodified cytosines (FIG. 54), with PCR and analysis shown in FIGS. 55A-55C. This extension with methylated-dCTP can also be paired with methods that convert only modified cytosines (FIG. 56), with PCR and analysis shown in FIGS. 57A-57C. This PCR conversion of U's to T's allows for sequencing by standard means.

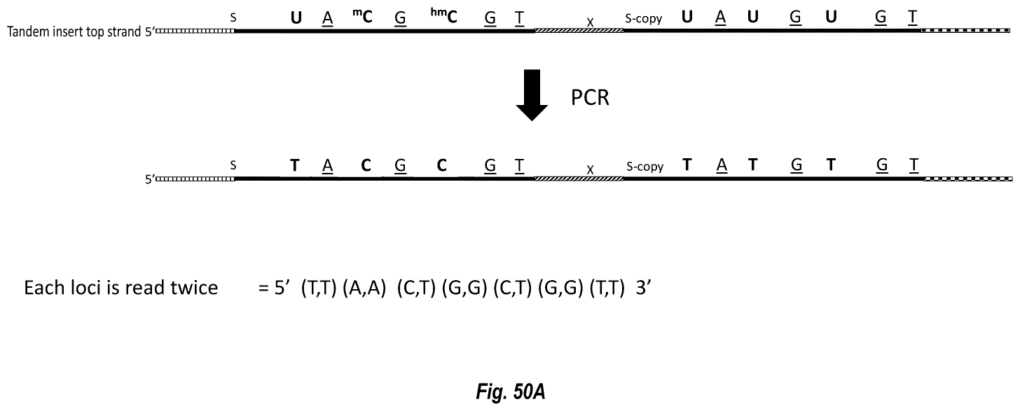

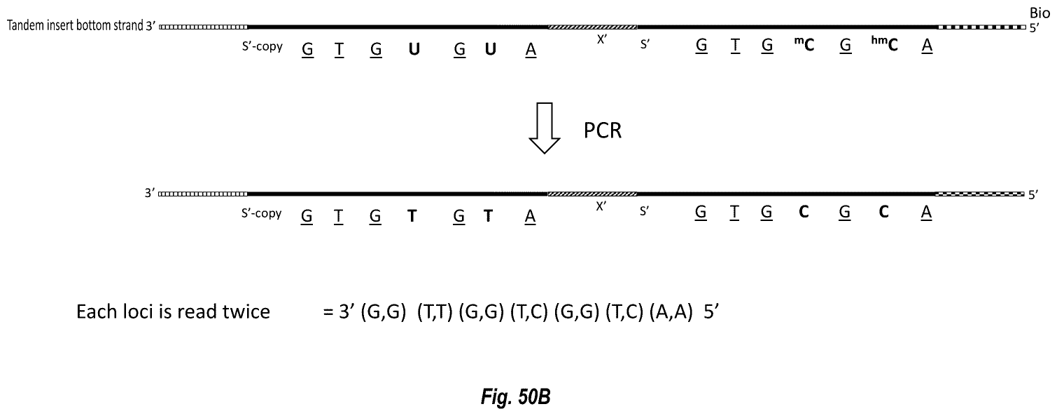

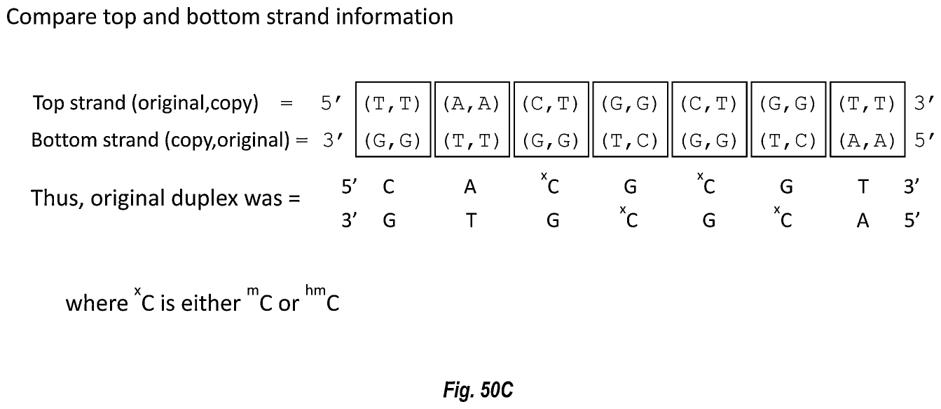

In some embodiments, uracils comprised in the concatenated sequencing templates are converted to thymines when preparing amplicons. This aspect is shown, for example, in FIGS. 50A and 50B, wherein the amplicons prepared by PCR have replaced T's, while the templates before PCR comprised U's.

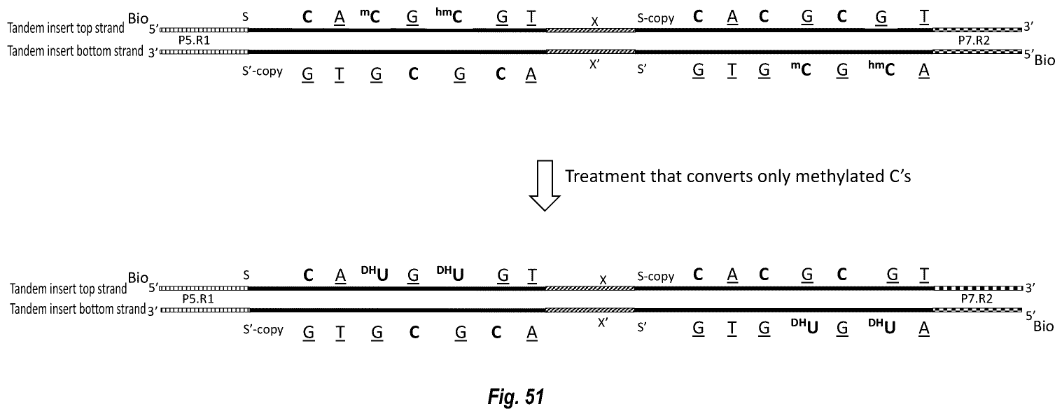

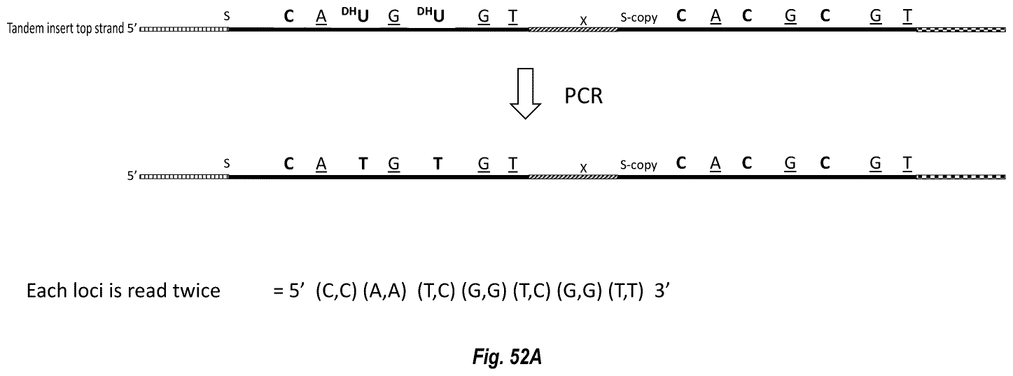

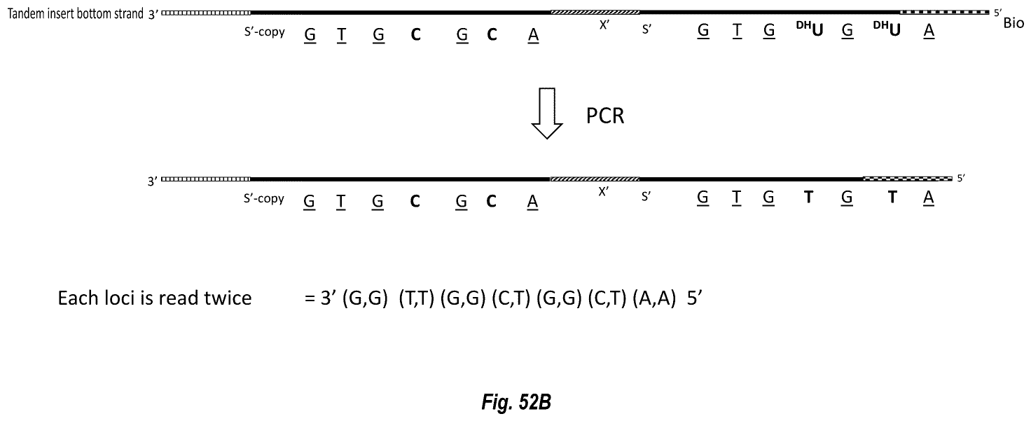

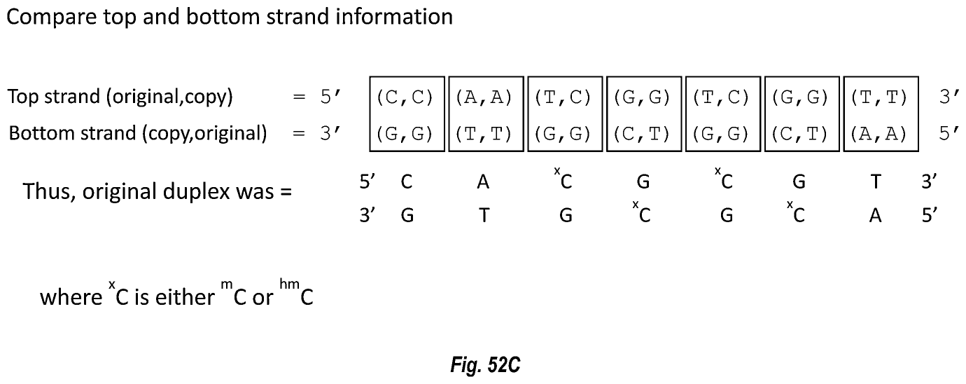

In some embodiments, modified cytosines are altered by TET-Assisted Pyridine Borane Sequencing (TAPS). A method comprising TAPS is shown in FIG. 51, wherein methylated cytosines (mC) and hydroxymethylated cytosines (h mC) are converted to dihydroxyuracil (DH U). DH U will be replaced by T during PCR amplification, as shown in FIGS. 52A and 52B, allowing for calling of (T,C) in an insert (i.e., “original”) and its copy, respectively, as positions with a methylated cytosine and (C,C) as positions with an unmodified cytosine. These (T,C) and (C,C) will all be paired with G's in the sequence of the complementary strand as shown in FIG. 52C.

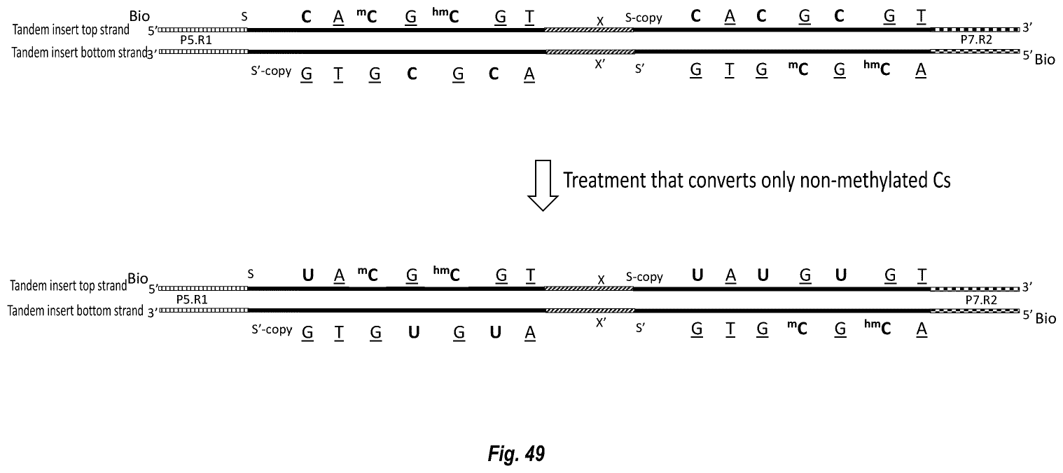

In some embodiments, unmodified cytosines are altered by a chemical or enzymatic reaction. In other words, modified cytosines may remain unaffected, but unmodified cytosines may be altered. In some embodiments, the chemical reaction is treatment with sodium bisulfite. In some embodiments, the enzymatic reaction comprises treatment with Tet methylcytosine dioxygenase 2 (TET2), T4-BGT, and APOBEC3A (using, for example, a method known as EM-seq, as described in Vaisvilas et al., Genome Res. 31(7): 1280-1289 (2021)). Such a method is shown in FIG. 49, wherein unmodified cytosines are converted to uracils. The uracils will be replaced by thymines during PCR amplification (as shown in FIGS. 50A and 50B), allowing for calling of (C,T) in an insert (i.e., “original”) and its copy, respectively, as positions with a modified cytosine and (T,T) as positions with an unmodified cytosine. In the complementary strand, these (C,T) and (T,T) will all be paired with G's, as shown in FIG. 50C. In this way, T positions in sequences of inserts that were originally C's in the target nucleic can be differentiated from positions that were originally T's in the target nucleic acid (as T's that occurred in the target nucleic acid would be paired with A's in the complementary strand). Modified C's will be retained as C since they were not altered by the treatment.

In some embodiments, the method differentiates positions of methylated cytosines from hydroxymethylated cytosines. In some embodiments, additional reaction steps allow for reactions to differentiate methylated cytosines from hydroxymethylated cytosines.

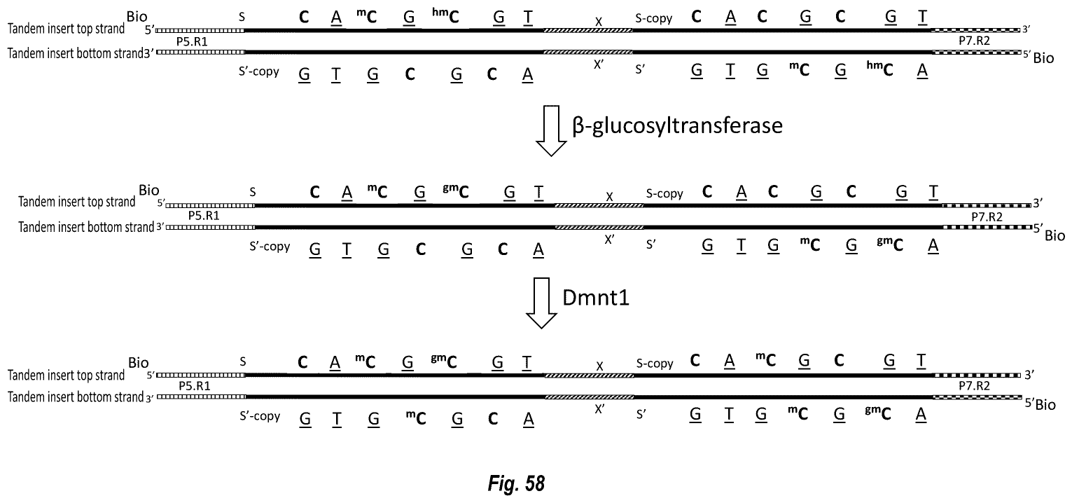

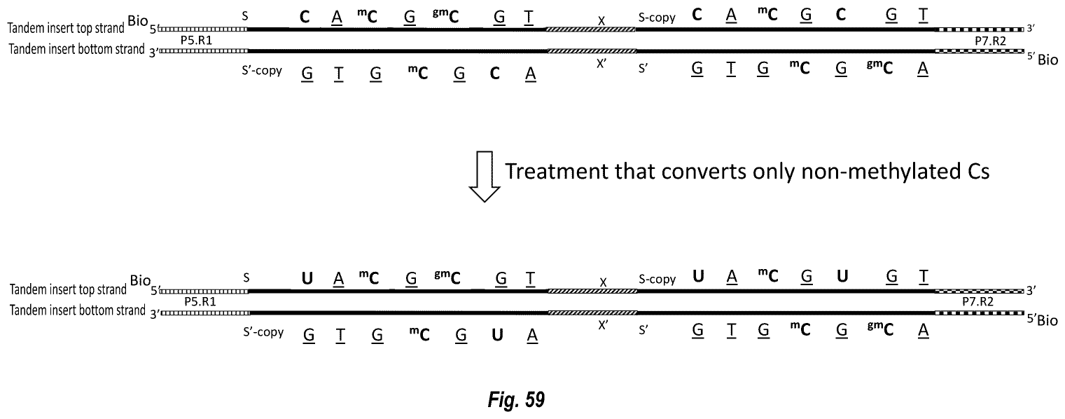

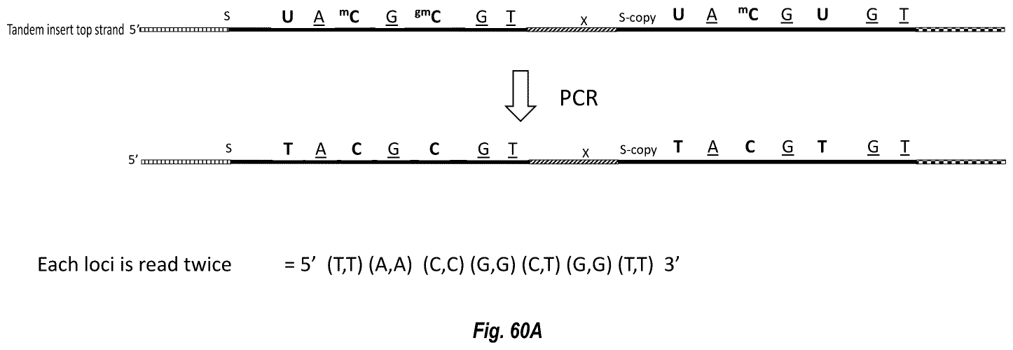

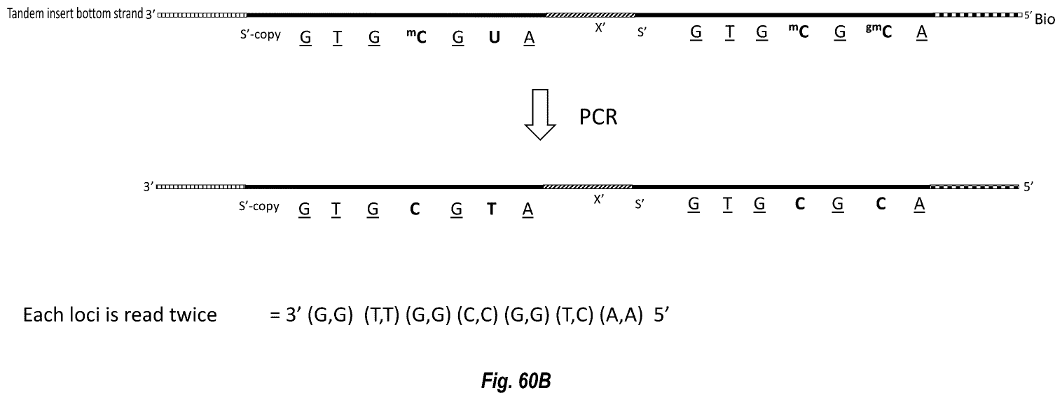

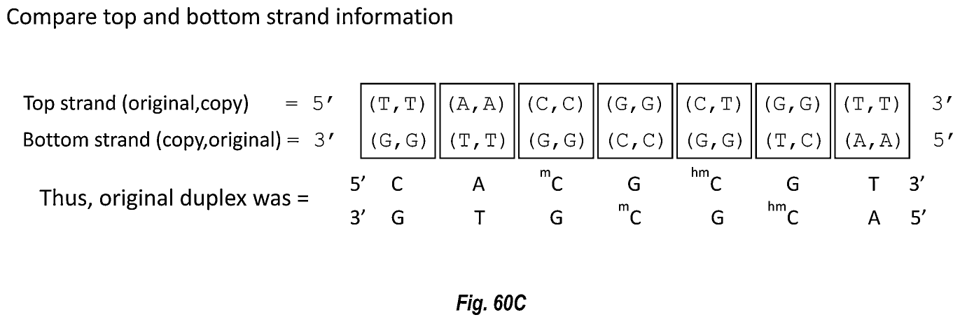

In some embodiments for differentiating positions of methylated cytosines from hydroxymethylated cytosines, the subjecting each strand to a condition for altering modified and/or unmodified cytosines comprises (a) reacting each strand with β-glycosyltransferase; (b) reacting each strand with a DNA methyltransferase (DNMT); and (c) reacting each strand with a condition that converts unmodified cytosines to uracils. Such a method is shown in FIGS. 58 and 59. Analysis of sequencing data from this method is shown in FIGS. 60A-60C. As shown in FIG. 60C using this method, cytosines from the original target nucleic acid present as (T,T) in the sequencing data, methylated cytosines present as (C,C), and hydroxymethylated cytosines present as (C,T), all of which will be paired with G's in the complementary strand.

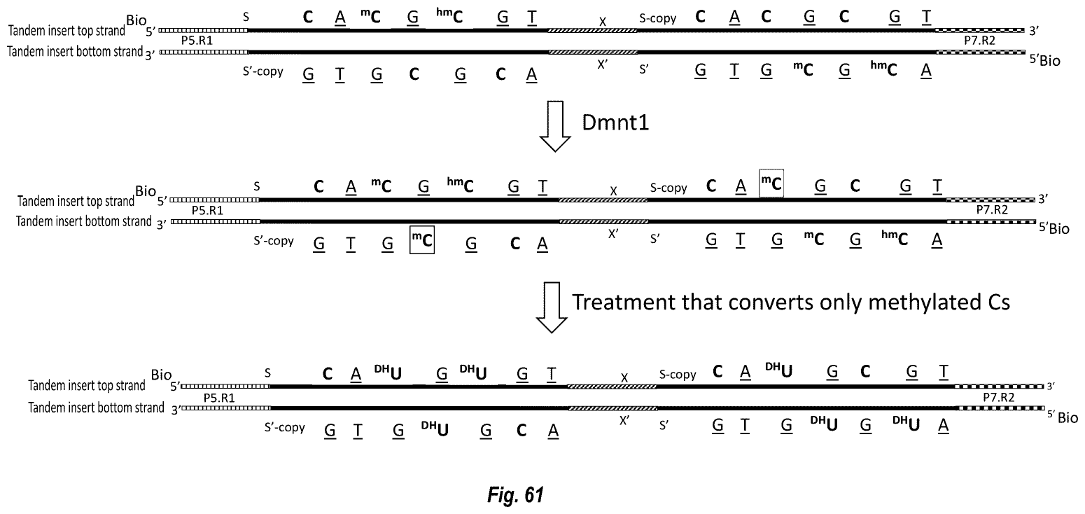

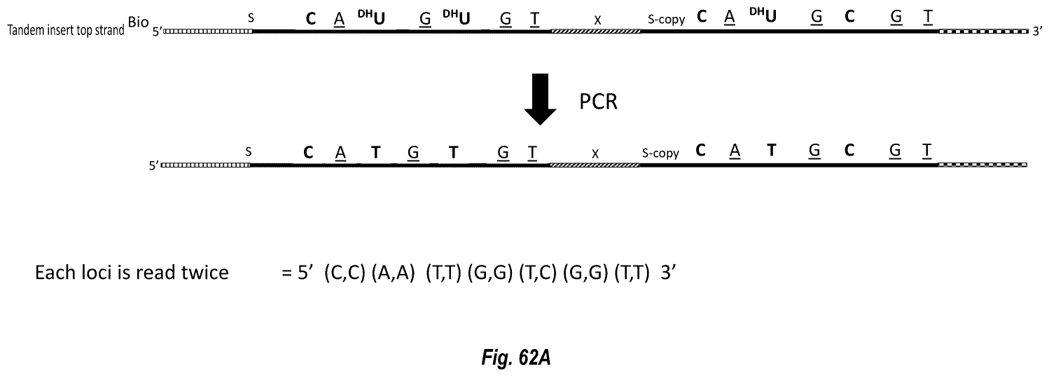

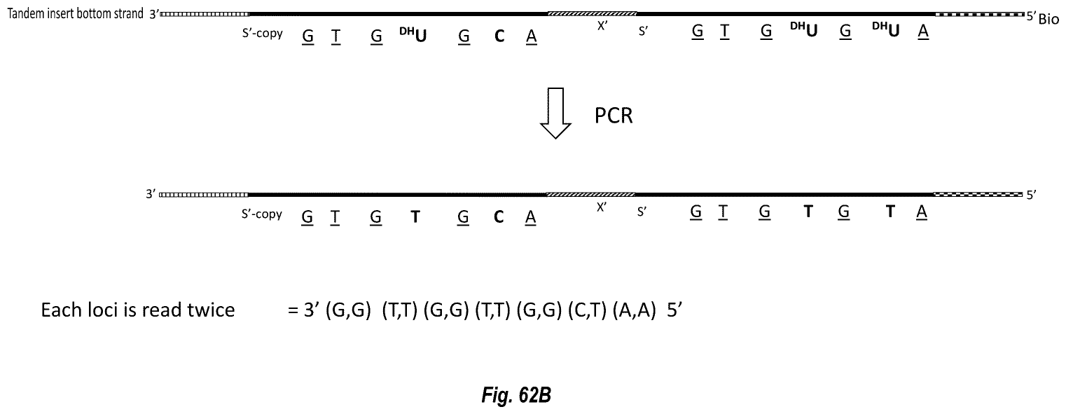

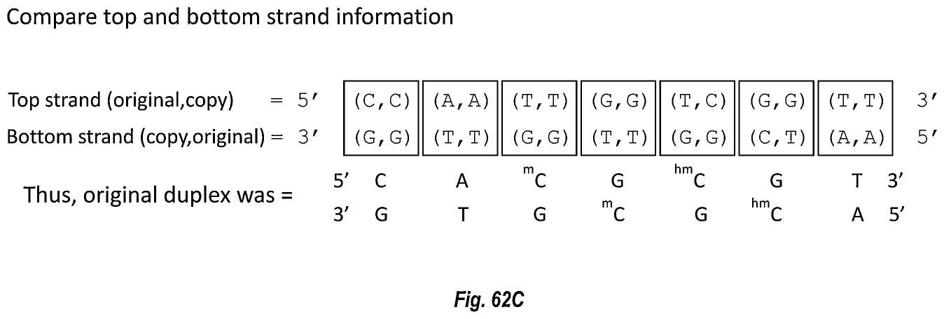

In some embodiments for differentiating positions of methylated cytosines from hydroxymethylated cytosines, the subjecting each strand to a condition for altering modified and/or unmodified cytosines comprises (1) reacting each strand with a DNMT; and (2) reacting each strand with a condition that converts methylated cytosines to dihydroxyuracil (DH U, such as using TAPS). Such a method is shown in FIG. 61. Analysis of sequencing data from this method is shown in FIGS. 62A-62C. As shown in FIG. 62C using this method, unmodified cytosines from the original target nucleic acid present as (C,C) in the sequencing data, methylated cytosines present as (T,T), and hydroxymethylated cytosines present as (T,C), all of which will be paired with G's in the complementary strand.

A. Methods Comprising Conversion of Unmodified C's to U's

In some embodiments, methylation analysis is performed with conversion of unmethylated cytosine to uracil while leaving 5-methylcytosine (5mC) and 5-hydroxymethylcytosine (5hmC) intact. An exemplary method is bisulfite sequencing. Since PCR amplification of the bisulfite-treated DNA reads uracil as thymine, the modification of each cytosine can be inferred at single base resolution, where C-to-T transitions provide the locations of the unmethylated cytosines.

B. Methods Comprising Conversion of Modified C's to U's

In some embodiments, a bisulfite-free method is used for methylation analysis. In some embodiments, TET Assisted Pic-borane Sequencing (TAPS) converts modified cytosine into dihydroxyuracil (DH U), a near natural base, which can be “read” as T by common polymerases. In some embodiments, TAPS detects cytosine modifications directly without affecting unmodified cytosines. In some embodiments, TAPS can be used to detect 5mC and 5hmC. Since PCR amplification of the TAPS-treated DNA reads DH U as thymine, the modification of each cytosine can be inferred at single base resolution, where C-to-T transitions provide the locations of the modified cytosines.

C. Methods Comprising Treatment with β-glucosyltransferase

In some embodiments, β-glucosyltransferase is used in methods to selectively convert hydroxymethylcytosines (hmC) to glucosylated-methylcytosines (gmC). In some embodiments, hydroxymethylated cytosines are “protected” from later reactions that alter methylated and hydroxymethylated cytosines. Such a method is shown in FIG. 58.

D. Methods Comprising Treatment with a DNMT

In some embodiments, a DNA methyltransferase (DNMT) is used. In some embodiments, the DNMT is DNA methyltransferase 1 (DNMT1). In some embodiments, a DNMT such as DNMT1 recognizes a hemi-methylated mCpG/GpC motif and methylates the unmethylated C to form mCpG/GpmC. DNMT1 has no activity on hemi-hydroxymethylated CpG sequences as described in Takahashi et al., FEBS Open Bio 5 (2015) 741-747. Accordingly, treatment with DNMT can be used in methods to differentiate methylated cytosines from hydroxymethylated cytosines, as shown in FIGS. 58-62C.

### EXAMPLES

### Example 1. Overview of Preparation of Polynucleotides Via Bead-Linked Transposomes

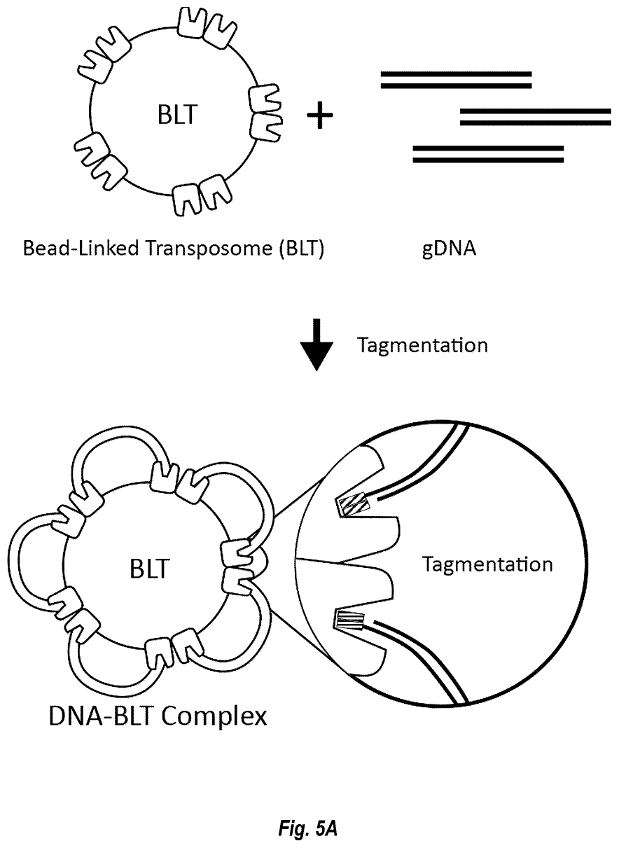

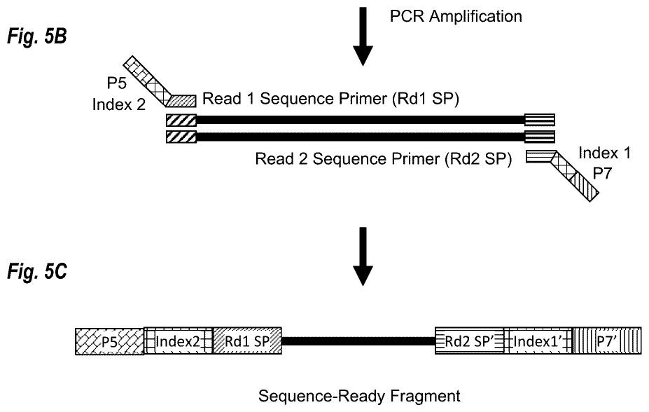

Polynucleotides comprising multiple insert sequences can be generated via methods based on bead-linked transposomes (BLTs). FIG. 5A-5C show a general methodology of generating fragments comprising insert sequences using tagmentation with BLTs, such as with the Nextera Flex workflow. As shown in FIG. 5C, however, a standard Nextera sequencing-ready fragment comprises a single insert sequence from one or more target nucleic acid. In contrast, polynucleotides described herein comprise multiple insert sequences.

Exemplary polynucleotides comprising two insert sequences can be generated by tagmentation followed PCR reactions to generate two libraries comprising different types of products: one library wherein the library products comprise P5-A14/Hyb-B15-ME sequences and one library wherein the library products comprise P7-B15/Hyb′-A14-ME sequences, as shown in FIGS. 6A-6E.

The resulting polynucleotides comprising multiple insert sequences can be used to generate a “tandem reads library,” which is a library of concatenated nucleic acid sequencing templates that can be sequenced. FIGS. 4A-4B highlight the differences between a standard Illumina pair-end library (FIG. 4A) and the present method with polynucleotides comprising multiple insert sequences (FIG. 4B). As shown in FIG. 4B, the read 1-A sequencing primer (first read primer) sequences the forward read of the first insert for this hybrid DNA library (i.e., the polynucleotide comprising multiple insert sequences). After 150 cycle SBS sequencing, the SBS synthesized strand can denature and then the read 1-B sequencing primer (second read primer) is hybridized and the forward read of the second insert. A paired-end turn around can then be performed to similarly carry out 150 cycles each for the reverse strand of second insert with the read 2-A sequencing primer (third read primer) followed by the reverse strand of the first insert with the read 2-B sequencing primer (fourth read primer).

The workflow of preparing the polynucleotide with multiple insert sequences leverages the well-established bead-linked transposome library preparation technology (e.g. Nextera flex) or adapter-based methods (e.g. Truseq).

### Example 2. Preparation of Polynucleotides Via Tagmentation and Subsequent Addition of P5/P7 and Hybridization and Complement of Hybridization Sequences

In an exemplary method, libraries products comprising A14 and B15 sequences were generated by tagmentation to add A14 and B15 sequences during a tagmentation reaction (FIG. 6A). This was followed by addition of P5/HYB sequences (in Tube 1) and P7/HYB′ (in Tube 2) by PCR, as shown in FIGS. 6B-6C.

After clean-up, libraries are mixed. Based on hybridized adducts generated between HYB and HYB′, extended products can then be prepared. Only those products that are boxed in FIG. 6D comprise a HYB or HYB′ sequence and can form a hybridized adduct with another library product based on HYB/HYB′ hybridization, after which extension can be used to generate a concatenated nucleic acid sequencing template. At least 1/9^{th} of the extended product is a sequenceable product capable of forming clusters (i.e., a concatenated nucleic acid sequencing template comprising one strand comprising HYB′ [H′] and P5 and one strand comprising P7 and HYB [H], FIG. 6E).

### Example 3. Preparation of Polynucleotides Via Tagmentation and Subsequent Addition of Hybridization and Complement of Hybridization Sequences

In an exemplary method, libraries products comprising insert, adapter, and hybridization sequences were generated via tagmentation by BLTs followed by addition of HYB and HYB′. In this exemplary method, one tube used bead-based tagmentation to form a P5-HYB′ forked library and another tube used solution-based tagmentation to form a P7-HYB forked library. HYB and HYB′ were added to the library products after tagmentation.

First, a P5/HYB′ library was generated using 10 μL of BLTs (10 fmole) and washed with 200 μL wash buffer. Next, 176 μL working buffer was mixed with 10 μL of single strand binding protein. Wash buffer was removed from the beads and 44 μL of working buffer plus SSB mix was added. The solution was incubated 1 min at RT. A total of 6 μL of 10× tagmentation buffer was then added to the beads, and tagmentation proceeded for 10 minutes at 37° C. Then, 124, 5% SDS was added and incubated at 37° C. for 10 minutes, followed by three washes with 200 μL wash buffer and resuspension in 200 μL wash buffer.

To add the hybridization sequence, fragments were incubated at ° C. for 5 mins to denature the ME′ sequence. After a quick wash with 200 μL wash buffer, beads were resuspended in 80 μL of 2 μM ME′-HYB′, and an Annealrt program was run starting from 60° C., going down to 20° C. (1° C. per cycle). Beads were washed with 200 μL wash buffer, resuspended in 804, ELM3, and then rotated for 30 minutes at RT. Beads were washed with 200 μL wash buffer and stored at 4° C. in wash buffer.

Separately, a P7/HYB library was prepared using an oligonucleotide (oligo) duplex comprising a P7-B8-ME/ME′. The oligonucleotide duplex comprised Oligo 1 and Oligo 2. Table 2 describes the components of the reaction solution for generating the oligonucleotide duplex.

Oligo 1: (20P7-B8-ME)
(SEQ ID NO: 9)
5′-CAG AAG ACG GCA TAC GAG ATG GGC TCG GAG ATG
TGT ATA AGA GAC AG-3′
Oligo 2: (ME′)
(SEQ ID NO: 3)
5′-/Phos/CTG TCT CTT ATA CAC ATC T-3′

TABLE 2
Components of reaction solution for generating the
oligonucleotide duplex

| Oligo duplex formation | stock | Final concentration | Amount |
|---|---|---|---|
| Water | | | 70 μL |
| Oligo 1 | 100 μM | 10 μM | 10 μL |
| Oligo 2 | 100 μM | 10 μM | 10 μL |
| Annealing Buffer | 10x | 1x | 10 μL |

After the oligonucleotide duplex solution was prepared, an Annealrt recipe was performed on PCR using the protocol in Table 3. The duplex was saved at −20° C. for long-term storage, and multiple freeze thaw cycles were avoided.

TABLE 3
PCR protocol for generating oligonucleotide duplex

| Temp | Time | |
|---|---|---|
| 95° C. | 1 minute | |
| 80° C. | 30 seconds | 60 cycles |
| Decrease temp by 1° C. every cycle | | |
| 20° C. | 60 minutes | |
| 10° C. | forever | |

The enzyme complex was assembled as outlined in Table 4, incubated overnight at 37° C., and then stored at 20° C.

TABLE 4
Reagents for assembly of enzyme complex

| Reagent | Stock conc | Final conc | Amount |
|---|---|---|---|
| Standard storage buffer | | | 77.65 μL |
| TsTn5 transposase enzyme | 85 μM | 2 μM | 2.35 μL |
| (Illumina) | | | |
| Transposons | 10 μM | 2 μM | 20 μL |
| Final volume | | | 100 μL |

The enzyme complex was diluted 1 into 5 in standard storage buffer to 400 nM. A tagmentation reaction was prepared based on Table 5, and the tagmentation proceeded for 5 minutes at 55° C.

TABLE 5
Tagmentation reaction preparation

| | Stock conc | Final conc | Volume to add |
|---|---|---|---|
| Tagmentation buffer | 2X | | 25 μL |
| DNA | | | 1.5 μL |
| Enzyme | 400 nM | | 6 μL |
| RSB | | | 17.5 |

Column clean-up was performed with zymo-kit and eluted in 20 μL of resuspension buffer (RSB). Then a total of 18 μL of tagmented library plus 2 μL of 100 μM HYB-ME′ oligo (final concentration 1004) was incubated at 75° C. incubation for 5 minutes, followed by a slow ramp to 20° C. to replace the ME′ oligo with HYB-ME′ using Oligo 3 and Oligo 4.

Oligo 3: (p-18ME′HYB′)
(SEQ ID NO: 10)
/5Phos/TGTCTCTTATACACATCTCTCTCTTCTCTCCTTCTTCTCTCT
Oligo 4: (p-18ME′HYB)
(SEQ ID NO: 11)
/5Phos/TGTCTCTTATACACATCTAGAGAGAAGAAGGAGAGAAGAGAG

A total of 180 μL of ELM3 was added, and the solution was rotated at RT for 30 minutes. A SPRI bead clean-up was performed.

At this step, the P5 library was on beads and the P7 library was in solution. Both libraries were mixed and an Annealrt program was started going from 40° C. going down to 20° C., followed by washing the beads and resuspending in 100 μL AMS1 extension buffer (comprising a strand-displacing polymerase such as Bst polymerase and nucleotides). The resuspended solution was washed with NaOH and library was amplified off the bead surface. In this example, the PCR was performed with P5/A14 and P7/B15 primers. Ampure bead clean-up was performed to remove unattached adapters.

The Qubit Concentration was measured as 0.849 μL/mL, which is approximately 2 nM. A 5 pM single-stranded library was made on a FC #CD79K, seeded miseq flowcell. The clusters did not appear consistent with 5 pM, as they were also dim, so another 24-cycle amplification was performed.

The protocol forms hybrid libraries, but may not have sufficient efficiency. For example, denaturing on beads with NaOH may cause sample loss and insufficient density on the flowcell for sequencing. Preparation of both libraries on beads may improve yields.

### Example 4. Preparation of DNA Libraries Via Bead-Linked Transposons Followed by Denaturation, Hybridization, and Strand Extension

The workflow for preparing hybrid DNA library can be performed with bead-linked transposons (BLTs). A difference from a standard protocol for library preparation is the presence of two types of beads (type I beads have BLTs comprising ME′-HYB′ and type II beads have BLTs comprising ME′-HYB at the non-inserted strand of transposon).

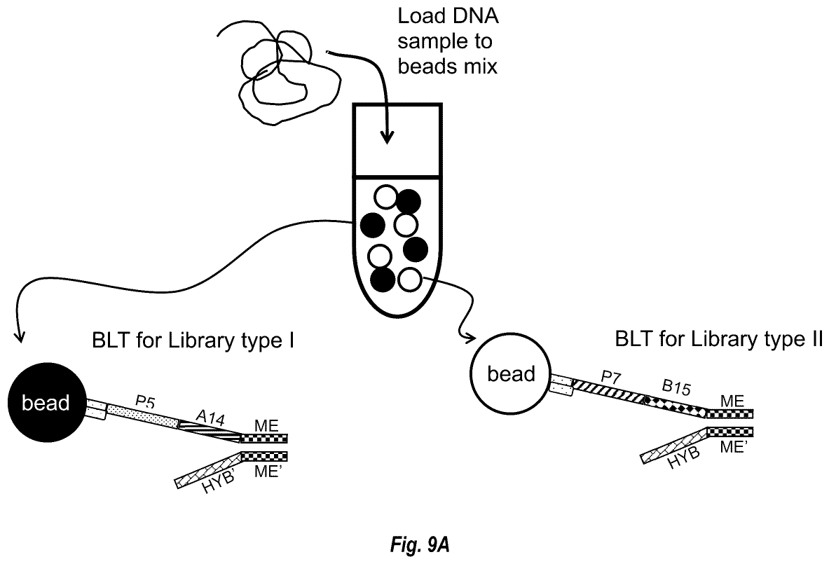

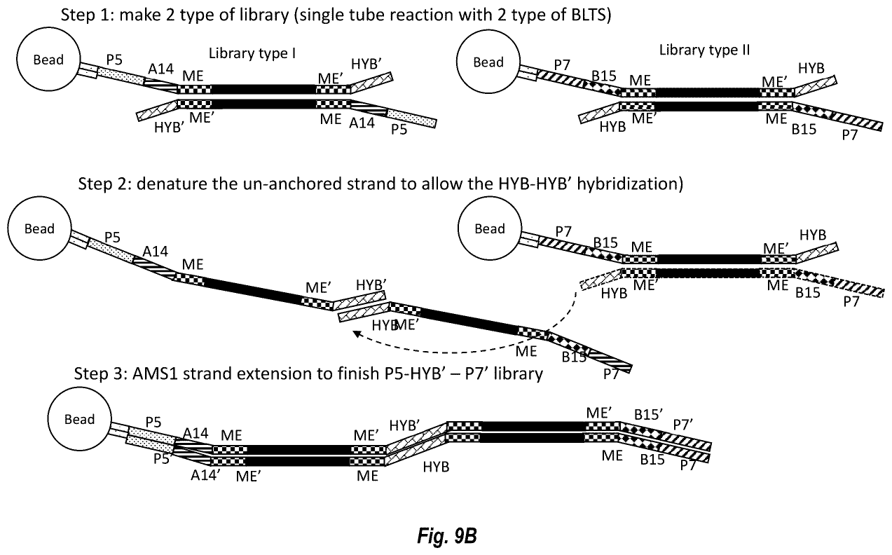

After BLT tagmentation and gap-fill ligation (using ELM3), there are two options for library preparation completion. As shown in FIG. 9B, the non-anchored strand can be denatured off the BLT to allow hybridization of the HYB-HYB′part of the library, and then AMS1 polymerase extension mix can be added to extend the strand to complete the library with P5-P7′ or P7-P5′ at the ends. The library can then be released from the beads via PCR or release buffer with biotin.

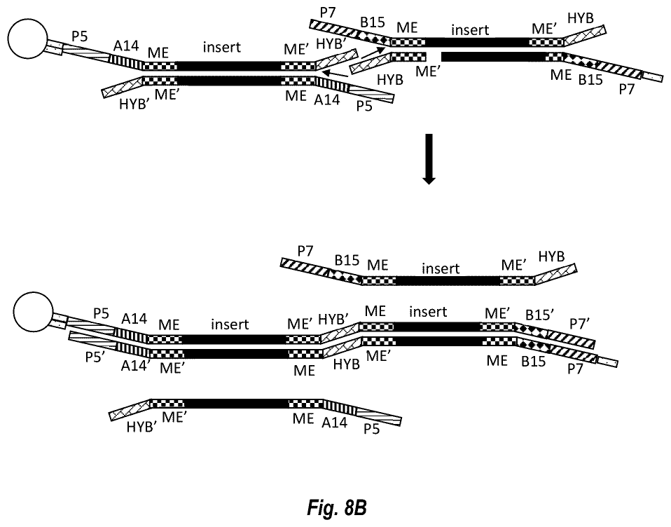

The alternate method is shown as FIGS. 8A-8B. Here, the P5 anchored transposomes are attached using biotin or chemical conjugation such that the library cannot be released with release buffers containing low concentration of biotin. The other bead type has P7 anchored to beads using single desthiobiotin, which can be easily removed off streptavidin using a release buffer. Therefore, the P7-HYB library can be selectively released and allowed to hybridize to P5-HYB′ library on the bead type I.

Again, AMS1 polymerase extension mix is added to extend the strand to make P5-P7′ or P7-P5′ library and then the libraries are collected from beads using PCR or other releasing conditions (such as denaturing buffer+high temperature).

These approaches for hybridization of HYB to HYB′ and extension to form concatenated nucleic acid sequencing templates can be used for library products from other sources, such as those generated by Truseq or other types of transposome reactions.

### Example 5. Preparation of Polynucleotides with Bead-Based Protocol Using Desthiobiotin-Tagged Oligonucleotides

A protocol was developed using desthiobiotin-tagged oligonucleotides. Desthiobiotin tagging can avoid the need for a NaOH denaturation step.

To generate the P5/HYB′ library, a total of 10 μL of BLTs (10 fmole) was washed with 200 μL wash buffer. 176 μL working buffer was mixed with 1 μL of single strand binding (SSB) protein. Wash buffer was removed from the beads and 44 μL of working buffer plus SSB mix was added and incubated for 1 minute at RT. Then, 6 μL of 10× tagmentation buffer was added to the beads and tagmentation proceeded for 10 minutes at 37° C. 12 μL of 5% SDS was added and incubated at 37° C. for 10 minutes. Beads were washed three times with 200 μL wash buffer and resuspended in 200 μL wash buffer. Beads were incubated at 60° C. for 5 minutes to denature ME′ and quickly washed with 200 μL wash buffer. Beads were resuspended in 80 μL of 2 μM ME′-HYB′. The Run Annealrt program was run starting from 60° C., going down to 20° C. (1° C. per cycle). Beads were washed with 200 μL wash buffer and resuspended in 80 μL ELM3 extension-ligation buffer and rotated for 30 minutes at RT, then washed with 200 μL wash buffer and saved in wash buffer at 4° C.

The P7/HYB library was generated using a single-desthiobiotin P7-B8-ME oligonucleotide to create an enzyme complex and was assembled to Dynabeads M280 streptavidin beads. In contrast, the P5/HYB′ were generated using BLTs having dual desthiobiotin. Therefore, the release conditions are different for the 2 libraries, with the P5/HYB′ library generated with BLTs having dual desthiobiotin having release conditions of 20 mM biotin at 60° C., while the P7/HYB library will have a single desthiobiotin with release conditions of 10 μM biotin at 70° C.

To prepare the P7/HYB library, an oligonucleotide (oligo) duplex was prepared as described in Table 6.

Oligo 1: (desthio20P7-B8-ME)
(SEQ ID NO: 12)
5′-/5deSBioTEG/CAGAAGACGGCATACGAGATGGGCTCGG
AGATGTGTATAAGAGACAG-3′
Oligo 2: (ME′)
(SEQ ID NO: 3)
5′-/Phos/CTG TCT CTT ATA CAC ATC T-3′

TABLE 6
Components of reaction solution for generating the
oligonucleotide duplex

| Oligo duplex formation | Stock | Final concentration | Amount |
|---|---|---|---|
| Water | | | 70 μL |
| Oligo 1 | 100 μM | 10 μM | 10 μL |
| Oligo 2 | 100 μM | 10 μM | 10 μL |
| Annealing Buffer | 10x | 1x | 10 μL |

After the oligonucleotide duplex solution was prepared, an Annealrt recipe was performed on PCR using the protocol in Table 7. The duplex was saved at −20° C. for long-term storage, and multiple freeze thaw cycles were avoided.

TABLE 7
PCR protocol for generating oligonucleotide duplex

| Temp | Time | |
|---|---|---|
| 95° C. | 1 minute | |
| 80° C. | 30 seconds | 60 cycles |
| Decrease temp by 1° C. every cycle | | |
| 20° C. | 60 minutes | |
| 10° C. | forever | |

The enzyme complex was assembled as outlined in Table 8, incubated overnight at 37° C., and then stored at 20° C.

TABLE 8
Reagents for assembly of enzyme complex

| Reagent | Stock conc | Final conc | Amount |
|---|---|---|---|
| Standard storage buffer | | | 77.65 μL |
| TsTn5 | 85 μM | 2 μM | 2.35 μL |
| TNP | 10 μM | 2 μM | 20 μL |
| Final volume | | | 100 μL |

40 μL of M280 beads was washed with 200 μL wash buffer, resuspended in 40 μL wash buffer, and 2 μL of 2 μM transposome complex (10 fmole per BLT) was added. The beads were rotated for 30 minutes at RT, washed, and resuspended in 40 μL of wash buffer. 10 μL of enzyme beads was washed with 200 μL wash buffer. 176 μL of the working buffer was mixed with 1 μL of single strand binding protein. Wash buffer was removed from the beads and 44 μL of working buffer plus SSB mix was added and incubated for 1 min at RT. 6 μL of 10× tagmentation buffer was added to beads and tagmentation proceeded for 10 minutes at 37° C. Then, 12 μL 5% SDS was added and incubated 37° C. for 10 minutes. Beads were washed three times with 200 μL wash buffer and resuspended in 200 μL wash buffer. Beads were then incubated at 60° C. for 5 minutes to denature ME′, quickly washed with 200 μL wash buffer, and resuspended beads in 80 μL of 2 μM ME′-HYB. A Run Annealrt program was run starting from 60° C., going down to 20° C. (1° C. per cycle). Beads were washed with 200 μL wash buffer and resuspended in 80 μL ELM3 extension ligation buffer and rotated for 30 mins at RT. Beads were washed with 200 μL wash buffer and saved in 4° C. in wash buffer.

At this point, 2 separate library sets on beads are ready. 15 cycle PCR was performed with each library set, and the supernatant of PCR product shows BA peaks on the expected location. In the PCR reaction, for P5/HYB′ library P5 and HYB were used as PCR primer 1 and for P7/HYB library P7 and HYB′ were used as PCR primer 2, as outlined in Table 9.

TABLE 9
PCR conditions

| | |
|---|---|
| beads | 2 μL |
| RSB | 8 μL |
| Water | 19 μL |
| PCR primer 1 | 0.5 μL from 100 μM |
| PCR primer 2 | 0.5 μL from 100 μM |
| Enhanced PCR Mix (EPM) | 20 μL |

P7/HYB beads were resuspended in 10 mM biotin in HT1 hybridization buffer and released at 60° C. for 10 minutes since Oligo 1 of the oligonucleotide duplex comprised a single desthiobiotin. The supernatant was added to P5/HYB beads and then a slow ramp down was started from 50° C. going down to 20° C. to hybridize the library products. Then, beads were washed with wash buffer, and AMS1 was added and incubated at 50° C. for 10 minutes. Polynucleotide comprising two insert sequences (one from each library) were loaded and released onto the flowcell with 20 mM biotin in HT1 hybridization buffer.

### Example 6. Updates to HYB/HYB′ Sequence

Initial experiments were performed with a HYB sequence that may be referred to as HYB1.

HYB1 (SEQ ID NO: 13):
5′-AGA GAG AAG AAG GAG AGA AGA GAG-3′

An updated HYB design, HYB2, involved additional A/T content, shuffling of A and G nucleotides, and a C/G lock on the 5′ end of the HYB sequence.

HYB2 (SEQ ID NO: 14):
5′-GAG TAA GTG GAA GAG ATA GGA AGG-3′

### Example 7. Preparation of Polynucleotides Using Truseq PCR Free

Polynucleotides comprising multiple insert sequences were also prepared using a Truseq PCR Free protocol.

1 μg of NA12878 genomic DNA was used as input for each forked library, followed by the Illumina Truseq PCR free protocol to sheer the DNA and to do end repair and A-tailing.

For ligation step used P5/HYB2′ adapters and P7/HYB2 adapters sets were used. The P7/HYB2 adapters (SEQ ID NOs: 24 and 25) were used for insert sequence 1, while the P5/HYB2′ adapters (SEQ ID NOs: 26 and 27) were used for insert sequence 2. In these adapters, C's were methylated.

Adapters sets were prepared (10 pM final concentration) using the Annealrt recipe in Table 10, with the duplex saved at −20 C for long-term and avoiding multiple freeze thaw cycles. The oligonucleotide stock concentration was 100 pM, with a final adapter concentration of 10 μM in 1× annealing buffer (20 mM Tris, 50 mM NaCl, 0.01 mM EDTA).

TABLE 10
Conditions for preparation of adapter sets

| Temp | Time | |
|---|---|---|
| 95° C. | 1 minute | |
| 80° C. | 30 seconds | 60 cycles |
| Decrease temp by 1° C. every cycle | | |
| 20° C. | 60 minutes | |
| 10° C. | forever | |

Ligation was performed following the Illumina PCR free Truseq protocol for ligation step using the custom adapter sets. Dual clean-up was performed as listed on the Truseq protocol, and final libraries were eluted in 22.5 μL Illumina resuspension buffer.

Forked libraries were then ready for stacking to prepare polynucleotides comprising two insert sequences. 6 μL of forked library product with P5/Hyb2′ and 6 μL of forked library product with p7/Hyb2 was mixed, and 1.3 μL of 10× annealing buffer was added. The annealing program on PCR listed in Table 11 was used to hybridize the two library products.

TABLE 11
Protocol for hybridizing library products to generate
polynucleotides comprising two insert sequences

| Temp | Time | |
|---|---|---|
| 70° C. | 1 minute | |
| 70° C. | 30 seconds | 50 cycles |
| Decrease temp by 1° C. every cycle | | |
| 20° C. | 60 minutes | |
| 10° C. | forever | |

After the annealing step, 1174, (9× the volume of annealed libraries) of AMS1 was added followed by incubation at 50° C. for 10 minutes. After extension, Illumina-compatible tandem libraries were formed. A 1×SPRI clean-up was performed and sample was eluted in 12 μL of Illumina resuspension buffer

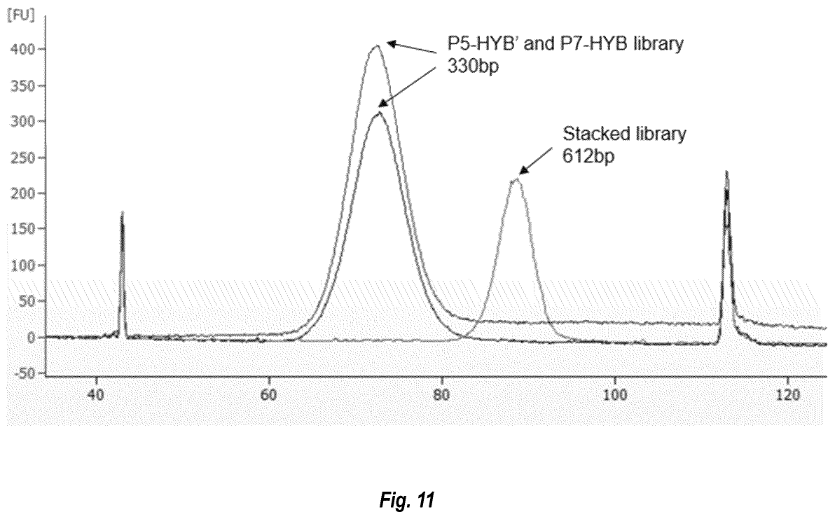

A Bioanalyzer run was done to confirm the size of the tandem library, and qPCR was used to quantify the final library product. As shown in FIG. 11, the tandem library showed an average size of 612 base pairs, which was approximately double that of the starting P5-HYB′ or P7-HYB library. These results show successfully paired of the tandem library using the Truseq method.

Tandem library can be sequenced on Illumina platforms with recipe modifications to have four reads instead of two. The location of sequencing primers was updated to use the correct sequencing primer for each sequencing read.

### Example 8. Sequencing of Polynucleotides Comprising Multiple Inserts

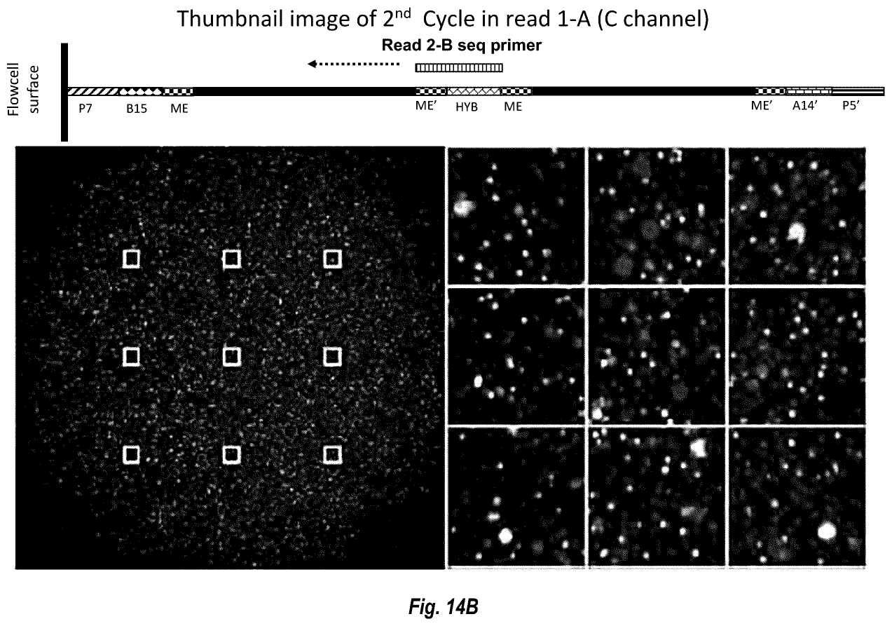

In these experiments, human genome library fragments were generated using bead-linked transposons followed by preparation of polynucleotides comprising multiple inserts. Polynucleotides were sequenced via Miseq FC. Data shown in FIG. 14A are the standard Read 1 sequencing (Read 1-A) using Read1 SBS3T sequencing primer. After finishing sequencing by Read 1-A, the synthesized strand was denatured and hybridized with a middle sequencing primer (Read1-B seq primer, which is a second read primer). Sequencing thumbnail images of the 2 read cycles are shown in FIG. 14B. There are some overamplified clusters to show data clearly.

Example reads from 10 clusters are shown in Table 12 to illustrate successful linking of two library fragments into a single cluster. 4×100 cycles of sequencing were performed and the resulting pairs of reads were mapped to the human genome. Table 12 shows the tile, x and y coordinate of the cluster as reported in BAM file. For a given cluster, the chromosome where each read mapped to is provided. As expected, the two paired reads from each library map to the same chromosome and the two library fragments map to different chromosomes. Thus, results in Table 12 show that the two inserts in a polynucleotide come from different regions in the human genome.

TABLE 12
Summary of reads from individual clusters

| | | | Lib1-Read1 | | Lib1-Read2 | | Lib2-Read1 | | Lib2-Read2 | |
|---|---|---|---|---|---|---|---|---|---|---|
| Tile | X | Y | Chr | Start | Chr | Start | Chr | Start | Chr | Start |
| 1101 | 23439 | 23439 | chr10 | 82,860,729 | chr10 | 82,860,729 | chr6 | 19,676,690 | chr6 | 19,676,690 |
| 1101 | 12443 | 12443 | chr17 | 40,802,772 | chr17 | 40,802,772 | chr2 | 193,086,113 | chr2 | 193,086,198 |
| 1101 | 17127 | 17127 | chr13 | 63,069,301 | chr13 | 63,069,192 | chr12 | 5,966,748 | chr12 | 5,966,657 |
| 1101 | 20585 | 20585 | chr11 | 50,205,348 | chr11 | 50,205,348 | chr8 | 28,763,665 | chr8 | 28,763,742 |
| 1101 | 23951 | 23951 | chr7 | 117,596,230 | chr7 | 117,596,230 | chr6 | 36,432,020 | chr6 | 36,432,021 |
| 1101 | 9397 | 9397 | chr10 | 83,626,902 | chr10 | 83,626,902 | chr10 | 489,691 | chr10 | 489,691 |
| 1101 | 18767 | 18767 | chr11 | 38,861,025 | chr11 | 38,861,005 | chr17 | 18,516,183 | chr17 | 18,516,245 |
| 1101 | 25608 | 25608 | chr15 | 54,279,093 | chr15 | 54,279,093 | chr10 | 127,364,865 | chr10 | 127,365,077 |
| 1101 | 25526 | 25526 | chr3 | 149,916,869 | chr3 | 149,916,869 | chr4 | 94,454,093 | chr4 | 94,454,162 |
| 1101 | 14474 | 14474 | chr2 | 33,141,302 | chr2 | 33,141,528 | chr2 | 33,141,302 | chr2 | 33,141,473 |

These results of reads from individual clusters demonstrates successful linking of two library fragments into a polynucleotide and sequencing of the two separate insert sequences.

### Example 9. Preparation of Polynucleotides from Starting Libraries with Sheared Genomic DNA Fragments Using a Ligation Method

Polynucleotides comprising multiple insert sequences were generated using a method comprising restriction enzyme digest and ligation. In the exemplary method described herein FIGS. 15A-F, a first library contained inserts that originated from sheared E. coli genomic DNA and a second library contained inserts that originated from sheared human genomic DNA. The first library was digested with BtgZI and the second library was digested with BgLII. The two digested libraries were ligated together to produce a tandem insert library wherein each polynucleotide contained one insert from the E. coli genome and another from the human genome (FIG. 19).

An 8-lane sequencing flow cell was prepared that contained polynucleotides from the tandem insert library polynucleotides at different concentrations: lane 1 had 2 pM, lane 2 had 10 pM, lane 3 had 20 pM, lane 6 had 2 pM, lane 7 had 10 pM, and lane 8 had 20 pM. Lanes 4 and 5 were lanes for control reactions: lane 4 had monotemplate control reaction and lane 5 had PhIX sequencing library control reaction (FIG. 19). Reads 1 and 4 were used to sequence inserts from the E. coli genome (FIG. 19). Reads 2 and 3 were used to sequence inserts from the human genome (FIG. 19).

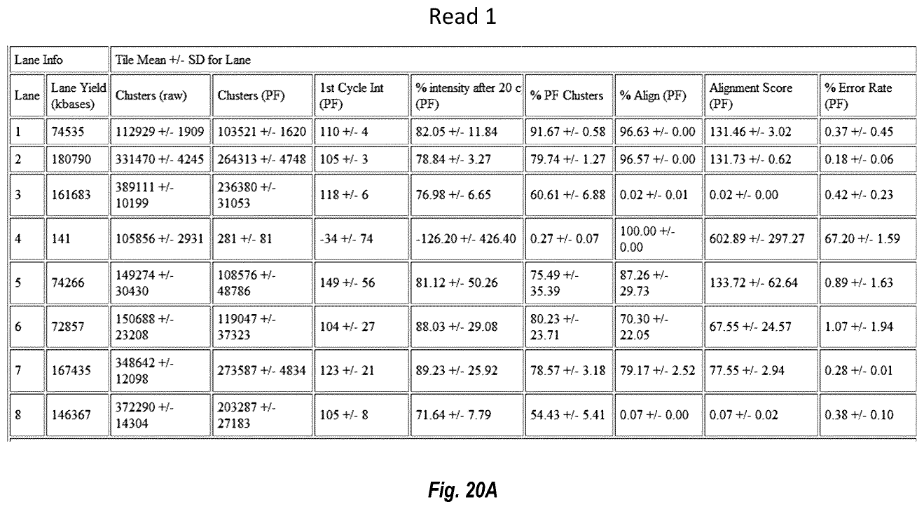

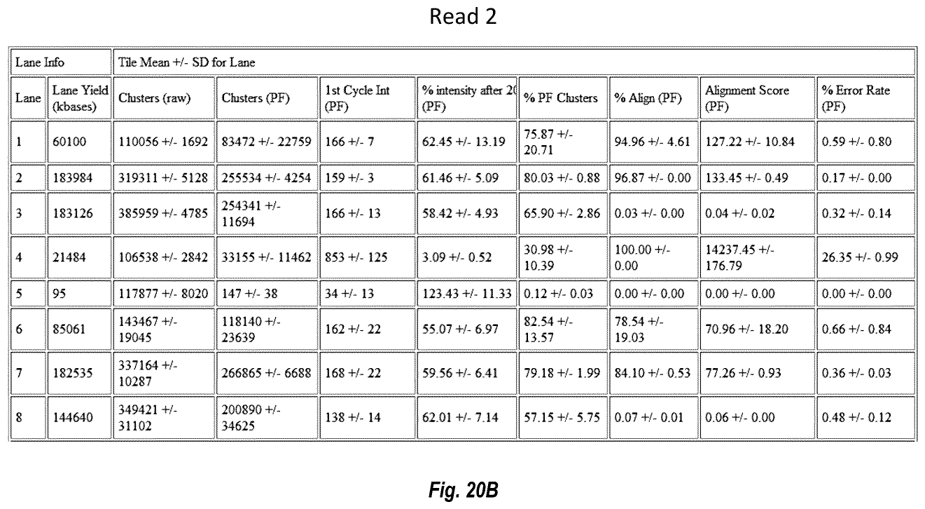

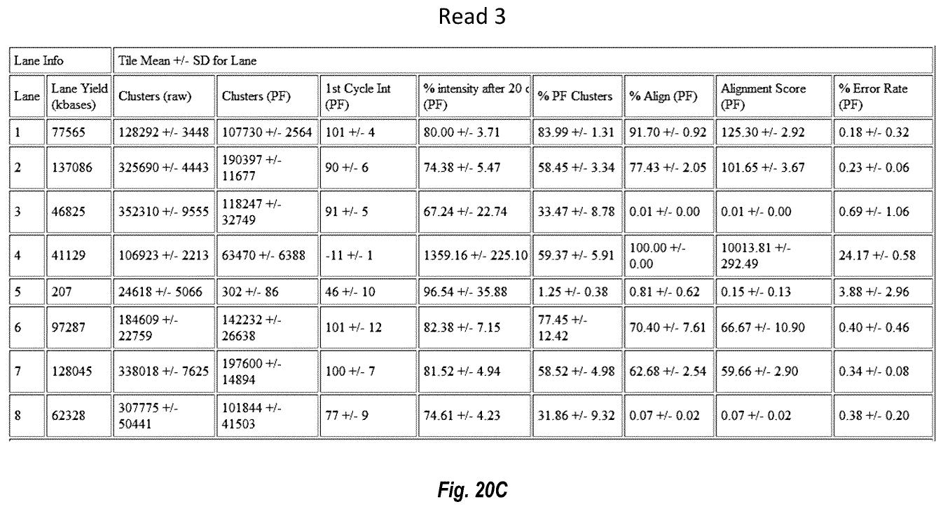

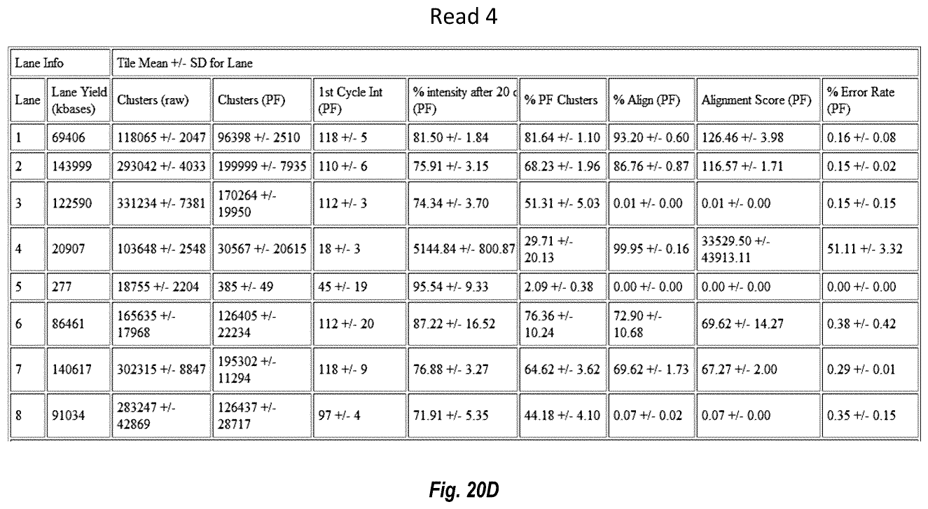

As shown in FIGS. 20A-D, lanes clustered at 2 pM or 10 pM generated a high percentage of pure clusters that passed purity filters (% PF) indicating a successful clustering and sequencing of correctly formed templates. Moreover, a high percentage of the reads when aligned to the expected reference genomes matched correctly indicating that the templates contained the expected inserts.

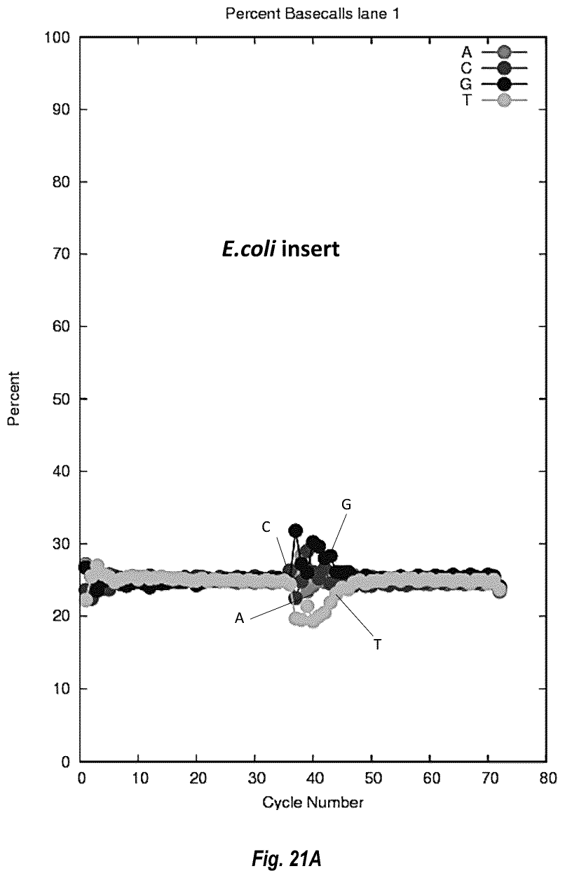

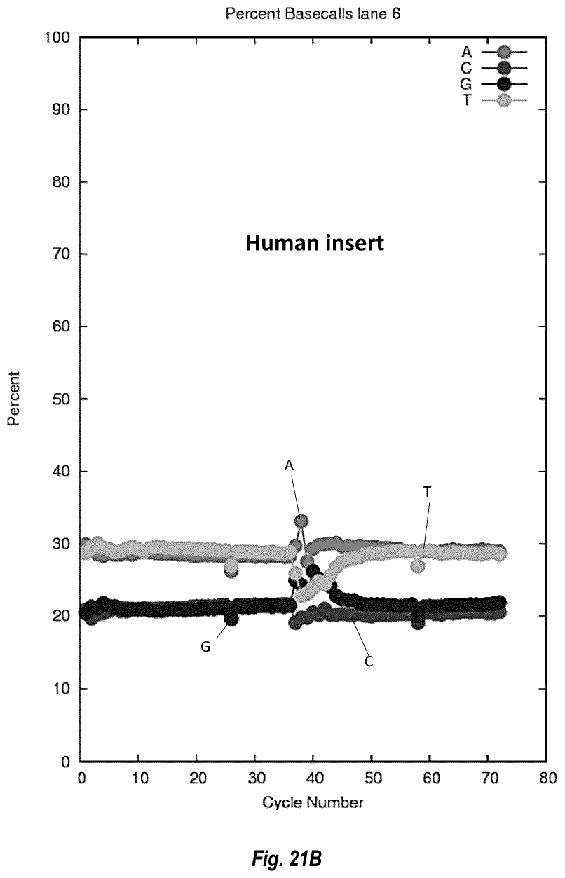

The proportion of each of the 4 bases detected at each cycle of sequencing for both inserts are represented in a % base-call per cycle plot in FIGS. 21A-B. A, T, G, and C were expected and observed to occur at a proportion of 25% for each cycle in the first insert which contained E. coli fragments. Similarly, A, T, C, and G were expected and observed to occur at a proportion of 30%, 30%, 20%, and 20% for each cycle in the second insert which contained human fragments. The data indicates that 4 reads were conducted that detected two inserts in the library as designed.

### Example 10. Preparation of Polynucleotides from Starting Libraries with Monotemplates Using the Strand Overlap Extension (SOE) Method

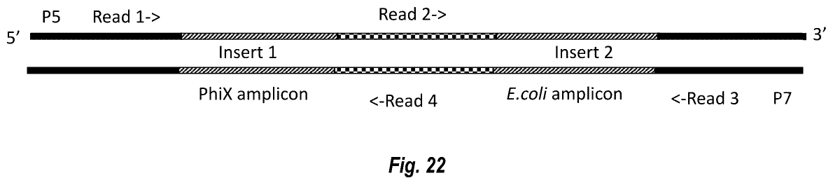

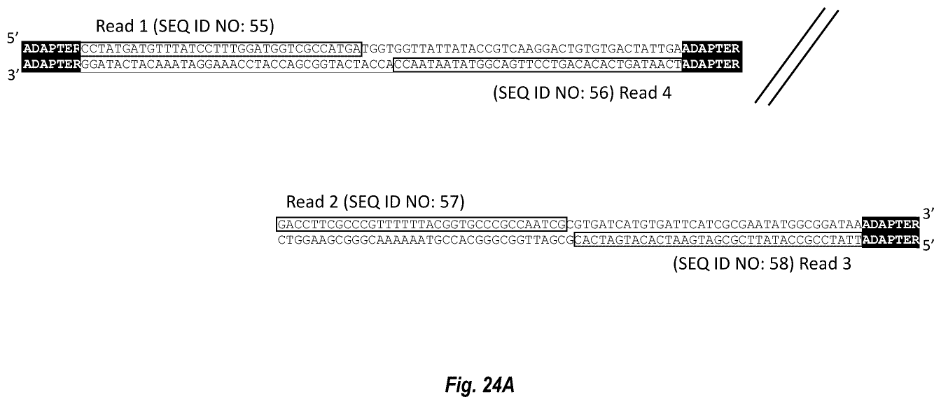

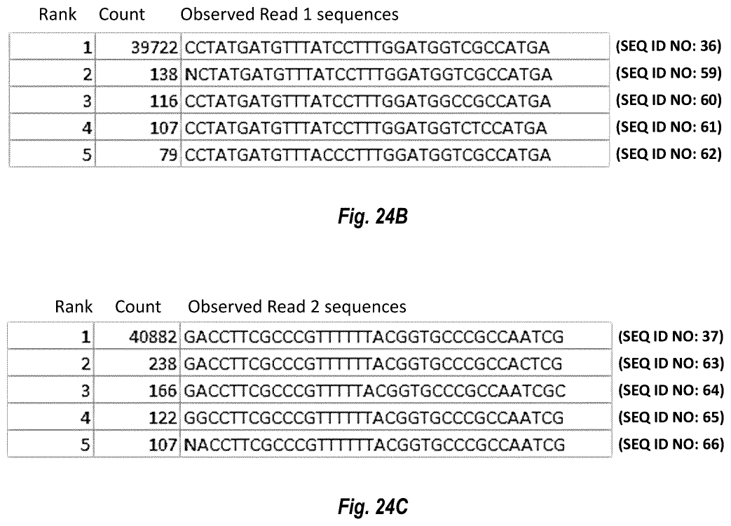

Polynucleotides comprising multiple insert sequences were generated using a method comprising strand overlap extension (SOE). In the exemplary method described herein (FIGS. 16A-B and 17), a first library contained inserts monotemplates (i.e., amplicons) from E. coli and a second library contained monotemplates from PhiX (FIGS. 22 and 24A-C). At least two different sets of amplicons were used. Adapters were ligated to the monotemplates and the tandem insert library was produced using the SOE method shown in (FIGS. 16A-B and 17).

A sequencing flow cell was prepared that contained polynucleotides from the tandem insert library polynucleotides in all lanes except for lane 5, which contained a single insert control PhiX library. Reads 1 and 4 were used to sequence inserts from the PhiX monotemplate (FIG. 22). Reads 2 and 3 were used to sequence inserts from E. coli monotemplate (FIG. 22).

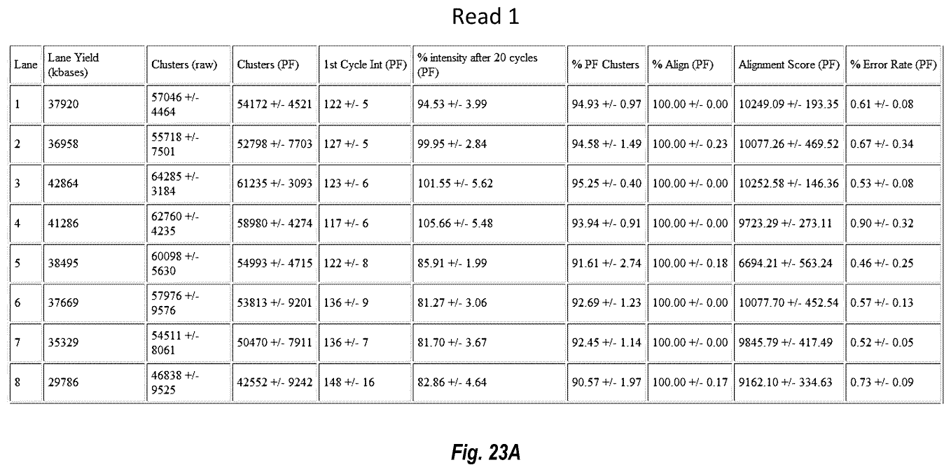

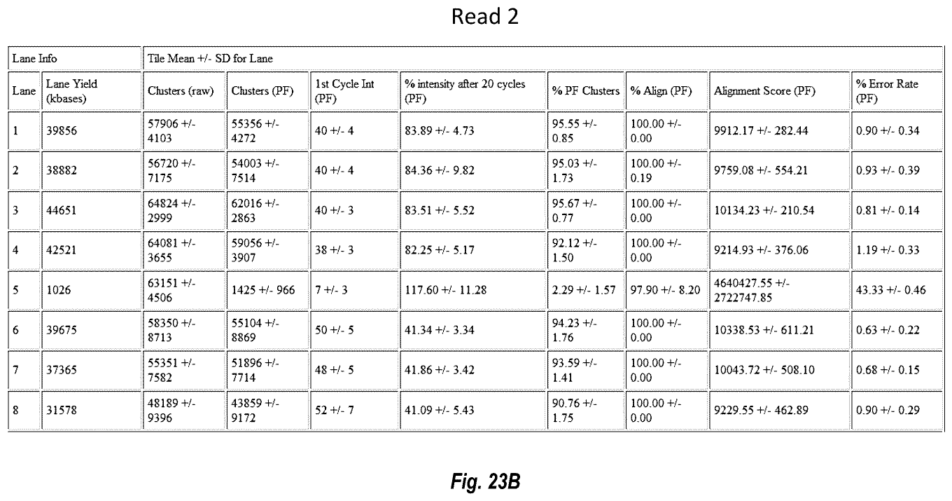

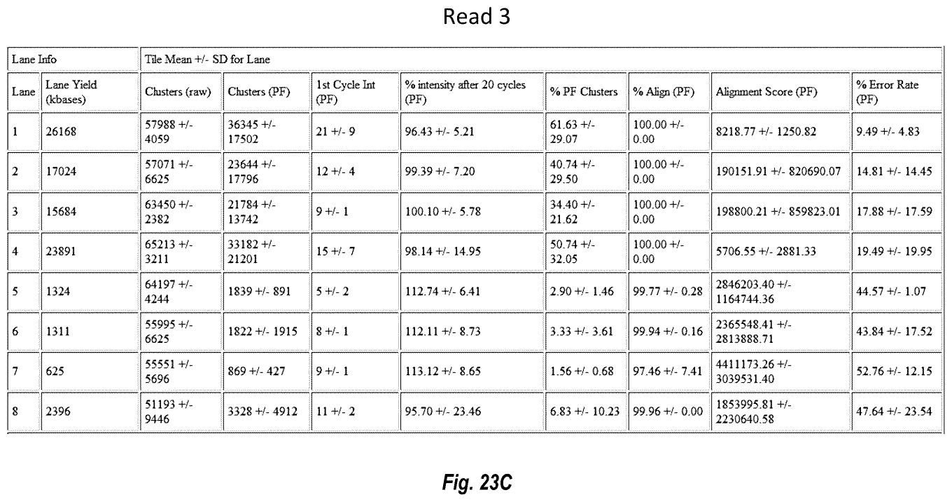

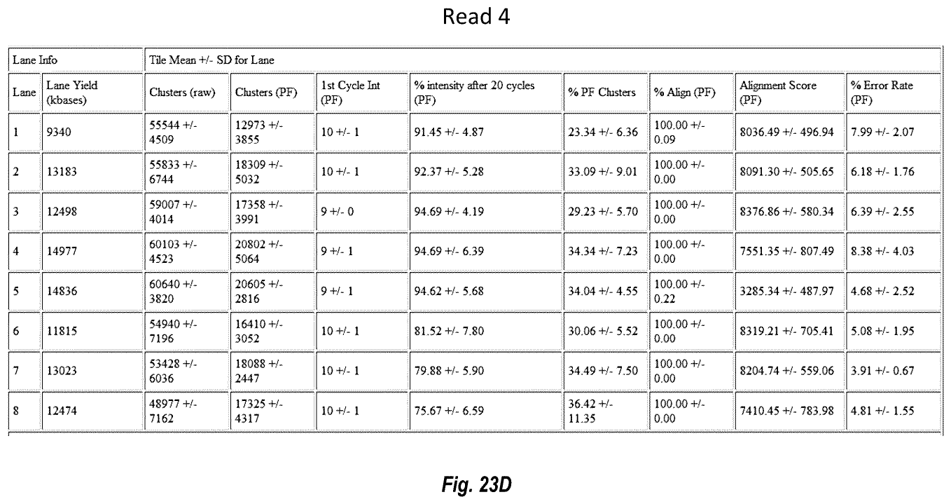

Primary metrics from the four-read sequencing run are shown in FIGS. 23A-D. Reads 1 and 2 which cover the first and second inserts, respectively, show cluster numbers, % PF, and % align, indicating that the presence of the two inserts in each polynucleotide. In contrast, lane 5, which contained the single insert control, yielded no meaningful data for read 2, indicating the absence of a second insert.

FIGS. 24A-C illustrates the complete amplicon sequence of the tandem insert polynucleotide produced using the method of this example. (The adapter sequences are marked as “ADAPTER” and their actual sequences are not shown.) FIGS. 24A-C show expected sequences from the sequencer instrument output, highlighting the top five most common read sequences for Read 1 and Read 2, and their counts. Read 1 read into the first insert and Read 2 read into the second insert. The data indicates the presence of both amplicons and confirms that a tandem insert polynucleotide was successfully generated.

The proportion of each of the 4 bases detected at each cycle of sequencing for both inserts are represented in a % base-call per cycle plot in FIGS. 21A-B. A, T, G, and C were expected and observed to occur at a proportion of 25% for each cycle in the first insert which contained E. coli fragments. Similarly, A, T, C, and G were expected and observed to occur at a proportion of 30%, 30%, 20%, and 20% for each cycle in the second insert which contained human fragments. The data indicates that 4 reads were conducted that detected two inserts in the library as designed.

### Example 11. Preparation of Sequencing Templates Comprising Two or More Inserts Using Forked Adapters and a Solid Support

A method of preparing sequencing templates comprising two or more inserts may be performed with forked adapters and a surface for immobilizing fragments with ligated adapters, with the solid support allowing hybridization of multiple fragments together to generate concatenated sequencing templates.

A first and a second adapter can be prepared, as shown in FIG. 25. The adapters can be “Y-shaped” or “forked” in structure, such that two adapters each comprise a first oligonucleotide and a second oligonucleotide that are partially hybridized to each other to form a double-stranded section and a single stranded section (i.e., each adapter is a forked adapter). Each forked adapters comprises a binding moiety for attaching the adapter to a surface. This moiety binding may be a biotin or other chemistries known to those skilled in the art. The moiety may be present on the 5′ end on one of the oligonucleotides in the forked adapter, which may be termed the “first stand” of the forked adapter. The first strand may comprise full or partial sequences corresponding to the “Read 1” sequences of Illumina's sequencing platform (referred to as P5.R1), and in the case of the second adapter, the ‘Read 2’ sequences of Illumina's sequencing platform (e.g. P7.R2). The second strand comprises two sections, a 5′ end section and a 3′ end section. The 5′ end section is complementary and hybridized to the 3′ end of the first strand. The 3′ end section of the second strand (X′) in the first adapter is complementary to the 3′ end section of the second oligonucleotide (X) in the second adapter. X and X′ may be a hybridization sequence and the complement of a hybridization sequence, respectively.

A blocking oligonucleotide may be hybridized to one or both forked adapter at the 3′ end of the second strand of either forked adapter (i.e., a blocking oligonucleotide is hybridized to the single-stranded section of the second strand of the forked adapter). This blocking oligonucleotide may be hybridized to either, or both, the first forked adapter or the second forked adapter (FIG. 26). The blocking oligonucleotide prevents the first forked adapter and the second adapter from hybridizing to one another via the 3′ complementary sections of each second strand (i.e., the X and X′ sequences shown in FIG. 26, which may correspond to a hybridization sequence and the complement of a hybridization sequence, respectively).

When a mixture of the first forked adapter and the second forked adapter is ligated to the ends of a double-stranded DNA fragment comprising a first strand (the top strand A in FIGS. 27A-27C) and a bottom strand (the bottom complement A′ in FIGS. 27A-27C), three different tagged library products can be formed: a fragment with a first forked adapter at one end and a second forked adapter at the other end (FIG. 27A), a fragment with a first forked adapter at both ends (FIG. 27B), or a fragment with a second forked adapter at both ends (FIG. 27C). The different fragments (as shown in FIGS. 27A-27C) will be formed in a ratio of 50 (FIG. 27A):25 (FIG. 27B):25 (FIG. 27C).

The fragments with ligated adapters can then be added to a surface and attached via the 5′ affinity moiety of the first strands of the forked adapters. The surface may be a bead, or a slide, or a wall of a vessel, or a nanowell on a flow cell. The fragments can next be denatured and subject to flow such that the blocking oligonucleotide is removed. Denaturation can occur by several ways known to those skilled in the art, including heat, pH, or chaotropic agents.

When the surface is subject to conditions that favor renaturation (such as cooling of the surface), the two single-stranded fragments may fully reanneal across their entire length. Alternatively, only single-stranded fragments that have an adapter sequence from a first forked adapter at one end and an adapter sequence from a second forked adapter at the other may reanneal just by their 3′ complementary ends (i.e., binding of the X sequence of the second strand of the second forked adapter with the X′ sequence of the second oligonucleotide of the first forked adapter, as shown in FIG. 28A). Polymerase, dNTPs and buffer can be added to extend the polynucleotide from the 3′ end to generate a new template comprising two inserts in tandem (FIG. 29).

Fragments that comprise a sequence from a first forked adapter at both ends cannot anneal to each other via their 3′ ends (FIG. 28B) and thus cannot be extended, because a X′ sequence will not anneal to another X′ sequence. Likewise, fragments that comprise a sequence from a second forked adapter at both ends cannot anneal to each other via their 3′ ends (FIG. 28C) and thus cannot be extended, because a X sequence will not anneal to another X sequence. The process of denaturation, reannealing, and extension can be performed multiple times until all the fragments comprising a sequence from a first forked adapter at one end and a sequence from a second adapter at the other end (FIG. 28A) have been converted into sequencing templates comprising tandem inserts (i.e., two or more inserts within the same polynucleotide).

As shown in FIG. 29, a sequencing template can comprise the original A top strand as an insert linked to a copy of the A top strand as a second insert. Any variants present in the original A strand will be reproduced in the copy A strand and thus will increase the confidence in the base-calling of the variant when both copies are sequenced. Likewise, a variant that only appears in the copy A strand can be dismissed with increased confidence as an artifact. In this manner, this embodiment improves the accuracy of base-calling in sequencing.

The concatenated sequencing template also comprises the complement the original A′ bottom strand linked to a copy of the A′ bottom strand. In the final stage of library preparation for sequencing, the top and bottom strands are harvested from the surface by disrupting the 5′ surface binding moiety, followed by denaturing the library. Thus, the top and bottom strand are sequenced independently of one another. They may also be replicated by PCR or other methods that copy DNA before sequencing.

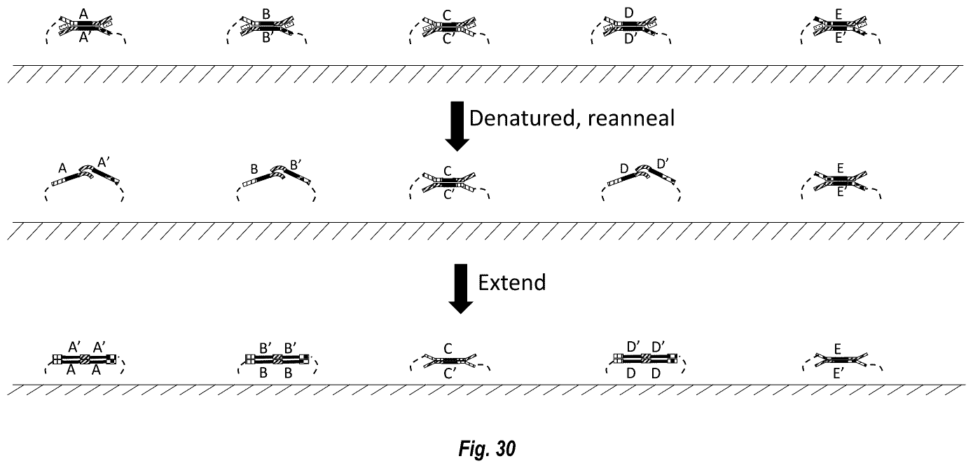

FIG. 30 illustrates an overview of a method where a multitude of library fragments, in this example represented by the 5 fragments A, B, C, D, and E, are bound to a surface, denatured, reannealed, and then extended to form concatenated sequencing templates. Templates that have a sequence from a first forked adapter at both ends or a sequence from a second forked adapter at both ends cannot reanneal via their 3′ ends (e.g., templates C and E in FIG. 30) and thus cannot be extended. The double-stranded fragments (which are then denatured to single-stranded fragments) may be added (and immobilized) to the surface at a density that favors reannealing of the two fragments from a double-stranded fragments to produce a concatenated sequencing template comprising two copies of the same insert, rather favoring annealing of two fragments from different double-stranded fragments.

In other cases, a sequencing template may comprise two insert of more inserts that are not copies of each other. Such sequencing templates can be generated by two fragments that anneal by binding of X to X′, without the inserts in the two fragments being complementary. In other words, some sequencing templates can have two copies of the same insert, while other sequencing templates can comprise two different inserts with unrelated sequences.

### Example 12. Preparation of Sequencing Templates Comprising Two or More Inserts Using a Compartmentalization

A method for preparing sequencing templates comprising two or more inserts may use forked adapters and a means of compartmentalization.

A pool of DNA molecules, for example, separate genomes, separate chromosomes, or large fragments of DNA (>1000 bp, preferably greater than 5000 bp) is aliquoted into multiple compartments by limiting dilution such that an individual compartment contains no DNA molecules, a single DNA molecule, or a limited number of DNA molecules equating to a fraction of one haploid copy whereby any position of the genome is likely to be represented by haploid DNA. Methods incorporating compartmentalization primarily capture contiguity information, but these methods can also produce concatenated sequencing templates with two copies of a given insert sequence (via hybridization of fragments comprising a sense strand and antisense strand of the same insert sequence).

Methods of compartmentalization (such as for use in preparing whole-genome haplotyping) are well-known in the art, such as those taught in Amini et al., Nat Genet. 46(12):1343-9 (2014); Kaper F, et al. Proc. Natl. Acad. Sci. USA. 110(14):5552-5557 (2013); Kitzman J O, et al. Nat. Biotechnol. 29(1):59-63 (2011); Peters B A, et al. Nature. 487(7406):190-195 (2012); Fan H C, et al. Nat. Biotechnol. 29(1):51-57 (2011); Levy S, et al. PLoS Biol. 5(10):e254 (2007); Duitama J, et al. Nucleic Acids Res. 40(5):2041-2053 (2012); Suk E K, et al. Genome Res. 21(10):1672-1685 (2011), each of which is incorporated by reference in its entirety herein. A user may choose a specific means of compartmentalization, such as emulsions, based on their preference and available equipment, and this method can be adapter to a variety of compartmentalization methods known in the art.

FIG. 31 illustrates a method wherein the compartment is a well on a plate or a number of tubes and the starting pool contains 3 molecules: f1, f2 and f3. Each compartment is subjected to library preparation (i.e., fragmentation of a starting double-stranded DNA molecule that may itself be a relatively large fragment, repair of the ends of the subfragments, and a ligation reaction using a mixture of a first forked adapter and a second forker adapter as described in Example 11 to form end-ligated subfragments). Next, the subfragments are denatured and reannealed via their 3′ complementary ends and extended to form tandem insert templates. As shown in the exemplary embodiment in FIG. 31, the molecule in the compartment that contained fragment molecule f1 was fragmented into three sub-fragments f1.1, f1.2, and f1.3. The resulting tandem insert templates are accordingly permutations of these three subfragments, e.g. f1.1-f1.2, f1.1-f1.3, and f1.2-f1.3. Other permutations of the same subfragment are also possible, e.g. f1.1-f1.1, f1.2-f1.2, and f1.3-f1.3.

It will be appreciated that a different compartment (e.g., a compartment comprising f2, f3, etc.) will also form tandem insert templates, but only from permutations of the starting molecules within those wells. In other words, only subfragments generated in the same compartment are available to hybridize together to generate concatenated sequencing templates. Accordingly, the presence of two insert sequences together in a concatenated sequencing template can be used to infer that these insert sequences were comprised in the same starting DNA molecule (such as fragment f1, f2, or f3 in FIG. 31), especially when conditions are optimized such that only a single DNA molecule is generally present in a compartment.

Accordingly, contiguity information is captured in the concatenated sequencing templates even when the tandem insert templates from all compartments are pooled together and sequenced. FIG. 31 shows a representative example of three fragments, more than three fragments from a starting double-stranded DNA molecule (before fragmenting) are also possible.

An advantage of using wells or tubes as compartments is that reagents can be added at each stage of the process. A potential disadvantage of using wells or tubes is the physical scale of the liquid handling and plasticware. Hence, alternative methods of compartmentalization using droplets of water in oil have been developed that use microfluidics. Droplets can be merged to add reagents such as endonucleases that fragment DNA. Droplet technology has been used to capture contiguity information (see, for example, exemplary methods outlined in “Everything you wanted to know about Linked-Reads,” 10× Genomics, Feb. 7, 2017), but such methods often require the addition of exogenous synthetic barcodes to link contiguous sequences.

FIG. 32 illustrates an exemplary method using a first forked adapter and a second forked adapter, wherein the first and second forked adapters comprise complementary 3′ ends, with the use of droplets for compartmentalizing the workflows. Similar to methods with compartments (such as wells or tubes), fragments f1, f2, and f3 may be comprised in separate droplets. After ligating forked adapters and generating concatenated sequencing templates, emulsions can then be merged together in a final step. The presence of different insert sequences in the same concatenated sequencing template can be used to infer that these insert sequences were comprised in the same starting nucleic acid, especially if emulsions are prepared where more starting nucleic acids are individually comprised in a droplet.

FIG. 33 illustrates an example of haplotype phasing wherein two or more variants in a gene can be ascribed to their originating chromosome haplotype. In this example, the starting sample has two unrelated genes, one on chromosome 1 and one on chromosome 2. Two variants, snp1 and snp2, are present in the gene on chromosome 1, but these two variants are only found on one of the two copies of the gene, i.e., that gene found on chromosome 1/Haplotype 1 (i.e., Chr1-Hap1) contains both variants. The second copy of this gene on the other chromosome 1/Haplotype 2 (i.e., Chr1-Hap2) bears no variants at these loci, and the sequences at these loci are wild-type (wt). Thus, the phased haplotypes for gene 1 are Chr1-Hap1-snp1-snp2 and Chr1-Hap2-wt-wt Likewise, the second gene on chromosome 2 also has two copies: Chr2-Hap1 and Chr2-Hap2, but in this case the two variants (snp3 and snp4) are on not in cis (i.e., both variants in the same copy) but instead a variant is found in either copy of the gene in the two haplotypes. Thus, the phased haplotypes are: Chr2-Hap1-snp3-wt and Chr2-Hap2-wt-snp4.

As a consequence of limiting dilution to sub-haploid concentrations and compartmentalization, two copies (haplotypes) of the same gene are unlikely to be present in the same compartment. For preparing haplotype data, however, dilutions need not limit to one or no target nucleic acid in a given compartment, but instead can allow for different chromosomes to be comprised in the same compartment. The dilution would only generally need to limit the probability of two haploid copies ending up in the same compartment.

As shown in FIG. 33, one compartment has Chr1-Hap1-snp1-snp2 and Chr2-Hap1-snp3-wt whereas another compartment has Chr1-Hap2-wt-wt and Chr2-Hap2-wt-snp4. Following denaturation, reannealing via the 3′ end of the templates, and extension, many permutations of tandem inserts are possible, including those that constitute the original haplotypes (as indicated by those encircled in a dashed line circles highlighted by the checked arrow in FIG. 33). However, because of the compartmentalization, permutations that scramble the haplotypes are not possible, e.g., Chr1-Hap1-snp1-Chr1-Hap2-wt or Chr2-Hap1-snp3-Chr2-Hap2-snp4 (shown as options highlighted with an arrow comprising “X” in FIG. 33). In this manner, phasing information is captured by the tandem insert approach without the necessity of barcoding.

### Example 13. Preparation of Sequencing Templates Comprising Two or More Inserts Using a Solid Support with Immobilized Transposomes

Sequencing templates comprising two or more inserts can also be prepared using a solid support with immobilized transposomes. A first and a second transposome are prepared as shown in FIG. 34. The first transposome comprises a complex of a transposase enzyme and a first adapter. The second transposome comprises a complex of a transposase enzyme and a second adapter. The adapters are ‘Y-shaped’ or ‘forked’ in structure as the two oligonucleotides, a first strand and a second strand, are partially hybridized to one another to form a forked adapter comprising double-stranded section and a single-stranded section. The first strand and second strand may also be termed the first transposon and the second transposon.

Both the first and second adapters comprise an affinity moiety that can bind to a binding moiety on a surface of a solid support to attach the first strands to the surface. In other words, association of the binding moiety on a surface with an affinity moiety in a transposome can be used to immobilize the transposomes on the surface. The affinity moiety may be a biotin or other chemistries known to those skilled in the art. The affinity moiety is present on the 5′ end of one of strands in a forked adapter comprised in the transposome. The first strand of the forked adapter comprised in the first transposome comprises full or partial sequences corresponding to the ‘Read 1’ sequences of Illumina's sequencing platform (e.g., P5.R1), and the first strand of the forked adapter comprised in the second transposome comprises full or partial sequences corresponding to the ‘Read 2’ sequences of Illumina's sequencing platform (e.g., P7.R2).

The second strand of each forked adapter can comprise two sections, a end section and a 3′ end section. The 5′ end section of the second strands is complementary and hybridized to the 3′ end of the first strands. The 3′ end section of the second strand (X′) of the forked adapter comprised in the first transposome adapter is complementary to the 3′ end section of the second strand (X) of the forked adapter comprised in the second transposome.

The transposomes are attached to a surface via the 5′ end of the first strand of the forked adapter comprised in the first and second transposome. Methods for attachment are known to those skilled in the art, for example, biotinylation of oligonucleotides to attach to streptavidin-coated surfaces. Attachment to the surface may result in a random arrangement of the two transposomes (FIG. 35) or in some embodiments the arrangement may be ordered in an array of fixed predetermined locations on the surface. A strand of double-stranded DNA added to this surface will undergo tagmentation by transposomes positioned by chance under the contact point of the DNA with the surface. Tagmentation results in the joining of the immobilized first transposon to the tagmented DNA, and the tagmented DNA is immobilized to the surface of the solid support.

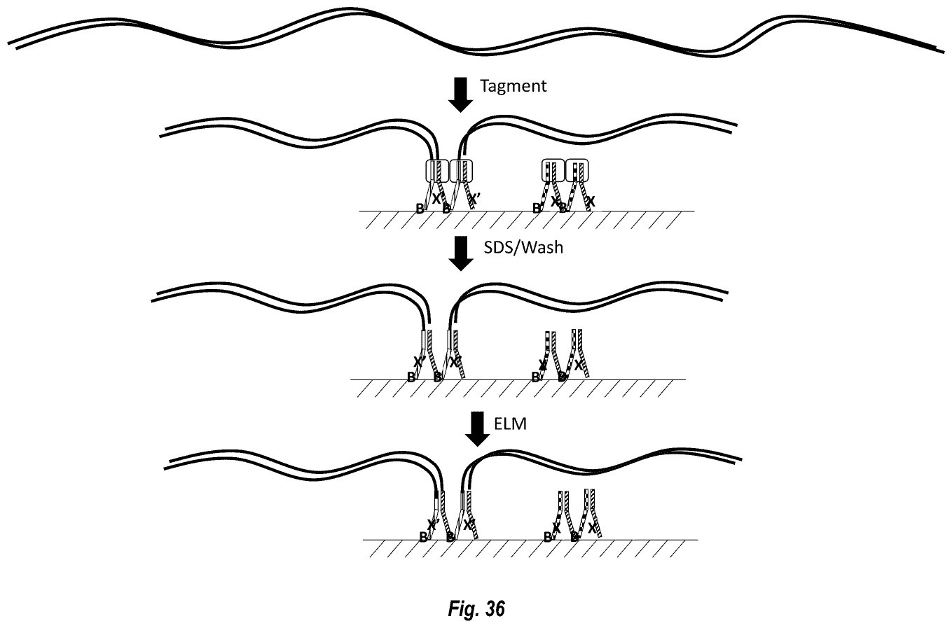

A strand of double-stranded DNA added to this surface with immobilized transposomes will undergo tagmentation by one or multiple transposomes positioned by chance under the contact point of the DNA with the surface (FIG. 35). An individual tagmentation reaction can be performed with a first transposome or a second transposome. Tagmentation cleaves DNA and covalently attaches the 3′OH end of the first strand of the adapter to the 5′ end of the cut DNA. The 5′ end of the second strand in the adapter is not attached and a nick/gap forms that is sealed by a polymerization/ligation reaction with reagent ELM (extension-ligation mix). In order for this reaction to succeed, the transposase enzyme must be removed by SDS and washing (FIG. 36).

The DNA to surface transposome ratio can be selected such that no more than two tagmentation events occur per double-stranded DNA molecule. Where two tagmentation reaction occur per double-stranded DNA, bridges are formed between neighboring transposomes.

Where a tagmentation reaction occurs with a first transposome and a second transposome, a bridge is formed comprising a segment of the starting DNA (e.g., segment A) with adapters appended at both ends. The bridges may be between a first transposome and a second transposome, or a first transposome and a first transposome, or a second transposome and a second transposome. Such permutations will occur in a ratio of 50:25:25, respectively.

When these bridges are processed to remove the Tn5 transposase (such as with SDS and washing), to seal the nicks/gaps, and then to denature the double-stranded fragments into single-stranded fragments, different combinations of templates can be formed.

For example, where the bridge is formed between a first transposome and a second transposome, two single stranded templates are formed, 5′-P5-R1-A-X-3′ and 5-′P7-R2-A′-X′-3′ (FIG. 38). Where the bridge is formed between a first transposome and a first transposome, two single stranded templates are formed, 5′-P5-R1-A-X′-3′ and 5′-P5-R1-A′-X′-3′. Where the bridge is formed between a second transposome and a second transposome, two single stranded templates are formed, 5′-P7-R2-A-X-3′ and 5′-P7-R2-A′-X-3′.

The single-stranded strands are then treated to promote reannealing by methods known to those skilled in the art, for example, cooling or conducive buffer conditions. One outcome is that single-stranded fragments simply reanneal to their complement. Alternatively, single-stranded fragments may reanneal by their 3′ complementary ends, i.e., via binding of an X sequence to an X′ sequence. This is only possible between the first transposome and second transposome adapters, i.e., 5′-P5-R1-A-X-3′ and 5-′P7-R2-A′-X′ (FIG. 39). 5′-P5-R1-A-X′-3′ and 5′-P5-R1-A′-X′-3′ cannot hybridize nor can 5′-P7-R2-A-X-3′ and 5′-P7-R2-A′-X-3′. When a polymerase and dNTPs are added and an extension reaction performed, a tandem insert template duplex is formed comprising two copies of the A-strand in tandem in the sense strand and two copies of the A′-strand in tandem in the antisense strand (FIG. 40). Two single-stranded inserts cannot pair if they both comprise a X′ sequence or both comprise a X sequence.

Where two bridges are formed by three tagmentation events, for example the two bridges represented by A and B in FIG. 41, then a larger number of permutations of cross insert hybridization is possible depending on the permutations of first and second transposome. These will produce chimeric template with two inserts that are permutations of the contiguous A and B segments from the starting DNA (FIG. 42). This contiguity association of inserts is revealed by sequencing the tandem templates. Some of these concatenated sequencing templates will not amplify due to suppression during PCR based on their complementary ends. Also, some concatenated sequencing templates will not produce a sequence on a NGS platform because of the complementarity between their 5′ and 3′ sequences. For example, P5-R1-A′-x-B′-R1′-P5′ and P5′-R1′-A-x′-B-R1-P5 would not produce sequences on an Illumina sequencer because they comprise P5/P5′ at both ends and would not be available for paired-end sequencing that require P5/P5′ at one end of fragments and P7/P7′ at the other end. Examples of concatenated sequencing templates that would not produce sequences on an Illumina sequencer are indicated on FIG. 42 in hashed line boxes.

It will be appreciated that two bridges may also form between three transposomes comprising a second forked adapter or three transposomes comprising a first forked adapter (FIG. 43). In these instances, no complementarity is present between the 3′ ends of the denatured templates (FIG. 44), and thus no tandem insert templates are produced.

Where more than two bridges are formed, for example the five bridges represented by A, B, C, D, E in FIG. 45, then multiple concatenated sequencing templates may form that share sequences. For example, insert A may hybridize with insert B; and insert B′ may hybridize with insert C′; insert C may hybridize with insert D; etc. The resulting extended templates when sequenced will enable contiguity information to be discovered as well as providing phasing of variants.

The process of denaturation, reannealing, and extension can be performed multiple times until all the templates comprising an adapter from the first strand of the forked adapter comprised in the first transposome at a first end and an adapter from the second strand of the forked adapter comprised in the second transposome at a second end are converted into sequencing templates comprising two inserts.

The sequencing templates can then be detached from the surface by disrupting the linkage joining the tag incorporated from the 5′ end of the first strand of the forked adapters with the surface, using means known to those skilled in the art, for instance by enzymatic digestion or chemical cleavage. The released templates can then be introduced to a sequencing platform directly or may first undergo further modification such as the addition of additional adapter sequences or amplification by PCR followed by sequencing.

The present method does not require barcodes to capture association information about contiguous and complementary sequences within the genome. However, where two or more libraries of templates from different samples are pooled before sequencing, a sample barcode may be desired. Sample barcodes may be included in the first strands of forked adapters (FIG. 46A), second strands of forked adapter (FIG. 46B), or both first and second strands of forked adapter (FIG. 46C). Sample indexes include i5-i8. Alternatively, unique molecular identifiers (UMIs) may be used to label different fragments prepared by different transposome complexes, wherein the UMIs can be comprised in the first and/or second strand of the forked adapters comprised in transposomes. Different sequencing runs using primers that bind A14, B15, or HYB (or their complements) may then be used to sequence inserts sequences as well as sample indexes and/or UMIs, as shown in FIG. 47.

### Example 14. Preparation of Sequencing Templates Comprising Two or More Inserts Using Transposomes Comprised in Compartments

Transposomes may also be used with methods of limited dilutions and/or compartmentalization as described in Example 12. The transposomes may be first and second transposomes as shown in FIG. 34, to allow for incorporation on X′ on some fragments and X on other fragments.

In such methods, transposomes may be in solution and may not be immobilized on a solid support. Transposomes may also be immobilized on a solid support (such as a bead) wherein most compartments only comprise a single solid support. DNA molecules within a compartment are tagmented with the first and second transposomes present in the compartment but not necessarily attached to a surface to produce double-stranded tagged fragments.

The tagged fragments can then be denatured to prepare single-stranded fragments, and hybridization may be allowed between a X sequence on one fragment and a X′ sequence on another fragment. After hybridization, extension may be performed to prepare concatenated sequencing templates. These concatenated sequencing templates can then be sequenced.

If solution-phase transposomes are used, this method may likely generate concatenated sequencing templates that comprise two different insert sequences (as opposed to concatenated sequencing templates comprising two copies of the same insert) since the single-stranded fragments will not be immobilized before the hybridizing. Since the compartments can be optimized to generally comprise one or no DNA molecules before tagmentation, the presence of a concatenated sequencing template with two different insert sequences in sequencing results can be used to infer that these two insert sequences originated from sequences comprised in a single DNA molecule (i.e., neighboring or proximal sequences within a DNA molecule).

### Example 15. Methylation Analysis Using Concatenated Sequencing Templates

Concatenated sequencing templates described herein may be used for methylation analysis.

FIG. 48 illustrates a method wherein a DNA fragment comprising methylated and hydroxymethylated cytosines is incorporated into a concatenated sequencing template. In this example, the ‘sense’ strand(s) of the original duplex contains a sequence that includes the following bases 5′-C.A.^{m}C.G.^{hm}C.G.T-3′, where C represents an unmethylated cytosine base, mC represents a methylated cytosine base, and h mC represents a hydroxymethylated cytosine. The ‘antisense strand’ (S′) is the complement of the sense strand and is also methylated thus: 3′-G.T.G.^{m}C.G.^{hm}C.A-5′. After conversion to a tandem insert template using unmethylated dCTP nucleotides, the ‘sense’ strand is linked in tandem to a copy of the ‘sense’ strand (s-copy) that bears no methylated cytosines and the sequence is as follows: 5′-C.A.^{m}C.G. ^{hm}C.G.T-x-C.A.C.G.C.G.C.T-3′. The ‘antisense strand’ (s′) is similarly linked in tandem to a copy of the ‘antisense’ strand (s′-copy) that bears no methylated cytosines and the sequence is as follows: 3′-G.T.G.C.G.C.A-x′-G.T.G.^{m}C.G.^{hm}C.A-5′.

The concatenated sequencing template may then undergo a conversion process to identify methylated C's.

As shown in FIG. 49, the concatenated sequencing template may be subjected to chemistries that convert non-methylated C's to U's, such as with sodium bisulfate chemical conversion or with an enzymatic reaction such as EM-Seq.

FIG. 50A illustrates the fate of the top strand of the concatenated sequencing template shown in FIG. 49 containing the ‘sense’ sequence(s) linked to a copy of the sense sequence (s-copy), after conversion of non-methylated C's to U's. After PCR, the U's are transformed to T's. When this single-stranded concatenated sequencing template is sequenced and the ‘sense’ sequence (s) compared to the copy of the sense sequence (s-copy), each base of the original template (prior to conversion to a tandem insert template) is represent by a ‘code’ of two ‘base-calls’. This ‘2-base’ code will depend upon the methylation status of the original template. Thus, in the example in FIG. 50A, the original sense strand (s) 5′-C.A.^{m}C.G.^{hm}C.G.T-3′ is encoded as: 5′-(T,T) (A,A) (C,T) (G,G) (C,T) (G,G) (T,T)-3′

FIG. 50B similarly illustrates the fate of the bottom strand of the concatenated sequencing template shown in FIG. 49 containing the ‘antisense’ sequence (s′) linked to a copy of the antisense sequence (s′-copy), after conversion of non-methylated C's to U's. After PCR, the U's are transformed to T's. When this single-stranded concatenated sequencing template is sequenced, the original antisense strand (s) 3′-G.T.G.^{m}C.G.^{hm}C.A-5′ is encoded as: 3′ (G,G) (T,T) (G,G) (T,C) (G,G) (T,C) (A,A) 5′.

The codification of the original bases is further developed and refined by collating the ‘2-base’ codes from the reads from the top strand and bottom strand of the tandem insert templates, using the method shown in FIG. 50C. This generates a ‘2×2-base’ code that enables the methylation status of the original duplex to be deciphered. For example, in the example in FIG. 50A where a chemistry such as bisulfate is used that converts non-methylated cytosines, a top strand/bottom strand ‘2×2-base’ code of (T,T)/(G,G) identifies that the original base pair was a unmethylated cytosine in the top strand and a guanine in the bottom strand. In contrast, a code of (C,T)/(G,G) identifies that the original base pair was a methylated cytosine in the top strand and a guanine in the bottom strand. Similarly a code of (G,G)/(T,C) identifies that the original base pair was a guanine in the top strand and a methylated cytosine in the bottom strand. In this workflow, methylated cytosines cannot be distinguished from hydroxymethylated cytosines.

Methylation analysis can also be performed wherein the conversion is performed on methylated cytosines, and not unmethylated cytosines, as shown in FIG. 51 using the TAPS workflow as described in Liu et al., Nature Biotechnology 37(4):424-429 (2019). TAPS converts modified cytosine into dihydroxyuracil (^{DH} U), a near natural base, which can be “read” as T by common polymerases. A ‘2×2-base’ code is generated as shown in FIGS. 52A and 52B and although the codes are different, they still enable the methylation status to be identified as described above (though methylated cytosines cannot be distinguished from hydroxymethylated cytosines). As shown in FIGS. 52A and 52B, PCR will convert ^{DH} U's into T and mismatch will be read as (C,T) as a specific locus. FIG. 52C shows a summary of evaluation of concatenated sequencing templates after conversion of methylated cytosines.

FIGS. 53-54C summarize a variety of different methods wherein the polymerase extension reaction to generate the concatenated sequencing templates is performed with dNTPs that include methylated-dCTP, as described in Wong et al., Nucleic Acids Research 19(5):1081-1085 (1991), which is incorporated herein in its entirety. The copied sequences prepared during extension can now bear methylated cytosines (FIG. 53). A s-copy or s′-copy will comprise a 5mC when the s or s′ strand comprises a 5hmC.

After preparation of a concatenated sequencing template using extensions with dNTPs that include methylated-dCTP, conversion of non-methylated C's to U's may be performed with any of the methods well-known in the art, such as sodium bisulfite conversion, enzymatic conversion, or borane-based conversion (FIG. 54). Following PCR, U's are then converted to T's, as shown for the top strand (FIG. 55A) and bottom strand (FIG. 55B). As shown in FIG. 55C, cytosines are sequenced as T from the original insert and C from the copy of the insert in a given strand, while methylated cytosines or hydroxymethylated cytosines are sequenced as C's from both the original insert and the copy of the insert in a given strand.

FIGS. 56 and 57A-C illustrate workflows that use chemistries or biochemistries (such as sodium bisulfite treatment) to convert non-methylated cytosines, together with extension with dNTPs that include methylated-dCTP. A new ‘2×2-base’ code is generated enables the methylation status to be identified (though methylated cytosines cannot be distinguished from hydroxymethylated cytosines). As shown in FIG. 57C, cytosines are sequenced as C from the original insert and T from the copy of the insert in a given strand, while methylated cytosines or hydroxymethylated cytosines are sequenced as T from both the original insert and the copy of the insert in a given strand.

Methods can also be used to separately identify cytosines, methylated cytosines, and hydroxymethylated cytosines. As shown in FIG. 58, concatenated sequencing templates generated with d-CTP during the polymerase extension step can be treated with enzymes such as β-glucosyltransferase that selectively converts hydroxymethylcytosines (^{hm}C) to glucosylated-methylcytosines (^{gm}C). This conversion reaction does not occur with unmethylated or methylated-cytosines. The product is further treated with a DNA methyltransferase enzyme such as DNMT1 which recognizes a hemi-methylated mCpG/GpC motif and methylates the unmethylated C to form ^{m}CpG/Gp^{m}C. DNMT1 has no activity on hemi-hydroxymethylated CpG sequences as described in Takahashi et al., FEBS Open Bio 5 (2015) 741-747. After DNMT1 treatment, a conversion may be performed that only converts non-methylated cytosines (such as bisulfite treatment), as shown in FIG. 59. After PCR and sequencing, analysis can be performed as outlined in FIGS. 60A-60C. As shown in FIG. 60C, cytosines from the target nucleic acid are sequenced as T's in the insert and the copy of the insert, methylated cytosines are sequenced as C's in the insert and the copy of the insert, and hydoxymethylated cytosines are sequenced as a C in the insert and a T in the copy of the insert.

Methods can also be used to identify cytosines, methylated cytosines, and hydroxymethylated cytosines using conversion of only methylated cytosines. As shown in FIG. 61, concatenated sequencing templates may be treated with DMNT1 to react with a hemi-methylated ^{m}CpG/GpC motif and methylate the unmethylated C to form ^{m}CpG/Gp^{m}C. The concatenated sequencing template can then be treated to convert only methylated C's to ^{DH}U's (such as by TAPS). The templates prepared after PCR are shown in FIGS. 62A and 62B. Using this method, cytosines from the target nucleic acid are sequenced as C's in the insert and the copy of the insert, methylated cytosines are sequenced as T's in the insert and the copy of the insert, and hydroxymethylated cytosines are sequenced as a T in the insert and a C in the copy of the insert, as shown in FIG. 62C.

Thus, the user can choose a decided means of methylation analysis based on the desired data and whether differentiation of methylated cytosines and hydroxymethylated cytosines is preferred.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A polynucleotide comprising:
a. a 5′ terminal polynucleotide comprising a first read primer binding sequence;
b. a first insert sequence located 3′ of the 5′ terminal polynucleotide, wherein the first insert sequence is derived from a target nucleic acid;
c. a concatenation sequence located 3′ of the first insert sequence comprising a second read primer binding sequence and a hybridization sequence;
d. a second insert sequence located 3′ of the concatenation sequence, wherein the second insert sequence is derived from a discontiguous sequence of the target nucleic acid or from a different target nucleic acid than the first insert sequence; and
e. a 3′ terminal polynucleotide sequence.

2. A polynucleotide comprising:
a. a 3′ terminal polynucleotide comprising a first read primer binding sequence;
b. a first insert sequence 5′ of the 3′ terminal polynucleotide that is derived from a target nucleic acid;
c. a concatenation sequence comprising a second read primer binding sequence that is orthogonal to the first read primer binding sequence, wherein the second read primer binding sequence comprises a hybridization sequence;
d. a second insert sequence 5′ of the concatenation sequence and derived from a discontiguous sequence of the target nucleic acid or from a different target nucleic acid than the first insert sequence; and
e. an attachment polynucleotide at the 5′ end of the polynucleotide and comprising an attachment sequence,
wherein the 3′ terminal polynucleotide, the concatenation sequence, and the attachment polynucleotide are not derived from the target nucleic acid.

3. The polynucleotide of claim 1, wherein
a. the two insert sequences are derived from different target nucleic acids;
b. the first insert sequence and the second insert sequence each independently comprise from 40 to 400 nucleotides, 100 to 200 nucleotides, or 150 nucleotides;
c. the first read primer binding sequence comprises a first adapter sequence;
d. the first read primer binding sequence further comprises the complement of a transposon end sequence; or
e. any combination of a-d.

4-6. (canceled)

7. The polynucleotide of claim 2, wherein
a. the concatenation sequence comprises (a) the hybridization sequence, and optionally comprises (b) a transposon end sequence 3′ of the hybridization unit and the complement of the transposon end sequence 5′ of the hybridization unit;
b. the attachment polynucleotide comprises a second adapter sequence and optionally a transposon end sequence;
c. the 3′ terminal polynucleotide and/or the attachment polynucleotide each independently comprise at least one of a barcode sequence, a unique molecular identifier (UMI) sequence, a capture sequence, or a cleavage sequence;
d. the polynucleotide is immobilized on a solid support; or
e. any combination of a-d.

8-10. (canceled)

11. The polynucleotide of claim 2, comprising, between the second insert sequence and the attachment polynucleotide, at least one insert unit comprising an insert sequence derived from a discontiguous sequence of the target nucleic acid or from a different target nucleic acid than the other insert sequences at the 5′ end and a concatenation sequence comprising a read primer binding sequence at the 3′ end, wherein the read primer binding sequence is orthogonal to the other read primer binding sequences.

12. A composition comprising the polynucleotide of claim 1 and its complement, wherein the complement comprises:
a. a 5′ terminal complement comprising a first complement read primer binding sequence;
b. a complement sequence of the second insert sequence located 3′ of the 5′ terminal complement;
c. a complement concatenation sequence located 3′ of the complement sequence of the second insert sequence comprising:
i. a second complement read primer binding sequence, and
ii. a complement hybridization sequence;
d. a complement sequence of the first insert sequence located 3′ of the complement concatenation sequence; and
e. a 3′ terminal complement.

13. A composition comprising the polynucleotide of claim 2 and its complement, wherein the complement comprises:
a. a 3′ terminal complement comprising a first complement read primer binding sequence, wherein the first complement read primer binding sequence is orthogonal to the first and second read primer binding sequences;
b. the complement of the second insert sequence 5′ of the 3′ terminal complement;
c. a complement concatenation sequence 5′ of the complement of the second insert sequence and comprising a 3′ to 5′ second complement read primer binding sequence, wherein the second complement read primer binding sequence is orthogonal to the first and second read primer binding sequences, and to the first complement read primer binding sequence;
d. the complement of the first insert sequence 5′ of the complement concatenation sequence; and
e. a complement attachment polynucleotide at the 5′ end comprising a complement attachment sequence.

14-17. (canceled)

18. An adapter composition or kit comprising a first forked adapter complex and a second forked adapter complex,
wherein the first forked adapter complex comprises:
a. a complement attachment polynucleotide comprising:
i. a 5′ portion comprising a complement attachment sequence; and
ii. a 3′ portion comprising an adapter; and
b. a hybridization polynucleotide comprising:
i. a 5′ portion comprising the complement of a portion of the adapter and hybridized thereto; and
ii. the complement of a hybridization sequence, wherein the complement of the hybridization sequence is not complementary to the complement attachment polynucleotide; and
the second forked adapter complex comprises:
a. an attachment polynucleotide comprising:
i. a 5′ portion comprising an attachment sequence; and
ii. a 3′ portion comprising the adapter; and
b. a hybridization polynucleotide comprising:
i. a 5′ portion comprising the complement of a portion of the adapter and hybridized thereto; and
ii. a hybridization sequence, wherein the hybridization sequence is not complementary to the attachment polynucleotide.

19-23. (canceled)

24. A method of generating a concatenated nucleic acid sequencing template comprising:
a. shearing or digesting a first source of nucleic acids and a second source of nucleic acids to generate a first library of nucleic acid fragments and a second library of nucleic acid fragments, respectively;
b. attaching the first forked adapter complex of claim 18 to each nucleic acid fragment from the first source of nucleic acids and attaching the second forked adapter complex of claim 18 to each nucleic acid fragment of the second source of nucleic acids, the attaching comprising:
i. contacting the nucleic acid fragments with a first polymerase to produce nucleic acid fragments with blunt ends;
ii. phosphorylating 5′-hydroxyl of the nucleic acid fragments with kinase;
iii. adding 3′ adenine to the nucleic acid fragments with a second polymerase; and
iv. ligating the first forked adapter complex to each nucleic acid fragment of the first library and ligating the second forked adapter complex to each nucleic acid fragment of the second library;
c. mixing and annealing the first and second libraries of nucleic acids, optionally by PCR, wherein
i. the nucleic acids denature at elevated temperatures; and
ii. A and A′ sequences hybridize to each other at lower temperatures; and
d. synthesizing a fully double-stranded concatenated nucleic acid sequencing template, optionally by PCR.

25. A method of sequencing a concatenated nucleic acid sequencing template comprising:
a. sequencing the first insert sequence of the polynucleotide of claim 1 by initiating sequencing with a first read sequencing primer complementary to the first read primer binding sequence; and
b. sequencing the second insert sequence by initiating sequencing with a second read sequencing primer complementary to the second read primer binding sequence.

26. The method of claim 24, comprising compartmentalizing a sample comprising one or more target double-stranded nucleic acid into a plurality of different compartments and generating concatenated nucleic acid sequencing templates is performed in the different compartments.

27-28. (canceled)

29. A forked adapter comprising two polynucleotide strands comprising:
a. a first strand comprising a sequencing primer sequence; and
b. a second strand comprising a 3′ hybridization sequence or its complement, wherein the 3′ end of the first strand is fully or partially complementary to the 5′ end of the second strand, and wherein the hybridization sequence or its complement is bound to a blocking oligonucleotide that is fully or partially complementary to the hybridization sequence or its complement.

30. The forked adapter of claim 29, wherein the first strand comprises a 5′ affinity element capable of binding to an affinity binding partner on a solid support or bead, optionally wherein the affinity element is connected via a linker attached to the first strand.

31. A composition or kit comprising two forked adapters of claim 29, wherein:
a. the first forked adapter comprises a first strand comprising a first read sequencing primer sequence and a second strand comprising a complement of a hybridization sequence; and
b. the second forked adapter comprises a first strand comprising a second read sequencing primer sequence and a second strand comprising a hybridization sequence, wherein one or both forked adapters comprise a blocking oligonucleotide.

32-34. (canceled)

35. A method of generating one or more concatenated nucleic acid sequencing templates comprising:
a. compartmentalizing a sample comprising target double-stranded nucleic acid into a plurality of different compartments;
b. preparing fragments each comprising an insert from the target double-stranded nucleic acid within the plurality of different compartments;
c. contacting the plurality of different compartments with the composition or kit of claim 18 comprising two forked adapters, wherein one or both forked adapters comprise a blocking oligonucleotide;
d. ligating the forked adapters to the double-stranded fragments to prepared tagged double-stranded fragments within the plurality of different compartments;
e. denaturing (1) the immobilized tagged double-stranded fragments to produce single-stranded fragments and (2) the blocking oligonucleotides to unblock hybridization sequences and complements of hybridization sequences within the plurality of different compartments;
f. hybridizing two single-stranded fragments within the same compartment to each other to form a bridge by binding of a hybridization sequence in a first fragment to the complement of the hybridization sequence in a second fragment; and
g. extending from the 3′ ends of each single-stranded fragment to produce a double-stranded concatenated nucleic acid sequencing template comprising inserts from both single-stranded fragments within the same compartment.

36. The method of claim 35, wherein
a. the compartments are wells, tubes, or droplets and/or wherein inserts comprised in the same concatenated sequencing templates were prepared from the same target nucleic acid; and/or
b. the compartmentalizing separates most different haplotypes into different compartments and the method is used for haplotype phasing.

37-54. (canceled)

55. A method of identifying modified cytosines comprised in an insert sequence comprised in a concatenated sequencing template, comprising:
a. preparing a double-stranded concatenated sequencing template by the method of claim 24, wherein each strand comprises an insert sequence and a copy of the insert sequence and the two strands are complementary to each other;
b. subjecting the double-stranded concatenated sequencing template to a condition for altering modified and/or unmodified cytosines;
c. preparing amplicons of each strand of the double-stranded concatenated sequencing template;
d. sequencing amplicons and evaluating sequencing results for the insert sequence and the copy of the insert sequence in the amplicons produced from each strand; and
e. determining positions of modified cytosines comprised in the insert sequence based on the sequences of each strand of the double-stranded concatenated sequencing template.

56. The method of claim 55, wherein the modified cytosines are methylated or hydroxymethylated cytosines.

57-66. (canceled)

67. A method of generating one or more concatenated nucleic acid sequencing templates comprising:
a. compartmentalizing a sample comprising target double-stranded nucleic acid into a plurality of different compartments;
b. preparing fragments each comprising an insert from the target double-stranded nucleic acid within the plurality of different compartments;
c. contacting the plurality of different compartments with the composition or kit of claim 29 comprising two forked adapters, wherein one or both forked adapters comprise a blocking oligonucleotide;
d. ligating the forked adapters to the double-stranded fragments to prepare tagged double-stranded fragments within the plurality of different compartments;
e. denaturing (1) the immobilized tagged double-stranded fragments to produce single-stranded fragments and (2) the blocking oligonucleotides to unblock hybridization sequences and complements of hybridization sequences within the plurality of different compartments;
f. hybridizing two single-stranded fragments within the same compartment to each other to form a bridge by binding of a hybridization sequence in a first fragment to the complement of the hybridization sequence in a second fragment; and
g. extending from the 3′ ends of each single-stranded fragment to produce a double-stranded concatenated nucleic acid sequencing template comprising inserts from both single-stranded fragments within the same compartment.


SEQUENCE LISTING (66 sequences)

SEQ ID NO: 1 - DNA, 33 bp, synthetic construct
  misc_feature 1..33: note = Synthetic
  source 1..33: mol_type = other DNA
tcgtcggcagcgtcagatgtgtataagagacag

SEQ ID NO: 2 - DNA, 34 bp, synthetic construct
  misc_feature 1..34: note = Synthetic
  source 1..34: mol_type = other DNA
gtctcgtgggctcggagatgtgtataagagacag

SEQ ID NO: 3 - DNA, 19 bp, synthetic construct
  misc_feature 1..19: note = Synthetic
  modified_base 1: mod_base = OTHER; note = 5' phosphate
  source 1..19: mol_type = other DNA
ctgtctcttatacacatct

SEQ ID NO: 4 - DNA, 14 bp, synthetic construct
  misc_feature 1..14: note = Synthetic
  source 1..14: mol_type = other DNA
tcgtcggcagcgtc

SEQ ID NO: 5 - DNA, 15 bp, synthetic construct
  misc_feature 1..15: note = Synthetic
  source 1..15: mol_type = other DNA
gtctcgtgggctcgg

SEQ ID NO: 6 - DNA, 19 bp, synthetic construct
  misc_feature 1..19: note = Synthetic
  source 1..19: mol_type = other DNA
agatgtgtataagagacag

SEQ ID NO: 7 - DNA, 29 bp, synthetic construct
  misc_feature 1..29: note = Synthetic
  source 1..29: mol_type = other DNA
  modified_base 23: mod_base = OTHER; note = uracil
aatgatacggcgaccaccgagatctacac

SEQ ID NO: 8 - DNA, 24 bp, synthetic construct
  misc_feature 1..24: note = Synthetic
  modified_base 22: mod_base = OTHER; note = 8-oxo-guanine
  source 1..24: mol_type = other DNA
caagcagaagacggcatacgagat

SEQ ID NO: 9 - DNA, 47 bp, synthetic construct
  misc_feature 1..47: note = Synthetic
  source 1..47: mol_type = other DNA
cagaagacggcatacgagatgggctcggagatgtgtataagagacag

SEQ ID NO: 10 - DNA, 42 bp, synthetic construct
  misc_feature 1..42: note = Synthetic
  modified_base 1: mod_base = OTHER; note = 5' phosphate
  source 1..42: mol_type = other DNA
tgtctcttatacacatctctctcttctctccttcttctctct

SEQ ID NO: 11 - DNA, 42 bp, synthetic construct
  misc_feature 1..42: note = Synthetic
  modified_base 1: mod_base = OTHER; note = 5' phosphate
  source 1..42: mol_type = other DNA
tgtctcttatacacatctagagagaagaaggagagaagagag

SEQ ID NO: 12 - DNA, 47 bp, synthetic construct; identical to SEQ ID NO: 9
  misc_feature 1..47: note = Synthetic
  modified_base 1: mod_base = OTHER; note = 5' Desthiobiotin-TEG
  source 1..47: mol_type = other DNA

SEQ ID NO: 13 - DNA, 24 bp, synthetic construct
  misc_feature 1..24: note = Synthetic
  source 1..24: mol_type = other DNA
agagagaagaaggagagaagagag

SEQ ID NO: 14 - DNA, 24 bp, synthetic construct
  misc_feature 1..24: note = Synthetic
  source 1..24: mol_type = other DNA
gagtaagtggaagagataggaagg

SEQ ID NO: 15 - DNA, 58 bp, synthetic construct
  misc_feature 1..58: note = Synthetic
  modified_base 57^58: mod_base = OTHER; note = phosphorothioate bond
  source 1..58: mol_type = other DNA
aatgatacggcgaccaccgagatctacactctttccctacacgacgctcttccgatct

SEQ ID NO: 16 - DNA, 36 bp, synthetic construct
  misc_feature 1..36: note = Synthetic
  modified_base 1: mod_base = OTHER; note = 5' phosphate
  source 1..36: mol_type = other DNA
gatcggaagagcccttcctatctcttccacttactc

SEQ ID NO: 17 - DNA, 64 bp, synthetic construct
  misc_feature 1..64: note = Synthetic
  modified_base 63^64: mod_base = OTHER; note = phosphorothioate bond
  source 1..64: mol_type = other DNA
caagcagaagacggcatacgagatacatcggtgactggagttcagacgtgtgctcttccgatct

SEQ ID NO: 18 - DNA, 36 bp, synthetic construct
  misc_feature 1..36: note = Synthetic
  modified_base 1: mod_base = OTHER; note = 5' phosphate
  source 1..36: mol_type = other DNA
gatcggaagagcgagtaagtggaagagataggaagg

SEQ ID NO: 19 - DNA, 28 bp, synthetic construct
  misc_feature 1..28: note = Synthetic
  source 1..28: mol_type = other DNA
tctttccctacacgacgctcttccgatc

SEQ ID NO: 20 - DNA, 12 bp, synthetic construct
  misc_feature 1..12: note = Synthetic
  modified_base 1: mod_base = OTHER; note = 5' phosphate
  source 1..12: mol_type = other DNA
gatcggaagagc

SEQ ID NO: 21 - DNA, 39 bp, synthetic construct
  misc_feature 1..39: note = Synthetic
  source 1..39: mol_type = other DNA
acatcggtgactggagttcagacgtgtgctcttccgatc

SEQ ID NO: 22 - DNA, 29 bp, synthetic construct
  misc_feature 1..29: note = Synthetic
  source 1..29: mol_type = other DNA
tctttccctacacgacgctcttccgatct

SEQ ID NO: 23 - DNA, 37 bp, synthetic construct
  misc_feature 1..37: note = Synthetic
  source 1..37: mol_type = other DNA
cggtctcggcattcctgctgaaccgctcttccgatct

SEQ ID NO: 24 - DNA, 36 bp, synthetic construct; identical to SEQ ID NO: 18
  misc_feature 1..36: note = Synthetic
  modified_base 1: mod_base = OTHER; note = 5' phosphate
  source 1..36: mol_type = other DNA

SEQ ID NO: 25 - DNA, 64 bp, synthetic construct; identical to SEQ ID NO: 17
  misc_feature 1..64: note = Synthetic
  modified_base 63^64: mod_base = OTHER; note = phosphorothioate bond
  source 1..64: mol_type = other DNA

SEQ ID NO: 26 - DNA, 36 bp, synthetic construct; identical to SEQ ID NO: 16
  misc_feature 1..36: note = Synthetic
  modified_base 1: mod_base = OTHER; note = 5' phosphate
  source 1..36: mol_type = other DNA

SEQ ID NO: 27 - DNA, 58 bp, synthetic construct; identical to SEQ ID NO: 15
  misc_feature 1..58: note = Synthetic
  modified_base 57^58: mod_base = OTHER; note = phosphorothioate bond
  source 1..58: mol_type = other DNA

SEQ ID NO: 28 - DNA, 20 bp, synthetic construct
  misc_feature 1..20: note = Synthetic
  source 1..20: mol_type = other DNA
aatgatacggcgaccaccga

SEQ ID NO: 29 - DNA, 21 bp, synthetic construct
  misc_feature 1..21: note = Synthetic
  source 1..21: mol_type = other DNA
caagcagaagacggcatacga

SEQ ID NO: 30 - DNA, 28 bp, synthetic construct
  misc_feature 1..28: note = Synthetic
  source 1..28: mol_type = other DNA
tgcaggtgcgatggcgctcttccgatct

SEQ ID NO: 31 - DNA, 42 bp, synthetic construct
  misc_feature 1..42: note = Synthetic
  source 1..42: mol_type = other DNA
gatcgaagatctcccgagaattccagataggaggaacttagt

SEQ ID NO: 32 - DNA, 33 bp, synthetic construct
  misc_feature 1..33: note = Synthetic
  source 1..33: mol_type = other DNA
acactctttccctacacgacgctcttccgatct

SEQ ID NO: 33 - DNA, 35 bp, synthetic construct
  misc_feature 1..35: note = Synthetic
  source 1..35: mol_type = other DNA
gatctcccgagaattccagataggaggaacttagt

SEQ ID NO: 34 - DNA, 35 bp, synthetic construct
  misc_feature 1..35: note = Synthetic
  source 1..35: mol_type = other DNA
cagcccattctagcactctccaggaggaacttagt

SEQ ID NO: 35 - DNA, 36 bp, synthetic construct
  misc_feature 1..36: note = Synthetic
  source 1..36: mol_type = other DNA
actaagttcctcctatctggaattctcgggagatct

SEQ ID NO: 36 - DNA, 34 bp, synthetic construct
  misc_feature 1..34: note = Synthetic
  source 1..34: mol_type = other DNA
cctatgatgtttatcctttggatggtcgccatga

SEQ ID NO: 37 - DNA, 35 bp, synthetic construct
  misc_feature 1..35: note = Synthetic
  source 1..35: mol_type = other DNA
gaccttcgcccgttttttacggtgcccgccaatcg

SEQ ID NO: 38 - DNA, 35 bp, synthetic construct
  misc_feature 1..35: note = Synthetic
  source 1..35: mol_type = other DNA
ttatccgccatattcgcgatgaatcacatgatcac

SEQ ID NO: 39 - DNA, 35 bp, synthetic construct
  misc_feature 1..35: note = Synthetic
  source 1..35: mol_type = other DNA
tcaatagtcacacagtccttgacggtataataacc

SEQ ID NO: 40 - DNA, 15 bp, synthetic construct
  misc_feature 1..15: note = Synthetic
  source 1..15: mol_type = other DNA
tcactcaagaacagc

SEQ ID NO: 41 - DNA, 58 bp, synthetic construct; identical to SEQ ID NO: 15
  misc_feature 1..58: note = Synthetic
  source 1..58: mol_type = other DNA

SEQ ID NO: 42 - DNA, 28 bp, synthetic construct
  misc_feature 1..28: note = Synthetic
  source 1..28: mol_type = other DNA
agatcggaagagcgccatcgcacctgca

SEQ ID NO: 43 - DNA, 58 bp, synthetic construct
  misc_feature 1..58: note = Synthetic
  source 1..58: mol_type = other DNA
agatcggaagagcgtcgtgtagggaaagagtgtagatctcggtggtcgccgtatcatt

SEQ ID NO: 44 - DNA, 59 bp, synthetic construct
  misc_feature 1..59: note = Synthetic
  source 1..59: mol_type = other DNA
caagcagaagacggcatacgagatcagcccattctagcactctccaggaggaacttagt

SEQ ID NO: 45 - DNA, 42 bp, synthetic construct
  misc_feature 1..42: note = Synthetic
  source 1..42: mol_type = other DNA
actaagttcctcctatctggaattctcgggagatcttcgatc

SEQ ID NO: 46 - DNA, 59 bp, synthetic construct
  misc_feature 1..59: note = Synthetic
  source 1..59: mol_type = other DNA
actaagttcctcctggagagtgctagaatgggctgatctcgtatgccgtcttctgcttg

SEQ ID NO: 47 - DNA, 31 bp, synthetic construct
  misc_feature 1..31: note = Synthetic
  source 1..31: mol_type = other DNA
actaagttcctcctatctggaattctcggga

SEQ ID NO: 48 - DNA, 35 bp, synthetic construct; identical to SEQ ID NO: 33
  misc_feature 1..35: note = Synthetic
  source 1..35: mol_type = other DNA

SEQ ID NO: 49 - DNA, 58 bp, synthetic construct; identical to SEQ ID NO: 15
  misc_feature 1..58: note = Synthetic
  source 1..58: mol_type = other DNA

SEQ ID NO: 50 - DNA, 36 bp, synthetic construct
  misc_feature 1..36: note = Synthetic
  source 1..36: mol_type = other DNA
agatctcccgagaattccagataggaggaacttagt

SEQ ID NO: 51 - DNA, 59 bp, synthetic construct; identical to SEQ ID NO: 46
  misc_feature 1..59: note = Synthetic
  source 1..59: mol_type = other DNA

SEQ ID NO: 52 - DNA, 58 bp, synthetic construct; identical to SEQ ID NO: 43
  misc_feature 1..58: note = Synthetic
  source 1..58: mol_type = other DNA

SEQ ID NO: 53 - DNA, 36 bp, synthetic construct; identical to SEQ ID NO: 35
  misc_feature 1..36: note = Synthetic
  source 1..36: mol_type = other DNA

SEQ ID NO: 54 - DNA, 59 bp, synthetic construct; identical to SEQ ID NO: 44
  misc_feature 1..59: note = Synthetic
  source 1..59: mol_type = other DNA

SEQ ID NO: 55 - DNA, 73 bp, synthetic construct
  misc_feature 1..73: note = Synthetic
  source 1..73: mol_type = other DNA
cctatgatgtttatcctttggatggtcgccatgatggtggttattataccgtcaaggactgtgtgactattga

SEQ ID NO: 56 - DNA, 73 bp, synthetic construct
  misc_feature 1..73: note = Synthetic
  source 1..73: mol_type = other DNA
tcaatagtcacacagtccttgacggtataataaccaccatcatggcgaccatccaaaggataaacatcatagg

SEQ ID NO: 57 - DNA, 71 bp, synthetic construct
  misc_feature 1..71: note = Synthetic
  source 1..71: mol_type = other DNA
gaccttcgcccgttttttacggtgcccgccaatcgcgtgatcatgtgattcatcgcgaatatggcggataa

SEQ ID NO: 58 - DNA, 71 bp, synthetic construct
  misc_feature 1..71: note = Synthetic
  source 1..71: mol_type = other DNA
ttatccgccatattcgcgatgaatcacatgatcacgcgattggcgggcaccgtaaaaaacgggcgaaggtc

SEQ ID NO: 59 - DNA, 34 bp, synthetic construct
  misc_feature 1..34: note = Synthetic
  source 1..34: mol_type = other DNA
nctatgatgtttatcctttggatggtcgccatga

SEQ ID NO: 60 - DNA, 34 bp, synthetic construct
  misc_feature 1..34: note = Synthetic
  source 1..34: mol_type = other DNA
cctatgatgtttatcctttggatggtctccatga

SEQ ID NO: 61 - DNA, 34 bp, synthetic construct
  misc_feature 1..34: note = Synthetic
  source 1..34: mol_type = other DNA
cctatgatgtttatcctttggatggccgccatga

SEQ ID NO: 62 - DNA, 34 bp, synthetic construct
  misc_feature 1..34: note = Synthetic
  source 1..34: mol_type = other DNA
cctatgatgtttaccctttggatggtcgccatga

SEQ ID NO: 63 - DNA, 35 bp, synthetic construct
  misc_feature 1..35: note = Synthetic
  source 1..35: mol_type = other DNA
gaccttcgcccgttttttacggtgcccgccactcg

SEQ ID NO: 64 - DNA, 35 bp, synthetic construct
  misc_feature 1..35: note = Synthetic
  source 1..35: mol_type = other DNA
gaccttcgcccgtttttacggtgcccgccaatcgc

SEQ ID NO: 65 - DNA, 35 bp, synthetic construct
  misc_feature 1..35: note = Synthetic
  source 1..35: mol_type = other DNA
ggccttcgcccgttttttacggtgcccgccaatcg

SEQ ID NO: 66 - DNA, 35 bp, synthetic construct
  misc_feature 1..35: note = Synthetic
  source 1..35: mol_type = other DNA
naccttcgcccgttttttacggtgcccgccaatcg